(19) United States
(12) Patent Application Publication
(10) Pub. No.: US 2005/0049192 A1
(43) Pub. Date: Mar. 3, 2005

(54) Therapeutic polypeptides, nucleic acids encoding same, and methods of use

(76) Inventors: Mei Zhong, Branford, CT (US); Weizhen Ji, Branford, CT (US); Xiaojia (Sasha) Guo, Branford, CT (US); Daniel Rieger, Branford, CT (US); Muralidhara Padigaru, Branford, CT (US); Rachel Malcolm, Orange, CT (US); Kimberly Spytek, Ellington, CT (US); David Anderson, Plantsville, CT (US); Linda Gorman, Branford, CT (US); Elina Catterton, Milford, CT (US); John MacDougall, Hamden, CT (US); David Stone, Guilford, CT (US); Shlomit Edinger, New Haven, CT (US)

Correspondence Address: Jenell Lawson;Intellectual Property, CuraGen Corporation, 555 Long Wharf Drive, New Haven, CT, 06511, US

(21) Appl. No.: 10/635,149

(22) Filed: Aug. 6, 2003

Related U.S. Application Data
(63) Continuation-in-part of application No. 10/236,392, filed on Sep. 6, 2002.
(63) Continuation-in-part of application No. 09/635,949, filed on Aug. 10, 2000.
(63) Continuation-in-part of application No. 10/023,634, filed on Dec. 17, 2001.
(63) Continuation-in-part of application No. 10/242,943, filed on Sep. 13, 2002, now abandoned.
Application No. 10/242,943 is a continuation of application No. 09/970,944, filed on Oct. 4, 2001.
Application No. 10/242,943 is a continuation-in-part of application No. 10/187,975, filed on Jul. 2, 2002.
Application No. 10/242,943 is a continuation-in-part of application No. 10/136,826, filed on May 1, 2002.
Application No. 10/242,943 is a continuation-in-part of application No. 09/584,411, filed on May 31, 2000, now abandoned.
Application No. 10/242,943 is a continuation-in-part of application No. 09/569,269, filed on May 11, 2000.
(60) Provisional application No. 60/390,155, filed on Jun. 19, 2002.
(60) Provisional application No. 60/318,765, filed on Sep. 12, 2001.
(60) Provisional application No. 60/357,303, filed on Feb. 15, 2002.
(60) Provisional application No. 60/367,753, filed on Mar. 25, 2002.
(60) Provisional application No. 60/369,479, filed on Apr. 2, 2002.
(60) Provisional application No. 60/318,120, filed on Sep. 7, 2001.
(60) Provisional application No. 60/318,130, filed on Sep. 7, 2001.
(60) Provisional application No. 60/381,672, filed on May 17, 2002.
(60) Provisional application No. 60/318,219, filed on Sep. 7, 2001.
(60) Provisional application No. 60/318,430, filed on Sep. 10, 2001.
(60) Provisional application No. 60/322,781, filed on Sep. 17, 2001.
(60) Provisional application No. 60/322,816, filed on Sep. 17, 2001.
(60) Provisional application No. 60/323,519, filed on Sep. 19, 2001.
(60) Provisional application No. 60/384,012, filed on May 29, 2002.
(60) Provisional application No. 60/323,631, filed on Sep. 20, 2001.
(60) Provisional application No. 60/323,636, filed on Sep. 20, 2001.
(60) Provisional application No. 60/360,973, filed on Feb. 28, 2002.
(60) Provisional application No. 60/366,131, filed on Mar. 20, 2002.
(60) Provisional application No. 60/324,969, filed on Sep. 25, 2001.
(60) Provisional application No. 60/383,651, filed on May 28, 2002.
(60) Provisional application No. 60/325,091, filed on Sep. 25, 2001.
(60) Provisional application No. 60/324,990, filed on Sep. 26, 2001.
(60) Provisional application No. 60/381,664, filed on May 17, 2002.
(60) Provisional application No. 60/379,532, filed on May 10, 2002.
(60) Provisional application No. 60/148,433, filed on Aug. 11, 1999.
(60) Provisional application No. 60/303,046, filed on Jul. 5, 2001.
(60) Provisional application No. 60/303,828, filed on Jul. 9, 2001.
(60) Provisional application No. 60/304,502, filed on Jul. 11, 2001.
(60) Provisional application No. 60/305,011, filed on Jul. 12, 2001.
(60) Provisional application No. 60/305,262, filed on Jul. 13, 2001.
(60) Provisional application No. 60/305,673, filed on Jul. 16, 2001.
(60) Provisional application No. 60/306,085, filed on Jul. 17, 2001.
(60) Provisional application No. 60/307,536, filed on Jul. 24, 2001.
(60) Provisional application No. 60/308,228, filed on Jul. 27, 2001.
(60) Provisional application No. 60/308,877, filed on Jul. 30, 2001.
(60) Provisional application No. 60/312,203, filed on Aug. 14, 2001.
(60) Provisional application No. 60/322,640, filed on Sep. 17, 2001.
(60) Provisional application No. 60/323,484, filed on Sep. 19, 2001.
(60) Provisional application No. 60/323,821, filed on Sep. 21, 2001.
(60) Provisional application No. 60/323,948, filed on Sep. 21, 2001.
(60) Provisional application No. 60/324,711, filed on Sep. 25, 2001.
(60) Provisional application No. 60/327,893, filed on Oct. 9, 2001.
(60) Provisional application No. 60/331,768, filed on Nov. 21, 2001.
(60) Provisional application No. 60/359,191, filed on Feb. 21, 2002.
(60) Provisional application No. 60/358,939, filed on Feb. 22, 2002.
(60) Provisional application No. 60/360,923, filed on Feb. 28, 2002.
(60) Provisional application No. 60/360,830, filed on Mar. 1, 2002.
(60) Provisional application No. 60/361,178, filed on Mar. 1, 2002.
(60) Provisional application No. 60/361,748, filed on Mar. 5, 2002.
(60) Provisional application No. 60/363,429, filed on Mar. 12, 2002.
(60) Provisional application No. 60/363,683, filed on Mar. 12, 2002.
(60) Provisional application No. 60/372,141, filed on Apr. 12, 2002.
(60) Provisional application No. 60/372,967, filed on Apr. 16, 2002.
(60) Provisional application No. 60/373,051, filed on Apr. 16, 2002.
(60) Provisional application No. 60/373,063, filed on Apr. 16, 2002.
(60) Provisional application No. 60/373,280, filed on Apr. 17, 2002.
(60) Provisional application No. 60/373,287, filed on Apr. 17, 2002.
(60) Provisional application No. 60/373,881, filed on Apr. 19, 2002.
(60) Provisional application No. 60/288,395, filed on May 3, 2001.
(60) Provisional application No. 60/308,901, filed on Jul. 31, 2001.
(60) Provisional application No. 60/330,292, filed on Oct. 18, 2001.
(60) Provisional application No. 60/288,063, filed on May 2, 2001.
(60) Provisional application No. 60/289,087, filed on May 7, 2001.
(60) Provisional application No. 60/289,818, filed on May 9, 2001.
(60) Provisional application No. 60/313,416, filed on Aug. 17, 2001.
(60) Provisional application No. 60/289,817, filed on May 9, 2001.
(60) Provisional application No. 60/290,194, filed on May 11, 2001.
(60) Provisional application No. 60/325,683, filed on Sep. 27, 2001.
(60) Provisional application No. 60/336,909, filed on Dec. 3, 2001.
(60) Provisional application No. 60/290,753, filed on May 14, 2001.
(60) Provisional application No. 60/291,181, filed on May 15, 2001.
(60) Provisional application No. 60/291,243, filed on May 16, 2001.
(60) Provisional application No. 60/292,001, filed on May 18, 2001.
(60) Provisional application No. 60/292,374, filed on May 21, 2001.
(60) Provisional application No. 60/312,270, filed on Aug. 14, 2001.
(60) Provisional application No. 60/292,587, filed on May 22, 2001.
(60) Provisional application No. 60/359,245, filed on Feb. 21, 2002.
(60) Provisional application No. 60/293,107, filed on May 23, 2001.
(60) Provisional application No. 60/294,110, filed on May 29, 2001.
(60) Provisional application No. 60/304,879, filed on Jul. 12, 2001.
(60) Provisional application No. 60/293,747, filed on May 25, 2001.
(60) Provisional application No. 60/294,434, filed on May 30, 2001.
(60) Provisional application No. 60/318,463, filed on Sep. 10, 2001.
(60) Provisional application No. 60/294,109, filed on May 29, 2001.
(60) Provisional application No. 60/333,873, filed on Nov. 28, 2001.
(60) Provisional application No. 60/337,552, filed on Dec. 3, 2001.
(60) Provisional application No. 60/294,827, filed on May 31, 2001.
(60) Provisional application No. 60/137,322, filed on Jun. 3, 1999.
(60) Provisional application No. 60/189,810, filed on Mar. 16, 2000.
(60) Provisional application No. 60/191,158, filed on Mar. 22, 2000.
(60) Provisional application No. 60/193,086, filed on Mar. 30, 2000.
(60) Provisional application No. 60/201,388, filed on May 3, 2000.
(60) Provisional application No. 60/134,315, filed on May 14, 1999.
(60) Provisional application No. 60/175,744, filed on Jan. 12, 2000.
(60) Provisional application No. 60/188,274, filed on Mar. 10, 2000.
(60) Provisional application No. 60/401,597, filed on Aug. 7, 2002.
(60) Provisional application No. 60/406,202, filed on Aug. 27, 2002.
(60) Provisional application No. 60/403,517, filed on Aug. 13, 2002.
(60) Provisional application No. 60/402,205, filed on Aug. 9, 2002.
(60) Provisional application No. 60/402,209, filed on Aug. 9, 2002.
(60) Provisional application No. 60/403,696, filed on Aug. 15, 2002.
(60) Provisional application No. 60/403,548, filed on Aug. 13, 2002.
(60) Provisional application No. 60/406,318, filed on Aug. 26, 2002.
(60) Provisional application No. 60/423,138, filed on Nov. 1, 2002.

(51) Int. Cl. A61K 38/17; C07H 21/04; C07K 14/47

(52) U.S. Cl. 514/12; 435/69.1; 435/320.1; 435/325; 530/350; 536/23.5

(57) ABSTRACT

Disclosed herein are nucleic acid sequences that encode novel polypeptides. Also disclosed are polypeptides encoded by these nucleic acid sequences, and antibodies that immunospecifically bind to the polypeptide, as well as derivatives, variants, mutants, or fragments of the novel polypeptide, polynucleotide, or antibody specific to the polypeptide. Vectors, host cells, antibodies and recombinant methods for producing the polypeptides and polynucleotides, as well as methods for using same are also included. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. Ser. No. 10/236392, filed Sep. 6, 2002; which claims the benefit of U.S. Ser. No. 09/540,763, filed Mar. 30, 2000; U.S. Ser. No. 60/390,155, filed Jun. 19, 2002;U.S. Ser. No. 09/635,949, filed Aug. 10, 2000; U.S. Ser. No. 60/318,765, filed Sep. 12, 2001; U.S. Ser. No. 60/357,303, filed Feb. 15, 2002; U.S. Ser. No. 60/367,753, filed Mar. 25, 2002; U.S. Ser. No. 60/369,479, filed Apr. 2, 2002; U.S. Ser. No. 09/659,634, filed Sep. 12, 2000; U.S. Ser. No. 60/318,120, filed Sep. 7, 2001; U.S. Ser. No. 60/318,130, filed Sep. 7, 2001; U.S. Ser. No. 60/381,672, filed May 17, 2002; U.S. Ser. No. 60/318,219, filed Sep. 7, 2001; U.S. Ser. No. 60/318,430, filed Sep. 10, 2001; U.S. Ser. No. 60/322,781, filed Sep. 17, 2001; U.S. Ser. No. 60/322,816, filed Sep. 17, 2001; U.S. Ser. No. 60/323,519, filed Sep. 19, 2001; U.S. Ser. No. 60/384,012, filed May 29, 2002; U.S. Ser. No. 60/323,631, filed Sep. 20, 2001; U.S. Ser. No. 60/323,636, filed Sep. 20, 2001; U.S. Ser. No. 60/360,973, filed Feb. 28, 2002; U.S. Ser. No. 60/366,131, filed Mar. 20, 2002; U.S. Ser. No. 60/324,969, filed Sep. 25, 2001; U.S. Ser. No. 60/383,651, filed May 28, 2002; U.S. Ser. No. 60/325,091, filed Sep. 25, 2001; U.S. Ser. No. 60/324,990, filed Sep. 26, 2001; U.S. Ser. No. 60/381,664, filed May 17, 2002; U.S. Ser. No. 60/379,532, filed May 10, 2002; a continuation-in-part of U.S. Ser. No. 09/635949, filed Aug. 10, 2000, which claims the benefit of U.S. Ser. No. 60/148,433, filed Aug. 11, 1999; a continuation-in-part of U.S. Ser. No. 10/023634, filed Dec. 17, 2001; a continuation-in-part of U.S. Ser. No. 10/242, 943 filed on 13-Sep. 13, 2002; which is a continuation of U.S. Ser. No. 09/970944, filed Oct. 4, 2001; a continuation-in-part of U.S. Ser. No. 10/187975, filed Jul. 2, 2002, which claims the benefit of U.S. Ser. No. 60/303,046, filed Jul. 5, 2001; U.S. Ser. No. 60/303,828, filed Jul. 9, 2001; U.S. Ser. No. 60/304,502, filed Jul. 11, 2001; U.S. Ser. No. 60/305,011, filed Jul. 12, 2001; U.S. Ser. No. 60/305,262, filed Jul. 13, 2001; U.S. Ser. No. 60/305,673, filed Jul. 16, 2001; U.S. Ser. No. 60/306,085, filed Jul. 17, 2001; U.S. Ser. No. 60/307,536, filed Jul. 24, 2002; U.S. Ser. No. 60/308,228, filed Jul. 27, 2001; U.S. Ser. No. 60/308,877, filed Jul. 30, 2001; U.S. Ser. No. 60/312,203, filed Aug. 14, 2001; U.S. Ser. No. 60/322,640, filed Sep. 17, 2001; U.S. Ser. No. 60/323,484, filed Sep. 19, 2001; U.S. Ser. No. 60/323,821, filed Sep. 21, 2001; U.S. Ser. No. 60/323,948, filed Sep. 21, 2001; U.S. Ser. No. 60/324,711, filed Sep. 25, 2001; U.S. Ser. No. 60/327,893, filed Oct. 9, 2001; U.S. Ser. No. 60/331,768, filed Nov. 21, 2001; U.S. Ser. No. 60/359,191, filed Feb. 21, 2002; U.S. Ser. No. 60/358,939, filed Feb. 22, 2002; U.S. Ser. No. 60/360,923, filed Feb. 28, 2002; U.S. Ser. No. 60/360,830, filed Mar. 1, 2002; U.S. Ser. No. 60/361,178, filed Mar. 1, 2002; U.S. Ser. No. 60/361,748, filed Mar. 5, 2002; U.S. Ser. No. 60/363,429, filed Mar. 12, 2002; U.S. Ser. No. 60/363,683, filed Mar. 12, 2002; U.S. Ser. No. 60/372,141, filed Apr. 12, 2002; U.S. Ser. No. 60/372,967, filed Apr. 16, 2002; U.S. Ser. No. 60/373,051, filed Apr. 16, 2002; U.S. Ser. No. 60/373,063, filed Apr. 16, 2002; U.S. Ser. No. 60/373,280, filed Apr. 17, 2002; U.S. Ser. No. 60/373,287, filed Apr. 17, 2002; and U.S. Ser. No. 60/373,881, filed Apr. 19, 2002; a continuation-in-part of U.S. Ser. No. 10/136,826, filed May 1, 2002, which claims benefit of U.S. Ser. No. 60/288,395, filed May 3, 2001; U.S. Ser. No. 60/308,901, filed Jul. 31, 2001; U.S. Ser. No. 60/330,292, filed Oct. 18, 2001; U.S. Ser. No. 60/288,063, filed May 2, 2001; U.S. Ser. No. 60/289,087, filed May 7, 2001; U.S. Ser. No. 60/289,818, filed May 9, 2001; U.S. Ser. No. 60/313,416, filed Aug. 17, 2001; U.S. Ser. No. 60/289,817, filed May 9, 2001; U.S. Ser. No. 60/290,194, filed May 11, 2001; U.S. Ser. No. 60/325,683, filed Sep. 27, 2001; U.S. Ser. No. 60/336,909, filed Dec. 3, 2001; U.S. Ser. No. 60/290,753, filed May 14, 2001; U.S. Ser. No. 60/291,181, filed May 15, 2001; U.S. Ser. No. 60/291,243, filed May 16, 2001; U.S. Ser. No. 60/292,001, filed May 18, 2001; U.S. Ser. No. 60/292,374, filed May 21, 2001; U.S. Ser. No. 60/312,270, filed Aug. 14, 2001; U.S. Ser. No. 60/292,587, filed May 22, 2001; U.S. Ser. No. 60/359,245, filed Feb. 21, 2002; U.S. Ser. No. 60/293,107, filed May 23, 2001; U.S. Ser. No. 60/294,110, filed May 29, 2001; U.S. Ser. No. 60/304,879, filed Jul. 12, 2001; U.S. Ser. No. 60/293,747, filed May 25, 2001; U.S. Ser. No. 60/294,434, filed May 30, 2001; U.S. Ser. No. 60/318,463, filed Sep. 10, 2001; U.S. Ser. No. 60/294,109, filed May 29, 2001; U.S. Ser. No. 60/333,873, filed Nov. 28, 2001; U.S. Ser. No. 60/337,552, filed Dec. 3, 2001; and U.S. Ser. No. 60/294,827, filed May 31, 2001; a continuation-in-part of U.S. Ser. No. 09/584411, filed May 31, 2000; which claims benefit of U.S. Ser. No. 60/137,322, filed Jun. 3, 1999; U.S. Ser. No. 60/189,810, filed Mar. 16, 2000; U.S. Ser. No. 60/191,158, filed Mar. 22, 2000; U.S. Ser. No. 60/193,086, filed Mar. 30, 2000; and U.S. Ser. No. 60/201,388, filed May 3, 2000; and a continuation-in-part of U.S. Ser. No. 09/569269, filed May 11, 2000; which claims benefit of U.S. Ser. No. 60/134,315 filed May 14, 1999, U.S. Ser. No. 60/175,744, filed Jan. 12, 2000; and U.S. Ser. No. 60/188,274, filed March 10, 2000; and this application claims priority to U.S. Ser. No. 60/401,597, filed Aug. 7, 2002; U.S. Ser. No. 60/406,202, filed Aug. 27, 2002; U.S. Ser. No. 60/403,517, filed Aug. 13, 2002; U.S. Ser. No. 60/402,205, filed Aug. 9, 2002; U.S. Ser. No. 60/402,209, filed Aug. 9, 2002; U.S. Ser. No. 60/403,696, filed Aug. 15, 2002; U.S. Ser. No. 60/403,548, filed Aug. 13, 2002; U.S. Ser. No. 60/406,318, filed Aug. 26, 2002; U.S. Ser. No. 60/423,138, filed Nov. 1, 2002; and each of which is incorporated herein by reference, in its entirety.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins, and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed -levels of the protein effector of interest. In addition, there is a need for a method of treatment of a pathological condition brought on by a increased or up-regulated levels of the protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 38. The novel nucleic acids and polypeptides are referred to herein as NOV1a, NOV1b, NOV1b, NOV1c, NOV2a, NOV2b, NOV2c, NOV2d, NOV3a, NOV3b, etc. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature-form of the amino acid sequence selected from the group consisting of SEQ ID NO:2n, wherein n is an integer between 1 and 38, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 38. In another embodiment, the invention also comprises variants of the amino acid sequence selected from the group consisting of SEQ ID NO:2n, wherein n is an integer between 1 and 38 wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 38, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 38. These allelic variants include amino acid sequences that are the translations of nucleic acid sequences differing by a single nucleotide from nucleic acid sequences selected from the group consisting of SEQ ID NOS: 2n-1, wherein n is an integer between 1 and 38. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 38 and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 38 wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 38 in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 38 in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 38, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 38, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO:2n, wherein n is an integer between 1 and 38, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 38, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 38, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO:2n, wherein n is an integer between 1 and 38 or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 38; a variant of a mature form of the amino acid sequence selected from the group consisting of SEQ ID NO:2n, wherein n is an integer between 1 and 38 wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 38; a variant of the amino acid sequence selected from the group consisting of SEQ ID NO:2n, wherein n is an integer between 1 and 38, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO:2n, wherein n is an integer between 1 and 38 or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 38, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO:2n, wherein n is an integer between 1 and 38 that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 38, wherein the nucleic acid molecule differs by a single nucleotide from a nucleic acid sequence selected from the group consisting of SEQ ID NOS: 2n-1, wherein n is an integer between 1 and 38.

In another embodiment, the invention includes an isolated nucleic acid molecule having a nucleic acid sequence encoding a polypeptide including an amino acid sequence selected from the group-consisting of a mature form of the amino acid sequence given SEQ ID NO:2n, wherein n is an integer between 1 and 38, wherein the nucleic acid molecule comprises a nucleotide sequence selected from the group consisting of the nucleotide sequence selected from the group consisting of SEQ ID NO:2n-1, wherein n is an integer between 1 and 38; a nucleotide sequence wherein one or more nucleotides in the nucleotide sequence selected from the group consisting of SEQ ID NO:2n-1, wherein n is an integer between 1 and 38 is changed from that selected from the group consisting of the chosen sequence to a different nucleotide provided that no more than 15% of the nucleotides are so changed; a nucleic acid fragment of the sequence selected from the group consisting of SEQ ID NO:2n-1, wherein n is an integer between 1 and 38; and a nucleic acid fragment wherein one or more nucleotides in the nucleotide sequence selected from the group consisting of SEQ ID NO:2n-1, wherein n is an integer between 1 and 38 is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 38, wherein the nucleic acid molecule hybridizes under stringent conditions to the nucleotide sequence selected from the group consisting of SEQ ID NO:2n-1, wherein n is an integer between 1 and 38, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 38, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO:2n, wherein n is an integer between 1 and 38. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 38 in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 38 in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

The invention further provides an antibody that binds immunospecifically to a NOVX polypeptide. The NOVX antibody may be monoclonal, humanized, or a fully human antibody. Preferably, the antibody has a dissociation constant for the binding of the NOVX polypeptide to the antibody less than 1×10⁻⁹ M. More preferably, the NOVX antibody neutralizes the activity of the NOVX polypeptide.

In a further aspect, the invention provides for the use of a therapeutic in the manufacture of a medicament for treating a syndrome associated with a human disease, associated with a NOVX polypeptide. Preferably the therapeutic is a NOVX antibody.

In yet a further aspect, the invention provides a method of treating or preventing a NOVX-associated disorder, a method of treating a pathological state in a mammal, and a method of treating or preventing a pathology associated with a polypeptide by administering a NOVX antibody to a subject in an amount sufficient to treat or prevent the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ ID | |
|---|---|---|---|---|
| NOVX | | NO | NO | |
| Assign- | Internal | (nucleic | (amino | |
| ment | Identification | acid) | acid) | Homology |
| NOV1a | CG121992-03 | 1 | 2 | Chordin precursor - |
| | | | | Homo sapiens |
| NOV1b | CG121992-02 | 3 | 4 | Chordin precursor - |
| | | | | Homo sapiens |
| NOV1c | CG121992-04 | 5 | 6 | Chordin precursor - |
| | | | | Homo sapiens |
| NOV2a | CG186275-03 | 7 | 8 | ADAM 22 precursor (A |
| | | | | disintegrin and |
| | | | | metalloproteinase |
| | | | | domain 22) |
| | | | | (Metalloproteinase-like, |
| | | | | disintegrin-like, and |
| | | | | cysteine-rich protein 2) |
| | | | | (Metalloproteinase- |
| | | | | disintegrin ADAM22- |
| | | | | 3) - Homo sapiens |
| NOV3a | 260368272 | 9 | 10 | Beta-secretase - Homo |
| | | | | sapiens |
| NOV3b | 260368280 | 11 | 12 | Beta-secretase - Homo |
| | | | | sapiens |
| NOV3c | 267441066 | 13 | 14 | Beta-secretase - Homo |
| | | | | sapiens |
| NOV3d | CG50586-03 | 15 | 16 | Beta-secretase - Homo |
| | | | | sapiens |
| NOV4a | CG50637-01 | 17 | 18 | Transmembrane protein |
| | | | | AMIGO - Homo |
| | | | | sapiens |
| NOV4b | 277577082 | 19 | 20 | Transmembrane protein |
| | | | | AMIGO - Homo |
| | | | | sapiens |
| NOV4c | 277577094 | 21 | 22 | Transmembrane protein |
| | | | | AMIGO - Homo |
| | | | | sapiens |
| NOV4d | 277577141 | 23 | 24 | Transmembrane protein |
| | | | | AMIGO - Homo |
| | | | | sapiens |
| NOV5a | 306433917 | 25 | 26 | Nephronectin - Homo |
| | | | | sapiens |
| NOV5b | 306447063 | 27 | 28 | Nephronectin - Homo |
| | | | | sapiens |
| NOV5c | 306447071 | 29 | 30 | Nephronectin - Homo |
| | | | | sapiens |
| NOV5d | 306447075 | 31 | 32 | Nephronectin - Homo |
| | | | | sapiens |
| NOV5e | CG51117-09 | 33 | 34 | Nephronectin - Homo |
| | | | | sapiens |
| NOV5f | CG51117-14 | 35 | 36 | Nephronectin - Homo |
| | | | | sapiens |
| NOV5g | SNP13382208 | 37 | 38 | Nephronectin - Homo |
| | | | | sapiens |
| NOV6a | CG51923-01 | 39 | 40 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6b | 305869563 | 41 | 42 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6c | 305869567 | 43 | 44 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6d | 306076041 | 45 | 46 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6e | 317868343 | 47 | 48 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6f | 317868367 | 49 | 50 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6g | 317871203 | 51 | 52 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6h | 317871219 | 53 | 54 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6i | 317871243 | 55 | 56 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6j | 317871246 | 57 | 58 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6k | 317999764 | 59 | 60 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6l | 318176301 | 61 | 62 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6m | CG51923-02 | 63 | 64 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV6n | CG51923-03 | 65 | 66 | Protocadherin Fat 2 |
| | | | | precursor (hFat2) |
| | | | | (Multiple epidermal |
| | | | | growth factor-like |
| | | | | domains 1) - Homo |
| | | | | sapiens |
| NOV7a | CG52919-06 | 67 | 68 | SEZ-6 - Homo sapiens |
| NOV7b | 298521010 | 69 | 70 | SEZ-6 - Homo sapiens |
| NOV7c | CG52919-09 | 71 | 72 | SEZ-6 - Homo sapiens |
| NOV8a | CG94946-01 | 73 | 74 | AGRIN precursor - |
| | | | | Homo sapiens |
| | | | | (Human), 2026 aa |
| NOV8b | 308909220 | 75 | 76 | AGRIN precursor - |
| | | | | Homo sapiens |
| | | | | (Human), 2026 aa |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to: e.g., cardiomyopathy, atherosclerosis, hypertension, congenital heart defects, aortic stenosis, atrial septal defect (ASD), vascular calcification, fibrosis, atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, osteoarthritis, rheumatoid arthritis, osteochondrodysplasia, adrenoleukodystrophy, congenital adrenal hyperplasia, prostate cancer, diabetes, metabolic disorders, neoplasm; adenocarcinoma, lymphoma, uterus cancer, fertility, glomerulonephritis, hemophilia, hypercoagulation, idiopathic thrombocytopenic purpura, immunodeficiencies, psoriasis, skin disorders, graft versus host disease, AIDS, bronchial asthma, lupus, Crohn's disease; inflammatory bowel disease, ulcerative colitis, multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, schizophrenia, depression, asthma, emphysema, allergies, the metabolic syndrome X and wasting disorders associated with chronic diseases and various cancers, as well as conditions such as transplantation, neuroprotection, fertility, or regeneration (in vitro and in vivo).

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 38; (b) a variant of a mature form of the amino acid sequence selected from the group consisting of SEQ ID NO: 2n, wherein n is an integer between 1 and 38, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 38; (d) a variant of the amino acid sequence selected from the group consisting of SEQ ID NO:2n, wherein n is an integer between 1 and 38 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 38; (b) a variant of a mature form of the amino acid sequence selected from the group consisting of SEQ ID NO: 2n, wherein n is an integer between 1 and 38 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 38; (d) a variant of the amino acid sequence selected from the group consisting of SEQ ID NO: 2n, wherein n is an integer between 1 and 38, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 38 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n-1, wherein n is an integer between 1 and 38; (b) a nucleotide sequence wherein one or more nucleotides in the nucleotide sequence selected from the group consisting of SEQ ID NO: 2n-1, wherein n is an integer between 1 and 38 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n-1, wherein n is an integer between 1 and 38; and (d) a nucleic acid fragment wherein one or more nucleotides in the nucleotide sequence selected from the group consisting of SEQ ID NO: 2n-1, wherein n is an integer between 1 and 38 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid-is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n-1, wherein n is an integer between 1 and 38, or a complement of this nucleotide sequence, can be isolated using standard molecular biology techniques and the sequence information provided herein. Using all or a portion of the nucleic acid sequence of SEQ ID NO:2n-1, wherein n is an integer between 1 and 38, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, NY, 1989; and Ausubel, et al., (eds.), CURRENT PROTOCOLS IN MOLECULAR BIOLOGY, John Wiley & Sons, New York, NY, 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n-1, wherein n is an integer between 1 and 38, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n-1, wherein n is an integer between 1 and 38, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n-1, wherein n is an integer between 1 and 38, is one that is sufficiently complementary to the nucleotide sequence of SEQ ID NO:2n-1, wherein n is an integer between 1 and 38, that it can hydrogen bond with few or no mismatches to the nucleotide sequence shown in SEQ ID NO:2n-1, wherein n is an integer between 1 and 38, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3═ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, NY, 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n-1, wherein n is an integer between 1 and 38, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n-1, wherein n is an integer between 1 and 38; or an anti-sense strand nucleotide sequence of SEQ ID NO:2n-1, wherein n is an integer between 1 and 38; or of a naturally occurring mutant of SEQ ID NO:2n-1, wherein n is an integer between 1 and 38.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n-1, wherein n is an integer between 1 and 38, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Single Nucleotide Polymorphisms

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOVX Nucleic Acid and Polypeptide Variants

The invention further encompasses nucleic acid molecules that differ from the nucleotide sequences of SEQ ID NO:2n-1, wherein n is an integer between 1 and 38, due to degeneracy of the genetic code and thus encode the same NOVX proteins as that encoded by the nucleotide sequences of SEQ ID NO:2n-1, wherein n is an integer between 1 and 38. In another embodiment, an isolated nucleic acid molecule of the invention has a nucleotide sequence encoding a protein having an amino acid sequence of SEQ ID NO:2n, wherein n is an integer between 1 and 38.

In addition to the human NOVX nucleotide sequences of SEQ ID NO:2n-1, wherein n is an integer between 1 and 38, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n-1, wherein n is an integer between 1 and 38, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n-1, wherein n is an integer between 1 and 38. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n-1, wherein n is an integer between 1 and 38, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n-1, wherein n is an integer between 1 and 38, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n-1, wherein n is an integer between 1 and 38, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n-1, wherein n is an integer between 1 and 38, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 38. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n-1, wherein n is an integer between 1 and 38, yet retain biological activity. In one embodiment, the isolated nucleic acid molecule comprises a nucleotide sequence encoding a protein; wherein the protein comprises an amino acid sequence at least about 40% homologous to the amino acid sequences of SEQ ID NO:2n, wherein n is an integer between 1 and 38. Preferably, the protein encoded by the nucleic acid molecule is at least about 60% homologous to SEQ ID NO:2n, wherein n is an integer between 1 and 38; more preferably at least about 70% homologous to SEQ ID NO:2n, wherein n is an integer between 1 and 38; still more preferably at least about 80% homologous to SEQ ID NO:2n, wherein n is an integer between 1 and 38; even more preferably at least about 90% homologous to SEQ ID NO:2n, wherein n is an integer between 1 and 38; and most preferably at least about 95% homologous to SEQ ID NO:2n, wherein n is an integer between 1 and 38.

An isolated nucleic acid molecule encoding a NOVX protein homologous to the protein of SEQ ID NO:2n, wherein n is an integer between 1 and 38, can be created by introducing one or more nucleotide substitutions, additions or deletions into the nucleotide sequence of SEQ ID NO:2n-1, wherein n is an integer between 1 and 38, such that one or more amino acid substitutions, additions or deletions are introduced into the encoded protein.

Mutations can be introduced any one of SEQ ID NO:2n-1, wherein n is an integer between 1 and 38, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n-1, wherein n is an integer between 1 and 38, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (mRNA) mediated gene silencing where expression products of a NOVX gene are targeted by specific double stranded NOVX derived mRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate mRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N 19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMNE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell *culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n-1, wherein n is an integer between 1 and 38, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 38, or antisense nucleic acids complementary to a NOVX nucleic acid sequence of SEQ ID NO:2n-1, wherein n is an integer between 1 and 38, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine,. 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic. DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (ie., SEQ ID NO:2n-1, wherein n is an integer between 1 and 38). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-'OKeefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 38. The invention also includes a mutant or variant protein any of whose residues may be changed from the corresponding residues shown in any one of SEQ ID NO:2n, wherein n is an integer between 1 and 38, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 38) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 38. In other embodiments, the NOVX protein is substantially homologous to SEQ ID NO:2n, wherein n is an integer between 1 and 38, and retains the functional activity of the protein of SEQ ID NO:2n, wherein n is an integer between 1 and 38, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 38, and retains the functional activity of the NOVX proteins of SEQ ID NO:2n, wherein n is an integer between 1 and 38.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n-1, wherein n is an integer between 1 and 38.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 38, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to-treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 38, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays including radioligand binding assays or similar assays known to skilled artisans.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, NY, incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17,2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 lmmunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison ( Nature 368, 812-13 (1994)); Fishwild et al,( Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD 16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mnitogellin, restrictocin, phenomycin, enomycin, and the tricothecenes. A variety of radionuclides are available for the production of radioconjugated antibodies. Examples include ²¹²Bi, ¹³¹I, ¹³¹ In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-.diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin imlunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as imnimunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences selected on the basis of the host cells to be used for expression, that is operatively-linked to the nucleic acid sequence to be expressed. Within a recombinant expression vector,. “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kujan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1.) 1986.

Another aspect of the invention pertains-to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n-1, wherein n is an integer between 1 and 38, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n-1, wherein n is an integer between 1 and 38), but more preferably, is a non-human homologue of a human NOVX gene. For example, a mouse homologue of human NOVX gene of SEQ ID NO:2n-1, wherein n is an integer between 1 and 38, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTCAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the creAoxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut; et al.; 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of-a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal-extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678;. Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in -a complex. For example, test compounds can be labeled with ¹²⁵ I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity-of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-1 14, Thesit®, Isotridecypoly(ethylene glycol ether)ₙ, N-dodecyl-N,N-dimethyl-3-ammonio-1-propane sulfonate, 3-(3-cholamidopropyl) dimethylammniniol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test-compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater.(i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n-1, wherein n is an integer between 1 and 38, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., H CHROMOSOMES: A MANUAL OF BASIC TECHNCQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent-genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n-1, wherein n is an integer between 1 and 38, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n-1, wherein n is an integer between 1 and 38, or a portion thereof, such as an oligonucleotide of at least 15, 30, 50, 100, 250 or 500 nucleotides in length and sufficient to specifically hybridize under stringent conditions to NOVX mRNA or genormic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein-precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide and Polypeptide Sequences, and Homology Data

### Example 1

### NOV1, CG121992, CHORDIN

The NOV1 clone was analyzed, and the nucleotide and encoded polypeptide sequences are shown in Table 1A.

TABLE 1A

NOV1 Sequence Analysis

| | | |
|---|---|---|
| NOV1a, CG121992-03 | SEQ ID NO: 1 | 3628 bp |
| DNA Sequence | ORF Start: ATG at 247 | ORF Stop: TAG at 3193 |

CCCGGGTCAGCGCCCGCCCGCCCGCGCTCCTCCCGGCCGCTCCTCCCGCCCCGCCCGGCCCGGCGCCG
ACTCTGCGGCCGCCCGACGAGCCCCTCGCGGCACTGCCCCGGCCCCGGCCCCGCCCCCGGCCCCCTCC
CGCCGCACCGCCCCCGGCCCGGCCCTCCCCCCTCCGCACTCCCGCCTCCCTCCCTCCGCCCCCTCCCG
CGCCCTCCTCCCTCCCTCCTCCCCAGCTGTCCCGTTCGCGTCPB ATGCCGAGCCTCCCGGCCCCGCCGGC
CCCGCTGCTGCTCCTCGGGCTGCTGCTGCTCGGCTCCCGGCCGGCCCGCGGCGCCGGCCCCGAGCCCC
CCGTGCTGCCCATCCGTTCTGAGAAGGAGCCGCTGCCCGTTCGGGGAGCGGCAGGCTGCACCTTCGGC
GGGAAGGTCTATGCCTTGGACGAGACGTGGCACCCGGACCTAGGGGAGCCATTCGGGGTGATGCGCTG
CGTGCTGTGCGCCTGCGAGGCGCCTCAGTGGGGTCGCCGTACCAGGGGCCCTGGCAGGGTCAGCTGCA
AGAACATCAAACCAGAGTCCCCAACCCCGGCCTGTGGGCAGCCGCGCCAGCTGCCGGGACACTGCTGC
CAGACCTGCCCCCAGGAGCGCAGCAGTTCGGAGCGGCAGCCGAGCGGCCTGTCCTTCGAGTATCCGCG
GGACCCCGAGCATCGCAGTTATAGCGACCGCGGGGAGCCAGGCGCTGAGGAGCGGGCCCGTGGTGACG
GCCACACGGACTTCGTGGCGCTGCTGACAGGGCCGAGGTCGCAGGCGGTGGCACGAGCCCGAGTCTCG
CTGCTGCGCTCTAGCCTCCGCTTCTCTATCTCCTACAGGCGGCTGGACCGCCCTACCAGGATCCGCTT
CTCAGACTCCAATGGCAGTGTCCTGTTTGAGCACCCTGCAGCCCCCACCCAAGATGGCCTGGTCTGTG
GGGTGTGGCGGGCAGTGCCTCGGTTGTCTCTGCGGCTCCTTAGGGCAGAACAGCTGCATGTGGCACTT
GTGACACTCACTCACCCTTCAGGGGAGGTCTGGGGGCCTCTCATCCGGCACCGGGCCCTGGCTGCAGA
GACCTTCAGTGCCATCCTGACTCTAGAAGGCCCCCCACAGCAGGGCGTAGGGGGCATCACCCTGCTCA
CTCTCAGTGACACAGAGGACTCCTTGCATTTTTTGCTGCTCTTCCGAGGGCTGCTGGAACCCAGGAGT
GGGGGTAAGTGGGATGGGGGCAAAACACGTGAGAAGGTTAGGGAGAGCACCTGTCTCAGAAAGGCCCA
CATGTGCGGCCTTGCAGGACTAACCCAGGTTCCCTTGAGGCTCCAGATTCTACACCAGGGGCAGCTAC
TGCGAGAACTTCAGGCCAATGTCTCAGCCCAGGAACCAGGCTTTGCTGAGGTGCTGCCCAACCTGACA
GTCCAGGAGATGGACTGGCTGGTGCTGGGGGAGCTGCAGATGGCCCTGGAGTGGGCAGGCAGGCCAGG
GCTGCGCATCAGTGGACACATTGCTGCCAGGAAGAGCTGCGACGTCCTGCAAAGTGTCCTTTGTGGGG
CTGATGCCCTGATCCCAGTCCAGACGGGTGCTGCCGGCTCAGCCAGCCTCACGCTGCTAGGAAATGGC
TCCCTGATCTATCAGGTGCAAGTGGTAGGGACAAGCAGTGAGGTGGTGGCCATGACACTGGAGACCAA
GCCTCAGCGGAGGGATCAGCGCACTGTCCTGTGCCACATGGCTGGACTCCAGCCAGGAGGACACACGG
CCGTGGGTATCTGCCCTGGGCTGGGTGCCCGAGGGGCTCATATGCTGCTGCAGAATGAGCTCTTCCTG
AACGTGGGCACCAAGGACTTCCCAGACGGAGAGCTTCGGGGGCACGTGGCTGCCCTGCCCTACTGTGG
GCATAGCGCCCGCCATGACACGCTGCCCGTGCCCCTAGCAGGAGCCCTGCTGGTACCCCCTGTGAAGA
GCCAAGCAGCAGGGCACGCCTGGCTTTCCTTGGATACCCACTGTCACCTGCACTATGAAGTGCTGCTG
GCTGGGCTTGGTGGCTCAGAACAAGGCACTGTCACTGCCCACCTCCTTGGGCCTCCTGGAACGCCAGG
GCCTCGGCGGCTGCTGAAGGGATTCTATGGCTCAGAGGCCCAGGGTGTGGTGAAGGACCTGGAGCCGG
AACTGCTGCGGCACCTGGCAAAAGGCATGGCCTCCCTGATGATCACCACCAAGGGTAGCCCCAGAGGG
GAGCTCCGAGGGCAGGTGCACATAGCCAACCAATGTGAGGTTGGCGGACTGCGCCTGGAGGCGGCCGG
GGCCGAGGGGGTGCGGGCGCTGGGGGCTCCGGATACAGCCTCTGCTGCGCCGCCTGTGGTGCCTGGTC
TCCCGGCCCTAGCGCCCGCCAAACCTGGTGGTCCTGGGCGGCCCCGAGACCCCAACACATGCTTCTTC
GAGCGGCAGCAGCGCCCCCACGGGGCTCGCTGGGCGCCCAACTACGACCCGCTCTGCTCACTCTGCAC
CTGCCAGAGACGAACGGTGATCTGTGACCCGGTGGTGTGCCCACCGCCCAGCTGCCCACACCCGGTGC
AGGCTCCCGACCAGTGCTGCCCTGTTTGCCCTGAGAAACAAGATGTCAGAGACTTGCCAGGGCTGCCA
AGGAGCCGGGACCCAGGAGAGGGCTGCTATTTTGATGGTGACCGGAGCTGGCGGGCAGCGGGTACGCG
GTGGCACCCCGTTGTGCCCCCCTTTGGCTTAATTAAGTGTGCTGTCTGCACCTGCAAGGGGGGCACTG
GAGAGGTGCACTGTGAGAAGGTGCAGTGTCCCCGGCTGGCCTGTGCCCAGCCTGTGCGTGTCAACCCC
ACCGACTGCTGCAAACAGTGTCCAGTGGGGTCGGGGGCCCACCCCCAGCTGGGGGACCCCATGCAGGC
TGATGGGCCCCGGGGCTGCCGTTTTGCTGGGCAGTGGTTCCCAGAGAGTCAGAGCTGGCACCCCTCAG
TGCCCCCTTTTGGAGAGATGAGCTGTATCACCTGCAGATGTGGGGCAGGGGTGCCTCACTGTGAGCGG
GATGACTGTTCACTGCCACTGTCCTGTGGCTCGGGGAAGGAGAGTCGATGCTGTTCCCGCTGCACGGC
CCACCGGCGGCCAGCCCCAGAGACCAGAACTGATCCAGAGCTGGAGAAAGAAGCCGAAGGCTCTTAGG
GAGCAGCCAGAGGGCCAAGTGACCAAGAGGATGGGGCCTGAGCTGGGGAAGGGGTGGCATCGAGGACC
TTCTTGCATTCTCCTGTGGGAAGCCCAGTGCCTTTGCTCCTCTGTCCTGCCTCTACTCCCACCCCCAC
TACCTCTGGGAACCACAGCTCCACAAGGGGGAGAGGCAGCTGGGCCAGACCGAGGTCACAGCCACTCC
AAGTCCTGCCCTGCCACCCTCGGCCTCTGTCCTGGAAGCCCCACCCCTTTCCTCCTGTACATAATGTC
ACTGGCTTGTTGGGATTTTTAATTTATCTTCACTCAGCACCAAGGGCCCCCGACACTCCACTCCTGCT
GCCCCTGAGCTGAGCAGAGTCATTATTGGAGAGTTTTGTATTTATTAAAACATTTCTTTTTCAGTCAA
AAAAAAAAAAAAAAAAAAAAAAAA

| | | | |
|---|---|---|---|
| NOV1a, CG121992-03 | | | |
| Protein Sequence | SEQ ID NO: 2 | 982 aa | MW at 105031.2kD |

MPSLPAPPAPLLLLGLLLLGSRPARGAGPEPPVLPIRSEKEPLPVRGAAGCTFGGKVYALDETWHPDL
GEPFGVMRCVLCACEAPQWGRRTRGPGRVSCKNIKPECPTPACGQPRQLPGHCCQTCPQERSSSERQP
SGLSFEYPRDPEHRSYSDRGERPAEERARGDGHTDFVALLTGPRSQAVARARVSLLRSSLRFSISYRR
LDRPTRIRFSDSNGSVLFEHPAAPTQDGLVCGVWRAVPRLSLRLLRAEQLHVALVTLTHPSGEVWGPL
IRHRALAAETFSAILTLEGPPQQGVGGITLLTLSDTEDSLHFLLLFRGLLEPRSGGKWDGGKTREKVR
ESTCLRKAHMCGLAGLTQVPLRLQILHQGQLLRELQANVSAQEPGFAEVLPNLTVQEMDWLVLGELQM
ALEWAGRPGLRISGHIAARKSCDVLQSVLCGADALIPVQTGAAGSASLTLLGNGSLIYQVQVVGTSSE
VVAMTLETKPQRRDQRTVLCHMAGLQPGGHTAVGICPGLGARGAHMLLQNELFLNVGTKDFPDGELRG
HVAALPYCGHSARHDTLPVPLAGALVLPPVKSQAAGHAWLSLDTHCHLHYEVLLAGLGGSEQGTVTAH
LLGPPGTPGPRRLLKGFYGSEAQGVVKDLEPELLRHLAKGMASLMITTKGSPRGELRGQVHIANQCEV
GGLRLEAAGAEGVRALGAPDTASAAPPVVPGLPALAPAKPGGPGRPRDPNTCFFEGQQRPHGARWAPN
YDPLCSLCTCQRRTVICDPVVCPPPSCPHPVQAPDQCCPVCPEKQDVRDLPGLPRSRDPGEGCYFDGD
RSWRAAGTRWHPVVPPFGLIKCAVCTCKGGTGEVHCEKVQCPRLACAQPVRVNPTDCCKQCPVGSGAH
PQLGDPMQADGPRGCRFAGQWFPESQSWHPSVPPFGEMSCITCRCGAGVPHCERDDCSLPLSCGSGKE
SRCCSRCTAHRRPAPETRTDPELEKEAEGS

| | | |
|---|---|---|
| NOV1b, CG121992-02 | SEQ ID NO: 3 | 2829 bp |
| DNA Sequence | ORF Start: ATG at 40 | ORF Stop: TGA at 2410 |

CCTCCTCCCTCCCTCCTCCCCAGCTGTCCCGTTCGCGTCATGCCGAGCCTCCCGGCCCCGCCGGCCCC
GCTGCTGCTCCTCGGGCTGCTGCTGCTCGGCTCCCGGCCGGCCCGCGGCGCCGGCCCCGAGCCCCCCG
TGCTGCCCATCCGTTCTGAGAAGGAGCCGCTGCCCGTTCGGGGAGCGGCAGGCTGCACCTTCGGCGGG
AAGGTCTATGCCTTGGACGAGACGTGGCACCCGGACCTAGGGGAGCCATTCGGGGTGATGCGCTGCGT
GCTGTGCGCCTGCGAGGCGCCTCAGTGGGGTCGCCGTACCAGGGGCCCTGGCAGGGTCAGCTGCAAGA
ACATCAAACCAGAGTGCCCAACCCCGGCCTGTGGGCAGCCGCGCCAGCTGCCGGGACACTGCTGCCAG
ACCTGCCCCCAGGAGCGCAGCAGTTCGGAGCGGCAGCCGAGCGGCCTGTCCTTCGAGTATCCGCGGGA
CCCGGAGCATCGCAGTTATAGCGACCGCGGGGAGCCAGGCGCTGAGGAGCGGGCCCGTGGTGACGGCC
ACACGGACTTCGTCGCGCTGCTGACAGGGCCGAGGTCGCAGGCGGTGGCACGAGCCCGAGTCTCGCTG
CTGCGCTCTAGCCTCCGCTTCTCTATCTCCTACAGGCGGCTGGACCGCCCTACCAGGATCCGCTTCTC
AGACTCCAATGGCAGTGTCCTGTTTGAGCACCCTGCAGCCCCCACCCAAGATGGCCTGGTCTGTGGGG
TGTGGCGGGCAGTGCCTCGGTTGTCTCTGCGGCTCCTTAGGGCAGAACAGCTGCATGTGGCACTTGTG
ACACTCACTCACCCTTCAGGGGAGGTCTGGGGGCCTCTCATCCGGCACCGGGCCCTGGCTGCAGAGAC
CTTCAGTGCCATCCTGACTCTAGAAGGCCCCCCACAGCAGGGCGTAGGGGGCATCACCCTGCTCACTC
TCAGTGACACAGAGGACTCCTTGCATTTTTTGCTGCTCTTCCGAGGGCTGCTGGAACCCAGGAGTGGG
GGACTAACCCAGGTTCCCTTGAGGCTCCAGATTCTACACCAGGGGCAGCTACTGCGAGAACTTCAGGC
CAATGTCTCAGCCCAGGAACCAGGCTTTGCTGAGGTGCTGCCCAACCTGACAGTCCAGGAGATGGACT
GGCTGGTGCTGGGGGAGCTGCAGATGGCCCTGGAGTGGGCAGGCAGGCCAGGGCTGCGCATCAGTGGA
CACATTGCTGCCAGGAAGAGCTGCGACGTCCTGCAAAGTGTCCTTTGTGGGGCTGATGCCCTGATCCC
AGTCCAGACGGGTGCTGCCGGCTCAGCCAGCCTCACGCTGCTAGGAAATGGCTCCCTGATCTATCAGG
TGCAAGTGGTAGGGACAAGCAGTGAGGTGGTGGCCATGACACTGGAGACCAAGCCTCAGCGGAGGGAT
CAGCGCACTGTCCTGTGCCACATGGCTGGACTCCAGCCAGGAGGACACACGGCCGTGGGTATCTGCCC
TGGGCTGGGTGCCCGAGGGGCTCATATGCTGCTGCAGAATGAGCTCTTCCTGAATGTGGGCACCAAGG
ACTTCCCAGACGGAGAGCTTCGGGGGCACGTGGCTGCCCTGCCCTACTGTGGGCATAGCGCCCGCCAT
GACACGCTGCCCGTGCCCCTAGCAGGAGCCCTGGTGCTACCCCCTGTGAAGAGCCAAGCAGCAGGGCA
CGCCTGGCTTTCCTTGGATACCCACTGTCACCTGCACTATGAAGTGCTGCTGGCTGGGCTTGGTGGCT
CAGAACAAGGCACTGTCACTGCCCACCTCCTTGGGCCTCCTGGAACGCCAGGGCCTCGGCGGCTGCTG
AAGGGATTCTATGGCTCAGAGGCCCACGGTGTGGTGAAGGACCTGGAGCCGGAACTGCTGCGGCACCT
GGCAAAAGGCATGGCCTCCCTGCTGATCACCACCAAGGGTAGCCCCAGAGGGGAGCTCCGAGGGCAGG
TGCACATAGCCAACCAATGTGAGGTTGGCGGACTGCGCCTGGAGGCGGCCGGGGCCGAGGGGGTGCGG
GCGCTGGGGGCTCCGGATACAGCCTCTGCTGCGCCGCCTGTGGTGCCTGGTCTCCCGGCCCTAGCGCC
CGCCAAACCTGGTGGTCCTGGGCGGCCCCGAGACCCCAACACATGCTTCTTCGAGGGGCAGCAGCGCC
CCCACGGGGCTCGCTGGGCGCCCAACTACGACCCGCTCTGCTCACTCTGCACCTGCCAGAGACGAACG
GTGATCTGTGACCCGGTGGTGTGCCCACCGCCCAGCTGCCCACACCCGGTGCAGGCTCCCGACCAGTG
CTGCCCTGTTTGCCCTGAGAAACAAGATGTCAGAGACTTGCCAGGGCTGCCAAGGAGCCGGGACCCAG
GAGAGGGGGGGCACTGGAGAGGTGCACTGTGAGAAGGTGCACTGTCCCCGGCTGGCCTGTGCCCAGCC
TGTGCGTGTCAACCCCACCGACTGCTGCAAACAGTCTCCAGTGGGGTCGGCGGCCCACCCCCAGCTGG
GGGACCCCATGCAGGCTGATGGCCCCCGGGGCTGCCGTTTTGCTGGGCAGTGGTTCCCAGAGAGTCAC
AGCTCGCACCCCTCAGTGCCCCCGTTTGGAGACATGAGCTGTATCACCTGCAGATGTGGGCCAGGGGT
GCCTCACTGTGAGCGGGATGACTGTTCACTGCCACTGTCCTCTGGCTCGGGGAAGGAGAGTCGATGCT
GTTCCCGCTGCACGGCCCACCGGCGGCCAGCCCCACAGACCAGAACTGATCCAGAGCTGGAGAAAGAA
GCCGAAGCCTCTTAGGGAGCAGCCAGAGGGCCAAGTGACCA

| | | | |
|---|---|---|---|
| NOV1b, CG121992-02 | | | |
| Protein Sequence | SEQ ID NO: 4 | 790 aa | MW at 84215.7kD |

MPSLPAPPAPLLLLGLLLLCSRPARGAGPEPPVLPIRSEKEPLPVRGAAGCTFGGKVYALDETWHPDL
GEPFGVMRCVLCACEAPQWGRRTRGPGRVSCKNIKPECPTPACGQPRQLPGHCCQTCPQERSSSERQP
SGLSFEYPRDPEHRSYSDRGEPGAEERARGDGHTDFVALLTGPRSGAVARARVSLLRSSLRFSISYRR
LDRPTRIRFSDSNGSVLFEHPAAPTQDGLVCGVWRAVPRLSLRLLRAEQLHVALVTLTHPSGEVWGPL
IRHRALAAETFSAILTLEGPPQQGVGGITLLTLSDTEDSLHFLLLFRGLLEPRSCGLTQVPLRLQILH
QGQLLRELQANVSAQEPGFAEVLPNLTVQEMDWLVLGELQMALEWAGRPGLRISGHIAARKSCDVLQS
VLCGADALIPVQTGAAGSASLTLLGNGSLIYQVQVVGTSSEVVAMTLETKPQRRDQRTVLCHMAGLQP
GGHTAVGICPGLGARGAHMLLQNELFLNVGTKDFPDGELRGHVAALPYCGHSARHDTLPVPLAGALVL
PPVKSQAAGHAWLSLDTHCHLHYEVLLAGLGGSEQGTVTAHLLGPPGTPGPRRLLKGFYGSEAQGVVK
DLEPELLRHLAKGMASLLITTKGSPRGELRGQVHIANQCEVGGLRLEAAGAEGVRALGAPDTASAAPP
VVPGLPALAPAKPGGPGRPRDPNTCFFEGQQRPHGARWAPNYDPLCSLCTCQRRTVICDPVVCPPPSC
PHPVQAPDQCCPVCPEKQDVRDLPGLPRSRDPGEGGHWRGAL

| | | |
|---|---|---|
| NOV1c, CG121992-04 | SEQ ID NO: 5 | 2319 bp |
| DNA Sequence | ORF Start: at 1 | ORF Stop: at end of sequence |

GTTCGGGGAGCGGCAGGCTGCACCTTCGGCGGGAAGGTCTATGCCTTGGACGAGACGT
CGCACCCGGACCTAGGGGAGCCATTCGGGGTGATGCGCTGCGTGCTQTGCGCCTGCGAGGCGCCTCAG
TGGGGTCGCCGTACCAGGGGCCCTGGCACGGTCAGCTGCAAGAACATCAAACCAGAGTCCCCAACCCC
GGCCTGTGGGCAGCCGCGCCAGCTGCCGGGACACTGCTGCCAGACCTGCCCCCAGCAGCGCAGCAGTT
CGGAGCGGCAGCCGAGCGGCCTGTCCTTCGAGTATCCGCGGGACCCCGAGCATCGCAGTTATAGCGAC
CGCGGGGAGCCAGGCGCTGAGGACCGGGCCCGTGGTGACGCCCACACGGACTTCGTGGCGCTGCTCAC
AGGGCCGAGGTCGCAGGCGGTGGCACGAGCCCGAGTCTCGCTGCTGCGCTCTAGCCTCCGCTTCTCTA
TCTCCTACAGGCGGCTGGACCGCCCTACCAGGATCCGCTTCTCAGACTCCAATGGCAGTGTCCTGTTT
GAGCACCCTGCAGCCCCCACCCAAGATGGCCTGGTCTGTGGGGTGTGGCGGGCAGTGCCTCGGTTGTC
TCTGCGGCTCCTTAGGGCAGAACAGCTGCATGTGGCACTTGTGACACTCACTCACCCTTCAGGGGAGG
TCTGGGGGCCTCTCATCCGGCACCGGGCCCTGGCTGCAGAGACCTTCAGTGCCATCCTGACTCTAGAA
GGCCCCCCACAGCAGGGCGTAGGGGGCATCACCCTGCTCACTCTCAGTGACACAGAGGACTCCTTGCA
TTTTTTGCTGCTCTTCCGAGGGCTGCTGGAACCCAGGAGTGGCGGTAAGTGGGATGGGGGCAAAACAC
GTGAGAAGGTTAGGGAGAGCACCTGTCTCAGAAAGCCCCACATGTGCGGCCTTGCAGCACTAACCCAG
GTTCCCTTGAGGCTCCAGATTCTACACCAGGGGCAGCTACTGCGAGAACTTCAGGCCAATGTCTCAGC
CCAGGAACCAGGCTTTGCTGAGGTGCTGCCCAACCTGACAGTCCAGGAGATGGACTGGCTGGTGCTGC
GGGAGCTGCAGATCGCCCTGGAGTGGGCAGGCAGGCCAGGGCTGCGCATCAGTCGACACATTGCTGCC
AGGAAGAGCTGCGACGTCCTGCAAAGTGTCCTTTGTGGGGCTGATGCCCTGATCCCAGTCCAGACGGG
TGCTGCCGGGTCAGCCAGCCTCACGCTGCTAGGAAATGGCTCCCTGATCTATCAGGTGCAAGTGGTAG
GGACAAGCAGTGAGGTGGTGGCCATCACACTGGAGACCAAGCCTCAGCGGAGCGATCAGCGCACTGTC
CTGTGCCACATGGCTGGACTCCAGCCACGAGGACACACGGCCGTGGGTATCTGCCCTGGGCTGGGTGC
CCGAGGGCCTCATATGCTGCTGCAGAATGAGCTCTTCCTGAACGTGGGCACCAAGGACTTCCCAGACG
GAGAGCTTCGGGGGCACGTGGCTGCCCTGCCCTACTGTGGGCATAGCGCCCGCCATGACACGCTGCCC
GTGCCCCTAGCAGGACCCCTGGTGCTACCCCCTGTGAAGAGCCAAGCAGCAGGGCACGCCTGGCTTTC
CTTGGATACCCACTGTCACCTGCACTATGAAGTGCTGCTGGCTGGGCTTGGTGGCTCAGAACAAGGCA
CTGTCACTGCCCACCTCCTTGGGCCTCCTGGAACGCCAGGGCCTCGGCGGCTGCTGAAGGGATTCTAT
GGCTCAGAGGCCCAGCGTGTGGTGAAGGACCTGGAGCCGGAACTGCTGCGGCACCTGGCAAAAGGCAT
GGCCTCCCTGATGATCACCACCAAGGGTACCCCCACAGGGGAGCTCCGAGGGCAGGTGCACATAGCCA
ACCAATGTGAGGTTGGCCGACTGCGCCTGCAGGCGGCCGGGGCCGAGGGGGTGCGGGCGCTGGGGGCT
CCGGATACAGCCTCTGCTGCGCCGCCTGTGGTGCCTGGTCTCCCGGCCCTAGCGCCCGCCAAACCTGG
TGGTCCTGGGCGGCCCCGAGACCCCAACACATGCTTCTTCGAGGGGCAGCAGCGCCCCCACGGGCCTC
GCTGGGCGCCCAACTACGACCCGCTCTGCTCACTCTGCACCTGCCAGAGACGAACGGTGATCTGTGAC
CCGGTGGTGTGCCCACCGCCCAGCTGCCCACACCCGGTGCAGCCTCCCGACCAGTGCTGCCCTGTTTG
CCCTGAGAAACAAGATGTCAGAGACTTGCCAGGGCTGCCAAGGAGCCGGGACCCAGGAGAGGGGGGGC
ACTGGAGAGGTGCACTG

| | | | |
|---|---|---|---|
| NOV1c, CG121992-04 | | | |
| Protein Sequence | SEQ ID NO: 6 | 773 aa | MW at 82722.9kD |

VRGAAGCTFGGKVYALDETWHPDLGEPFCVMRCVLCACEAPQWGRRTRGPGRVSCKNIKPECPTPACG
QPRQLPCHCCQTCPQERSSSERQPSGLSFEYPRDPEHRSYSDRGEPGAEERARGDGHTDFVALLTGPR
SQAVARARVSLLRSSLRFSISYRRLDRPTRIRFSDSNGSVLFEHPAAPTQDGLVCCVWRAVPRLSLRL
LRAEQLHVALVTLTHPSGEVWGPLIRHRALAAETFSAILTLEGPPQQGVGGITLLTLSDTEDSLHFLL
LFRGLLEPRSGGKWDGGKTREKVRESTCLRKAHMCGLAGLTQVPLRLQILHQGQLLRELQANVSAQEP
GFAEVLPNLTVQEMDWLVLGELQMALEWAGRPGLRISGHIAARKSCDVLQSVLCGADALIPVQTGAAC
SASLTLLGNGSLIYQVQVVGTSSEVVAMTLETKPQRRDQRTVLCHMAGLQPGGHTAVGICPGLGARGA
HMLLQNELFLNVGTKDFPDGELRGHVAALPYCGHSARHDTLPVPLAGALVLPPVKSQAAGHAWLSLDT
HCHLHYEVLLAGLGGSEQGTVTAHLLCPPGTPGPRRLLKGFYGSEAQGVVKDLEPELLRHLAKGMASL
MITTKGSPRGELRGQVHIANQCEVGGLRLEAAGAEGVRALGAPDTASAAPPVVPGLPALAPAKPGGPG
RPRDPNTCFFEGQQRPHGARWAPNYDPLCSLCTCQRRTVICDPVVCPPPSCPHPVQAPDQCCPVCPEK
QDVRDLPGLPRSRDPGEGGHWRGAL

TABLE 1B
Comparison of the NOV1 protein sequences.

| | |
|---|---|
| NOV1a | MPSLPAPPAPLLLLGLLLLGSRPARGAGPEPPVLPIRSEKEPLPVRGAAGCTFGGKVYAL |
| NOV1b | MPSLPAPPAPLLLLGLLLLGSRPARGAGPEPPVLPIRSEKEPLPVRGAAGCTFGGKVYAL |
| NOV1c | --------------------------------------------VRGAAGCTFGGKVYAL |
| NOV1a | DETWHPDLGEPFGVMRCVLCACEAPQWGRRTRGPGRVSCKNIKPECPTPACGQPRQLPGH |
| NOV1b | DETWHPDLGEPFGVMRCVLCACEAPQWGRRTRGPGRVSCKNIKPECPTPACGQPRQLPGH |
| NOV1c | DETWHPDLGEPFGVMRCVLCACEAPQWGRRTRGPGRVSCKNIKPECPTPACGQPRQLPGH |
| NOV1a | CCQTCPQERSSSERQPSGLSFEYPRDPEHRSYSDRGEPGAEERARGDGHTDFVALLTGPR |
| NOV1b | CCQTCPQERSSSERQPSGLSFEYPRDPEHRSYSDRGEPGAEERARGDGHTDFVALLTGPR |
| NOV1c | CCQTCPQERSSSERQPSGLSFEYPRDPEHRSYSDRGEPGAEERARGDGHTDFVALLTGPR |
| NOV1a | SQAVARARVSLLRSSLRFSISYRRLDRPTRIRFSDSNCSVLFEHPAAPTQDGLVCGVWRA |
| NOV1b | SQAVARARVSLLRSSLRFSISYRRLDRPTRIRFSDSNGSVLFEHPAAPTQDGLVCGVWRA |
| NOV1c | SQAVARARVSLLRSSLRFSISYRRLDRPTRIRFSDSNGSVLFEHPAAPTQDGLVCGVWRA |
| NOV1a | VPRLSLRLLRAEQLHVALVTLTHPSGEVWGPLIRHRALAAETFSAILTLEGPPQQGVGGI |
| NOV1b | VPRLSLRLLRAEQLHVALVTLTHPSGEVWGPLIRHRALAAETFSAILTLEGPPQQGVGGI |
| NOV1c | VPRLSLRLLRAEQLHVALVTLTHPSGEVWGPLIRHRALAAETFSAILTLEGPPQQGVGGI |
| NOV1a | TLLTLSDTEDSLHFLLLFRGLLEPRSGGKWDGGKTREKVRESTCLRKAHMCGLAGLTQVP |
| NOV1b | TLLTLSDTEDSLHFLLLFRGLLEPRSGG---------------------------LTQVP |
| NOV1c | TLLTLSDTEDSLHFLLLFRGLLEPRSGGKWDGGKTREKVRESTCLRKAHMCGLAGLTQVP |
| NOV1a | LRLQILHQGQLLRELQANVSAQEPGFAEVLPNLTVQEMDWLVLGELQMALEWAGRPGLRI |
| NOV1b | LRLQILHQGQLLRELQANVSAQEPGFAEVLPNLTVQEMDWLVLGELQMALEWAGRPGLRI |
| NOV1c | LRLQILHQGQLLRELQANVSAQEPGFAEVLPNLTVQEMDWLVLGELQMALEWAGRPGLRI |
| NOV1a | SGHIAARKSCDVLQSVLCGADALIPVQTGAAGSASLTLLGNGSLIYQVQVVGTSSEVVAM |
| NOV1b | SGHIAARKSCDVLQSVLCGADALIPVQTGAAGSASLTLLGNGSLIYQVQVVGTSSEVVAM |
| NOV1c | SGHIAARKSCDVLQSVLCGADALIPVQTGAAGSASLTLLGNGSLIYQVQVVGTSSEVVAM |
| NOV1a | TLETKPQRRDQRTVLCHMAGLQPGGHTAVGICPGLGARGAHMLLQNELFLNVGTKDFPDG |
| NOV1b | TLETKPQRRDQRTVLCHMAGLQPGGHTAVGICPGLGARGAHNLLQNELFLNVGTKDFPDG |
| NOV1c | TLETKPQRRDQRTVLCHMAGLQPGGHTAVGICPGLGARGAHMLLQNELFLNVGTKDFPDG |
| NOV1a | ELRGHVAALPYCGHSARHDTLPVPLAGALVLPPVKSQAAGHAWLSLDTHCHLHYEVLLAG |
| NOV1b | ELRGHVAALPYCGHSARHDTLPVPLAGALVLPPVKSQAAGHAWLSLDTHCHLHYEVLLAG |
| NOV1c | ELRGHVAALPYCGHSARHDTLPVPLAGALVLPPVKSQAAGHAWLSLDTHCHLHYEVLLAG |
| NOV1a | LGGSEQGTVTAHLLGPPGTPGPRRLLKGFYGSEAQGVVKDLEPELLRHLAKGMASLMITT |
| NOV1b | LGGSEQGTVTAHLLGPPGTPCPRRLLKGFYGSEAQGVVKDLEPELLRHLAKGMASLLITT |
| NOV1c | LGGSEQGTVTAHLLGPPGTPGPRRLLKGFYGSEAQGVVKDLEPELLRHLAKGMASLMITT |
| NOV1a | KGSPRGELRGQVHIANQCEVGGLRLEAAGAEGVRALGAPDTASAAPPVVPGLPALAPAK |
| NOV1b | KGSPRGELRGQVHIANQCEVGGLRLEAAGAEGVRALGAPDTASAAPPVVPGLPALAPAK |
| NOV1c | KGSPRGELRGQVHIANQCEVGGLRLEAAGAEGVRALGAPDTASAAPPVVPGLPALAPAK |
| NOV1a | PGGPGRPRDPNTCFFEGQQRPHGARWAPNYDPLCSLCTCQRRTVICDPVV |
| NOV1b | PGGPGRPRDPNTCFFEGQQRPHGARWAPNYDPLCSLCTCQRRTVICDPVV |
| NOV1c | PGGPGRPRDPNTCFFEGQQRPHGARWAPNYDPLCSLCTCQRRTVICDPVV |
| NOV1a | CPPPSCPHPVQAPDQCCPVCPEKQDVRDLPGLPRSRDPGEGCYFDGDRSWRAAGTRW |
| NOV1b | CPPPSCPHPVQAPDQCCPVCPEKQDVRDLPGLPRSRDPGEG------GHWRGAL--- |
| NOV1c | CPPPSCPHPVQAPDQCCPVCPEKQDVRDLPGLPRSRDPGEG------GHWRGAL--- |
| NOV1a | HPVVPPFGLIKCAVCTCKGGTGEVHCEKVQCPRLACAQPVRVNPTDCCKQCPVGSGAHPQ |
| NOV1b | ------------------------------------------------------------ |
| NOV1c | ------------------------------------------------------------ |
| NOV1a | LGDPMQADGPRGCRFAGQWFPESQSWHPSVPPFGEMSCITCRCGAGVPHCERDDCSLPLS |
| NOV1b | ------------------------------------------------------------ |
| NOV1c | ------------------------------------------------------------ |
| NOV1a | CGSGKESRCCSRCTAHRRPAPETRTDPELEKEAEGS |
| NOV1b | ------------------------------------ |
| NOV1c | ------------------------------------ |
| NOV1a | (SEQ ID NO: 2) |
| NOV1b | (SEQ ID NO: 4) |
| NOV1c | (SEQ ID NO: 6) |

NOV1a, 1b have a cleavable signal peptide corresponding to amino acid residues 1 to 23 of SEQ ID NO:2 and 4 respectively. NOV1a mature protein corresponds to amino acid residues 24-982 of SEQ ID NO:2. NOV1b mature protein corresponds to amino acid residues 24-790 of SEQ ID NO:4. NOV1 sequences contain von Willebrand factor type C domains corresponding to amino acid residues 51-125 and 705-762 of NOV1b, SEQ ID NO:4; amino acid residues 51-125, 732-789, 811-877 and 899-959 of NOV1a SEQ ID NO:2; and amino acid residues 7-81 and 688-745 of NOV1c SEQ ID NO:6. NOV1a and NOV1c have a novel insertion at amino acid residues 329-355 of SEQ ID NO:2 and residues 285-311 of SEQ ID NO:6 respectively.

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABG31265 | Human chordin (CHRD) protein - | 1 . . . 982 | 954/982 (97%) | 0.0 |
| | Homo sapiens, 955 aa. | 1 . . . 955 | 955/982 (97%) | |
| | [WO200254940-A2, 18 JUL. | | | |
| | 2002] | | | |
| AAE12889 | Human chordin protein - Homo | 1 . . . 982 | 954/982 (97%) | 0.0 |
| | sapiens, 955 aa. [WO200164885- | 1 . . . 955 | 955/982 (97%) | |
| | A1, 07 SEP. 2001] | | | |
| AAW48978 | Mature human chordin protein - | 1 . . . 982 | 954/982 (97%) | 0.0 |
| | Homo sapiens, 954 aa. | 1 . . . 954 | 954/982 (97%) | |
| | [WO9821335-A1, 22 MAY 1998] | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9H2X0 | Chordin precursor - Homo | 1 . . . 982 | 954/982 (97%) | 0.0 |
| | sapiens (Human), 955 aa. | 1 . . . 955 | 955/982 (97%) | |
| Q9Z0E2 | Chordin precursor - Mus | 1 . . . 982 | 824/985 (83%) | 0.0 |
| | musculus (Mouse), 948 aa. | 1 . . . 948 | 863/985 (86%) | |
| O57465 | Chordin - Gallus gallus | 11 . . . 966 | 523/985 (53%) | 0.0 |
| | (Chicken), 940 aa. | 5 . . . 927 | 651/985 (65%) | |
| Q8N2W7 | Hypothetical protein - Homo | 570 . . . 982 | 413/413 (100%) | 0.0 |
| | sapiens (Human), 413 aa | 1 . . . 413 | 413/413 (100%) | |
| | (fragment). | | | |
| Q8TEH7 | FLJ00220 protein - Homo | 382 . . . 815 | 430/434 (99%) | 0.0 |
| | sapiens (Human), 503 aa | 68 . . . 501 | 432/434 (99%) | |
| | (fragment). | | | |

Chordin is a bone morphogenetic protein (BMP) antagonist. BMPs were originally identified by an ability of demineralized bone extract to induce endochondral osteogenesis in vivo in an extraskeletal site. To date, 15 BMPs have been identified and all are members of the transforming growth factor-beta superfamily of secreted signaling molecules and regulate tissue differentiation and maintenance. They play roles in embryogenesis by binding to specific serine/threonine kinase receptors, which transduce the signal to the nucleus. In contrast, there are proteins that antagonize the BMP functions by specifically binding to BMPs and preventing their binding to specific receptors or their signaling.

Chordin can interfere with normal embryogenesis by binding to TGF-beta-likeBMPs and sequestering them in latent complexes. It has been shown that BMP1 and TLL1 counteracted the effects of chordin upon overexpression in Xenopus embryos (Scott et al. “Mammalian BMP-1/Tolloid-related metalloproteinases, including novel family member mammalian Tolloid-like 2, have differential enzymatic activities and distributions of expression relevant to patterning and skeletogenesis.” Dev. Biol. 213: 283-300, 1999). They suggested that BMP1 is the major chordin antagonist in early mammalian embryogenesis and in pre- and postnatal skeletogenesis. It also directly binds BMP-4 and BMP-2, and interferes with the binding of these proteins to their receptors.

Bone metastases are a frequent clinical problem in patients with breast, prostate, and other cancers. Formation of these lesions is a site-specific process determined by multiple cellular and molecular interactions between the cancer cells and the bone microenvironment. BMP has been shown to be one of the significant factors in the prognosis of bone tumors. The overexpression of BMP2, BMP4, and BMP6 were found in most osteosarcomas or prostate cancers with metastases (Hamdy, F., Autzen, P., Robinson, MC., Wilson Horne, C H., Neal, D E. and Robson C N. “Immunolocalization and messenger RNA expression of bone morphogenetic protein-6 in human bening and malignant prostatic tissue.” Cancer Research 57: 44274431, 1997; Guo, W., Gorlick, R., Ladanyi, M., Meyers, P A., Huvos, A G., Bertino, J R., and Healey, J H.

“Expression of bone morphogenetic proteins and receptors in sarcomas.” Clinical Orthopaedics and Related Research 365: 175-183, 1999.) suggesting a close association between BMPs and skeletal metastases. BMP-2, -4, -6 may be responsible, in part, for osteoblastic changes in metastatic lesions secondary to prostate cancer. NOV1 has a role in the regulation of morphogenesis and cancer development. It is an important antibody or protein therapeutic target for the related diseases.

NOV1a has a nucleic acid of 3628 nucleotides (designated CuraGen Acc. No. CG121992-03) encoding a novel CHORDIN-like splice variant with deletion of exon 19 causing a frameshift staring from 784 aa. An open reading frame was identified beginning at nucleotides 247-249 and ending at nucleotides 3193-3195. This sequence represents a splice form of CHORDIN as indicated with 1 amino acid change L630M and insertion in frame of 27 amino acids KWDGGKTREKVRESTCLRKAMCGLAG (SEQ ID NO:77). The encoded protein having 982 amino acid residues contains 2 of 4 repeated von Willebrand factor type C domains compared to full length chordin. The von Willebrand factor (VWF) type C domain is found in multidomain protein/multifunctional proteins involved in maintaining homeostasis. The duplicated VWFC domain participates in oligomerization, but not in the initial dimerization step. The presence of this region in a number of other complex-forming proteins points to involvment of the VWFC domain in complex formation.

The CHORDIN-like genes disclosed in this invention map to chromosome 3. The PSORT, SignalP results for the CHORDIN-like protein NOV1a predict that this sequence has a signal peptide and is likely to be localized extracellularly with a certainty of 0.5469. The signal peptide is predicted by SignalP to be cleaved at amino acid between position 26 and 27: ARG-AG.

### Example 2

### NOV2, CG186275, ADAM 22

The NOV2 clone was analyzed, and the nucleotide and encoded polypeptide sequences are shown in Table 2A.

TABLE 2A

NOV2 Sequence Analysis

| | | |
|---|---|---|
| NOV2a, CG186275-03 | SEQ ID NO: 7 | 2847 bp |
| DNA Sequence | ORF Start: ATG at 47 | ORF Stop: TAA at 2795 |

CATGAGGAGCTGAGCGTCTCGGGCGAGGCGGGCTCACGGCAGCACCATGCAGCCGGCAGTGGCTGTGT
CCGTGCCCTTCTTGCTGCTCTGTGTCCTGCGGACCTGCCCTCCGGCGCGCTGCGGCCAGGCAGGAGAC
GCCTCATTGATGGAGCTAGAGAAGAGGAAGGAAAACCGCTTCGTGGAGCGCCAGAGCATCGTGCCACT
GCGCCTCATCTACCGCTCGGGCGGCGAAGACGAAAGTCGGCACGACGCGCTCGACACGCGGGTGCGGG
GCGACCTCGGTCGCCGGCAGATTCAGATGTTTTTGAAGTCACAATCCCAGAAGACCATATACCAGATA
CAGTTGACTCATGTTGACCAAGCAAGCTTCCAGGTTGATGCCTTTGGAACGTCATTCATTCTCGATGT
CGTGCTAAATCATGATTTGCTGTCCTCTGAATACATAGAGAGACACATTGAACATGGAGCCAAGACTG
TGGAAGTTAAAGGAGGAGAGCACTGTTACTACCACGGCCATATCCGAGGAAACCCTGACTCATTTGTT
GCATTGTCAACATGCCACGGACTTCATGGGATGTTCTATGACGGGAACCACACATATCTCATTGAGCC
AGAAGAAAATGACACTACTCAAGAGGATTTCCATTTTCATTCAGTTTACAAATCCAGACTGTTTGAAT
TTTCCTTGGATGATCTTCCATCTGAATTTCAGCAAATAAACATTACTCCATCAAAATTTATTTTGAAG
CCAAGACCAAAAAGGAGTAAACGGCACCTTCGTCGATATCCTCGTAATGTAGAAGAAGAAACCAAATA
CATTGAACTGATGATTGTGAATGATCACCTTATGTTTAAAAAACATCCGCTTTCCGTTGTACATACCA
ATACCTATGCGAAATCTGTGGTGAACATGGCAGATTTAATATATAAAGACCAACTTAAGACCAGGATA
GTATTGGTTGCTATGGAAACCTGGGCGACTGACAACAAGTTTGCCATATCTGAAAATCCATTGATCAC
CCTACGTGAGTTTATGAAATACAGGAGGGATTTTATCAAAGAGAAAAGTGATGCAGTTCACCTTTTTT
CGGGAAGTCAATTTGAGAGTAGCCGGAGCGGGGCAGCTTATATTGGTGGGATTTGCTCGTTGCTGAAA
GGAGGAGGCGTGAATGAATTTGGGAAAACTGATTTAATGGCTGTTACACTTGCCCAGTCATTAGCCCA
TAATATTGGTATTATCTCAGACAAAAGAAAGTTAGCAAGTGGTGAATGTAAATGCGAGGACACGTGGT
CCGGCTGCATAATGGGAGACACTGGCTATTATCTTCCTAAAAAGTTCACCCAGTGTAATATTGAAGAG
TATCATGACTTCCTGAATAGTGGAGGTGGTGCCTGCCTTTTCAACAAACCTTCTAAGCTTCTTGATCC
TCCTGAGTGTGGCAATGGCTTCATTGAAACTGGAGAGGAGTGTGATTGTGGAACCCCGGCCGAATGTG
TCCTTGAAGGAGCAGAGTGTTGTAACAAATGCACCTTGACTCAAGACTCTCAATGCAGTGACGGTCTT
TGCTGTAAAAAGTGCAAGTTTCAGCCTATGGGCACTGTGTGCCGACAAGCAGTAAATGATTGTGATAT
TCGTGAAACGTGCTCAGGAAATTCAAGCCAGTGTGCCCCTAATATTCATAAAATQGATGGATATTCAT
GTGATGGTGTTCAGGGAATTTCCTTTGGAGGAAGATGCAAAACCAGAGATAGACAATGCAAATACATT
TGGGGGCAAAAGGTGACAGCATCAGACAAATATTGCTATGAGAAACTGAATATTGAAGGGACGGAGAA
GGGTAACTGTGGGAAAGACAAAGACACATGGATACAGTGCAACAAACGGGATGTGCTTTGTGGTTACC
TTTTGTGTACCAATATTGGCAATATCCCAAGGCTTGGAGAACTCGATGGTGAAATCACATCTACTTTA
GTTGTGCAGCAAGGAAGAACATTAAACTGCAGTGGTGGCCATGTTAAGCTTGAAGAAGATGTAGATCT
TGGCTATGTGGAAGATGGGACACCTTGTGGTCCCCAAATGATGTGCTTAGAACACAGGTGTCTTCCTG
TGGCTTCTTTCAACTTTAGTACTTGCTTGAGCAGTAAAGAAGGCACTATTTGCTCAGGAAATGGAGTT
TGCAGTAATGAGCTGAAGTGTGTGTGTAACAGACACTGGATAGGTTCTGATTGCAACACTTACTTCCC
TCACAATGATGATGCAAAGACTGGTATCACTCTGTCTGGCAATGGTGTTCCTGGCACCAATATCATAA
TAGGCATAATTGCTGGCACCATTTTAGTGCTGGCCCTCATATTAGGAATAACTGCGTGGGGTTATAAA
AACTATCGAGAACAGAGGTCAAATGGGCTCTCTCATTCTTGGAGTGAAAGGATTCCAGACACAAAACA
TATTTCAGACATCTGTGAAAATGGGCGACCTCGAAGTAACTCTTGGCAAGGTAACCTGGGAGGCAACA
AAAAGAAAATCAGAGGCAAAAGATTTAGACCTCGGTCTAATTCAACTGAGTATTTAAACCCATGGTTC
AAAAGAGACTATAATGTAGCTAAGTGGGTAGAAGATGTGAATAAAAACACTGAAGAACCATACTTTAG
GACTTTATCTCCTGCCAAGTCTCCTTCTTCATCAACTGGGTCTATTGCCTCCAGCAGAAAATACCCTT
ACCCAATGCCTCCACTTCCTGATGAGGACAAGAAAGTGAACCGACAAAGTGCCAGGCTATGGGAGACA
TCCATTTAAGATCAACTGTTTACATGTGATACATCGAAAACTGTTTACTTCAACTTTTA

| | | | |
|---|---|---|---|
| NOV2a, CG186275-03 | | | |
| Protein Sequence | SEQ ID NO: 8 | 916 aa | MW at 102480.1kD |

MQAAVAVSVPFLLLCVLGTCPPARCGQAGDASLMELEKRKENRFVERQSIVPLRLIYRSGGEDESRHD
ALDTRVRGDLGGRQIQMFLKSESQKTIYQIQLTHVDQASFQVDAFGTSFILDVVLNHDLLSSEYIERH
IEHGGKTVEVKGGEHCYYQGHIRGNPDSFVALSTCHGLHGMFYDGNHTYLIEPEENDTTQEDFHFHSV
YKSRLFEFSLDDLPSEFGGINITPSKFILKPRPKRSKRGLRRYPRNVEEETKYIELMIVNDHLMFKKH
RLSVVHTNTYAKSVVNMADLIYKDQLKTRIVLVAMETWATDNKFAISENPLITLREFMKYRRDFIKEK
SDAVHLFSGSQFESSRSGAAYIGGICSLLKGGGVNEFGKTDLMAVTLAQSLAHNIGIISDKRKLASGE
CKCEDTWSGCIMGDTGYYLPKKFTQCNIEEYHDFLNSGGGACLFNKPSKLLDPPECGNGFIETGEECD
CGTPAECVLEGAECCKKCTLTQDSQCSDGLCCKKCKFQPMGTVCREAVNDCDIRETCSGNSSQCAPNI
HKMDGYSCDGVQGICFGGRCKTRDRQCKYIWGQKVTASDKYCYEKLNIEGTEKGNCGKDKDTWIQCNK
RDVLCGYLLCTNIGNIPRLGELDGEITSTLVVQQGRTLNCSGGHVKLEEDVDLGYVEDGTPCGPQMMC
LEHRCLPVASFNFSTCLSSKEGTICSGNGVCSNELKCVCNRHWIGSDCNTYFPHNDDAKTGITLSGNG
VAGTNIIIGIIAGTILVLALILGITAWGYKNYREQRSNGLSHSWSERIPDTKHISDICENGkPRSNSW
QGNLGGNKKKIRGKRFRPRSNSTEYLNPWFKRDYNVAKWVEDVNKNTEEPYFRTLSPAKSPSSSTGSI
ASSRKYPYPMPPLPDEDKKVNRQSARLWETSI

Further analysis of the NOV2a protein yielded the following properties. NOV2a has a cleavable signal peptide corresponding to amino acid residues 1-25 of SEQ ID NO:8. NOV2a has a novel insertions at amino acid residues 81-98 and 841-871 as well as a deletion of 36 amino acids between residues 784-784 of SEQ ID NO: 8.

A search of the NOV2a protein against the Geneseq database, a proprietary database that contains sequences published in patents and patent publication, yielded several homologous proteins shown in Table 2B.

TABLE 2B
Geneseq Results for NOV2a

| | | NOV2a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAY25119 | Human MDC2-beta protein - Homo | 1 . . . 840 | 821/840 (97%) | 0.0 |
| | sapiens, 823 aa. [JP11155574-A, | 1 . . . 823 | 822/840 (97%) | |
| | 15 JUN. 1999] | | | |
| AAY30208 | Amino acid sequence of the human | 40 . . . 916 | 830/913 (90%) | 0.0 |
| | SVPH3-13 protein - Homo sapiens, | 1 . . . 867 | 831/913 (90%) | |
| | 867 aa. [WO9941388-A2, 19 AUG. 1999] | | | |
| AAY25118 | Human MDC2-alpha protein - | 1 . . . 840 | 821/876 (93%) | 0.0 |
| | Homo sapiens, 859 aa. | 1 . . . 859 | 822/876 (93%) | |
| | [JP11155574-A, 15 JUN. 1999] | | | |
| AAR75352 | Human fetal brain MDC protein - | 52 . . . 787 | 407/742 (54%) | 0.0 |
| | Homo sapiens, 769 aa. [EP633268- | 50 . . . 768 | 518/742 (68%) | |
| | A2, 11 JAN. 1995] | | | |
| AAR67759 | Human fetal brain MDC protein - | 123 . . . 787 | 382/670 (57%) | 0.0 |
| | Homo sapiens, 670 aa. [EP633268- | 4 . . . 669 | 485/670 (72%) | |
| | A2, 11 JAN. 1995] | | | |

In a BLAST search of public sequence databases, the NOV2a protein was found to have homology to the proteins shown in the BLASTP data in Table 2C.

TABLE 2C
Public BLASTP Results for NOV2a

| | | NOV2a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9P0K1 | ADAM 22 precursor (A disintegrin | 1 . . . 916 | 868/952 (91%) | 0.0 |
| | and metalloproteinase domain 22) | 1 . . . 906 | 869/952 (91%) | |
| | (Metalloproteinase-like, disintegrin- | | | |
| | like, and cysteine-rich protein 2) | | | |
| | (Metalloproteinase-disintegrin | | | |
| | ADAM22-3) - Homo sapiens | | | |
| | (Human), 906 aa. | | | |
| Q9R1V6 | ADAM 22 precursor (A disintegrin | 1 . . . 840 | 751/876 (85%) | 0.0 |
| | and metalloproteinase domain 22) - | 1 . . . 857 | 783/876 (88%) | |
| | Mus musculus (Mouse), 857 aa. | | | |
| O42596 | ADAM 22 precursor (A disintegrin | 13 . . . 916 | 613/970 (63%) | 0.0 |
| | and metalloproteinase domain 22) | 12 . . . 935 | 709/970 (72%) | |
| | (Metalloprotease-disintegrin | | | |
| | MDC11b) (MDC11.2) - Xenopus | | | |
| | laevis (African clawed frog), 935 aa. | | | |
| O75078 | ADAM 11 precursor (A disintegrin | 52 . . . 787 | 408/742 (54%) | 0.0 |
| | and metalloproteinase domain 11) | 50 . . . 768 | 518/742 (68%) | |
| | (Metalloproteinase-like, disintegrin- | | | |
| | like, and cysteine-rich protein) (MDC)- | | | |
| | Homo sapiens (Human), 769 aa. | | | |
| Q9R1V4 | ADAM 11 precursor (A disintegrin | 52 . . . 787 | 409/743 (55%) | 0.0 |
| | and metalloproteinase domain 11) | 54 . . . 772 | 517/743 (69%) | |
| | (Metalloproteinase-like, disintegrin- | | | |
| | like, and cysteine-rich protein) (MDC)- | | | |
| | Mus musculus (Mouse), 773 aa. | | | |

PFam analysis predicts that the NOV2a protein contains the domains shown in the Table 3F.

TABLE 2D
Domain Analysis of NOV2a

| | NOV2a Match | Identities/ | |
|---|---|---|---|
| | Region Amino | Similarities | |
| | Acid residues of | for the | Expect |
| Pfam Domain | SEQ ID NO: 8 | Matched Region | Value |
| Pep_M12B_propep | 120 . . . 228 | 31/119 (26%) | 6.3e−17 |
| | | 76/119 (64%) | |
| Reprolysin | 256 . . . 455 | 69/206 (33%) | 1.4e−89 |
| | | 172/206 (83%) | |
| disintegrin | 470 . . . 546 | 35/79 (44%) | 4.2e−17 |
| | | 52/79 (66%) | |
| EGF | 696 . . . 728 | 10/48 (21%) | 0.4 |
| | | 21/48 (44%) | |

The cellular disintegrins, also known as ADAM (a disintegrin and metalloproteinase) and MDC (metalloproteinase-like, disintegrin-like, and cysteine-rich) proteins, are regulators of cell-cell and cell-matrix interactions. They contain multiple regions, including pro-, metalloproteinase-like, disintegrin-like, cysteine-rich, epidermal growth factor-like, transmembrane, and cytoplasmic domains.

NOV 2a has a nucleic acid of 2847 nucleotides (designated CuraGen Acc. No. CG186275-03) encoding a novel ADAM 22-like protein. An open reading frame was identified beginning at nucleotides 47-49 and ending at nucleotides 2795-2797. The encoded protein has 916 amino acid residues and is a splice form of ADAM 22 as indicated in position 81 with one exon insertion of 18 amino acids RQIQMFLKSESQKTIYQI (SEQ ID NO:79). NOV3 genes disclosed in this invention map to chromosome 7q21

The presence of identifiable domains in the protein was determined by searches of domain databases such as Pfam, PROSITE, ProDom, Blocks or Prints and then identified by the Interpro domain accession number. Significant domains include reprolysin, disintegrin and metalloendopeptidase domains.

Reprolysin, found in CD156 (also called ADAM8 (EC 3.4.24.-) or MS2 human) has been implicated in extravasation of leukocytes. The members of this family are enzymes that cleave peptides. These proteases require zinc for catalysis. Members of this family are also known as adamalysins. Most members of this family are snake venom endopeptidases, but there are also some mammalian proteins such as P78325, and fertilin Q28472. Fertilin and closely related proteins appear to not have some active site residues and may not be active enzymes.

Metalloendopeptidase M12B contains a sequence motif similar to the ‘cysteine switch’ of the matrixins. Many of the proteins with this domain are zinc proteases that may mediate cell-cell or cell-matrix interactions. The adhesion of platelets to the extracellular matrix, and platelet-platelet interactions, are essential in thrombosis and haemostasis. Platelets adhere to damaged blood vessels, release biologically active chemicals, and aggregate, a function that is inhibited in normal blood. The binding of fibrinogen to the glycoprotein IIb/IIIa complex of activated platelets is essential to platelet aggregation and is induced by many agonists, including ADP, collagen, thrombin, epinephrine and prostaglandin endoperoxide analogue. Snake venoms affect blood coagulation and platelet function in a complex manner: some induce aggregation and release reactions, and some inhibit them. Disintegrin, a component of some snake venoms, rather than inhibiting the release reactions, operates by inhibiting platelet aggregation, blocking the binding of fibrinogen to the receptor-glyco-protein complex of activated platelets. They act by binding to the integrin glycoprotein IIb-IIIa receptor on the platelet surface and inhibit aggregation induced by ADP, thrombin, platelet-activating factor and collagen. The role of disintegrin in preventing blood coagulation renders it of medical interest, particularly with regard to its use as an anti-coagulant.

Disintegrins are peptides of about 70 amino acid residues that contain many cysteines all involved in disulfide bonds. Disintegrins contain an Arg-Gly-Asp (RGD) sequence, a recognition site of many adhesion proteins. The RGD sequence of disintegrins interacts with the glycoprotein IIb-IIIa complex.

### Example 3

### NOV3 CG50586, Beta-Secretase

The NOV3 clone was analyzed, and the nucleotide and encoded polypeptide sequences are shown in Table 3A.

TABLE 3A

NOV3 Sequence Analysis

| | | |
|---|---|---|
| NOV3a, 260368272 | SEQ ID NO: 9 | 1140 bp |
| DNA Sequence | ORF Start: at 1 | ORF Stop: end of sequence |

AAGAATAAAGTTAAAGGCAGCCAAGGGCAGTTTCCACTAACACAGAATCTAACCGTTG
TTGAAGGTGGAACTGCAATTTTGACCTGCAGGGTTGATCAAAATGATAACACCTCCCTCCAGTGGTCA
AATCCAGCTCAACAGACTCTGTACTTTGACGACAAGAAAGCTTTAAGGGACAATAGGATCGAGCTGGT
TCGCGCTTCCTCGCATGAATTGAGTATTAGTGTCAGTAATGTGTCTCTCTCTGATGAAGGACAGTACA
CCTGTTCTTTATTTACAATGCCTGTCAAAACTTCCAACGCATATCTCACCGTTCTGGGTGTTCCTGAA
AAGCCTCAGATTAGTGGATTCTCATCACCAGTTATGGAGGGTGACTTGATGCAGCTGACTTGCAAAAC
ATCTGGTAGTAAACCTGCAGCTGATATAAGATGGTTCAAAAATGACAAAGAGATTAAAGATGTAAAAT
ATTTAAAAGAAGAGGATGCAAATCGCAAGACATTCACTGTCAGCAGCACACTGGACTTCCGAGTGGAC
CGGAGTGATGATGGAGTCGCGGTCATCTGCAGAGTAGATCACGAATCCCTCAATGCCACCCCTCAGGT
AGCCATGCAGGTGCTAGAAATACACTATACACCATCAGTTAAGATTATACCATCGACTCCTTTTCCAC
AAGAAGGACAGCCTTTAATTTTGACTTGTGAATCCAAAGGAAAACCACTGCCAGAACCTGTTTTGTGG
ACAAAGGATGGCGGAGAATTACCAGATCCTGACCGAATGGTTGTGAGTGGTAGGGAGCTAAACATTCT
TTTCCTGAACAAAACGGATAATGGTACATATCGATGTGAAGCCACAAACACCATTGGCCAAAGCAGTG
CGGAATATGTTCTCATTGTGCATGATCCTAATGCTTTGGCTGGCCAGAATGGCCCTGACCATGCTCTC
ATAGGAGGAATAGTGGCTGTAGTTGTATTTGTCACGCTGTGTTCTATCTTTCTGCTTCGTCGATATCT
GGCAAGGCATAAAGGAACGTATTTAACAAATGAAGCTAAAGGAGCTGAAGATGCACCAGATGCTGATA
CAGCCATTATCAATGCTGAACGCAGCCAAGTCAATGCTGAAGAGAAAAAACAGTATTTCATT

| | | | |
|---|---|---|---|
| NOV3a, 260368272 | | | |
| Protein Sequence | SEQ ID NO: 10 | 381 aa | MW at 42300.3kD |

SKNKVKGSQGQFPLTQNVTVVEGGTAILTCRVDQNDNTSLQWSNPAQQTLYFDDKKALRDNRIELV
RASWHELSISVSNVSLSDEGQYTCSLFTMPVKTSKAYLTVLGVPEKPQISGFSSPVMEGDLMQLTCKT
SGSKPAADIRWFKNDKEIKDVKYLKEEDANRKTFTVSSTLDFRVDRSDDGVAVICRVDHESLNATPQV
AMQVLEIHYTPSVKIIPSTPFPQEGQPLILTCESKGKPLPEPVLWTKDGGELPDPDRMVVSGRELNIL
FLNKTDNGTYRCEATNTIGQSSAEYVLIVHDPNALAGQNGPDHALIGGIVAVVVFVTLCSIFLLGRYL
ARHKGTYLTNEAKGAEDAPDADTAIINAEGSQVNAEEKKEYFI

| NOV3b, 260368280 | | |
|---|---|---|
| DNA Sequence | SEQ ID NO: 11 | 786 bp |
| NOV3b, 260368280 | | |
| DNA Sequence | ORF Start: at 1 | ORF Stop: end of sequence |

GGAACTGCAATTTTCACCTGCAGGGTTGATCAAAATCATAACACCTCCCTCCAGT
GGTCAAATCCAGCTCAACAGACTCTGTACTTTGACGACAAGAAAGCTTTAAGGGACAATAGGATCGAG
CTGGTTCGCGCTTCCTGGCATGAATTGAGTATTAGTGTCAGTGATGTGTCTCTCTCTGATGAAGGACA
GTACACCTGTTCTTTATTTACAATGCCTGTCAAAACTTCCAAGGCATATCTCACCCTTCTGGGTGTTC
CTGAAAACCCTCAGATTAGTCGATTCTCATCACCAGTTATGGAGGGTGACTTGATGCAGCTGACTTGC
AAAACATCTGGTAGTAAACCTGCAGCTGATATAAGATGGTTCAAAAATGACAAAGAGATTAAAGATGT
AAAATATTTAAAAGAAGAGGATGCAAATCGCAAGACATTCACTGTCAGCAGCACACTGGACTTCCGAG
TGGACCGCAGTGATGATGCACTGGCGGTCATCTGCAGAGTAGATCACGAATCCCTCAATGCCACCCCT
CAGGTAGCCATGCAGGTGCTAGAAATACACTATACACCATCAGTTAAGATTATACCATCGACTCCTTT
TCCACAAGAAGGACAGCCTTTAATTTTGACTTGTGAATCCAAAGGAAAACCACTGCCAGAACCTGTTT
TGTGGACAAAGGATGGCGGAGAATTACCAGATCCTGACCGAATGGTTGTGAGTGGTAGGGAGCTAAAC
ATTCTTTTCCTGAACAAAACGGATAATGGTACATATCGATGTGAAGCC

| | | | |
|---|---|---|---|
| NOV3b, 260368280 | | | |
| Protein Sequence | SEQ ID NO: 12 | 262 aa | MW at 29748.3kD |

GGTAILTCRVDQNDNTSLQWSNPAQQTLYFDDKKALRDNRIELVRASWHELSISVSDVSLSDEGQ
YTCSLFTMPVKTSKAYLTVLGVPEKPQISGFSSPVMEGDLMQLTCKTSGSKPAADTRWFKNDKEIKDV
YLKEEDAIRKTFTVSSTLDFRVDRSDDGVAVICRVDHESLNATPQVAMQKTLEIHYTPSVKIIPSTPF
PQEGQPLILTCESKGKPLPEPVLWTKDGGELPDPDRMVVSGRELNILFLNKTDNGTYRCE

| | | |
|---|---|---|
| NOV3c, 267441066 | SEQ ID NO: 13 | 1074 bp |
| DNA Sequence | ORF Start: at 1 | ORF Stop: end of sequence |

ATGATTTGGAAACGCAGCGCCGTTCTCCGCTTCTACAGTGTCTGCGGGCTCCTGG
TACAAGCGGCTGCTTCAAAGAATAAAGTTAAAGGCAGCCAAGGGCAGTTTCCACTAACACAGAATGTA
ACCGTTGTTGAAGGTGGAACTGCAATTTTGACCTGCAGGGTTGATCAAAATGATAACACCTCCCTCCA
GTGGTCAAATCCAGCTCAACAGACTCTGTACTTTGACGACAAGAAAGCTTTAAGGCACAATAGGATCG
AGCTGGTTCGCGCTTCCTGGCATGAATTGAGTATTAGTGTCAGTGATGTGTCTCTCTCTGATGAAGGA
CAGTACACCTGTTCTTTATTTACAATGCCTGTCAAAACTTCCAAGGCATATCTCACCGTTCTGGATGT
AAAATATTTAAAAGAAGAGGATGCAAATCGCAAGACATTCACTGTCAGCAGCACACTGGACTTCCGAG
TGGACCGGAGTGATGATGGAGTGGCGGTCATCTGCAGAGTAGATCACGAATCCCTCAATGCCACCCCT
CAGGTAGCCATGCAGGTGCTAGAAATACACTATACACCATCAGTTAACATTATACCATCGACTCCTTT
TCCACAAGAAGGACAGCCTTTAATTTTGACTTGTGAATCCAAAGGAAAACCACTGCCAGAACCTGTTT
TGTGGACAAAGGATGGCGGAGAATTACCAGATCCTGACCGAATGGTTGTGAGTGGTAGGGAGCTAAAC
ATTCTTTTCCTGAACAAAACGGATAATGGTACATATCGATGTGAAGCCACAAACACCATTGGCCAAAG
CAGTGCGGAATATGTTCTCATTGTGCATGATCCTAATGCTTTGCCTGGCCAGAATGGCCCTGACCATG
CTCTCATAGGAGGAATAGTGGCTGTAGTTGTATTTGTCACGCTGTGTTCTATCTTTCTGCTTGGTCGA
TATCTGGCAAGGCATAAAGGAACGTATTTAACAAATGAAGCTAAAGGAGCTGAAGATGCACCAGATGC
TGATACAGCCATTATCAATGCTGAAGGCAGCCAAGTCAATGCTGAAGAGAAAAAAGAGTATTTCATT

| | | | |
|---|---|---|---|
| NOV3c, 267441066 | | | |
| Protein Sequence | SEQ ID NO: 14 | 358 aa | MW at 40019.9kD |

MIWKRSAVLRFYSVCGLLVQAAASKNKVKGSQGQFPLTQNVTVVEGGTAILTCRVDQNDNTSLQ
WSNPAQQTLYFDDKKALRDNRTELVRASWHELSISVSDVSLSDEGQYTCSLFTMPVKTSKAYLTVLDV
KYLKEEDANRKTFTVSSTLDFRVDRSDDGVAVICRVDHESLNATPQVAMQVLEIHYTPSVKIIPSTPF
PQEGQPLILTCESKGKPLPEPVLWTKDGGELPDPDRMVVSGRELNILFLNKTDNGTYRCEATNTIGQS
SAEYVLIVHDPNALAGQNGPDHALIGGIVAVVVFVTLCSIFLLGRYLARHKGTYLTNEAKGAEDAPDA
DTAIINAEGSQVNAEEKKEYFI

| | |
|---|---|
| SEQ ID NO: 15 | 918 bp |
| ORF Start: a 1 | ORF Stop: end of sequence |

AAGAATAAAGTTAAAGGCAGCCAAGGGCAGTTTCCACTAACACACAATGTAACCGTTGTTGAAGGTGG
AACTGCAATTTTGACCTGCAGGGTTGATCAAAATGATAACACCTCCCTCCAGTGGTCAAATCCAGCTC
AACAGACTCTGTACTTTGACGACAAGAAAGCTTTAAGGGACAATACGATCGAGCTGGTTCGCGCTTCC
TGGCATGAATTGAGTATTAGTGTCAGTGATGTGTCTCTCTCTGATGAAGGACAGTACACCTGTTCTTT
ATTTACAATGCCTGTCAAAACTTCCAAGGCATATCTCACCGTTCTGGGTGTTCCTGAAAAGCCTCAGA
TTAGTGGATTCTCATCACCAGTTATGGAGGGTGACTTGATGCAGCTGACTTGCAAAACATCTGGTAGT
AAACCTGCAGCTCATATAAGATGGTTCAAAAATGACAAAGAGATTAAAGATGTAAAATATTTAAAAGA
AGAGGATGCAAATCGCAAGACATTCACTGTCAGCAGCACACTGGACTTCCGAGTGGACCGGAGTGATG
ATGGAGTGGCGGTCATCTGCAGAGTAGATCACGAATCCCTCAATGCCACCCCTCAGGTACCCATGCAG
GTGCTAGAAATACACTATACACCATCAGTTAAGATTATACCATCGACTCCTTTTCCACAAGAAGGACA
GCCTTTAATTTTGACTTGTGAATCCAAAGGAAAACCACTGCCAGAACCTGTTTTGTGGACAAAGGATG
GCGGAGAATTACCAGATCCTGACCGAATGGTTGTGAGTGGTAGGGAGCTAAACATTCTTTTCCTGAAC
AAAACGGATAATGGTACATATCGATGTGAAGCCACAAACACCATTGGCCAAAGCAGTGCGGAATATGT
TCTCATTGTGCATGATCCTAATGCTTTCGCTGGC

| | | | |
|---|---|---|---|
| NOV3d, CG50586-03 | | | |
| Protein Sequence | SEQ ID NO: 16 | 306 aa | MW at 33839.9kD |

KNKVKGSQGQFPLTQNVTVVEGGTAILTCRVDQNDNTSLQWSNPAQQTLYFDDKKALRDNRIELVRAS
WHELSISVSDVSLSDEGQYTCSLFTMPVKTSKAYLTVLGVPEKPQISGFSSPVMEGDLMQLTCKTSGS
KPAADIRWFKNDKEIKDVKYLKEEDANRKTFTVSSTLDFRVDRSDDGVAVICRVDHESLNATPQVAMQ
VLEIHYTPSVKIIPSTPFPQEGQPLILTCESKGKPLPEPVLWTKDGGELPDPDRMVVSGRELNILFLN
KTDNGTYRCEATNTIGQSSAEYVLIVHDPNALAG

A ClustalW comparison of the above protein sequences yields the following sequence alignment shown in Table 3B.

[image]

Further analysis of the NOV3c protein yielded the following properties shown in Table 3C.

TABLE 3C
Protein Sequence Properties NOV3a

| SignalP | Cleavage site between pos. 28 and 29 | | | | |
|---|---|---|---|---|---|
| analysis: | PSG: a new signal peptide prediction method | | | | |
| PSORT II | N-region: length 9; pos. chg 2; neg. chg 0 | | | | |
| analysis: | H-region: length 4; peak value −0.57 | | | | |
| | PSG score: −4.98 | | | | |
| | GvH: von Heijne's method for signal seq. recognition | | | | |
| | GvH score (threshold: −2.1): −4.54 | | | | |
| | possible cleavage site: between 27 and 28 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 2 | | | | |
| | Number of TMS(s) for threshold 0.5: 1 | | | | |
| | INTEGRAL | Likelihood = | −11.94 | Transmembrane | 299-315 |

| PERIPHERAL | Likelihood = | 6.26 (at 183) | |
|---|---|---|---|
| ALOM score: −11.94 (number of TMSs: 1) | | | |
| MTOP: Prediction of membrane topology (Hartmann et al.) | | | |
| Center position for calculation: 306 | | | |
| Charge difference: 3.5 C(2.5)-N(−1.0) | | | |
| C > N: C-terminal side will be inside | | | |
| >>> Single TMS is located near the C-terminus | | | |
| >>> membrane topology: type Nt (cytoplasmic tail 1 to 298) | | | |
| MITDISC: discrimination of mitochondrial targeting seq | | | |
| R content: | 2 | Hyd Moment(75): | 7.03 |
| Hyd Moment(95): | 5.62 | G content: | 4 |
| D/E content: | 1 | S/T content: | 9 |

Score: −2.29
Gavel: prediction of cleavage sites for mitochondrial preseq
R-3 motif at 17 LRFY|S
NUCDISC: discrimination of nuclear localization signals
pat4: none
pat7: none
bipartite: none
content of basic residues: 9.6%
NLS Score: −0.47
KDEL: ER retention motif in the C-terminus: none
ER Membrane Retention Signals: none
SKL: peroxisomal targeting signal in the C-terminus: none
SKL2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 21
checking 63 PROSITE DNA binding motifs: none
checking 71 PROSITE ribosomal protein motifs: none
checking 33 PROSITE prokaryotic DNA binding motifs: none
NNCN: Reinhardt's method for Cytplasmic/Nuclear discrimination
Prediction: cytoplasmic
Reliability: 89
COIL: Lupas's algorithm to detect coiled-coil regions
total: 0 residues

A search of the NOV3c protein against the Geneseq database, a proprietary database that contains sequences published in patents and patent publication, yielded several homologous proteins shown in Table 3D.

TABLE 3D
Geneseq Results for NOV3a

| | | NOV3a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB61142 | Human NOV12 protein - Homo | 105 . . . 362 | 236/262 (90%) | 0.0 |
| | sapiens, 404 aa. [WO200075321- | 143 . . . 404 | 240/262 (91%) | |
| | A2, 14 DEC. 2000] | | | |
| ABG66677 | Human novel polypeptide #12 - | 14 . . . 444 | 236/262 (90%) | 0.0 |
| | Homo sapiens, 404 aa. | 6 . . . 437 | 240/262 (91%) | |
| AAY33741 | Beta-secretase - Homo sapiens, | 105 . . . 286 | 159/187 (85%) | 0.0 |
| | 444 aa. | 143 . . . 329 | 163/187 (87%) | |

In a BLAST search of public sequence databases, the NOV3a protein was found to have homology to the proteins shown in the BLASTP data in Table 3E.

TABLE 3E
Public BLASTP Results for NOV3a

| | | NOV3a | Identities/ | |
|---|---|---|---|---|
| Protein | | Residues/ | Similarities | |
| Accession | | Match | for the | Expect |
| Number | Protein/Organism/Length | Residues | Matched Portion | Value |
| CAC22523 | Sequence 23 from Patent | 105 . . . 362 | 236/262 (90%) | 0.0 |
| | WO0075321 - Homo sapiens | 143 . . . 404 | 240/262 (91%) | |
| | (Human), 404 aa. | | | |
| Q8BLQ9 | Weakly similar to BK134P22.1 - | 105 . . . 362 | 232/262 (88%) | 0.0 |
| | Mus musculus (Mouse), 404 aa. | 143 . . . 404 | 237/262 (90%) | |
| Q8BYP1 | Weakly similar to BK134P22.1 - | 105 . . . 362 | 232/262 (88%) | 0.0 |
| | Mus musculus (Mouse), 404 aa. | 143 . . . 404 | 237/262 (90%) | |

PFam analysis predicts that the NOV3c protein contains the domains shown in the Table 3F.

TABLE 3F
Domain Analysis of NOV3a

| | NOV3a Match Region | | |
|---|---|---|---|
| | Amino acid residues | | |
| Pfam Domain | of SEQ ID NO: 10 | Score | E-Value |
| ig | 50 . . . 119 | 26.1 | 0.00081 |
| ig | 208 . . . 265 | 38.3 | 1.7e−07 |
| Adeno_E3_CR1 | 211 . . . 280 | −20.2 | 3.3 |

### Example 4

### NOV4, CG50637, T-Cell Surface Glycoprotein CD1B Precursor

The NOV4 clone was analyzed, and the nucleotide and encoded polypeptide sequences are shown in Table 4A.

TABLE 4A

NOV4 Sequence Analysis

| | | |
|---|---|---|
| NOV4a, CG50637-01 | SEQ ID NO: 17 | 1680 bp |
| DNA Sequence | ORF Start: ATG at 177 | ORF Stop: TGA at 1656 |

TGACAGGCCGGCCGGTGAGGCCCCGCCGCGAGAGGCCGCGACGCAGCTCCCAGACCGGCCATGGGCTC
AGACACGTCCTCGCCGAGCAGTGACCCTTCCGTACCCCACCAGAACATGCCCGGGTCACCTCCTCCCA
GATCTTCCTTGTGGCCTTCCTCGCCCACTCCAGTGACACTATGCACCCCCACCGTGACCCGAGAGGCC
TCTGGCTCCTCCTGCCGTCCTTGTCCCTGCTGCTTTTTGAGGTGGCCACAGCTGGCCGAGCCGTGGTT
AGCTGTCCTGCCGCCTCCTTGTGCGCCAGCAACATCCTCAGCTGCTCCAAGCAGCAGCTGCCCAATGT
GCCCCATTCCTTGCCCAGTTACACAGCACTACTGGACCTCAGTCACAACAACCTGAGCCGCCTGCGGG
CCGAGTGGACCCCCACGCGCCTGACCCAACTGCACTCCCTGCTGCTGAGCCACAACCACCTGAACTTC
ATCTCCTCTGAGGCCTTTTCCCCGGTACCCAACCTGCGCTACCTGGACCTCTCCTCCAACCAGCTGCG
TACACTGGATGAGTTCCTGTTCAGTCACCTGCAACTACTGGAGCTGCTGCTGCTCTACAATAACCACA
TCATGGCGGTGGACCGGTGCGCCTTCGATGACATGCCCCAGCTGCAGAAACTCTACTTGAGCCAGAAC
CAGATCTCTCGCTTCCCTCTGGAACTGGTCAAGGAAGGAGCCAAGCTACCCAAACTAACGCTCCTGGA
TCTCTCTTCTAACAAGCTGAAGAACTTGCCATTGCCTGACCTGCAGAAGCTGCCGGCCTGGATCAACA
ATGGGCTCTACCTACATAACAACCCCCTGAACTGCGACTGTGAGCTCTACCAGCTGTTTTCACACTGG
CAGTATCGGCAGCTGAGCTCCGTGATGGACTTTCAAGAGGATCTGTACTGCATGAACTCCAAGAAGCT
GCACAATGTCTTCAACCTGAGTTTCCTCAACTGTGGCGAGTACAAGGACCGTGCCTGGGAGGCCCACC
TGGGTGACACCTTGATCATCAAGTGTGACACCAAGCAGCAAGGGATGACCAAGGTGTGGGTGACACCA
AGTAATGAACGGGTGCTAGATGAGGTGACCAATGGCACACTGAGTGTGTCTAAGGATGGCAGTCTTCT
TTTCCAGCAGGTGCAGGTCGAGGACGGTGGTGTGTATACCTGCTATGCCATGGGAGAGACTTTCAATG
AGACACTGTCTGTGGAATTGAAAGTCCACAATTTCACCTTGCACGGACACCATGACACCCTCAACACA
GCCTATACCACCCTAGTGGGCTGTATCCTTAGTGTGGTCCTGGTCCTCATATACCTATACCTCACCCC
TTGCCGCTCCTGGTGCCGGGGTGTAGAGAAGCCTTCCAGCCATCAAGGAGACAGCCTCAGCTCTTCCA
TGCTTAGTACCACACCCAACCATGATCCTATCGCTGGTGGGGACAAAGATGATGGTTTTGACCGGCGG
GTGGCTTTCCTCGAACCTGCTGGACCTGGGCAGGGTCAAAACGGCAAGCTCAAGCCACGCAACACCCT
GCCAGTGCCTGAGGCCACAGGCAACGGCCAACGGAGGATGTCCGATCCAGAATCAGTCAGCTCGGTCT
TCTCTGATACGCCCATTGTGGTGTGAGCAGGATGGGTTGGTGGGGAGA

| | | | |
|---|---|---|---|
| NOV4a, CG50637-01 | | | |
| Protein Sequence | SEQ ID NO: 18 | 493 aa | MW at 55238.6kD |

MHPHRDPRGLWLLLPSLSLLLFEVARAGRAVVSCPAACLCASNILSCSKQQLPNVPHSLPSYTALLDL
SHNNLSRLRAEWTPTRLTQLHSLLLSHNHLNFISSEAFSPVPNLRYLDLSSNQLRTLDEFLFSDLQVL
EVLLLYNNHTMAVDRCAFDDMAQLQKLYLSQNQISRFPLELVKEGAKLPKLTLLDLSSNKLKNLPLPD
LQKLPAWIKNGLYLHNNPLNCDCELYQLFSHWQYRQLSSVMDFQEDLYCMNSKKLHNVFNLSFLNCGE
YKERAWEAHLGDTLIIKCDTKQQGMTKVWVTPSNERVLDEVTNGTVSVSKDGSLLFQQVQVEDGGVYT
CYAMGETFNETLSVELKVHNFTLHGHHDTLNTAYTTLVGCILSVVLVLIYLYLTPCRCWCRGVEKPSS
HQGDSLSSSMLSTTPNHDPMAGGDKDDGFDRRVAFLEPAGPGQGQNGKLKPGNTLPVPEATGKGQRRM
SDPESVSSVFSDTPIVV

| | | |
|---|---|---|
| NOV4b, 277577082 | SEQ ID NO: 19 | 1476 bp |
| DNA Sequence | ORF Start: at 1 | ORF Stop: end of sequence |

ATGCACCCCCACCGTGACCCGAGAGGCCTCTCGCTCCTGCTG
CCGTCCTTGTCCCTGCTGCTTTTTGAGGTGGCCAGAGCTGGCCGAGCCGTCGTTAGCTGTCCTCCCGC
CTCCTTGTGCGCCAACATCCTCAGCTGCTCCAAGCAGCAGCTGCCCAATGTGCCCCATTCCTTGCCCA
GTTACACAGCACTACTGGACCTCAGTCACAACAACCTGAGCCGCCTGCGGGCCGAGTGGACCCCCACG
CGCCTGACCCAACTGCACTCCCTGCTGCTGAGCCACAACCACCTGAACTTCATCTCCTCTGAGGCCTT
TTCCCCGGTACCCAACCTGCGCTACCTGGACCTCTCCTCCAACCAGCTGCGTACACTGGATGAGTTCC
TGTTCAGTGACCTGCAAGTACTCGAGGTGCTCCTGCTCTACAATAACCACATCATGGCGGTGGACCGG
TGCGCCTTCGATGACATGGCCCAGCTGCAGAAACTCTACTTGAGCCAGAACCAGATCTCTCCCTTCCC
TCTGGAACTGGTCAAGGAAGGAGCCAAGCTACCCAAACTAACGCTCCTGGATCTCTCTTCTAACAAGC
TGAAGAACTTGCCATTGCCTGACCTGCACAAGCTGCCGGCCTGGATCAAGAATGGGCTGTACCTACAT
AACAACCCCCTGAACTGCGACTGTGAGCTCTACCAGCTGTTTTCACACTGGCAGTATCGGCAGCTGAC
CTCCGTGATGGACTTTCAAGAGGATCTGTACTGCATGAACTCCAAGAAGCTGCACAATGTCTTCAACC
TGAGTTTCCTCAACTGTGGCGAGTACAAGGAGCGTGCCTGGGAGGCCCACCTGGGTGACACCTTGATC
ATCAAGTGTGACACCAAGCAGCAAGGGATGACCAAGGTGTGGGTGACACCAAGTAATGAACGGGTGCT
AGATGAGGTGACCAATGGCACAGTGAGTGTGTCTAACGATGGCAGTCTTCTTTTCCAGCAGGTGCAGG
TCGAGGACGGTGGTGTGTATACCTGCTATGCCATGGGACAGACTTTCAATGAGACACTGTCTGTGGAA
TTGAAAGTGCACAATTTCACCTTGCACGGACACCATGACACCCTCAACACAGCCTATACCACCCTAGT
GGGCTGTATCCTTAGTGTGGTCCTGGTCCTCATATACCTATACCTCACCCCTTGCCGCTGCTGGTGCC
GGGGTGTAGAGAAGCCTTCCAGCCATCAAGGAGACAGCCTCAGCTCTTCCATGCTTAGTACCACACCC
AACCATGATCCTATGGCTGGTGGGGACAAAGATGATGGTTTTGACCGGCGGGTGGCTTTCCTGGAACC
TGCTGGACCTGGGCACGGTCAAAACGGCAAGCTCAAGCCAGGCAACACCCTGCCAGTGCCTGAGGCCA
CAGGCAAGGGCCAACGGAGGATGTCGGATCCAGAATCAGTCAGCTCGGTCTTCTCTGATACGCCCATT
GTGGTG

| | | | |
|---|---|---|---|
| NOV4b, 277577082 | | | |
| Protein Sequence | SEQ ID NO: 20 | 492 aa | MW at 56294.8kD |

MNPHRDPRGLWLLLPSLSLLLFEVARAGRAVVSCPAACLCANILSCSKQQLPNVPHSLPS
YTALLDLSHNNLSRLRAEWTPTRLTQLHSLLLSHNHLNFISSEAFSPVPNLRYLDLSSNQLRTLDEFL
FSDLQVLEVLLLYNNHIMAVDRCAFDDMAQLQKLYLSQNQISRFPLELVKEGAKLPKLTLLDLSSNKL
KNLPLPDLQKLPAWIKNGLYLHNNPLNCDCELYQLFSHWQYRQLSSVMDFQEDLYCMNSKKLHNVFNL
SFLNCGEYKERAWEAHLGDTLIIKCDTKQQGMTKVWVTPSNERVLDEVTNGTVSVSKDCSLLFQQVQV
EDGGVYTCYAMGETFNETLSVELKVHNFTLHGHHDTLNTAYTTLVGCILSVVLVLIYLYLTPCRCWCR
GVEKPSSHQGDSLSSSMLSTTPNHDPMAGGDKDDGFDRRVAFLEPAGPGQGQNGKLKPGNTLPVPEAT
CKGQRRMSDPESVSSVFSDTPIVV

| NOV4c, 277577094 | | |
|---|---|---|
| DNA Sequence | SEQ ID NO: 21 | 1398 bp |
| NOV4c, 277577094 | | |
| DNA Sequence | ORF Start: at 1 | ORF Stop: end of sequence |

GGCCGAGCCGTGGTTAGCTGTCCTGCCGCCTGCTTGTGCGCCAGCAACATCCTCAGCT
GCTCCAAGCAGCAGCTGCCCAATGTGCCCCATTCCTTGCCCAGTTACACAGCACTACTGGACCTCAGT
CACAACAACCTGAGCCGCCTGCGGGCCGAGTGGACCCCCACGCGCCTGACCCAACTGCACTCCCTGCT
GCTGAGCCACAACCACCTGAACTTCATCTCCTCTGAGGCCTTTTCCCCGGTACCCAACCTGCGCTACC
TGCACCTCTCCTCCAACCAGCTGCGTACACTGGATGAGTTCCTGTTCAGTGACCTGCAAGTACTGGAG
GTGCTGCTGCTCTACAATAACCACATCATGGCCGTGGACCGGTGCGCCTTCGATGACATGGCCCACCT
GCAGAAACTCTACTTGAGCCAGAACCAGATCTCTCGCTTCCCTCTGGAACTGGTCAAGGAAGGACCCA
AGCTACCCAAACTAACGCTCCTCGATCTCTCTTCTAACAAGCTGAAGAACTTGCCATTCCCTGACCTG
CAGAAGCTGCCGGCCTGGATCAAGAATGGGCTGTACCTACATAACAACCCCCTGAACTGCGACTGTGA
GCTCTACCAGCTGTTTTCACACTGGCAGTATCGGCAGCTGAGCTCCGTGATGGACTTTCAAGAGGATC
TGTACTGCATGAACTCCAAGAAGCTGCACAATGTCTTCAACCTGAGTTTCCTCAACTGTGGCGAGTAC
AAGGAGCGTGCCTGGGAGGCCCACCTGGGTGACACCTTGATCATCAAGTGTGACACCAAGCAGCAAGG
GATGACCAAGGTGTGGGTGACACCAAGTAATGAACGGGTGCTAGATGAGGTGACCAATGGCACAGTGA
GTGTGTCTAAGGATGGCAGTCTTCTTTTCCAGCAGGTGCAGGTCGACGACGGTGGTGTGTATACCTGC
TATGCCATGGGAGAGACTTTCAATGAGACACTGTCTGTGGAATTGAAAGTGCACAATTTCACCTTGCA
CGGACACCATGACACCCTCAACACAGCCTATACCACCCTAGTGGGCTGTATCCTTAGTGTGGTCCTGG
TCCTCATATACCTATACCTCACCCCTTGCCGCTGCTGGTGCCGGGGTGTAGAGAAGCCTTCCAGCCAT
CAAGCAGACAGCCTCAGCTCTTCCATGCTTAGTACCACACCCAACCATGATCCTATGGCTGGTGGGGA
CAAAGATGATGGTTTTGACCGGCGGGTGGCTTTCCTGGAACCTGCTGGACCTGGGCAGGGTCAAAACG
GCAAGCTCAAGCCAGGCAACACCCTGCCAGTGCCTGAGGCCACAGGCAAGGGCCAACGGAGGATGTCG
GATCCAGAATCAGTCAGCTCGGTCTTCTCTGATACGCCCATTGTGGTG

| | | | |
|---|---|---|---|
| NOV4c, 277577094 | | | |
| Protein Sequence | SEQ ID NO: 22 | 466 aa | MW at 52731.6kD |

GRAVVSCPAACLCASNILSCSKQQLPNVPHSLPSYTALLDLSHNNLSRLRAEWTPTRLTQLHSLL
LSHNHLNFISSEAFSPVPNLRYLDLSSNQLRTLDEFLFSDLQVLEVLLLYNNHIMAVDRCAFDDMAQL
QKLYLSQNQISRFPLELVKEGAKLPKLTLLDLSSNKLKNLPLPDLQKLPAWIKNGLYLHNNPLNCDCE
LYQLFSHWQYRQLSSVMDFQEDLYCNNSKKLHNVFNLSFLNCGEYKERAWEAHLGDTLIIKCDTKQQG
MTKVWVTPSNERVLDEVTNGTVSVSKDGSLLFQQVQVEDGGVYTCYAMGETFNETLSVELKVHNFTLH
GHHDTLNTAYTTLVGCILSVVLVLIYLYLTPCRCWCRGVEKPSSHQGDSLSSSMLSTTPNHDPMAGGD
KDDGFDRRVAFLEPAGPGQCQNGKLKPGNTLPVPEATGKGQRRMSDPESVSSVFSDTPIVV

| | |
|---|---|
| SEQ ID NO: 23 | 717 bp |
| ORF Start: at 1 | ORF Stop: end of sequence |

AGCTGTCCTGCCGCCTGCTTGTGCGCCAGCAACATCCTCAGCTGCTCCAAGCAGCAGC
TGCCCAATGTGCCCCATTCCTTGCCCAGTTACACAGCACTACTGGACCTCAGTCACAACAACCTGAGC
CGCCTGCGGGCCGAGTGGACCCCCACGCGCCTGACCCAACTGCACTCCCTGCTCCTGAGCCACAACCA
CCTGAACTTCATCTCCTCTGACGCCTTTTCCCCGGTACCCAACCTCCGCTACCTGGACCTCTCCTCCA
ACCAGCTGCGTACACTGGATGAGTTCCTGTTCAGTGACCTGCAAGTACTGGAGGTGCTGCTGCTCTAC
AATAACCACATCATGGCCGTGGACCGGTGCGCCTTCGATGACATGGCCCAGCTGCAGAAACTCTACTT
GAGCCAGAACCAGATCTCTCGCTTCCCTCTGGAACTGGTCAAGGAAGGACCCAACCTACCCAAACTAA
CGCTCCTGGATCTCTCTTCTAACAAGCTGAAGAACTTGCCATTGCCTGACCTGCAGAAGCTGCCGGCC
TGGATCAAGAATGGGCTGTACCTACATAACAACCCCCTGAACTGCGACTGTGAGCTCTACCAGCTGTT
TTCACACTGGCAGTATCGGCAGCTGAGCTCCGTGATGGACTTTCAAGAGGATCTGTACTGCATGAACT
CCAAGAAGCTGCACAATGTCTTCAACCTGAGTTTCCTCAACTGTGGC

| | | | |
|---|---|---|---|
| NOV4d, 277577141 | | | |
| Protein Sequence | SEQ ID NO: 24 | 239 aa | MW at 28046.9.kD |

SCPAACLCASNILSCSKQQLPNVPHSLPSYTALLDLSHNNLSRLRAEWTPTRLTQLHSLLLSHNH
LNFISSEAFSPVPNLRYLDLSSNQLRTLDEFLFSDLQVLEVLLLYNNHIMAVDRCAFDDMAQLQKLYL
SQNQISRFPLELVKEGAKLPKLTLLDLSSNKLKNLPLPDLQKLPAWIKNGLYLHNNPLNCDCELYQLF
SNWQYRQLSSVMDFQEDLYCMNSKKLHNVFNLSFLNCG

A ClustalW comparison of the above protein sequences yields the following sequence alignment shown in Table 4B.

TABLE 4B
Comparison of the NOV4 protein sequences.

| | |
|---|---|
| NOV4a | MHPHRDPRGLWLLLPSLSLLLFEVARAGRAVVSCPAACLCASNILSCSKQQL |
| NOV4b | MHPHRDPRGLWLLLPSLSLLLFEVARAGRAVVSCPAACLCA-NILSCSKQQL |
| NOV4c | ---------------------------GRAVVSCPAACLCASNILSCSKQQL |
| NOV4d | -------------------------------SCPAACLCASNILSCSKQQL |
| NOV4a | PNVPHSLPSYTALLDLSHNNLSRLPAEWTPTRLTQLHSLLLSHNHLNFISSEAFSPVPNL |
| NOV4b | PNVPHSLPSYTALLDLSHNNLSRLRAEWTPTRLTQLHSLLLSHNHLNFISSEAFSPVPNL |
| NOV4c | PNVPHSLPSYTALLDLSHNNLSRLRAEWTPTRLTQLHSLLLSHNHLHFISSEAFSPVPNL |
| NOV4d | PNVPHSLPSYTALLDLSHNNLSRLRAEWTPTRLTQLHSLLLSHNHLNFISSEAFSPVPNL |
| NOV4a | RYLDLSSNQLRTLDEFLFSDLQVLEVLLLYNNHIMAVDRCAFDDMAQLQKLYLSQNQISR |
| NOV4b | RYLDLSSNQLRTLDEFLFSDLQVLEVLLLYNNHIMAVDRCAFDDMAQLQKLYLSQNQISR |
| NOV4c | RYLDLSSNQLRTLDEFLFSDLQVLEVLLLYNNHIMAVDRCAFDDMAQLQKLYLSQNQISR |
| NOV4d | RYLDLSSNQLRTLDEFLFSDLQVLEVLLLYNNHIMAVDRCAFDDMAQLQKLYLSQNQISR |
| NOV4a | FPLELVKEGAKLPKLTLLDLSSNKLKNLPLPDLQKLPAWIKNGLYLHNNPLNCDCELYQL |
| NOV4b | FPLELVKEGAKLPKLTLLDLSSNKLKNLPLPDLQKLPAWIKNGLYLHNNPLNCDCELYQL |
| NOV4c | FPLELVKEGAKLPKLTLLDLSSNKLKNLPLPDLQKLPAWIKNGLYLHNNPLNCDCELYQL |
| NOV4d | FPLELVKEGAKLPKLTLLDLSSNKLKNLPLPDLQKLPAWIKNGLYLHNNPLNCDCELYQL |
| NOV4a | FSHWQYRQLSSVMDFQEDLYCNNSKKLHNVFNLSFLNCGEYKERAWEAHLGDTLIIKCDT |
| NOV4b | FSHWQYRQLSSVMDFQEDLYCMNSKKLHNVFNLSFLNCGEYKERAWEAHLGDTLIIKCDT |
| NOV4c | FSHWQYRQLSSVMDFQEDLYCNNSKKLHNVFNLSFLNCGEYKERAWEAHLGDTLIIKCDT |
| NOV4d | FSHWQYRQLSSVMDFQEDLYCMNSKKLHNVFNLSFLNCG--------------------- |
| NOV4a | KQQGMTKVWVTPSNERVLDEVTNGTVSVSKDGSLLFQQVQVEDGGVYTCYAMGETFNETL |
| NOV4b | KQQGMTKVWVTPSNERVLDEVTNGTVSVSKDGSLLFQQVQVEDGGVYTCYAMGETFNETL |
| NOV4c | KQQGMTKVWVTPSNERVLDEVTNGTVSVSKDGSLLFQQVQVEDGGVYTCYAMGETFNETL |
| NOV4d | ------------------------------------------------------------ |
| NOV4a | SVELKVHNFTLHGHHDTLNTAYTTLVGCILSVVLVLIYLYLTPCRCWCRGVEKPSSHQGD |
| NOV4b | SVELKVHNFTLHGHHDTLNTAYTTLVGCILSVVLVLIYLYLTPCRCWCRGVEKPSSHQGD |
| NOV4c | SVELKVHNFTLHGHHDTLNTAYTTLVGCILSVVLVLIYLYLTPCRCWCRGVEKPSSHQGD |
| NOV4d | ------------------------------------------------------------ |
| NOV4a | SLSSSMLSTTPNHDPMAGGDKDDGFDRRVAFLEPAGPGQGQNGKLKPGNTLPVPEATGKG |
| NOV4b | SLSSSMLSTTPNHDPMAGGDKDDGFDRRVAFLEPAGPGQGQNGKLKPGNTLPVPEATGKG |
| NOV4c | SLSSSMLSTTPNHDPMAGGDKDDGFDRRVAFLEPAGPGQGQNGKLKPGNTLPVPEATGKG |
| NOV4d | ------------------------------------------------------------ |
| NOV4a | QRRMSDPESVSSVFSDTPIVV |
| NOV4b | QRRMSDPESVSSVFSDTPIVV |
| NOV4c | QRRMSDPESVSSVFSDTPIVV |
| NOV4d | --------------------- |
| NOV4a | (SEQ ID NO: 18) |
| NOV4b | (SEQ ID NO: 20) |
| NOV4c | (SEQ ID NO: 22) |
| NOV4d | (SEQ ID NO: 24) |

Further analysis of the NOV4a protein yielded the following properties shown in Table 4C.

TABLE 4C
Protein Sequence Properties NOV4a

| SignalP | Cleavage site between residues 35 and 36. | | | | |
|---|---|---|---|---|---|
| analysis: | PSG: a new signal peptide prediction method | | | | |
| PSORT II | N-region: length 5; pos. chg 1; neg. chg 0 | | | | |
| analysis: | H-region: length 7; peak value −5.92 | | | | |
| | PSG score: −10.32 | | | | |
| | GvH: von Heijne's method for signal seq. recognition | | | | |
| | GvH score (threshold: −2.1): −0.90 | | | | |
| | possible cleavage site: between 35 and 36 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 3 | | | | |
| | INTEGRAL | Likelihood = | −2.28 | Transmembrane | 17-33 |
| | INTEGRAL | Likelihood = | −2.18 | Transmembrane | 38-54 |
| | INTEGRAL | Likelihood = | −10.83 | Transmembrane | 384-400 |

| PERIPHERAL | Likelihood = | 2.44 (at 132) | |
|---|---|---|---|
| ALOM score: −10.83 (number of TMSs: 3) | | | |
| MTOP: Prediction of membrane topology (Hartmann et al.) | | | |
| Center position for calculation: 24 | | | |
| Charge difference: −2.0 C(1.0)-N(3.0) | | | |
| N >= C: N-terminal side will be inside | | | |
| >>> membrane topology: type 3a | | | |
| MITDISC: discrimination of mitochondrial targeting seq | | | |
| R content: | 2 | Hyd Moment(75): | 5.88 |
| Hyd Moment(95): | 6.17 | G content: | 1 |
| D/E content: | 2 | S/T content: | 5 |

Score: −4.21
Gavel: prediction of cleavage sites for mitochondrial preseq
R-2 motif at 47 GRA|VV
NUCDISC: discrimination of nuclear localization signals
pat4: none
pat7: none
bipartite: none
content of basic residues: 8.5%
NLS Score: −0.47
KDEL: ER retention motif in the C-terminus: none
ER Membrane Retention Signals: none
SKL: peroxisomal targeting signal in the C-terminus: none
SKL2: 2nd peroxisomal targeting signal: none
VAC: possible vacuolar targeting motif: found
KLPK at 190
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:
Leucine zipper pattern (PS00029): *** found ***
LSCSKQQLPNVPHSLPSYTALL at 52
LDLSSNQLRTLDEFLFSDLQVL at 122
LKVHNFTLHGHHDTLNTAYTTL at 363
none
checking 71 PROSITE ribosomal protein motifs: none
checking 33 PROSITE prokaryotic DNA binding motifs: none
NNCN: Reinhardt's method for Cytplasmic/Nuclear discrimination
Prediction: nuclear
Reliability: 55.5
COIL: Lupas's algorithm to detect coiled-coil regions
total: 0 residues

A search of the NOV4a protein against the Geneseq database, a proprietary database that contains sequences published in patents and patent publication, yielded several homologous proteins shown in Table 4D.

TABLE 4D
Geneseq Results for NOV4a

| | | NOV4a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB49650 | Human SEC2 protein sequence SEQ | 9 . . . 500 | 492/493 (99%) | 0.0 |
| | ID 4 - Homo sapiens, 493 aa. | 1 . . . 493 | 492/493 (99%) | |
| | [WO200070046-A2, 23 NOV. 2000] | | | |
| ABJ10921 | Human secreted protein (SECP) #17 - | 9 . . . 500 | 492/493 (99%) | 0.0 |
| | Homo sapiens, 493 aa. | 1 . . . 493 | 492/493 (99%) | |
| ABB17119 | Human nervous system related | 158 . . . 335 | 177/178 (99%) | 0.0 |
| | polypeptide SEQ ID NO 5776 - | 27 . . . 204 | 177/178 (99%) | |
| | Homo sapiens, 212 aa. | | | |
| | [WO200159063-A2, 16 AUG. 2001] | | | |

In a BLAST search of public sequence databases, the NOV4a protein was found to have homology to the proteins shown in the BLASTP data in Table 4E.

TABLE 4E
Public BLASTP Results for NOV4a

| | | NOV4a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q86WK6 | Transmembrane protein AMIGO - | 9 . . . 500 | 492/493 (99%) | 0.0 |
| | Homo sapiens (Human), 493 aa. | 1 . . . 493 | 492/493 (99%) | |
| Q8IW71 | Hypothetical protein - Homo | 9 . . . 500 | 491/493 (99%) | 0.0 |
| | sapiens (Human), 493 aa. | 1 . . . 493 | 493/493 (99%) | |
| Q80ZD8 | Transmembrane protein AMIGO - | 9 . . . 500 | 440/492 (89%) | 0.0 |
| | Mus musculus (Mouse), 492 aa. | 1 . . . 492 | 463/492 (94%) | |

PFam analysis predicts that the NOV4a protein contains the domains shown in the Table 4F.

TABLE 4F
Domain Analysis of NOV4a

| | NOV4a Match Region | | |
|---|---|---|---|
| | Amino acid residues | | |
| Pfam Domain | of SEQ ID NO: 18 | Score | E-Value |
| LRRNT | 41 . . . 67 | 11.6 | 1.2 |
| LRR | 69 . . . 91 | 4.2 | 3e+02 |
| LRR | 94 . . . 117 | 15.4 | 1.3 |
| LRR | 118 . . . 141 | 23.4 | 0.0053 |
| LRR | 142 . . . 165 | 8.3 | 78 |
| LRR | 166 . . . 185 | 11.2 | 24 |
| LRR | 193 . . . 216 | 19.3 | 0.093 |
| LRRCT | 228 . . . 278 | 30.0 | 5.6e−05 |
| Ig | 290 . . . 350 | 20.7 | 0.036 |

### Example 5

### NOV5, CG51117-09, Nephronectin

The NOV5 clone was analyzed, and the nucleotide and encoded polypeptide sequences are shown in Table 5A.

TABLE 5A

NOV5 Sequence Analysis

| | | |
|---|---|---|
| NOV5a, 306433917 | SEQ ID NO: 25 | 1413 bp |
| DNA Sequence | ORF Start: at 1 | ORF Stop: end of sequence |

TGCCAACCACGATGGAAACATGGTGAATGTATCGGGCCAAACAAGTGCAAGTGTCATC
CTGGTTATGCTGGAAAAACCTGTAATCAAGATCTAAATGAGTGTGGCCTGAAGCCCCGGCCCTGTAAG
CACAGGTGCATGAACACTTACGGCAGCTACAAGTGCTACTGTCTCAACGGATATATGCTCATGCCGGA
TGGTTCCTGCTCAAGTGCCCTGACCTGCTCCATGGCAAACTGTCAGTATGGCTGTGATGTTGTTAAAG
GACAAATACGGTGCCAGTGCCCATCCCCTGGCCTGCAGCTGGCTCCTGATGGGAGGACCTGTGTAGAT
GTTGATGAATGTCCTACAGGAAGAGCCTCCTGCCCTAGATTTAGGCAATGTGTCAACACTTTTGGCAG
CTACATCTGCAAGTGTCATAAAGGCTTCGATCTCATCTATATTGGAGACATAGACGAATGCTCACTTG
GTCAGTATCAGTGCAGCAGCTTTGCTCGATGTTATAACGTACGTGGGTCCTACAAGTGCAAATGTAAA
GAAGGATACCAGGGTGATGGACTGACTTGTGTGTATATCCCAAAAGTTATGATTGAACCTTCAGGTCC
AATTCATGTACCAAAGGGAAATCGTACCATTTTAAAGGGTGACACAGGAAATAATAATTGGATTCCTG
ATGTTGGAAGTACTTGGTGGCCTCCGAAGACACCATATATTCCTCCTATCATTACCAACAGGCCTACT
TCTAAGCCAACAACAAGACCTACACCAAAGCCAACACCAATTCCTACTCCACCACCACCACCACCCCT
GCCAACAGAGCTCAGAACACCTCTACCACCTACAACCCCAGAAAGGCCAACCACCGGACTGACAACTA
TAGCACCAGCTGCCAGTACACCTCCAGGAGGGATTACAGTTGACAACAGGGTACAGACAGACCCTCAG
AAACCCAGAGGAGATGTGTTCAGTGTTCTGGTACACAGTTGTAATTTTGACCATGGACTTTGTGGATG
GATCAGGGAGAAAGACAATGACTTGCACTGGGAACCAATCAGGGACCCAGCAGGTGGACAATATCTGA
CAGTGTCGGCAGCCAAAGCCCCAGGGGGAAAAGCTGCACGCTTGGTGCTACCTCTCGCCCGCCTCATG
CATTCAGGCGACCTGTGCCTGTCATTCAGGCACAAGGTGACGGGGCTGCACTCTGGCACACTCCACGT
GTTTGTGAGAAAACACGGTGCCCACGGAGCAGCCCTGTCGGGAAGAAATGGTGGCCATGGCTGGAGGC
AAACACAGATCACCTTGCGAGGGGCTGACATCAAGAGCGTCGTCTTCAAAGGTGAAAAAAGGCGTGGT
CACACTGGGGAGATTGGATTAGATGATGTGAGCTTGAAAAAAGGCCACTGCTCTGAAGAACGC

| | | | |
|---|---|---|---|
| NOV5a, 306433917 | | | |
| Protein Sequence | SEQ ID NO: 26 | 471 aa | MW at 51775.8kD |

CQPRCKHGECIGPNKCKCHPGYAGKTCNQDLNECGLKPRPCKHRCNNTYGSYKCYCLNGYMLMPD
GSCSSALTCSMANCQYGCDVVKGQIRCQCPSPGLQLAPDGRTCVDVDECATGRASCPRFRQCVNTFGS
YICKCHKGFDLMYIGDIDECSLGQYQCSSFARCYNVRGSYKCKCKEGYQGDGLTCVYIPKVMIEPSGP
IHVPKGNGTILKGDTGNNNWIPDVCSTWWPPKTPYIPPIITNRPTSKPTTRPTPKPTPIPTPPPPPPL
PTELRTPLPPTTPERPTTGLTTIAPAASTPPGGITVDNRVQTDPQKPRGDVFSVLVHSCNFDHGLCGW
IREKDNDLHWEPIRDPAGGQYLTVSAAKAPGGKAARLVLPLGRLMHSGDLCLSFRHKVTGLHSGTLQV
FVRKHGAHGAALWGRNGGHGWRQTQITLRGADIKSVVFKGEKRRGHTGEIGLDDVSLKKGHCSEER

| | | |
|---|---|---|
| NOV5b, 306447063 | SEQ ID NO: 27 | 1743 bp |
| DNA Sequence | ORF Start: at 1 | ORF Stop: end of sequence |

ATGGATTTTCTCCTGGCGCTGGTGCTGGTATCCTCGCTC
TACCTGCAGGCGGCCGCCGAGTTCGACGGGAGTAGGTGGCCCAGGCAAATAGTGTCATCGATTGGCCT
ATGTCGTTATGGTGGGAGCATTGACTGCTGCTGGGGCTGGGCTCCCCAGTCTTGGGGACAGTGTCAGC
CTTTCTACGTCTTAAGGCAGAGAATAGCCAGGATAAGGTGCCAGCTCAAAGCTGTGTGCCAACCACGA
TGCAAACATGGTGAATGTATCGGGCCAAACAAGTGCAAGTGTCATCCTGGTTATGCTGGAAAAACCTG
TAATCAAGATCTAAATGAGTGTCGCCTGAAGCCCCGGCCCTGTAAGCACAGGTGCATGAACACTTACC
GCAGCTACAAGTGCTACTGTCTCAACGGATATATGCTCATGCCGGATGGTTCCTGCTCAAGTGCCCTG
ACCTGCTCCATGGCAAACTGTCACTATGGCTGTGATGTTGTTAAAGGACAAATACGGTGCCAGTGCCC
ATCCCCTGGCCTGCAGCTGGCTCCTGATGGGAGGACCTGTGTAGATGTTCATGAATGTGCTACAGGAA
GAGCCTCCTGCCCTAGATTTAGGCAATGTGTCAACACTTTTGGGAGCTACATCTGCAAGTGTCATAAA
GGCTTCGATCTCATGTATATTGGAGGCAAATATCAATGTCATGACATAGACGAATGCTCACTTGGTCA
GTATCAGTGCAGCAGCTTTGCTCGATGTTATAACGTACGTGGGTCCTACAAGTGCAAATGTAAAGAAG
GATACCAGGGTCATGGACTGACTTGTGTGTATATCCCAAAAGTTATGATTGAACCTTCAGGTCCAATT
CATGTACCAAAGGGAAATGGTACCATTTTAAAGCGTGACACAGGAAATAATAATTGGATTCCTCATGT
TGGAAGTACTTGGTGGCCTCCGAAGACACCATATATTCCTCCTATCATTACCAACAGGCCTACTTCTA
AGCCAACAACAAGACCTACACCAAAGCCAACACCAATTCCTACTCCACCACCACCACCACCCCTGCCA
ACAGAGCTCAGAACACCTCTACCACCTACAACCCCAGAAAGGCCAACCACCGGACTGACAACTATAGC
ACCAGCTGCCAGTACACCTCCAGGAGGGATTACAGTTGACAACAGGGTACAGACAGACCCTCAGAAAC
CCAGAGGAGATGTGTTCATTCCACGGCAACCTTCAAATGACTTGTTTGAAATATTTGAAATAGAAACA
GGAGTCAGTGCAGACGATCAAGCAAAGGATGATCCAGGTGTTCTGGTACACAGTTGTAATTTTGACCA
TGGACTTTGTGGATGGATCAGGGAGAAAGACAATGACTTGCACTGGGAACCAATCAGGGACCCAGCAG
GTGGACAATATCTGACAGTGTCGGCAGCCAAAGCCCCAGGGGGAAAAGCTGCACGCTTGGTGCTACCT
CTCGGCCGCCTCATGCATTCAGGGGACCTGTGCCTGTCATTCAGGCACAAGGTGACGGGGCTGCACTC
TCGCACACTCCAGGTGTTTGTGAGAAAACACGGTGCCCACGGAGCAGCCCTGTCGGGAAGAAATGGTG
GCCATGGCTGGAGGCAAACACAGATCACCTTGCGAGGGGCTGACATCAAGAGCCTCGTCTTCAAAGGT
GAAAAAAGGCGTGGTCACACTGGGGAGATTGGATTAGATGATGTGAGCTTGAAAAAAGGCCACTGCTC
TGAA

| | | | |
|---|---|---|---|
| NOV5b, 306447063 | | | |
| Protein Sequence | SEQ ID NO: 28 | 581 aa | MW at 65022.9kD |

MDFLLALVLVSSLYLQAAAEFDGSRWPRQIVSSIGLCRYGGRIDCCWGWARQSWCQCQP
FYVLRQRIARIRCQLKAVCQPRCKHGECIGPNKCKCHPGYAGKTCNQDLNECGLKPRPCKHRCMNTYG
SYKCYCLNGYMLMPDGSCSSALTCSMANCQYGCDVVKGQIRCQCPSPGLQLAPDGRTCVDVDECATCR
ASCPRFRQCVNTFGSYICKCHKGFDLMYIGGKYQCHDIDECSLGQYQCSSFARCYNVRGSYKCKCKEG
YQGDGLTCVYIPKVMIEPSGPIHVPKCNGTILKGDTGNNNWIPDVGSTWWPPKTPYIPPIITNRPTSK
PTTRPTPKPTPIPTPPPPPPLPTELRTPLPPTTPERPTTGLTTIAPAASTPPGGITVDNRVQTDPQKP
RGDVFIPRQPSNDLFEIFEIERGVSADDEAKDDPGVLVHSCNFDHGLCGWIREKDNDLHWEPIRDPAG
GQYLTVSAAKAPGGKAARLVLPLGRLMHSCDLCLSFRHKVTGLHSGTLQVFVRKHGAHGAALWGRNGG
HCWRQTQITLRGADIKSVVFKGEKRRGHTGEIGLDDVSLKKGHCSE

| | | |
|---|---|---|
| NOV5c, 306447071 | SEQ ID NO: 29 | 1689 bp |
| DNA Sequence | ORF Start: at 1 | Stop: end of sequence |

ATGGATTTTCTCCTGGCGCTGGTGCTGGTATCCTCGCTCTACCTGCAGGCGCCCGCCG
AGTTCGACGGGAGGTGGCCCAGGCAAATAGTGTCATCGATTGGCCTATGTCGTTATGGTCGGAGGATT
GACTGCTGCTGGGGCTGGGCTCGCCAGTCTTGCGGACAGTGTCAGCCTGTGTGCCAACCACGATGCAA
ACATGGTGAATGTATCGGGCCAAACAAGTGCAAGTGTCATCCTGGTTATGCTGGAAAAACCTGTAATC
AAGATCTAAATGAGTGTGGCCTGAAGCCCCGGCCCTGTAAGCACAGGTGCATGAACACTTACGGCAGC
TACAAGTGCTACTGTCTCAACGGATATATGCTCATGCCGGATGGTTCCTGCTCAAGTGCCCTCACCTG
CTCCATGGCAAACTGTCAGTATGGCTGTGATGTTGTTAAAGGACAAATACGGTGCCAGTGCCCATCCC
CTGGCCTGCACCTGGCTCCTGATGGGAGGACCTGTGTAGATGTTGATGAATGTGCTACAGGAAGAGCC
TCCTGCCCTAGATTTAGGCAATGTGTCAACACTTTTGGGAGCTACATCTGCAAGTGTCATAAAGGGTT
CGATCTCATGTATATTGGACGCAAATATCAATGTCATGACATAGACGAATGCTCACTTGGTCAGTATC
AGTGCAGCAGCTTTGCTCGATGTTATAACGTACGTGGGTCCTACAAGTGCAAATGTAAAGAAGGATAC
CAGGGTGATGGACTGACTTGTGTGTATATCCCAAAAGTTATGATTGAACCTTCAGCTCCAATTCATGT
ACCAAAGGGAAATGGTACCATTTTAAAGGGTGACACAGGAAATAATAATTGGATTCCTGATGTTGGAA
GTACTTGGTGGCCTCCGAAGACACCATATATTCCTCCTATCATTACCAACAGGCCTACTTCTAAGCCA
ACAACAAGACCTACACCAAAGCCAACACCAATTCCTACTCCACCACCACCACCACCCCTGCCAACAGA
GCTCAGAACACCTCTACCACCTACAACCCCAGAAAGGCCAACCACCGGACTGACAACTATAGCACCAG
CTGCCAGTACACCTCCAGGAGGGATTACAGTTGACAACAGGGTACAGACAGACCCTCAGAAACCCAGA
GGAGATGTGTTCATTCCACGGCAACCTTCAAATGACTTGTTTGAAATATTTGAAATAGAAAGAGGAGT
CAGTGCAGACGATGAAGCAAAGGATGATCCAGGTGTTCTGGTACACAGTTGTAATTTTGACCATCGAC
TTTGTGGATGGATCAGGGAGAAAGACAATGACTTGCACTGGGAACCAATCAGGGACCCAGCAGGTGGA
CAATATCTGACAGTGTCGGCAGCCAAAGCCCCAGGGGGAAAAGCTGCACGCTTGGTGCTACCTCTCGG
CCGCCTCATGCATTCAGGGGACCTGTGCCTGTCATTCAGGCACAAGGTGACGGGGCTGCACTCTGGCA
CACTCCAGGTGTTTGTGAGAAAACACGGTGCCCACGGACCAGCCCTGTGGGGAAGAAATGGTGGCCAT
GGCTGGAGGCAAACACAGATCACCTTGCGAGGGGCTGACATCAAGAGCGTCGTCTTCAAAGGTGAAAA
AAGGCGTGGTCACACTGGGGAGATTGGATTAGATGATGTGAGCTTGAAAAAAGGCCACTGCTCTGAA

| | | | |
|---|---|---|---|
| NOV5c, 306447071 | | | |
| Protein Sequence | SEQ ID NO: 30 | 563 aa | MW at 62132.5kD |

MDFLLALVLVSSLYLQAAAEFDGRWPRQIVSSIGLCRYGGRIDCCWGWARQSWGQCQPVCQPRCK
HGECIGPNKCKCHPGYAGKTCNQDLNECGLKPRPCKHRCMNTYGSYKCYCLNGYMLMPDGSCSSALTC
SMANCQYGCDVVKGQIRCQCPSPGLHLAPDGRTCVDVDECATGRASCPRFRQCVNTFGSYICKCHKGF
DLMYIGGKYQCHDIDECSLGQYQCSSFARCYNVRGSYKCKCKEGYQGDGLTCVYIPKVMIEPSGPIHV
PKGNGTILKGDTGNNNWIPDVGSTWWPPKTPYIPPTITNRPTSKPTTRPTPKPTPIPTPPPPPPLPTE
LRTPLPPTTPERPTTGLTTIAPAASTPPGGITVDNRVQTDPQKPRGDVFIPRQPSNDLFEIFEIERGV
SADDEAKDDPGVLVHSCNFDHGLCGWIREKDNDLHWEPIRDPAGGQYLTVSAAKAPGGKAARLVLPLG
RLMHSGDLCLSFRHKVTGLHSGTLQVFVRKHGAHGAALWGRNGGHGWRQTQITLRGADIKSVVFKGEK
RRGHTGEIGLDDVSLKKGHCSE

| | | |
|---|---|---|
| NOV5d, 306447075 | SEQ ID NO: 31 | 1740 bp |
| DNA Sequence | ORF Start: at 1 | ORF Stop: end of sequence |

ATGGATTTTCTCCTGGCGCTGCTGCTGGTATCCTCGCTCTACCTGCAGGCGGCCCCCG
AGTTCGACGGGAGGTGGCCCAGGCAAATAGTGTCATCGATTGGCCTATGTCGTTATGGTGGGACGATT
GACTGCTGCTGCGGCTGGGCTCGCCAGTCTTGGGGACAGTGTCAGCCTTTCTACGTCTTAACGCAGAG
AATAGCCAGGATAAGGTGCCAGCTCAAAGCTGTGTGCCAACCACGATGCAAACATGGTGAATGTATCG
GGCCAAACAAGTGCAAGTGTCATCCTGGTTATGCTGGAAAAACCTGTAATCAAGATCTAAATGAGTGT
GGCCTGAAGCCCCGGCCCTGTAAGCACAGGTGCATGAACACTTACGGCAGCTACAAGTGCTACTGTCT
CAACGGATATATGCTCATGCCGGATGGTTCCTGCTCAAGTGCCCTGACCTGCTCCATGGCAAACTGTC
AGTATGGCTGTGATGTTGTTAAAGGACAAATACGGTCCCAGTGCCCATCCCCTGGCCTGCAGCTGGCT
CCTGATGGGAGGACCTGTGTAGATGTTGATGAATGTGCTACAGGAAGAGCCTCCTGCCCTAGATTTAC
GCAATGTGTCAACACTTTTGGGAGCTACATCTGCAAGTGTCATAAAGGCTTCGATCTCATGTATATTG
GAGGCAAATATCAATGTCATGACATAGACGAATGCTCACTTGGTCAGTATCAGTGCAGCAGCTTTGCT
CGATGTTATAACGTACGTGGGTCCTACAAGTGCAAATGTAAAGAAGGATACCAGGGTGATGCACTGAC
TTGTGTGTATATCCCAAAAGTTATGATTGAACCTTCAGGTCCAATTCATGTACCAAAGGGAAATGGTA
CCATTTTAAAGGGTGACACAGGAAATAATAATTGGATTCCTGATGTTGGAAGTACTTGGTGGCCTCCG
AAGACACCATATATTCCTCCTATCATTACCAACAGGCCTACTTCTAAGCCAACAACAAGACCTACACC
AAAGCCAACACCAATTCCTACTCCACCACCACCACCACCCCTGCCAACAGAGCTCAGAACACCTCTAC
CACCTACAACCCCACAAAGGCCAACCACCGGACTGACAACTATAGCACCAGCTGCCAGTACACCTCCA
GGAGGGATTACAGTTGACAACAGGGTACAGACAGACCCTCAGAAACCCAGAGGAGATGTGTTCATTCC
ACGGCAACCTTCAAATGACTTGTTTGAAATATTTGAAATAGAAAGAGGAGTCAGTGCAGACGATGAAG
CAAAGGATGATCCAGGTGTTCTGGTACACAGTTGTAATTTTGACCATGGACTTTGTGGATGGATCAGG
GAGAAAGACAATGACTTGCACTCGGAACCAATCAGGGACCCAGCAGGTGCACAATATCTCACAGTGTC
GGCAGCCAAAGCCCCAGGGGGAAAAGCTGCACGCTTGGTGCTACCTCTCGGCCGCCTCATGCATTCAG
GGGACCTGTGCCTGTCATTCAGGCACAAGGTGACGGGGCTGCACTCTGGCACACTCCAGGTGTTTGTG
AGAAAACACGGTGCCCACGGAGCAGCCCTGTGGGGAAGAAATGGTGGCCATGGCTGGAGGCAAACACA
GATCACCTTGCGAGGGGCTGACATCAAGAGCGTCGTCTTCAAAGGTGAAAAAAGGCGTGGTCACACTG
GGGAGATTGGATTAGATGATGTGAGCTTGAAAAAAGGCCACTGCTCTGAA

| | | | |
|---|---|---|---|
| NOV5d, 306447075 | | | |
| Protein Sequence | SEQ ID NO: 32 | 586 aa | MW at 64240.0kD |

MDFLLALVLVSSLYLQAAAEFDGRWPRQIVSSIGLCRYGGRIDCCWGWARQSWGQCQPFYVLRQR
IARIRCQLKAVCQPRCKHGECIGPNKCKCHPGYAGKTCNQDLNECGLKPRPCKHRCMNTYGSYKCYCL
NGYMLMPDGSCSSALTCSMANCQYGCDVVKGQIRCQCPSPGLQLAPDGRTCVDVDECATGRASCPRFR
QCVNTFGSYICKCHKGFDLMYIGGKYQCHDIDECSLCQYQCSSFARCYNVRGSYKCKCKEGYQGDGLT
CVYIPKVMIEPSGPIHVPKGNGTILKGDTGNNNWIPDVGSTWWPPKTPYIPPIITNRPTSKPTTRPTP
KPTPIPTPPPPPPLPTELRTPLPPTTPERPTTGLTTIAPAASTPPGGITVDNRVQTDPQKPRGDVFIP
RQPSNDLFEIFEIERGVSADDEAKDDPGVLVHSCNFDHGLCGWIREKDNDLHWEPIRDPACGQYLTVS
AAKAPGGKAARLVLPLGRLMHSGDLCLSFRHKVTGLHSGTLQVFVRKHGAHGAALWGRNGGHGWRQTQ
ITLRGADIKSVVFKGEKRRGHTGEIGLDDVSLKKGHCSEVDG

| | | |
|---|---|---|
| NOV5e, CG51117-09 | SEQ ID NO: 33 | 1839 bp |
| DNA Sequence | ORF Start: ATG at 1 | ORF Stop: at 1850 |

ATGGATTTTCTCCTGGCGCTGGTGCTGGTATCCTCGCTCTACCTGCAGGCGGCCGCCG
AGTTCGACGGGAGTAGGTGGCCCAGGCAAATAGTGTCATCGATTGGCCTATGTCGTTATGGTGGGAGG
ATTGACTGCTGCTGGGGCTGGGCTCGCCAGTCTTGGGGACAGTGTCAGCCTTTCTACGTCTTAAGGCA
GAGAATAGCCAGGATAAGGTGCCAGCTCAAAGCTGTGTGCCAACCACGATGCAAACATGGTGAATGTA
TCGGGCCAAACAAGTGCAAGTGTCATCCTGGTTATGCTCGAAAAACCTGTAATCAAGACGAGCACATC
CCAGCTCCTCTTGACCAAGGCAGTGAACAGCCTCTTTTCCAACCCCTGGATCACCAAGCCACAAGTTT
GCCTTCAAGGGATCTAAATGAGTGTGGCCTGAAGCCCCGGCCCTGTAAGCACAGGTGCATGAACACTT
ACGGCAGCTACAAGTGCTACTGTCTCAACGGATATATGCTCATGCCGGATGGTTCCTGCTCAAGTGCC
CTGAGCTGCTCCATGGCAAACTGTCAGTATGGCTGTGATGTTGTTAAAGGACAAATACGCTGCCAGTG
CCCATCCCCTGGCCTGCAGCTGGCTCCTGATGGGAGGACCTGTGTAGATGTTGATGAATGTGCTACAG
GAAGAGCCTCCTCCCCTAGATTTAGGCAATGTGTCAACACTTTTGGGAGCTACATCTGCAAGTGTCAT
AAAGGCTTCGATCTCATGTATATTGGAGGCAAATATCAATGTCATGACATAGACGAATGCTCACTTGG
TCAGTATCAGTGCAGCAGCTTTGCTCGATGTTATAACGTACGTGGGTCCTACAAGTGCAAATGTAAAG
AAGGATACCAGCGTGATGGACTGACTTGTGTGTATATCCCAAAAGTTATGATTGAACCTTCAGGTCCA
ATTCATGTACCAAAGGGAAATGGTACCATTTTAAAGGGTGACACAGGAAATAATAATTGCATTCCTGA
TGTTGGAAGTACTTGGTGGCCTCCGAAGACACCATATATTCCTCCTATCATTACCAACAGGCCTACTT
CTAACCCAACAACAAGACCTACACCAAAGCCAACACCAATTCCTACTCCACCACCACCACCACCCCTG
CCAACAGAGCTCAGAACACCTCTACCACCTACAACCCCAGAAAGGCCAACCACCGGACTGACAACTAT
AGCACCAGCTGCCAGTACACCTCCAGGAGGGATTACAGTTGACAACAGGGTACAGACAGACCCTCAGA
AACCCAGAGGAGATGTGTTCATTCCACGGCAACCTTCAAATGACTTGTTTGAAATATTTGAAATAGAA
AGAGGAGTCAGTGCAGACGATGAAGCAAAGGATGATCCAGGTGTTCTGGTACACAGTTGTAATTTTGA
CCATGGACTTTGTGGATGGATCAGGGAGAAAGACAATGACTTCCACTGGGAACCAATCAGGGACCCAG
CAGGTGGACAATATCTGACAGTGTCGGCAGCCAAAGCCCCAGGGGGAAAAGCTGCACGCTTGGTGCTA
CCTCTCGGCCGCCTCATGCATTCAGGGGACCTGTGCCTGTCATTCAGGCACAAGGTGACGGGGCTGCA
CTCTGGCACACTCCAGGTGTTTGTCAGAAAACACGGTGCCCAGGGAGCAGCCCTGTGGGGAACAAATG
GTGCCCATGGCTGGAGGCAAACACAGATCACCTTGCGAGGGGCTGACATCAACAGCGTCGTCTTCAAA
GGTGAAAAAAGGCGTGGTCACACTGGGGAGATTGGATTAGATGATGTGAGCTTGAAAAAAGGCCACTG
CTCTGAAGAACGC

| | | | |
|---|---|---|---|
| NOV5e, CG51117-09 | | | |
| Protein Sequence | SEQ ID NO: 34 | 613 aa | MW at 67402.4kD |

MDFLLALVLVSSLYLQAAAEFDGSRWPRQTVSSIGLCRYGGRIDCCWGWARQSWGQCQPFYVLRQRIA
RIRCQLKAVCQPRCKHGECIGPNKCKCHPGYAGKTCNQDEHIPAPLDQGSEQPLFQPLDHQATSLPSR
DLNECGLKPRPCKHRCMNTYGSYKCYCLNGYMLMPDGSCSSALTCSMANCQYGCDVVKGQIRCQCPSP
GLQLAPDGRTCVDVDECATGRASCPRFRQCVNTFGSYTCKCHKGFDLMYIGGKYQCHDIDECSLGQYQ
CSSFARCYNVRGSYKCKCKEGYQGDCLTCVYIPKVMIEPSGPIHVPKGNGTILKGDTGNNNWIPDVGS
TWWPPKTPYIPPIITNRPTSKPTTRPTPKPTPIPTPPPPPPLPTELRTPLPPTTPERPTTGLTTIAPA
ASTPPGGITVDNRVQTDPQKPRGDVFIPRQPSNDLFEIFEIERGVSADDEAKDDPGVLVHSCNFDHGL
CGWIREKDNDLHWEPIRDPAGGQYLTVSAAKAPGGKAARLVLPLGRLMHSGDLCLSFRHKVTGLHSGT
LQVFVRKHGAHGAALWGRNGGHGWRQTQITLRGADIKSVVFKGEKRRGHTGEIGLDDVSLKKGHCSEER

| | | |
|---|---|---|
| NOV5f, CG51117-14 | SEQ ID NO: 35 | 933 bp |
| DNA Sequence | ORF Start: at 1 | ORF Stop: at 944 |

CTGACTTGTGTGTATATCCCAAAAGTTATGATTGAACCTTCAGGTCCAATTCATGTAC
CAAAGGGAAATCGTACCATTTTAAAGGGTGACACAGGAAATAATAATTGGATTCCTGATGTTGGAAGT
ACTTGGTGGCCTCCGAAGACACCATATATTCCTCCTATCATTACCAACAGGCCTACTTCTAAGCCAAC
AACAAGACCTACACCAAAGCCAACACCAATTCCTACTCCACCACCACCACCACCCCTGCCAACAGAGC
TCAGAACACCTCTACCACCTACAACCCCAGAAAGGCCAACCACCGGACTGACAACTATAGCACCAGCT
GCCAGTACACCTCCAGGAGGGATTACAGTTGACAACAGGGTACAGACACACCCTCAGAAACCCAGAGG
AGATGTGTTCATTCCACGGCAACCTTCAAATGACTTGTTTGAAATATTTGAAATAGAAAGAGGAGTCA
GTGCAGACGATGAAGCAAACGATCATCCAGGTGTTCTGGTACACAGTTGTAATTTTGACCATGGACTT
TGTGGATGGATCAGGGAGAAAGACAATGACTTGCACTGCGAACCAATCAGGGACCCAGCAGGTGGACA
ATATCTGACAGTGTCGGCAGCCAAAGCCCCAGGGGGAAAAGCTGCACGCTTGGTGCTACCTCTCGGCC
GCCTCATGCATTCAGGGGACCTGTGCCTGTCATTCAGGCACAAGGTGACGGGGCTGCACTCTGGCACA
CTCCAGGTGTTTGTGAGAAAACACGGTGCCCACGGAGCAGCCCTGTGGGGAAGAAATGGTGGCCATGG
CTCGAGGCAAACACAGATCACCTTGCGAGGGGCTGACATCAACACCGTCGTCTTCAAAGGTGAAAAAA
GGCCTGGTCACACTGGGGAGATTCGATTAGATGATGTGAGCTTGAAAAAAGGCCACTGC

| | | | |
|---|---|---|---|
| NOV5f, CG51117-14 | | | |
| Protein Sequence | SEQ ID NO: 36 | 311 aa | MW at 33658.1kD |

LTCVYIPKVMIEPSGPIHVPKGNGTILKGDTGNNNWIPDVGSTWWPPKTPYIPPIITNRPTSKPTTRP
TPKPTPIPTPPPPPPLPTELRTPLPPTTPERPTTGLTTIAPAASTPPGGITVDNRVQTDPQKPRGDVF
IPRQPSNDLFEIFEIERGVSADDEAKDDPGVLVHSCNFDHGLCGWIREKDNDLHWEPIRDPAGGQYLT
VSAAKAPGGKAARLVLPLGRLMHSGDLCLSFRHKVTGLHSGTLQVFVRKHGAHGAALWGRNGGHGWRQ
TQITLRGADIKSVVFKGEKRRGHTGEIGLDDVSLKKGHC

| | | |
|---|---|---|
| NOV5g, SNP13382208 of | SEQ ID NO: 37 | 2112 bp |
| CG51117-03, | ORF Start: ATG at 203 | ORF Stop: TAA at 1949 |
| DNA Sequence | SNP Pos: 1794 | SNP Change: G to A |

GGGAGGGGGCTCCGGGCGCCGCGCAGCAGACCTGCTCCGCCCGCGCGCCTCGCCGCTGTCCTCCGGGA
GCGGCAGCAGTAGCCCGGGCGGCGAGGGCTGGGGGTTCCTCGAGACTCTCAGAGGGGCGCCTCCCATC
GGCCCCCACCACCCCAACCTGTTCCTCGCGCCCCACTGCGCTGCGCCCCAGGACCCGCTGCCCAACAT
GGATTTTCTCCTGGCGCTGGTGCTGGTATCCTCGCTCTACCTGCAGGCGGCCGCCGAGTTCGACGGGA
GGTGGCCCAGGCAAATAGTGTCATCGATTGGCCTATGTCGTTATGGTGGGAGGATTGACTGCTGCTGG
GGCTGGGCTCGCCAGTCTTGGCGACAGTGTCAGCCTTTCTACGTCTTAAGGCAGAGAATAGCCAGGAT
AAGGTGCCAGCTCAAAGCTGTGTGCCAACCACGATGCAAACATGGTCAATCTATCGGGCCAAACAAGT
GCAAGTGTCATCCTGGTTATGCTGGAAAAACCTGTATTCAAGTTTTAAATGAGTGTGGCCTGAAGCCC
CGGCCCTGTAAGCACAGGTGCATCAACACTTACGGCAGCTACAAGTGCTACTGTCTCAACGGATATAT
GCTCATGCCGGATGGTTCCTGCTCAAGTGCCCTGACCTGCTCCATGGCAAACTGTCAGTATGGCTGTG
ATGTTGTTAAAGGACAAATACGGTGCCAGTGCCCATCCCCTGGCCTGCAGCTGGCTCCTGATGGGAGG
ACCTGTGTAGATGTTGATGAATGTGCTACAGGAAGAGCCTCCTGCCCTAGATTTAGCCAATGTGTCAA
CACTTTTGGGAGCTACATCTGCAAGTGTCATAAAGGCTTCGATCTCATGTATATTGGAGGCAAATATC
AATGTCATGACATACACGAATGCTCACTTGGTCAGTATCAGTGCAGCAGCTTTGCTCGATGTTATAAC
GTACGTGGGTCCTACAAGTGCAAATGTAAAGAAGGATACCACGGTGATGGACTGACTTGTGTGTATAT
CCCAAAAGTTATGATTGAACCTTCAGGTCCAATTCATGTACCAAAGGGAAATGGTACCATTTTAAAGG
GTGACACAGGAAATAATAATTGGATTCCTGATGTTGGAAGTACTTGGTGGCCTCCGAAGACACCATAT
ATTCCTCCTATCATTACCAACAGGCCTACTTCTAAGCCAACAACAAGACCTACACCAAAGCCAACACC
AATTCCTACTCCACCACCACCACCACCCCTGCCAACAGAGCTCAGAACACCTCTACCACCTACAACCC
CAGAAAGGCCAACCACCGGACTGACAACTATAGCACCAGCTGCCAGTACACCTCCAGGAGGGATTACA
GTTGACAACAGGGTACAGACAGACCCTCAGAAACCCAGAGGAGATGTGTTCATTCCACGGCAACCTTC
AAATGACTTGTTTGAAATATTTCAAATAGAAACAGGAGTCAGTGCACACCATGAAGCAAAGGATGATC
CAGGTGTTCTGGTACACAGTTGTAATTTTGACCATGGACTTTGTGGATGGATCAGGGAGAAACACAAT
GACTTGCACTGGGAACCAATCAGGGACCCAGCAGGTGGACAATATCTGACAGTGTCGGCAGCCAAAGC
CCCAGGGGGAAAAGCTGCACGCTTGGTGCTACCTCTCGGCCGCCTTATGCATTCAGGGGACCTGTGCC
TGTCATTCAGGCACAAGGTGACGGGGCTGCACTCTGCCACACTCCAGGTGTTTGTGAGAAAACACGGT
GCCCACGGAGCAGCCCTGTGGGGAAAAAATGGTGGCCATGGCTGGAGGCAAACACAGATCACCTTGCG
AGGGGCTGACATCAAGAGCGTCGTCTTCAAAGGTGAAAAAAGGCGTGGTCACACTGGGGAGATTGGAT
TAGATGATGTGAGCTTGAAAAAAGGCCACTGCTCTGAAGAACGCTAACAACTCCAGAACTAACAATGA
ACTCCTATGTTGCTCTATCCTCTTTTTCCAATTCTCATCTTCTCTCCTCTTCTCCCTTTTATCAGGCC
TAGGAGAAGACTGGGTCAGTGCGTCACAAGCAAGTCTATTTGGTGACCCAGGTTCTTCTGGCCTGCTT
TTGT

| | | | |
|---|---|---|---|
| NOV5g, SNP13382208 of | | | |
| CG51117-03, Protein | SEQ ID NO: 38 | | MW at 63963.9kD |
| Sequence | SNP Pos: 531 | 582 aa | SNP Change: Arg to Lys |

MDFLLALVLVSSLYLQAAAEFDGRWPRQIVSSIGLCRYGGRIDCCWGWARQSWGQCQPFYVLRQRIAR
IRCQLKAVCQPRCKHGECIGPNKCKCHPGYACKTCIQVLNECGLKPRPCKHRCMNTYGSYKCYCLNGY
MLMPDGSCSSALTCSMANCQYGCDVVKGQIRCQCPSPGLQLAPDGRTCVDVDECATGRASCPRFRQCV
NTFGSYICKCHKGFDLMYIGGKYQCHDIDECSLGQYQCSSFARCYNVRCSYKCKCKEGYQGDGLTCVY
IPKVMIEPSGPIHVPKGNGTILKGDTGNNNWIPDVGSTWWPPKTPYIPPIITNRPTSKPTTRPTPKPT
PIPTPPPPPPLPTELRTPLPPTTPERPTTGLTTIAPAASTPPGGITVDNRVQTDPQKPRCDVFIPRQP
SNDLFEIFEIERGVSADDEAKDDPGVLVHSCNFDHGLCGWIREKDNDLHWEPIRDPAGGQYLTVSAAK
APGGKAARLVLPLGRLMHSGDLCLSFRHKVTGLHSGTLQVFVRKHGAHGAALWGKNGGHGWRQTQITL
RGADIKSVVFKGEKRRGHTGEIGLDDVSLKKGHCSEER

Further analysis of the NOV5a protein yielded the following properties shown in Table 5B.

TABLE 5C
Protein Sequence Properties NOV5e

| SignalP | Cleavage site between residues 19 and 20 | | | | |
|---|---|---|---|---|---|
| analysis: | PSG: a new signal peptide prediction method | | | | |
| PSORT II | N-region: length 2; pos. chg 0; neg. chg 1 | | | | |
| analysis: | H-region: length 17; peak value 0.00 | | | | |
| | PSG score: −4.40 | | | | |
| | GvH: von Heijne's method for signal seq. recognition | | | | |
| | GvH score (threshold: −2.1): −0.34 | | | | |
| | possible cleavage site: between 17 and 18 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 1 | | | | |
| | Number of TMS(s) for threshold 0.5: 1 | | | | |
| | INTEGRAL | Likelihood = | −4.19 | Transmembrane | 3-19 |

| PERIPHERAL | Likelihood = | 5.67 (at 516) | |
|---|---|---|---|
| ALOM score: −4.19 (number of TMSs: 1) | | | |
| MTOP: Prediction of membrane topology (Hartmann et al.) | | | |
| Center position for calculation: 10 | | | |
| Charge difference: 0.0 C(0.0)-N(0.0) | | | |
| N >= C: N-terminal side will be inside | | | |
| >>> membrane topology: type 2 (cytoplasmic tail 1 to 3) | | | |
| MITDISC: discrimination of mitochondrial targeting seq | | | |
| R content: | 0 | Hyd Moment(75): | 4.41 |
| Hyd Moment(95): | 7.23 | G content: | 0 |
| D/E content: | 2 | S/T content: | 2 |

Score: −6.55
Gavel: prediction of cleavage sites for mitochondrial preseq
cleavage site motif not found
NUCDISC: discrimination of nuclear localization signals
pat4: none
pat7: none
bipartite: none
content of basic residues: 11.9%
NLS Score: −0.47
KDEL: ER retention motif in the C-terminus: none
ER Membrane Retention Signals: none
SKL: peroxisomal targeting signal in the C-terminus: none
SKL2: 2nd peroxisomal targeting signal: found
RIARIRCQL at 66
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytplasmic/Nuclear discrimination
Prediction: nuclear
Reliability: 89
COIL: Lupas's algorithm to detect coiled-coil regions
total: 0 residues

A search of the NOV5e protein against the Geneseq database, a proprietary database that contains sequences published in patents and patent publication, yielded several homologous proteins shown in Table 5D.

TABLE 5C
Geneseq Results for NOV5e

| | | NOV5e | Identities/ | |
|---|---|---|---|---|
| | | Residues/ | Similarities | |
| Geneseq | Protein/Organism/Length | Match | for the | Expect |
| Identifier | [Patent #,Date] | Residues | Matched Region | Value |
| ABR47621 | Breast cancer associated protein | 138 . . . 613 | 476/476 (100%) | 0.0 |
| | sequence SEQ ID NO: 484 - Homo | 107 . . . 582 | 476/476 (100%) | |
| | sapiens, 582 aa. [WO2003004989- | | | |
| | A2, 16 JAN. 2003] | | | |
| AAB70547 | Human PRO 17 protein sequence | 138 . . . 613 | 476/476 (100%) | 0.0 |
| | SEQ ID NO: 34 - Homo sapiens, | 107 . . . 582 | 476/476 (100%) | |
| | 582 aa. [WO200110902-A2, 15 FEB. 2001] | | | |
| ABG69659 | Human secreted protein SCEP-39 - | 135 . . . 613 | 477/479 (99%) | 0.0 |
| | Homo sapiens, 566 aa. | 88 . . . 566 | 479/479 (100%) | |
| | [WO200248337-A2, 20 JUN. 2002] | | | |
| ABJ37055 | Human breast cancer/ovarian | 103 . . . 613 | 506/511 (99%) | 0.0 |
| | cancer related protein #31 - Homo | 51 . . . 560 | 509/511 (99%) | |
| | sapiens, 560 aa. [WO2003000012- | | | |
| | A2, 03 JAN. 2003] | | | |

In a BLAST search of public sequence databases, the NOV5e protein was found to have homology to the proteins shown in the BLASTP data in Table 5D.

TABLE 5D
Public BLASTP Results for NOV5e

| | | NOV5e | Identities/ | |
|---|---|---|---|---|
| Protein | | Residues/ | Similarities | |
| Accession | | Match | for the | Expect |
| Number | Protein/Organism/Length | Residues | Matched Portion | Value |
| CAC33425 | Sequence 33 from Patent | 138 . . . 613 | 476/476 (100%) | 0.0 |
| | WO0110902 - Homo sapiens | 107 . . . 582 | 476/476 (100%) | |
| | (Human), 582 aa. | | | |
| Q923T5 | Nephronectin - Mus musculus | 1 . . . 578 | 536/609 (88%) | 0.0 |
| | (Mouse), 609 aa. | 1 . . . 609 | 566/609 (93%) | |
| Q91XL5 | Nephronectin - Mus musculus | 25 . . . 609 | 503/585 (85%) | 0.0 |
| | (Mouse), 592 aa. | 24 . . . 592 | 534/585 (91%) | |

PFam analysis predicts that the NOV5e protein contains the domains shown in the Table 5E.

TABLE 5E
Domain Analysis of NOV5e

| | NOV5e Match Region | | |
|---|---|---|---|
| | Amino acid residues | | |
| Pfam Domain | of SEQ ID NO: 34 | Score | E-Value |
| EGF | 78 . . . 104 | 18.9 | 0.12 |
| EGF | 141 . . . 175 | 15.6 | 0.26 |
| EGF | 181 . . . 215 | 20.7 | 0.035 |
| EGF | 221 . . . 260 | 2.7 | 6.1 |
| MAM | 470 . . . 611 | 102.4 | 8.6e−27 |

The epithelial-mesenchymal interactions required for kidney organogenesis are disrupted in mice lacking the integrin alpha8beta1. None of this integrin's known ligands, however, appears to account for this phenotype. To identify a more relevant ligand, Brandenberger et al. (2001) used a soluble integrin alpha8beta1 heterodimer fused to alkaline phosphatase (AP) to probe blots and cDNA libraries. In newborn mouse kidney extracts, alpha8beta1-AP detects a novel ligand of 70-90 kD. This protein, named nephronectin, is an extracellular matrix protein with five EGF-like repeats, a mucin region containing a RGD sequence, and a COOH-terminal MAM domain. Integrin alpha8beta1 and several additional RGD-binding integrins bind nephronectin. Nephronectin mRNA is expressed in the ureteric bud epithelium, whereas alpha8beta1 is expressed in the metanephric mesenchyme. Nephronectin is localized in the extracellular matrix in the same distribution as the ligand detected by alpha8beta1-AP and forms a complex with alpha8beta1 in vivo. Thus, these results strongly suggest that nephronectin is a relevant ligand mediating alpha8beta1 function in the kidney. Nephronectin is expressed at numerous sites outside the kidney, so it may also have wider roles in development. (Brandenberger et al. J Cell Biol Jul. 23, 2001;154(2):447-58)

NOV5e is a novel nucleic acid of 613 nucleotides (designated CuraGen Acc. No. CG51117-09) encoding a novel Nephronectin-like protein. This sequence represents a splice form of Nephronectin as indicated in positions with one exon insertion 30 amino acids and one amino acid S insertion at position 24 and maps to chromosome 6

The presence of identifiable domains in the protein disclosed herein was determined by searches versus domain databases such as Pfam, PROSITE, ProDom, Blocks or Prints and then identified by the Interpro domain accession number. A 170 amino acid domain, the so-called MAM domain, has been recognised in the extracellular region of functionally diverse proteins. These proteins have a modular, receptor-like architecture comprising a signal peptide, an N-terminal extracellular domain, a single transmembrane domain and an intracellular domain. Such proteins include meprin (a cell surface glycoprotein); A5 antigen (a developmentally-regulated cell surface protein); and receptor-like tyrosine protein phosphatase. The MAM domain is thought to have an adhesive function. It contains 4 conserved cysteine residues, which probably form disulphide bridges.

A sequence of about thirty to forty amino-acid residues long found in the sequence of epidermal growth factor (EGF) has been shown to be present, in a more or less conserved form, in a large number of other, mostly animal proteins. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in disulfide bonds. The main structure is a two-stranded beta-sheet followed by a loop to a C-terminal short two-stranded sheet. Subdomains between the conserved cysteines vary in length.

NOV5a, clone 306433917 is a splice variant with deletion of amino acid sequences GKYQCH, EHIPAPLDQGSEQPLFQPLDHQATSLPSR (SEQ ID NO:79) and PRQPSNDLFEIFEIERGVSADDEAKDDPG (SEQ ID NO:80) one deleted exon 29 amino acids, plus 1 amino acid changes I322S compared to NOV5e. NOV5b, 5c, 5d, assemblies 306447063, 306447071, 306447075 respectively were found to encode an open reading frame between residues 1 to 611 of NOV5e, CG51117-09. The cloned insert NOV5c 306447071 is a splice variant of parent with one exon deletion 17 amino acids FYVLRQRIARIRCQLKA (SEQ ID NO:81), deletion of amino acid sequence EHIPAPLDQGSEQPLFQPLDHQATSLPSR (SEQ ID NO:82) plus 1 amino acid S deletion at position 24, amino acid changes Q159H compare to NOV5e. The cloned insert NOV5d 306447075 has a deletion of amino acid sequence EHIPAPLDQGSEQPLFQPLDHQATSLPSR (SEQ ID NO:83) plus 1 amino acid S deletion at position 24 compared to NOV5e NOV5b, 306447063 has has a deletion of amino acid sequence EHIPAPLDQGSEQPLFQPLDHQATSLPSR (SEQ ID NO:84) compared to NOV5e

### Example 6

### NOV6, CG51923, Protocadherin Fat 2 Precursor

The NOV6 clone was analyzed, and the nucleotide and encoded polypeptide sequences are shown in Table 6A.

TABLE 6A

NOV6 Sequence Analysis

| | | |
|---|---|---|
| NOV6a, CG51923-01 | SEQ ID NO: 39 | 14536 bp |
| DNA Sequence | ORF Start: ATG at 14 | ORF Stop: TAG at 13061 |

GGAGTTTTCCACCATGACTATTGCCCTGCTGGGTTTTGCCATATTCTTGCTCCATTGTGCGACCTGTG
AGAAGCCTCTAGAAGGGATTCTCTCCTCCTCTGCTTGGCACTTCACACACTCCCATTACAATGCCACC
ATCTATGAAAATTCTTCTCCCAACACCTATGTGGAGAGCTTCGAGAAAATGGGCATCTACCTCGCGGA
GCCACAGTGGGCAGTGAGGTACCCGATCATCTCTGGGGATGTGGCCAATGTATTTAAAACTGAGGAGT
ATGTGGTGGGCAACTTCTGCTTCCTAAGAATAAGGACAAAGAGCAGCAACACAGCTCTTCTGAACAGA
GAGGTGCGAGACAGCTACACCCTCATCATCCAAGCCACAGAGAAGACCTTGGAGTTGGAAGCTTTGAC
CCCTGTGGTGGTCCACATCCTGGACCAGAATGACCTGAAGCCTCTCTTCTCTCCACCTTCGTACAGAC
TCACCATCTCTGAGGACATGCCCCTGAAGAGCCCCATCTGCAAGGTGACTGCCACAGATGCTGATCTA
GGCCAGAATGCTGAGTTCTATTATGCCTTTAACACAAGGTCAGAGATGTTTGCCATCCATCCCACCAG
CGGTGTGGTCACTGTGGCTGGGAAGCTTAACGTCACCTGGCGAGGAAAGCATGAGCTCCAGGTGCTAG
CTGTGGACCGCATGCGGAAAATCTCTGAGGGCAATGGGTTTGGCAGCCTGGCTGCACTTGTGGTTCAT
GTGGAGCCTGCCCTCAGGAAGCCCCCACCCATTGCTTCGGTGGTGGTGACTCCACCAGACAGCAATGA
TGGTACCACCTATGCCACTGTACTGGTCGATGCAAATAGCTCACGAGCTGAAGTGGAGTCAGTGGAAG
TTGTTGGTGGTGACCCTGGAAAGCACTTCAAAGCCATCAAGTCTTATGCCCGGAGCAATCAGTTCAGT
TTGGTGTCTGTCAAAGACATCAACTGGATCGAGTACCTTCATGGGTTCAACCTCAGCCTCCACGCCAG
GAGTGGGAGCGGCCCTTATTTTTATTCCCAGATCAGGGGCTTTCACCTACCACCTTCCAAACTGTCTT
CCCTCAAATTCGAGAAGGCTGTTTACAGAGTGCAGCTTAGTGAGTTTTCCCCTCCTGGCACCCGCGTG
GTCATGGTGAGAGTCACCCCAGCCTTCCCCAACCTGCAGTATGTTCTAAAGCCATCTTCAGAGAATGT
AGGATTTAAACTTAATGCTCGAACTGGGTTGATCACCACCACAAACCTCATGGACTTCCACGACAGAG
CCCACTATCAGCTACACATCAGAACCTCACCGGGCCAGGCCTCCACCGTGGTGGTCATTGACATTGTG
GACTGCAACAACCATGCCCCCCTCTTCAACAGGTCTTCCTATGATGGTACCTTGGATGAGAACATCCC
TCCAGGCACCAGTGTTTTGGCTGTGACTGCCACTGACCGGGATCATGGGGAAAATGGATATGTCACCT
ATTCCATTCCTGGACCAAAAGCTTTGCCATTTTCTATTGACCCCTACCTGGGGATCATCTCCACCTCC
AAACCCATGGACTATGAACTCATGAAAAGAATTTATACCTTCCGGGTAAGAGCATCAGACTGGGGATC
CCCTTTTCGCCGGGAGAAGGAAGTGTCCATTTTTCTTCAGCTCAGGAACTTGAATGACAACCAGCCTA
TGTTTGAAGAAGTCAACTGTACAGGGTCTATCCGCCAAGACTGGCCAGTAGGGAAATCGATAATGACT
ATGTCAGCCATAGATGTGGATGAGCTTCAGAACCTAAAATACCAGATTGTATCAGGCAATGAACTAGA
GTATTTTGATCTAAATCATTTCTCCGGAGTGATATCCCTCAAACGCCCTTTTATCAATCTTACTGCTG
GTCAACCCACCAGTTATTCCCTGAACATTACAGCCTCAGATGGCAAAAACTATGCCTCACCCACAACT
TTGAATATTACTGTGGTGAAGGACCCTCATTTTGAAGTTCCTGTAACATGTGATAAAACAGGGGTATT
GACACAATTCACAAAGACTATCCTCCACTTTATTGGGCTTCACAACCAGGAGTCCAGTGATGAGGAAT
TCACTTCTTTAAGCACATATCAGATTAATCATTACACCCCACAGTTTGAGGACCACTTCCCCCAATCC
ATTGATGTCCTTGAGAGTGTCCCTATCAACACCCCCTTGGCCCGCCTAGCAGCCACTCACCCTGATGC
TGGTTTTAATGGCAAACTGGTCTATGTGATTCCAGATGGCAATGAGGAGCGCTGCTTTGACATAGAGC
TGGAGACAGGGCTGCTCACTGTAGCTGCTCCCTTGGACTATGAAGCCACCAATTTCTACATCCTCAAT
GTAACAGTATATGACCTGGGCACACCCCAGAAGTCCTCCTCGAAGCTGCTGACAGTGAATGTGAAAGA
CTGGAATGACAACGCACCCAGATTTCCTCCCGGTGGGTACCAGTTAACCATCTCGGAGGACACAGAAG
TTGGAACCACAATTGCAGAGCTGACAACCAAAGATGCTGACTCGGAAGACAATGGCAGGGTTCGCTAC
ACCCTGCTAAGTCCCACAGAGAAGTTCTCCCTCCACCCTCTCACTGGGGAACTGGTTGTTACAGGACA
CCTGGACCGCGAATCAGAGCCTCGGTACATACTCAAGGTGGAGGCCAGGCATCAGCCCAGCAAAGGCC
ACCAGCTCTTCTCTGTCACTGACCTGATAATCACATTGGAGGATGTCAACGACAACTCTCCCCAGTGC
ATCACAGAACACAACAGGCTGAAGGTTCCAGAGGACCTGCCCCCCGGGACTGTCTTGACATTTCTGGA
TGCCTCTGATCCTGACCTGGGCCCCGCAGGTGAAGTGCGATATGTTCTGATCGATGGCGCCCATGGGA
CCTTCCGGGTGGACCTGATCACAGGGGCGCTCATTCTGGAGAGAGAGCTGGACTTTGAGAGGCGAGCT
GGGTACAATCTGAGCCTGTGGGCCAGTGATGGTGGGAGGCCCCTAGCCCGCAGGACTCTCTGCCATGT
GGAGGTGATCGTCCTGGATGTGAATGAGAATCTCCACCCTCCCCACTTTGCCTCCTTCGTGCACCAGG
GCCAGGTGCAGGAGAACAGCCCCTCGGGAACTCAGGTCATTGTAGTGGCTGCCCAGGACGATGACAGT
GGCTTGGATGGGGAGCTCCAGTACTTCCTGCGTGCTGGCACTGGACTCGCAGCCTTCAGCATCAACCA
AGATACAGHAATGATTCAGACTCTGGCACCCCTGGACCGAGAATTTGCATCTTACTACTGGTTGACGG
TATTAGCAGTGGACAGCGGTTCTGTGCCCCTCTCTTCTGTAACTCAAGTCTACATCGAGGTTACGGAT
GCCAATGACAACCCACCCCAGATGTCCCAAGCTGTGTTCTACCCCTCCATCCAGGAGGATGCTCCCGT
GGGCACCTCTGTGCTTCAACTGGATGCCTGGGACCCAGACTCCAGCTCCAAACGGAAGCTGACCTTCA
ACATCACCAGTGGGAACTACATGGGATTCTTTATGATTCACCCTGTTACAGGTCTCCTATCTACAGCC
CAGCAGCTGGACAGAGAGAACAAGGATGAACACATCCTGGAGGTGACTGTGCTGGACAATGGGGAACC
CTCACTGAAGTCCACCTCCAGGGTGGTGGTAGGCATCTTGGACGTCAATGACAATCCACCTATATTCT
CCCACAAGCTCTTCAATGTCCGCCTTCCAGAGAGGCTGAGCCCTGTGTCCCCTGGGCCTGTGTACAGG
CTGGTGGCTTCAGACCTGGATGAGGGTCTTAATGGCAGAGTCACCTACAGTATCGACGACAGCTATGA
GGAGGCCTTCAGTATCGACCTGGTCACAGGTGTGGTTTCATCCAACAGCACTTTTACAGCTGGAGAGT
ACAACATCCTAACGATCAAGGCAACAGACAGTGGGCAGCCACCACTCTCAGCCAGTGTCCGGCTACAC
ATTGAGTGGATCCCTTGGCCCCGGCCGTCCTCCATCCCTCTGGCCTTTGATGAGACCTACTACAGCTT
TACGGTCATGGAGACGGACCCTGTGAACCACATGGTGGGGGTCATCAGCGTAGAGGGCAGACCCGGAC
TCTTCTGGTTCAACATCTCAGGTGGGGATAAGGACATGGACTTTGACATTGAGAAGACCACAGGCAGC
ATCGTCATTGCCAGGCCTCTTGATACCAGGAGAAGGTCGAACTATAACTTGACTGTTCAGGTGACAGA
TGGGTCCCGCACCATTGCCACACAGGTCCACATCTTCATGATTCCCAACATTAACCACCATCGGCCCC
AGTTTCTGGAAACTCGTTATGAAGTCAGAGTTCCCCAGGACACCGTGCCAGGGGTAGAGCTCCTGCGA
GTCCAGGCCATAGATCAAGACAAGGGCAAAAGCCTCATCTATACCATACATGGCAGCCAAGACCCAGG
AAGTGCCAGCCTCTTCCAGCTGGACCCAAGCAGTGGTGTCCTGGTAACGGTGGGAAAATTGGACCTCG
GCTCGGGGCCCTCCCAGCACACACTGACAGTCATGGTCCGAGACCAGGAAATACCTATCAAGAGGAAC
TTCGTGTGGGTGACCATTCATGTGGAGGATGGAAACCTCCACCCACCCCCCTTCACTCAGCTCCATTA
TGAGGCAAGTGTTCCTGACACCATAGCCCCCGGCACAGAGCTGCTGCAGGTCCGAGCCATGGATGCTG
ACCGGGGAGTCAATGCTGAGGTCCACTACTCCCTCCTGAAAGGGAACAGCGAAGGTTTCTTCAACATC
AATGCCCTGCTAGGCATCATTACTCTAGCTCAAAAGCTTGATCAGGCAAATCATGCCCCACATACTCT
GACAGTGAAGGCAGAAGATCAAGGCTCCCCACAATGGCATGACCTGGCTACAGTGATCATTCATCTCT
ATCCCTCAGATAGGAGTGCCCCCATCTTTTCAAAATCTGAGTACTTTGTAGAGATCCCTGAATCAATC
CCTGTTGGTTCCCCAATCCTCCTTGTCTCTGCTATGAGCCCCTCTGAAGTTACCTATGAGTTAAGAGA
GGCAAATAAGGATGGAGTCTTCTCTATGAACTCATATTCTGGCCTTATTTCCACCCAGAAGAAATTGG
ACCATGAGAAAATCTCGTCTTACCAGCTGAAAATCCGAGGCAGCAATATGGCAGGTCCATTTACTGAT
GTCATGGTGGTGGTTGACATAATTGATGAAAATCACAATGCTCCTATGTTCTTAAACTCAACTTTTGT
GGGCCAAATTAGTGAAGCAGCTCCACTGTATAGCATGATCATGCATAAAAACAACAACCCCTTTGTGA
TTCATGCCTCTGACAGTGACAAAGAAGCTAATTCCTTGTTGGTCTATAAAATTTTGGAGCCGGAGGCC
TTGAAGTTTTTCAAAATTGATCCCAGCATGGGAACCCTAACCATTGTATCAGAGATGGATTATGAGAG
CATGCCCTCTTTCCAATTCTGTGTCTATGTCCATCACCAAGGAAGCCCTGTATTATTTGCACCCAGAC
CTGCCCAAGTCATCATTCATGTCAGACATGTGAATGATTCCCCTCCCAGATTCTCAGAACAGATATAT
GAGGTAGCAATAGTCGGGCCTATCCATCCAGGCATGGAGCTTCTCATGGTGCGGGCCAGCGATGAAGA
CTCAGAAGTCAATTATAGCATCAAAACTGGCAATGCTGATGAAGCTGTTACCATCCATCCTGTCACTG
GTAGCATATCTGTGCTCAATCCTGCTTTCCTGGGACTCTCTCGGAAGCTCACCATCAGGGCTTCTGAT
GGCTTGTATCAAGACACTGCGCTGCTAAAAATTTCTTTGACCCAAGTGCTTGACAAAAGCTTGCAGTT
TGATCAGGATGTCTACTGGGCAGCTGTGAAGGAGAACTTGCAGGACAGAAAGGCACTGGTGATTCTTG
GTGCCCAGGGCAATCATTTGAATGACACCCTTTCCTACTTTCTCTTGAATGGCACAGATATGTTTCAT
ATGGTCCAGTCAGCAGGTGTGTTGCAGACAAGAGGTGTGGCGTTTGACCGGGAGCAGCAGGACACTCA
TGAGTTGGCAGTGCAAGTGAGGGACAATCGGACACCTCAGCGGGTGGCTCAGGGTTTGGTCAGAGTCT
CTATTCAGGATGTCAATGACAATCCCCCCAAATTTAAGCATCTGCCCTATTACACAATCATCCAAGAT
GGCACAGAGCCAGGGGATGTCCTCTTTCAGGTATCTQCCACTGATGAGGACTTGGGGACAAATGGGGC
TGTTACATATGAATTTGCAGAAGATTACACATATTTCCGAATTGACCCCTATCTTGGGGACATATCAC
TCAAGAAACCCTTTGATTATCAAGCTTTAAATAAATATCACCTCAAAGTCATTGCTCGGGATGGAGGA
ACGCCATCCCTCCAGAGTGAGGAAGAGGTACTTGTCACTGTGAGAAATAAATCCAACCCACTGTTTCA
GAGTCCTTATTACAAAGTCAGAGTACCTGAAAATATCACCCTCTATACCCCAATTCTCCACACCCAGG
CCCGGAGTCCAGAGGGACTCCGGCTCATCTACAACATTGTGCAGGAAGAACCCTTGATCCTGTTCACC
ACTGACTTCAAGACTGGTGTCCTAACAGTAACAGGGCCTTTGCACTATGAGTCCAAGACCAAACATGT
GTTCACAGTCAGAGCCACGCATACACCTCTGGGCTCATTTTCTGAAGCCACAGTGGAAGTCCTAGTGG
AGGATGTCAATGATAACCCTCCCACTTTTTCCCAATTGGTCTATACCACTTCCATCTCAGAAGGCTTG
CCTGCTCAGACCCCTGTGATCCAACTGTTGGCTTCTGACCAGGACTCAGGGCGGAACCGTGACGTCTC
TTATCAGATTGTGGAGGATGGCTCAGATGTTTCCAAGTTCTTCCAGATCAATGGGAGCACAGGGGAGA
TGTCCACAGTTCAAGAACTGGATTATGAAGCCCAACAACACTTTCATGTGAAAGTCAGGGCCATGGAT
AAAGGAGATCCCCCACTCACTGGTGAAACCCTTGTGGTTCTCAATGTGTCTGATATCAATGACAACCC
CCCAGAGTTCAGACAACCTCAATATGAAGCCAATGTCAGTGAACTGCCAACCTGTGGACACCTGGTTC
TTAAAGTCCAGGCTATTGACCCTGACAGCAGAGACACCTCCCGCCTGGAGTACCTGATTCTTTCTGGC
AATCAGGACAGGCACTTCTTCATTAACACCTCATCGGGAATAATTTCTATGTTCAACCTTTGCAAAAA
GCACCTGGACTCTTCTTACAATTTGAGGGTAGGTGCTTCTGATGGAGTCTTCCGAGCAACTGTGCCTG
TGTACATCAACACTACAAATGCCAACAAGTACAGCCCAGAGTTCCAGCAGCACCTTTATGAGGCAGAA
TTAGCAGAGAATGCAATQGTTGGAACCAAGGTGATTGATTTGCTAGCCATAGACAAAGATAGTGGTCC
CTATGGCACTATAGATTATACTATCATCAATAAACTAGCAAGTGAGAAGTTCTCCATAAACCCCAATG
GCCAGATTGCCACTCTGCAGAAACTGGATCGGGAAAATTCAACAGAGAGAGTCATTGCTATTAAGGTC
ATGGCTCGGGATGGAGGAGGAAGAGTACCCTTCTGCACGCTGAAGATCATCCTCACAGATGAAAATGA
CAACCCCCCACAGTTCAAAGCATCTGAGTACACAGTATCCATTCAATCCAATGTCAGTAAAGACTCTC
CGGTTATCCAGGTGTTGGCCTATGATGCAGATGAAGGTCAGAACGCAGATGTCACCTACTCAGTGAAC
CCAGAGGACCTAGTTAAAGATGTCATTGAAATTAACCCAGTCACTGGTGTGGTCAAGGTGAAAGACAG
CCTGGTGGGATTGGAAAATCAGACCCTTGACTTCTTCATCAAAGCCCAAGATGGACGCCCTCCTCACT
GGAACTCTCTGGTGCCAGTACCACTTCAGGTGGTTCCTAAAAAAGTATCCTTACCGAAATTTTCTGAA
CCTTTGTATACTTTCTCTGCACCTGAAGACCTTCCAGAGGGGTCTGAAATTGGGATTGTTAAAGCAGT
GGCAGCTCAAGATCCAGTCATCTACAGTCTAGTCCGGCGCACTACACCTGAGAGCAACAAGGATGGTG
TCTTCTCCCTAGACCCAGACACACGCGTCATAAAGGTGAGGAAGCCCATGGACCACGAATCCACCAAA
TTGTACCAGATTGATGTGATGGCACATTGCCTTCAGAACACTGATGTGGTGTCCTTGGTCTCTGTCAA
CATCCAAGTGGGAGACGTCAATGACAATAGGCCTGTATTTGACGCTGATCCATATAAGGCTGTCCTCA
CTGAGAATATGCCAGTGGGGACCTCAGTCATTCAAGTGACTGCCATTGACAAGGACACTGGGAGAGAT
GGCCAGGTGAGCTACAGGCTGTCTGCAGACCCTGGTAGCAATGTCCATGAGCTCTTTGCCATTGACAG
TGAGAGTGGTTGGATCACCACACTCCAGGAACTTGACTGTGAGACCTCCCAGACTTATCATTTTCATG
TGGTGGCCTATGACCACGGACAGACCATCCAGCTATCCTCTCAGGCCCTGGTTCACGTCTCCATTACA
GATGAGAATGACAATGCTCCCCGATTTGCTTCTGAAGAGTACAGAGGATCTGTGGTTGAGAACAGTGA
GCCTGGCGAACTGGTCGCGACTCTAAAGACCCTGGATGCTCACATTTCTGAGCAGAACAGGCAGGTCA
CCTGCTACATCACAGAGGGAGACCCCCTGGGCCAGTTTGGCATCAGCCAAGTTGGAGATGAGTGGAGG
ATTTCCTCAAGGAAGACCCTGGACCGCGAGCATACAGCCAAGTACTTGCTCAGAGTCACAGCATCTGA
TGGCAAGTTCCAGGCTTCGGTCACTGTGGAGATCTTTGTCCTGGACGTCAATGATAACAGCCCACAGT
GTTCACAGCTTCTCTATACTGGCAAGGTTCATGAAGATGTATTTCCAGGACACTTCATTTTCAAGGTT
TCTGCCACAGACTTGGACACTGATACCAATGCTCAGATCACATATTCTCTGCATGGCCCTGGGGCGCA
TGAATTCAAGCTGGATCCTCATACAGGCGAGCTGACCACACTCACTGCCCTAGACCGAGAAAGGAAGG
ATGTGTTCAACCTTGTTGCCAAGGCGACGGATGGAGGTGGCCGATCGTGCCAGGCAGACATCACCCTC
CATGTGGAGGATGTGAATGACAATGCCCCGCGGTTCTTCCCCAGCCACTGTGCTGTCGCTGTCTTCGA
CAACACCACAGTGAACACCCCTCTGGCTGTAGTATTTGCCCGGGATCCCGACCAAGGCGCCAATGCCC
AGGTGGTTTACTCTCTCCCGGATTCAGCCGAAGGCCACTTTTCCATCGACGCCACCACGGGGGTGATC
CGCCTGGAAAAGCCGCTGCAGGTCAGGCCCCAGGCACCACTGGAGCTCACGGTCCGTGCCTCTCACCT
GGGCACCCCAATACCGCTGTCCACGCTGGGCACCGTCACAGTCTCGGTGGTCGGCCTAGAAGACTACC
TGCCCGTGTTCCTGAACACCGAGCACAGCGTGCAGGTGCCCGAGGACGCCCCACCTGGCACGGAGGTG
CTGCAGCTGGCCACCCTCACTCGCCCGGGCGCAGAGAAGACCGGCTACCGCGTGGTCAGCGGGAACGA
GCAAGGCACGTTCCGCCTGGATGCTCGCACAGGGATCCTGTATGTCAACGCAAGCCTCGACTTTGAGA
CAAGCCCCAAGTACTTCCTGTCCATTGAGTGCAGCCGGAAGACCTCCTCTTCCCTCAGTGACGTGACC
ACAGTCATGGTCAACATCACTGATGTCAATGAACACCGGCCCCAATTCCCCCAAGATCCATATAGCAC
AAGGGTCTTAGAGAATGCCCTTGTGCGTGACGTCATCCTCACGGTATCAGCGACTGATGAAGATGGAC
CCCTAAATAGTGACATTACCTATAGCCTCATAGGAGGGAACCAGCTTGGGCACTTCACCATTCACCCC
AAAAAGGGGGAGCTACAGGTGGCCAAGCCCCTGGACCGGGAACAGGCCTCTAGTTATTCCCTGAAGCT
CCGAGCCACAGACAGTGGCCAGCCTCCACTGCATGAGGACACAGACATCGCTATCCAAGTGGCTGATG
TCAATGATAACCCACCGAGATTCTTCCAGCTCAACTACAGCACCACTGTCCAGGAGAACTCCCCCATT
CGCAGCAAAGTCCTGCACCTGATCCTGAGTGACCCAGATTCTCCAGAGAATGGCCCCCCCTACTCGTT
TCGAATCACCAAGGGGAACAACGGCTCTGCCTTCCGAGTCACCCCGGATGGATGGCTGGTGACTGCTG
AGGGCCTAACCAGGAGGGCTCAGGAATGGTATCAGCTTCAGATCCAGGCGTCAGACACTGGCATCCCT
CCCCTCTCGTCTTTGACGTCTGTCCGTGTCCATGTCACAGAGCAGAGCCACTATGCACCTTCTGCTCT
CCCACTGGACATCTTCATCACTGTTGGAGAGGATGAGTTCCAGGGTGGCATGGTGGGTAAGATCCATG
CCACAGACCGAGACCCCCAGGACACGCTGACCTATAGCCTGGCAGAAGAGGAGACCCTGGGCAGGCAC
TTCTCAGTGGGTCCGCCTCATGGCAAGATTATCGCCGCCCAGGGCCTGCCTCGTGGCCACTACTCGTT
CAACGTCACGGTCAGCGATGGGACCTTCACCACGACTGCTGGGGTCCATGTGTACGTGTGGCATGTGG
GGCAGGAGGCTCTGCAGCAGGCCATGTGGATGGGCTTCTACCAGCTCACCCCCGAGGAGCTGGTGAGT
GACCACTGGCGGAACCTGCAGACGTTCCTCAGCCATAAGCTGGACATCAAACGGGCTAACATTCACTT
GGCCAGCCTCCAGCCTGCAGAGGCCGTGGCTGGTGTGGATGTCCTCCTGGTCTTTGAGGGGCATTCTG
GAACCTTCTACGAGTTTCAGGAGCTAGCATCCATCATCACTCACTCAGCCAAGGAGATGGAGCATTCA
GTGGGGGTTCAGATGCGGTCAGCTATGCCCATGGTGCCCTGCCAGGGCCCAACCTGCCAGGGTCAAAT
CTGCCATAACACAGTGCATCTGGACCCCAAGGTTGGGCCCACGTACAGCACCGCCAGGCTCAGCATCC
TAACCCCGCGGCACCACCTGCAGAGGAGCTGCTCCTGCAATGGTACTGCTACAAGGTTCAGTGGTCAG
AGCTATGTGCGGTACAGGGCCCCAGCGGCTCGGAACTGGCACATCCATTTCTATCTGAAAACACTCCA
GCCACACGCCATTCTTCTATTCACCAATGAAACAGCGTCCGTCTCCCTGAAGCTGGCCAGTGGAGTGC
CCCAGCTGGAATACCACTGTCTGGGTGGTTTCTATGGAAACCTTTCCTCCCAGCGCCATGTGAATGAC
CACGAGTGGCACTCCATCCTGGTGGAGGAGATGGACGCTTCCATTCGCCTGATGGTTGACAGCATGGG
CAACACCTCCCTTGTGGTCCCAGAGAACTGCCGTGGTCTGAGGCCCGAAAGGCACCTCTTGCTGGGCG
GCCTCATTCTGTTGCATTCTTCCTCGAATGTCTCCCAGGGCTTTGAAGGCTGCCTGCATCCTGTCGTG
GTCAACGAAGAGGCTCTAGATCTGCTGGCCCCTGGCAAGACGGTGGCAGGCTTGCTGGAGACACAAGC
CCTCACCCAGTGCTGCCTCCACAGTGACTACTGCAGCCAGAACACATGCCTCAATGGTGGGAAGTGCT
CATGGACCCATGGGGCAGGCTATGTCTGCAAATGTCCCCCACAGTTCTCTGGGAAGCACTGTGAACAA
GGAAGGGAGAACTGTACTTTTGCACCCTGCCTGGAAGGTGGAACTTGCATCCTCTCCCCCAAAGGAGC
TTCCTGTAACTGCCCTCATCCTTACACAGGAGACAGGTGTGAAATGGAGGCCAGGGGTTGTTCAGAAG
GACACTGCCTAGTCACTCCCGAGATCCAAACGGGGGACTGGGGGCAGCAGGAGTTACTGATCATCACA
GTGGCCGTGGCGTTCATTATCATAAGCACTGTCGGGCTTCTCTTCTACTGCCGGCGTTGCAAGTCTCA
CAAGCCTGTGGCCATGGAGGACCCAGACCTCCTGGCCAGGAGTGTTGGTGTTGACACCCAAGCCATGC
CTGCCATCGAGCTCAACCCATTGAGTCCCAGCTCCTGCAACAACCTCAACCAACCGGAACCCAGCAAG
GCCTCTGTTCCAAATGAACTCGTCACATTTGGACCCAATTCTAAGCAACGGCCAGTGGTCTGCAGTGT
GCCCCCCAGACTCCCGCCAGCTGCGGTCCCTTCCCACTCTGACAATGAGCCTGTCATTAAGAGAACCT
GGTCCAGCGAGGAGATGGTGTACCCTGGCGGAGCCATGGTCTGGCCCCCTACTTACTCCAGGAACGAA
CGCTGGGAATACCCCCACTCCGAAGTGACTCAGGGCCCTCTGCCGCCCTCGGCTCACCGCCACTCAAC
CCCAGTCGTGATGCCAGAGCCTAATGGCCTCTATCGGGGCTTCCCCTTCCCCCTGGAGATGGAAAACA
AGCGGGCACCTCTCCCACCCCGTTACACCAACCAGAACCTGGAAGATCTGATGCCCTCTCGGCCCCCT
AGTCCCCGGGAGCGCCTGGTTGCCCCCTGTCTCAATGAGTACACGGCCATCAGCTACTACCACTCGCA
GTTCCGGCAGGGAGGGGGAGCGCCCTGCCTGGCAGACGGGGGCTACAAGGGGGTGGGTATGCGCCTCA
GCCGAGCTGGGCCCTCTTATGCTGTCTGTGAGGTGGAGGGGGCACCTCTTGCAGGCCAGCGCCAGCCC
CGGGTGCCCCCCAACTATGAGCGCTCTGACATGGTGGAGAGTGATTATGGCACCTCTGAGGAGGTCAT
GTTCTAGCTTCCCATTCCCAGAGCAAGGCAGGCGGCAGGCCAACGACTGGACTTGGCTTATTTCTTCC
TGTCTCGTAGGGGGTGAGTTGAGTGTGGCTGGGAGAGTGGGAGGGAAGCCCTCAGCCCAGGCTGTTGT
CCCTTGAAATGTGCTCTTCCAATCCCCCACCTAGTCCCTGAGGGTGGAGCGAAGCTGAGGATAGAGCT
CCAGAAACAGCACTAGGGTCCCAGGAGAGGGGCATTTCTAGAGCAGTGACCCTGGAAAACCAGGAACA
ATTGACTCCTGGGGTGGGCGACAGACAGCAGGGCTCCCTGATCTGCCGGCTCTCACTCCCCGGCGCAA
AGCCTGATTGACTGTGCTGGCTCAACTTCACCAAGATGCATTCTCATACCTGCCCACAGCTCCATTTT
GGAGGCAGGCAGGTTGGTGCCTGACAGACAACCACTACGCGGGCCGTACAGAGGAGCTCTAGAGGGCT
GCGTCGCATCCTCCTAGGGGCTGAGAGGTGAGCAGCAGCGGAGCGGGCACAGTCCCCTCTGCCCCTGC
CTCAGTCGAGCACTCACTGTGTCTTTGTCAAGTGTCTGCTCCACGTCAGGCACTGTGCTTTGCACCGG
GGAGAAAATGGTGATGGACGGCAACAACGACTCCGAGGAGCACCACCAGGCCTCGGGCCCCAGACGTC
CCGCTCCTCAGCCTACACGCAGAGGAACGCGCCCACCTCAGAGTCACACCACTCGCTGCCAGTCAGGC
CCTGCCAGGAGTCTACACAGCTCTGAACCTTCTTTGTTAAAGAATTCAGACCTCATGGAACTCTCGGT
TCTTCATCCCAAGTTTCCCAGGCACTTTTGGCCAAAGGAAGCAAGGAACTAATTCTTCATTTTAAAAA
TTCTTAGGCACTTTTTGACCTTGCTGTCTGGATGAGTTTCCTCAATGGGATTTTTCTTCCCTAGACAC
AAGCAAGTCTGAACTCCTATTTAGGGCCGGTTGGAAGCAGGGAGCTGGACCGCAGTGTCCAGGCTGGA
CACCTCCCATTGCCTCCTCTCCACTGCAGACGCCTGCCCATCAAGTATTACCTGCACCCACTCAACCC
TATGCATGGAGGGTCAATGTGGGCACATGTCTACACATCTGGGTGCCCATGGATAGTACGTGTGTACA
CATGTGTAGAGTGTATGTAGCCAGGACTGGTGGGGACCAGAAGCCTCTGTGGCCTTTCGTGACCTCAC
CACTCCCTCCCACCCAGTCCCTCCCTCTGGTCCACTGCCTTTTCATATGTGTTGTTTCTGGAGACAGA
AGTCAAAACGAAGAGCAGTGGAGCCTTGCCCACAGGGCTGCTGCTTCATGCGAGAGGGAGATGTGTGG
GCGAGAGCCAATTTGTGTGAGTGGTTTGTGGCTGTGTGTGTGACTGTGAGTGTGAGTGACACATACAT
AGTTTCATTCGTCATTTTTTTTTTTAACAATAAAGTATCTTTTTTTACTGTT

| | | | |
|---|---|---|---|
| NOV6a, CG51923-01 | | | |
| Protein Sequence | SEQ ID NO: 40 | 4349 aa | MW at 479387.3kD |

MTIALLGFAIFLLHCATCEKPLEGILSSSAWHFTHSHYNATIYENSSPKTYVESFEKMGIYLAEPQWA
VRYRIISGDVANVFKTEEYVVGNFCFLRIRTKSSNTALLNREVRDSYTLIIQATEKTLELEALTRVVV
HILDQNDLKPLFSPPSYRVTISEDMPLKSPICKVTATDADLGQNAEFYYAFNTRSEMFAIHPTSCVVT
VAGKLNVTWRGKHELQVLAVDRMRKISEGNGFGSLAALVVHVEPALRKPPAIASVVVTPPDSNDGTTY
ATVLVDANSSGAEVESVEVVGGDPGKHFKAIKSYARSNEFSLVSVKDINWMEYLHGFNLSLQARSGSG
PYFYSQIRGFHLPPSKLSSLKFEKAVYRVQLSEFSPPGSRVVMVRVTPAFPNLQYVLKPSSENVGFKL
NARTGLITTTKLMDFHDRAHYQLHIRTSPGQASTVVVIDIVDCNNHAPLFNRSSYDGTLDENIPPGTS
VLAVTATDRDHGENGYVTYSIAGPKALPFSIDPYLGIISTSKPMDYELMKRIYTFRVRASDWGSPFRR
EKEVSIFLQLRNLNDNQPMFEEVNCTGSIRQDWPVGKSIMTMSAIDVDELQNLKYEIVSGNELEYFDL
NHFSGVISLKRPFINLTAGQPTSYSLKITASDGKNYASPTTLNITVVKDPHFEVPVTCDKTGVLTQFT
KTILHFIGLQNQESSDEEFTSLSTYQINHYTPQFEDHFPQSIDVLESVPINTPLARLAATDPDAGFNG
KLVYVIADGNEEGCFDIELETGLLTVAAPLDYEATNFYILNVTVYDLGTPQKSSWKLLTVNVKDWNDN
APRFPPGGYQLTISEDTEVGTTIAELTTKDADSEDNGRVRYTLLSPTEKFSLHPLTGELVVTGHLDRE
SEPRYILKVEARDQPSKGHQLFSVTDLIITLEDVNDNSPQCITEHNRLKVPEDLPPGTVLTFLDASDP
DLGPAGEVRYVLMDGAHGTFRVDLMTGALILERELDFERRAGYNLSLWASDGGRPLARRTLCHVEVIV
LDVNENLHPPHFASFVHQGQVQENSPSGTQVIVVAAQDDDSGLDGELQYFLRAGTGLAAFSINQDTGM
IQTLAPLDREFASYYWLTVLAKTDRGSVPLSSVTEVYIEVTDANDNPPQMSQAVFYPSIQEDAPVGTS
LQLDAWDPDSSSKGKLTFNITSGNYMGFFMIHPVTGLLSTAQQLDRENKDEHILEVTVLDNGEPSLKS
TSRVVVGILDVNDNPPIFSHKLFNVRLPERLSPVSPGPVYRLVASDLDEGLNGRVTYSIEDSYEEAFS
IDLVTGVVSSNSTFTAGEYNILTIKATDSGQPPLSASVRLHIEWIPWPRPSSIPLAFDETYYSFTVME
TDPVNHMVGVISVEGRPGLFWFNISGGDKDMDFDIEKTTGSIVIARPLDTRRRSNYNLTVEVTDGSRT
IATQVHIFMIANINHHRPQFLETRYEVRVPQDTVPGVELLRVQAIDQDKGKSLIYTIHGSQDPGSASL
FQLDPSSCVLVTVGKLDLGSGPSQHTLTVMVRDQEIPIKRNFVWVTIHVEDGNLHPPRFTQLHYEASV
PDTIAPGTELLQVRAMDADRGVNAEVHYSLLKGNSEGFFNINALLGIITLAQKLDQANHAPHTLTVKA
EDQGSPQWHDLATVIIHVYPSDRSAPIFSKSEYFVEIPESIPVGSPILLVSAMSPSEVTYELREGNKD
GVFSMNSYSGLISTQKKLDHEKISSYQLKIRGSNMAGAFTDVMVVVDIIDENDNAPMFLKSTFVGQIS
EAAPLYSMIMDKNNNPFVIHASDSDKEANSLLVYKILEPEALKFFKIDPSMGTLTIVSEMDYESMPSF
QFCVYVHDQGSPVLFAPRPAQVIIHVRDVNDSPPRFSEQIYEVAIVGPIHPGMELLMVRASDEDSEVN
YSIKTGNADEAVTIHPVTGSISVLNPAFLGLSRKLTIRASDGLYQDTALVKISLTQVLDKSLQFDQDV
YWAAVKENLQDRKALVILCAQGNHLNDTLSYFLLNGTDMFHMVQSAGVLQTRGVAFDREQQDTHELAV
EVRDNRTPQRVAQGLVRVSIEDVNDNPPKFKHLPYYTIIQDGTEPGDVLFQVSATDEDLGTNGAVTYE
FAEDYTYFRIDPYLGDISLKKPFDYQALNKYHLKVIARDGGTPSLQSEEEVLVTVRNKSNPLFQSPYY
KVRVPENITLYTPILHTQARSPEGLRLIYNIVEEEPLMLFTTDFKTGVLTVTGPLDYESKTKHVFTVR
ATDTALGSFSEATVEVLVEDVNDNPPTFSQLVYTTSISEGLPAQTPVIQLLASDQDSGRNRDVSYQIV
EDGSDVSKFFQINGSTGEMSTVQELDYEAQQHFHVKVRAMDKGDPPLTGETLVVVNVSDINDNPPEFR
QPQYFANVSELATCGHLVLKVQAIDPDSRDTSRLEYLILSGNQDRHFFINSSSGIISMFNLCKKHLDS
SYNLRVGASDGVFRATVPVYINTTNANKYSPEFQQHLYEAELAENAMVGTKVIDLLAIDKDSGPYGTI
DYTIINKLASEKFSINPNGQIATLQKLDRENSTERVIAIKVMARDGGGRVAFCTVKIILTDENDNPPQ
FKASEYTVSIQSNVSKDSPVIQVLAYDADEGQNADVTYSVNPEDLVKDVIEINPVTGVVKVKDSLVGL
ENQTLDFFIKAQDGGPPHWNSLVPVRLQVVPKKVSLPKFSEPLYTFSAPEDLPEGSEIGIVKAVAAQD
PVIYSLVRGTTPESNKDGVFSLDPDTGVIKVRKPMDHESTKLYQIDVMAHCLQNTDVVSLVSVNIQVG
DVNDNRPVFEADPYKAVLTENMPVGTSVIQVTAIDKDTGRDGQVSYRLSADPGSNVHELFAIDSESGW
ITTLQELDCETCQTYHFHVVAYDHGQTIQLSSQALVQVSITDENDNAPRFASEEYRGSVVENSEPGEL
VATLKTLDADISEQNRQVTCYITEGDPLGQFGISQVGDEWRISSRKTLDREHTAKYLLRVTASDGKFQ
ASVTVEIFVLDVNDNSPQCSQLLYTGKVHEDVFPGHFILKVSATDLDTDTNAQITYSLHGPGAHEFKL
DPHTCELTTLTALDRERKDVFNLVAKATDGGGRSCQADITLHVEDVNDNAPRFFPSHCAVAVFDNTTV
KTPVAVVFARDPDQGANAQVVYSLPDSAEGHFSIDATTGVIRLEKPLQVRPQAPLELTVRASDLGTPI
PLSTLGTVTVSVVGLEDYLPVFLNTEHSVQVPEDAPPGTEVLQLATLTRPGAEKTGYRVVSGNEQGRF
RLDARTGILYVNASLDFETSPKYFLSIECSRKSSSSLSDVTTVMVNITDVNEHRPQFPQDPYSTRVLE
NALVGDVILTVSATDEDGPLNSDITYSLIGGNQLGHFTIHPKKGELQVAKALDREQASSYSLKLRATD
SGQPPLHEDTDIAIQVADVNDNPPRFFQLNYSTTVQENSPIGSKVLQLILSDPDSPENGPPYSFRITK
GNNGSAFRVTPDGWLVTAEGLSRRAQEWYQLQIQASDSGIPPLSSLTSVRVHVTEOSHYAPSALPLEI
FITVGEDEFQGGMVGKIHATDRDPQDTLTYSLAEEETLGRHFSVGAPDGKIIAAQGLPRGHYSFNVTV
SDGTFTTTAGVHVYVWHVGQEALQQAMWMGFYQLTPEELVSDHWRNLQRFLSHKLDIKRANIHLASLQ
PAEAVAGVDVLLVFEGHSGTFYEFQELASIITHSAKEMEHSVGVQMRSAMPMVPCQGPTCQGQICHNT
VHLDPKVGPTYSTARLSILTPRHHLQRSCSCNGTATRFSGQSYVRYRAPAARNWHIHFYLKTLQPQAI
LLFTNETASVSLKLASGVPQLEYHCLGGFYGNLSSQRHVNDHEWHSILVEEMDASIRLMVDSMGNTSL
VVPENCRGLRPERHLLLGGLILLHSSSNVSQGFEGCLDAVVVNEEALDLIAPGKTVAGLLETQALTQC
CLHSDYCSQNTCLNGGKCSWTHGAGYVCKCPPQFSGKHCEQGRENCTFAPCLEGGTCILSPKGASCNC
PHPYTGDRCEMEARGCSEGHCLVTPEIQRGDWGQQELLIITVAVAFIIISTVGLLFYCRRCKSHKPVA
MEDPDLLARSVGVDTQAMPAIELNPLSASSCNNLNQPEPSKASVPNELVTFGPNSKQRPVVCSVPPRL
PPAAVPSHSDNEPVIKRTWSSEEMVYPGGAMVWPPTYSRNERWEYPHSEVTQGPLPPSAHRHSTPVVM
PEPNGLYGGFPFPLEMENKRAPLPPRYSNQNLEDLMPSRPPSPRERLVAPCLNEYTAISYYHSQFRQG
GGGPCLADGGYKGVGMRLSRAGPSYAVCEVEGAPLAGQGQPRVPPNYEGSDMVESDYGSCEEVMF

| | | |
|---|---|---|
| NOV6b, 305869563 | SEQ ID NO: 41 | 2019 bp |
| DNA Sequence | ORF Start: at 1 | ORF Stop: end of sequence |

GATGGAGGAGGAACAGTAGCCTTCTGCACGGTGAAGATCATCCTCACAGATGAAAATGA
CAACCCCCCACAGTTCAAAGCATCTGAGTACACAGTATCCATTCAATCCAATGTCAGTAAAGACTCTC
CGGTTATCCAGGTGTTGGCCTATGATGCAGATGAAGGTCAGAACGCAGATGTCACCTACTCAGTGAAC
CCAGACGACCTAGTTAAAGATGTCATTGAAATTAACCCAGTCACTGCTGTGGTCAAGGTGAAAGACAG
CCTGGTGGGATTGGAAAATCAGACCCTTGACTTCTTCATCAAAGCCCAAGATGGAGGCCCTCCTCACT
GGAACTCTCTGGTGCCAGTACGACTTCAGGTGGTTCCTAAAAAAGTATCCTTACCCAAATTTTCTGAA
CCTTTGTATACTTTCTCTGCACCTGAAGACCTTCCAGAGGGGTCTGAAATTGGGATTGTTAAAGCAGT
GGCAGCTCAACATCCAGTCATCTACAGTCTAGTGCGGGGCACTACACCTGAGAGCAACAAGGATGGTG
TCTTCTCCCTAGACCCAGACACAGGGGTCATAAAGGTGAGGAAGCCCATGGACCACGAATCCACCAAA
TTGTACCAGATTGATGTGATGGCACATTGCCTTCAGAACACTGATGTGGTGTCCTTGGTCTCTGTCAA
CATCCAAGTGGGAGACGTCAATGACAATAGGCCTGTATTTGAGGCTGATCCATATAAGGCTGTCCTCA
CTGAGAATATGCCAGTGGGGACCTCAGTCATTCAAGTGACTGCCATTGACAAGGACACTGGGAGAGAT
GGCCAGGTGAGCTACAGGCTGTCTGCAGACCCTGGTAGCAATGTCCATGAGCTCTTTGCCATTGACAG
TGAGAGTGGTTGGATCACCACACTCCAGGAACTTGACTGTGAGACCTGCCAGACTTATCATTTTCATG
TGGTGGCCTATCACCACGGACAGACCATCCAGCTATCCTCTCAGGCCCTCGTTCAGGTCTCCATTACA
GATGAGAATGACAATGCTCCCCGATTTGCTTCTGAAGAGTACAGAGGATCTGTGGTTGAGAACAGTGA
GCCTGGCGAACTCGTGGCGACTCTAAAGACCCTGGATGCTGACATTTCTGAGCAGAACAGGCAGGTCA
CCTGCTACATCACAGAGGGAGACCCCCTGGGCCAGTTTGGCATCAGCCAAGTTGGAGATGAGTGGAGG
ATTTCCTCAAGGAAGACCCTGGACCGCGAGCATACAGCCAAGTACTTGCTCAGAGTCACAGCATCTGA
TGCCAAGTTCCATGCTTCGGTCACTGTGGAGATCTTTGTCCTGGACGTCAATGATAACACCCCACAGT
GTTCACAGCTTCTCTATACTGGCAAGGTTCATGAAGATGTATTTCCAGGACACTTCATTTTGAAGGTT
TCTGCCACAGACTTGGACACTGATACCAATGCTCAGATCACATATTCTCTGCATGGCCCTGGGGCGCA
TGAATTCAAGCTGGATCCTCATACAGGGGAGCTGACCACACTCACAGCCCTAGACCGACAAAGGAAGG
ATGTGTTCAACCTTGTTGCCAAGGCGACGGATGGAGGTGGCCGATCGTGCCAGGCAGACATCACCCTC
CATGTGGAGGATGTGAATGACAATGCCCCGCGGTTCTTCCCCAGCCACTGTGCTGTGGCTGTCTTCGA
CAACACCACAGTGAAGACCCCTGTGGCTGTAGTATTTGCCCGGGATCCCGACCAAGGCGCCAATGCCC
AGGTGGTTTACTCTCTGCCGGATTCAGCCGAAGGCCACTTTTCGATCGACGCCACCACGGGGGTGATC
CGCCTGGAAAAGCCGCTGCAGGTCAGGCCCCAGGCACCACTGGAGCTCACGGTCCGTGCCTCTGACCT
GGGCACCCCAATACCGCTCTCCACGCTGGGCACCGTCACAGTCTCGGTGGTGGGCCTAGAAGACTACC
TGCCCGTGTTCCTGAACACCGAGCACAGCGTGCAGGTGCCCGAGGACGCCCCACCT

| | | | |
|---|---|---|---|
| NOV6b, 305869563 | | | |
| Protein Sequence | SEQ ID NO: 42 | 679 aa | MW at ˜73948kD |

DGGGRVAFCTVKIILTDENDNPPQFKASEYTVSIQSNVSKDSPVIQVLAYDADEGQNADVTYSVN
PEDLVKDVIEINPVTGVVKVKDSLVGLENQTLDFFIKAQDGGPPHWNSLVPVRLQVVPKKVSLPKFSE
PLYTFSAPEDLPEGSEIGIVKAVAAQDPVIYSLVRGTTPESNKDCVFSLDPDTGVIKVRKPMDHESTK
LYQIDVMAHCLQNTDVVSLVSVNIQVGDVNDNRPVFEADPYKAVLTENMPVGTSVIQVTAIDKDTGRD
GQVSYRLSADPGSNVHELFAIDSESGWITTLQELDCETCQTYHFHVVAYDHGQTIQLSSQALVQVSIT
DENDNAPRFASEEYRGSVVENSEPGELVATLKTLDADISEQNRQVTCYITEGDPLGQFGISQVGDEWR
ISSRKTLDREHTAKYLLRVTASDGKFHASVTVEIFVLDVNDNSPQCSQLLYTCKVHEDVFPGHFILKV
SATDLDTDTNAQITYSLHGPCAHEFKLDPHTGELTTLTALDRERKDVFNLVAKATDGGCRSCQADITL
HVEDVNDNAPRFFPSHCAVAVFDNTTVKTPVAVVFARDPDQGANAQVVYSLPDSAEGHFSIDATTGVI
RLEKPLQVRPQAPLELTVRASDLGTPIPLSTLGTVTVSVVGLEDYLPVFLNTEHSVQVPEDAPP

| | | |
|---|---|---|
| NOV6c, 305869567 | SEQ ID NO:43 | 2037 bp |
| DNA Sequence | ORF Start: at 1 | ORF Stop: end of sequence |

GATGGAGGAGGAAGAGTAGCCTTCTGCACGGTGAAGATCATCCTCACAGATGAAAATGA
CAACCCCCCACAGTTCAAAGCATCTGAGTACACAGTATCCATTCAATCCAATGTCAGTAAAGACTCTC
CGGTTATCCAGGTGTTGGCCTATGATGCAGATGAAGGTCAGAACGCAGATGTCACCTACTCAGTGAAC
CCAGAGGACCTAGTTAAAGATGTCATTGAAATTAACCCAGTCACTGGTGTGGTCAAGGTGAAAGACAG
CCTGGTGGGATTGGAAAATCAGACCCTTGACTTCTTCATCAAAGCCCAACATGGAGGCCCTCCTCACT
GGAACTCTCTGGTGCCAGTACGACTTCAGGTGGTTCCTAAAAAAGTATCCTTACCGAAATTTTCTGAA
CCTTTGTATACTTTCTCTGCACCTGAAGACCTTCCAGAGGGGTCTGAAATTGGGATTGTTAAAGCAGT
GGCAGCTCAAGATCCAGTCATCTACAGTCTAGTGCGGGGCACTACACCTGAGAGCAACAAGGATGGTG
TCTTCTCCCTAGACCCAGACACAGGGGTCATAAAGGTGAGGAAGCCCATGGACCACGAATCCACCAAA
TTGTACCAGATTGATGTGATGGCACATTGCCTTCAGAACACTGATGTGGTGTCCTTGGTCTCTGTCAA
CATCCAAGTGGGAGACGTCAATGACAATAGGCCTGTATTTGAGGCTGATCCATATAAGGCTGTCCTCA
CTGAGAATATGCCAGTGGGGACCTCAGTCATTCAAGTGACTGCCATTGACAAGGACACTGGGACAGAT
GGCCAGGTGAGCTACAGGCTGTCTGCAGACCCTGGTAGCAATGTCCATGAGCTCTTTGCCATTGACAG
TGAGAGTGGTTGGATCACCACACTCCAGGAACTTGACTGTGAGACCTGCCAGACTTATCATTTTCATC
TGGTGCCCTATGACCACGGACAGACCATCCAGCTATCCTCTCAGGCCCTGGTTCAGGTCTCCATTACA
GATGAGAATGACAATGCTCCCCGATTTGCTTCTGAAGAGTACAGAGGATCTGTGGTTGAGAACAGTGA
GCCTGGCCAACTGGTGGCGACTCTAAAGACCCTGGATGCTGACATTTCTGAGCAGAACAGGCAGGTCA
CCTGCTACATCACAGAGGGAGACCCCCTCGGCCAGTTTGGCATCAGCCAAGTTGGAGATGAGTGGAGG
ATTTCCTCAAGGAAGACCCTGGACCGCGAGCATACACCCAAGTACTTGCTCAGAGTCACAGCATCTGA
TGGCAAGTTCCAGGCTTCGGTCACTGTGGAGATCTTTGTCCTGGACGTCAATGATAACAGCCCACAGT
GTTCACAGCTTCTCTATACTGGCAAGGTTCATGAAGATGTATTTCCAGGACACTTCATTTTGAAGGTT
TCTGCCACAGACTTGGACACTGATACCAATGCTCAGATCACATATTCTCTGCATGGCCCTGCGGCGCA
TGAATTCAAGCTGGATCCTCATACAGGGGAGCTGACCACACTCACTGCCCTACACCGAGAAAGCAAGG
ATGTGTTCAACCTTGTTGCCAAGGCGACGGATGGAGGTGGCCGATCGTGCCAGGCAGACATCACCCTC
CATGTGGACGATGTGAATGACAATGCCCCGCGGTTCTTCCCCAGCCACTGTGCTGTGGCTGTCTTCGA
CAACACCACAGTGAAGACCCCTGTGGCTCTAGTATTTGCCCGGGATCCCGACCAACGCGCCAATGCCC
AGGTCCTTTACTCTCTGCCGGATTCAGCCGAAGCCCAGTTTTCCATCGACGCCACCACGGGGGTGATC
CGCCTGGAAAAGCCGCTCCAGGTCAGGCCCCACGCACCACTGGAGCTCACGGTCCGTGCCTCTGACCT
GGGCACCCCAATACCGCTGTCCACGCTGGGCACCGTCACAGTCTCGGTGGTGGGCCTAGAACACTACC
TGCCCGTGTTCCTGAACACCCAGCACAGCGTGCAGGTGCCCGAGGACGCCCCACCT

| | | | |
|---|---|---|---|
| NOV6c, 305869567 | | | |
| Protein Sequence | SEQ ID NO: 44 | 679 aa | MW at ˜73939 kD |

DGGGRVAFCTVKIILTDENDNPPQFKASEYTVSIQSNVSKDSPVTQVLAYDADEGQNADVTYSVN
PEDLVKDVIEINPVTGVVKVKDSLVGLENQTLDFFIKAQDGGPPHWNSLVPVRLQVVPKKVSLPKFSE
PLYTFSAPEDLPEGSEIGIVKAVAAQDPVIYSLVRGTTPESNKDGVFSLDPDTGVIKVRKPMDHESTK
LYQIDVMAHCLQNTDVVSLVSVNIQVGDVNDNRPVFEADPYKAVLTENMPVGTSVIQVTAIDKDTGRD
GQVSYRLSADPGSNVHELFAIDSESGWITTLQELDCETCQTYHFHVVAYDHGQTIQLSSQALVQVSIT
DENDNAPRFASEEYRGSVVENSEPGELVATLKTLDADISEQNRQVTCYITEGDPLGQFGISQVGDEWR
ISSRKTLDREHTAKYLLRVTASDGKFQASVTVEIFVLDVNDNSPQCSQLLYTGKVHEDVFPGHFILKV
SATDLDTDTNAQITYSLHGPGAHEFKLDPHTGELTTLTALDRERKDVFNLVAKATDGGGRSCQADITL
HVEDVNDNAPRFFPSHCAVAVFDNTTVKTPVAVVFARDPDQGANAQVVYSLPDSAEGHFSIDATTGVI
RLEKPLQVRPQAPLELTVRASDLGTPIPLSTLGTVTVSVVGLEDYLPVFLNTEHSVQVPEDAPP

| | | |
|---|---|---|
| NOV6d, 306076041 | SEQ ID NO: 45 | 1455 bp |
| DNA Sequence | ORF Start: at 1 | ORF Stop: end of sequence |

GACCCCCAGGACACGCTGACCTATAGCCTGGCAGAAGAGGAGACCCTGGGCAGGCACTT
CTCAGTGGGTGCGCCTGATGGCAAGATTATCGCCGCCCAGGGCCTGCCTCGTGGCCACTACTCGTTCA
ACGTCACGGTCAGCGATGGGACCTTCACCACGACTGCTGGGGTCCATGTGTATCTGTGGCATGTGGGG
CACGAGGCTCTGCAGCAGGCCATATGGATGGGCTTCTACCAGCTCACCCCCGAGGAGCTGGTGAGTGA
CCACTGGCGGAACCTGCAGAGGTTCCTCAGCCATAAGCTGGACATCAAACGGGCTAACATTCACTTGG
CCAGCCTCCAGCCTGCAGAGGCCGTCGCTGGTGTGGATGTGCTCCTGGTCTTTGAGGGGCATTCTGGA
ACCTTCTACGAGTTTCAGGAGCTAGCATCCATCATCACTCACTCAGCCAAGGACATGGAGCATTCAGT
GGGGGTTCAGATGCGGTCAGCTATGCCCATGGTGCCCTGCCACGCGCCAACCTGCCAGGGTCAAATCT
GCCATAACACAGTGCATCTGGACCCCAAGGTTGGGCCCACCTACAGCACCGCCAGGCTCAGCATCCTA
ACCCCGCGGCACCACCTGCAGAGGAGCTGCTCCTGCAATGCTACTGCTACAAGGTTCAGTGGTCAGAG
CTATGTGCGGTACAGGGCCCCAGCGGCTCGGAACTGGCACATCCATTTCTATCTGAAAACACTCCAGC
CACAGGCCATTCTTCTATTCACCAATGAAACAGCGTCCGTCTCCCTGAAGCTGGCCAGTGGAGTGCCC
CAGCTGGAATACCACTGTCTGGGTGGTTTCTATGGAAACCTTTCCTCCCAGCGCCATGTGAATGACCA
CGAGTGGCACTCCATCCTGGTGGAGGAGATGGACGCTTCCATTCGCCTGATGGTTGACAGCATGGGCA
ACACCTCCCTTGTGGTCCCAGACAACTGCCGTGGTCTGAGGCCCGAAAGGCACCTCTTGCTGGGCGGC
CTCATTCTGTTGCATTCTTCCTCGAATGTCTCCCAGGGCTTTCAAGGCTGCCTGGATGCTGTCGTGGT
CAACGAAGAGGCTCTAGATCTGCTGGCCCCTGGCAAGACGCTGGCAGGCTTCCTGGAGACACAAGCCC
TCACCCAGTGCTGCCTCCACAGTGACTACTGCAGCCAGAACACATGCCTCAATGGTGGGAAGTGCTCA
TGGACCCATGGGGCAGGCTATGTCTGCAAATGTCCCCCACAGTTCTCTGGGAAGCACTGTGAACAACG
AACGGAGAACTGTACTTTTGCACCCTGCCTGGAAGGTGGAACTTGCATCCTCTCCCCCAAAGGAGCTT
CCTGTAACTGCCCTCATCCTTACACAGGAGACAGGTGTGAAATGGAGGCGAGGGGTTGTTCAGAAGGA
CACTGCCTAGTCACTCCCGAGATCCAAAGGGGGGAC

| | | | |
|---|---|---|---|
| NOV6d, 306076041 | | | |
| Protein Sequence | SEQ ID NO: 46 | 485 aa | MW at ˜53871kD |

DPQDTLTYSLAEEETLGRHFSVGAPDGKIIAAQGLPRGHYSFNVTVSDGTFTTTAGVHVYVWHVG
OEALGGAIWMGFYGLTPEELVSDHWRNLGRFLSHKLDIKRANIHLASLGPAEAVAGVDVLLVFEGHSG
TFYEFQELASTITHSAKEMEHSVGVQMRSAMPMVPCQGPTCQGQICHNTVHLDPKVGPTYSTARLSIL
TPRHHLQRSCSCNGTATRFSGQSYVRYRAPAARNWHIHFYLKTLQPQAILLFTNETASVSLKLASGVP
QLEYHCLGGFYGNLSSQRHVNDHEWHSTLVEEMDASIRLMVDSMGNTSLVVPENCRGLRPERHLLLGG
LILLHSSSNVSQGFEGCLDAVVVNEEALDLLAPGKTVAGLLETQALTQCCLHSDYCSQNTCLNGGKCS
WTHGAGYVCKCPPQFSGKHCEQGRENCTFAPCLEGGTCILSPKGASCNCPHPYTGDRCEMEARGCSEG
HCLVTPEIQRGD

| | | |
|---|---|---|
| NOV6e, 317868343 | SEQ ID NO: 47 | 1977 bp |
| DNA Sequence | ORF Start: at 1 | ORF Stop: end of sequence |

ATGACTATTGCCCTGCTGGGTTTTGCCATATTCTTGCTCCATTGTGCGACCTGTGAGAA
GCCTCTAGAAGGGATTCTCTCCTCCTCTGCTTGGCACTTCACACACTCCCATTACAATGCCACCATCT
ATGAAAATTCTTCTCCCAAGACCTATGTGGAGAGCTTCGAGAAAATGGGCATCTACCTCGCGGAGCCA
CAGTGGGCAGTGAGGTACCGGATCATCTCTGGGGATGTGGCCAATGTATTTAAAACTGAGGAGTATGT
GGTGGGCAACTTCTGCTTCCTAAGAATAAGGACAAAGAGCAGCAACACAGCTCTTCTGAACAGAGAGG
TGCGAGACAGCTACACCCTCATCATCCAAGCCACAGAGAAGACCTTGGAGTTGGAAGCTTTGACCCGT
GTGGTGGTCCACATCCTGGACCAGAATCACCTGAAGCCTCTCTTCTCTCCACCTTCGTACAGAGTCAC
CATCTCTGAGGACATGCCCCTGAAGAGCCCCATCTGCAAGGTGACTGCCACAGATCCTGATCTAGGCC
AGAATGCTGAGTTCTATTATGCCTTTAACACAAGGTCAGAGATGTTTGCCATCCATCCCACCAGCGGT
GTGGTCACTGTGGCTGGGAAGCTTAACGTCACCTGGCGAGGAAAGCATGAGCTCCAGGTGCTAGCTGT
GGACCGCATGCGGAAAATCTCTGAGGGCAATGGGTTTGGCAGCCTGGCTGCACTTGTGGTTCATGTGG
AGCCTGCCCTCAGGAAGCCCCCAGCCATTGCTTCAGTGGTGGTGACTCCACCAGACAGCAATGATGGT
ACCACCTATGCCACTGTACTGGTCGATGCAAATAGCTCAGGAGCTGAAGTGGAGTCAGTGGAAGTTGT
TGGTGGTGACCCTGGAAAGCACTTCAAAGCCATCAAGTCTTATGCCCGGAGCAATGAGTTCAGTTTGG
TGTCTGTCAAAGACATCAACTGGATGGAGTACCTTCATGGGTTCAACCTCAGCCTCCAGGCCAGGAGT
GGGAGCGGCCCTTATTTTTATTCCCAGATCAGGGGCTTTCACCTACCACCTTCCAAACTGTCTTCCCT
CAAATTCGAGAAGGCTGTTTACAGAGTGCAGCTTAGTGAGTTTTCCCCTCCTGGCAGCCGCGTGGTGA
TGGTGAGAGTCACCCCAGCCTTCCCCAACCTGCAGTATGTTCTAAAGCCATCTTCAGAGAATGTAGGA
TTTAAACTTAATGCTCGAACTGGGTTGATCACCACCACAAAGCTCATGCACTTCCACGACAGAGCCCA
CTATCAGCTACACATCAGAACCTCACCGGGCCAGGCCTCCACCGTGGTGGTCATTGACATTGTGGACT
GCAACAACCATGCCCCCCTCTTCAACAGGTCTTCCTATGATGGTACCTTGGATGAGAACATCCCTCCA
GGCACCAGTGTTTTGGCTGTGACTGCCACTGACCGGGATCATGGGGAAAATGGATATGTCACCTATTC
CATTGCTGGACCAAAAGCTTTGCCATTTTCTATTGACCCTTACCTGGGGATCATCTCCACCTCCAAAC
CCATGGACTATGAACTCATGAAAAGAATTTATACCTTCCGGGTAAGAGCATCAGACTGGGGATCCCCT
TTTCGCCGGGAGAAGGAAGTGTCCATTTTTCTTCAGCTCAGGAACTTGAATGACAACCAGCCTATGTT
TGAAGAAGTCAACTGTACAGGTTCTATCTGCCAAGACTGGCCAGTAGGGAAATCGATAATGACTATGT
CAGCCATAGATGTGGATGAGCTTCAGAACCTAAAATACGAGATTGTATCAGGCAATGAACTAGAGTAT
TTTGATCTAAATCATTTCTCCCGAGTGATATCCCTCAAACGCCCTTTTATCAATCTTACTGCTGGTCA
ACCCACCAGTTATTCCCTGAAGATTACAGCCTCAGATGGCAAAAACTATGCCTCACCCACAACTTTGA
ATATTACTGTGGTG

| | | | |
|---|---|---|---|
| NOV6e, 317868343 | | | |
| Protein Sequence | SEQ ID NO: 48 | 606 aa | MW at ˜73978kD |

MTIALLGFAIFLLHCATCEKPLEGILSSSAWHFTHSHYNATIYENSSPKTYVESFEKMGIYLAEP
QWAVRYRIISGDVANVFKTEEYVVGNFCFLRIRTKSSNTALLNREVRDSYTLIIQATEKTLELEALTR
VVVHILDQNDLKPLFSPPSYRVTISEDMPLKSPICKVTATDADLGQNAEFYYAFNTRSEMFAIHPTSG
VVTVAGKLNVTWRGKHELQVLAVDRMRKISEGNGFGSLAALVVHVEPALRKPPATASVVVTPPDSNDG
TTYATVLVDANSSGAEVESVEVVGGDPGKHFKAIKSYARSNEFSLVSVKDINWMEYLHGFNLSLQARS
GSGPYFYSQIRGFHLPPSKLSSLKFEKAVYRVQLSEFSPPGSRVVMVRVTPAFPNLQYVLKPSSENVG
FKLNARTGLITTTKLMDFHDRAHYQLHIRTSPGQASTVVVIDIVDCNNHAPLFNRSSYDGTLDENIPP
GTSVLAVTATDRDHGENGYVTYSIAGPKALPFSIDPYLGIISTSKPMDYELMKRIYTFRVRASDWGSP
FRREKEVSIFLQLRNLNDNQPMFEEVNCTGSICQDWPVGKSIMTMSAIDVDELQNLKYEIVSGNE
FDLNHFSGVISLKRPFINLTAGQPTSYSLKITASDGKNYASPTTLNITVVLEA

| | | |
|---|---|---|
| NOV6f, 317868367 | SEQ ID NO: 49 | 1977 bp |
| DNA Sequence | ORF Start at 1 | ORF Stop: end of sequence |

ATGACTATTGCCCTGCTGGGTTTTGCCATATTCTTGCTCCATTGTGCGACCTGTGAGAA
GCCTCTAGAAGGGATTCTCTCCTCCTCTGCTTGGCACTTCACACACTCCCATTACAATGCCACCATCT
ATGAAAATTCTTCTCCCAAGACCTATGTGGAGAGCTTCGAGAAAATGGGCATCTACCTCGCGGAGCCA
CAGTGGGCAGTGAGGTACCGGATCATCTCTGGGGATGTGGCCAATGTATTTAAAACTGAGGAGTATGT
GGTGGGCAACTTCTGCTTCCTAAGAATAAGGACAAAGAGCAGCAACACAGCTCTTCTGAACAGAGAGG
TGCGAGACAGCTACACCCTCATCATCCAAGCCACAGAGAAGACCTTGGAGTTGGAAGCTTTGACCCGT
GTGGTGGTCCACATCCTGGACCAGAATGACCTCAAGCCTCTCTTCTCTCCACCTTCGTACAGAGTCAC
CATCTCTGAGGACATGCCCCTGAAGAGCCCCATCTGCAAGGTGACTGCCACAGATGCTGATCTAGGCC
AGAATGCTGAGTTCTATTATGCCTTTAACACAAGGTCAGAGATGTTTGCCATCCATCCCACCAGCGGT
GTGGTCACTGTGGCTGGGAAGCTTAACGTCACCTGGCGAGGAAAGCATGAGCTCCAGGTGCTAGCTGT
GGACCGCATGCGGAAAATCTCTGAGGGCAATCGGTTTGGCAGCCTGGCTGCACTTGTGGTTCATGTGG
AGCCTGCCCTCAGGAAGCCCCCAGCCATTGCTTCGGTGGTGGTGACTCCACCAGACAGCAATGATGGT
ACCACCTATGCCACTGTACTGGTCGATGCAAATAGCTCAGGAGCTGAAGTGGAGTCAGTGGAAGTTGT
TGGTGGTGACCCTGGAAAGCACTTCAAAGCCATCAAGTCTTATGCCCGGAGCAATGAGTTCAGTTTGG
TGTCTGTCAAAGACATCAACTGGATGGAGTACCTTCATGGGTTCAACCTCAGCCTCCAGGCCACGAGT
GGGAGCGGCCCTTATTTTTATTCCCAGATCAGGGGCTTTCACCTACCACCTTCCAAACTGTCTTCCCT
CAAATTCGAGAAGGCTGTTTACAGAGTGCAGCTTAGTGAGTTTTCCCCTCCTGGCAGCCGCGTGGTGA
TGGTGAGAGTCACCCCAGCCTTCCCCAACCTGCAGTATGTTCTAAAGCCATCTTCAGAGAATGTAGGA
TTTAAACTTAATGCTCGAACTGGGTTGATCACCACCACAAAGCTCATGGACTTCCACGACAGAGCCCA
CTATCAGCTACACATCAGAACCTCACCGGGCCAGGCCTCCACCGTGGTGGTCATTGACATTGTGGACT
GCAACAACCATGCCCCCCTCTTCAACAGGTCTTCCTATGATGGTACCTTGGATGAGAACATCCCTCCA
GGCACCAGTGTTTTGGCTGTGACTGCCACTGACCGGGATCATGGGGAAAATGGATATGTCACCTATTC
CATTGCTGGACCAAAAGCTTTGCCATTTTCTATTGACCCTTACCTGGGGATCATCTCCACCTCCAAAC
CCATGGACTATGAACTCATGAAAAGAATTTATACCTTCCGGGTAAGAGCATCAGACTGGGGATCCCCT
TTTCGCCGGGAGAAGGAAGTGTCCATTTTTCTTCAGCTCAGGAACTTGAATGACAACCAGCCTATGTT
TGAAGAAGTCAACTGTACAGGGTCTATCCGCCAAGACTGGCCAGTACGGAAATCGATAATGACTATGT
CAGCCATAGATGTGGATGAGCTTCAGAACCTAAAATACGAGATTGTATCAGGCAATGAACTAGAGTAT
TTTGATCTAAATCATTTCTCCGGAGTGATATCCCTCAAACGCCCTTTTATCAATCTTACTGCTGGTCA
ACCCACCAGTTATTCCCTGAAGATTACAGCCTCAGATCGCAAAAACTATGCCTCACCCACAACTTTGA
ATATTACTGTGGTG

| | | | |
|---|---|---|---|
| NOV6f, 317868367 | | | |
| Protein Sequence | SEQ ID NO: 50 | 659 aa | MW at 74031.4kD |

MTIALLCFAIFLLHCATCEKPLEGILSSSAWHFTHSHYNATIYENSSPKTYVESFEKMGIYLAEP
QWAVRYRIISGDVANVFKTEEYVVGNFCFLRIRTKSSNTALLNREVRDSYTLIIQATEKTLELEALTR
VVVHILDQNDLKPLFSPPSYRVTISEDMPLKSPICKVTATDADLGQNAEFYYAFNTRSEMFAIHPTSG
VVTVAGKLNVTWRGKHELQVLAVDRMRKISEGNGFGSLAALVVHVEPALRKPPAIASVVVTPPDSNDG
TTYATVLVDANSSGAEVESVEWGGDPGKHFKAIEKSYARSNEFSLVSVKDINWMEYLHGFNLSLQARS
GSGPYFYSQIRGFHLPPSKLSSLKFEKAVYRVQLSEFSPPGSRVVNVRVTPAFPNLQYVLKPSSENVG
FKLNARTGLITTTKLMDFHDRAHYQLHIRTSPGQASTVVVIDIVDCNNHAPLFNRSSYDGTLDENIPP
GTSVLAVTATDRDHGENGYVTYSIAGPKALPFSIDPYLGIISTSKPMDYELMKRIYTFRVRASDWGSP
FRREKEVSIFLQLRNLNDNQPMFEEVUCTGSIRQDWPVGKSIMTMSAIDVDELQNLKYEIVSGNELEY
FDLNHFSGVISLKRPFINLTAGQPTSYSLKITASDGKNYASPTTLNITVV

| | | |
|---|---|---|
| NOV6g, 317871203 | SEQ ID NO: 51 | 1923 bp |
| DNA Sequence | ORF Start: at 1 | ORF Stop: end of sequence |

GAGAAGCCTCTAGAAGGGATTCTCTCCTCCTCTGCTTGGCACTTCACACACTCCCATTA
CAATCCCACCATCTATGAAAATTCTTCTCCCAAGACCTATGTCCAGAGCTTCGAGAAAATGCGCATCT
ACCTCGCGGAGCCACAGTGGGCAGTGAGGTACCGGATCATCTCTGGGGATGTGGCCAATGTATTTAAA
ACTGAGCAGTATGTGGTGGGCAACTTCTGCTTCCTAAGAATAAGGACAAAGAGCAGCAACACAGCTCT
TCTGAACAGAGAGGTGCGAGACAGCTACACCCTCATCATCCAAGCCACAGAGAAGACCTTGGAGTTGG
AAGCTTTGACCCGTGTGGTGGTCCACATCCTGGACCAGAATGACCTGAAGCCTCTCTTCTCTCCACCT
TCGTACAGAGTCACCATCTCTGAGGACATGCCCCTGAAGAGCCCCATCTGCAAGGTGACTGCCACAGA
TGCTGATCTAGGCCAGAATGCTGAGTTCTATTATGCCTTTAACACAAGGTCAGAGATGTTTGCCATCC
ATCCCACCAGCGGTGTGGTCACTGTGGCTGGGAAGCTTAACGTCACCTGGCGAGGAAAGCATGAGCTC
CAGGTCCTAGCTGTGGACCGCATGCGGAAAATCTCTGAGGGCAATGGGTTTGGCAGCCTGGCTGCACT
TGTGGTTCATGTGGAGCCTGCCCTCAGGAAGCCCCCAGCCATTGCTTCAGTGGTGGTGACTCCACCAG
ACAGCAATGATGGTACCACCTATGCCACTGTACTGGTCGATGCAAATAGCTCAGGAGCTGAAGTGGAG
TCAGTGGAAGTTGTTGGTGGTCACCCTGGAAAGCACTTCAAAGCCATCAAGTCTTATGCCCCGAGCAA
TGAGTTCAGTTTGGTGTCTGTCAAAGACATCAACTGGATGGAGTACCTTCATGGGTTCAACCTCAGCC
TCCAGGCCAGGAGTGGGAGCGGCCCTTATTTTTATTCCCAGATCAGGGGCTTTCACCTACCACCTTCC
AAACTGTCTTCCCTCAAATTCGAGAAGGCTGTTTACAGAGTGCAGCTTAGTGAGTTTTCCCCTCCTGG
CAGCCGCGTGGTGATGGTGAGAGTCACCCCAGCCTTCCCCAACCTGCAGTATGTTCTAAAGCCATCTT
CAGACAATGTAGGATTTAAACTTAATGCTCGAACTGGGTTGATCACCACCACAAAGCTCATGGACTTC
CACGACAGAGCCCACTATCAGCTACACATCAGAACCTCACCGGGCCAGGCCTCCACCGTGGTGGTCAT
TGACATTGTGGACTGCAACAACCATGCCCCCCTCTTCAACAGGTCTTCCTATGATGGTACCTTGGATG
AGAACATCCCTCCAGGCACCAGTGTTTTGGCTGTGACTGCCACTGACCGGGATCATGGGGAAAATGGA
TATGTCACCTATTCCATTGCTGGACCAAAAGCTTTGCCATTTTCTATTGACCCTTACCTGGGGATCAT
CTCCACCTCCAAACCCATGGACTATGAACTCATGAAAAGAATTTATACCTTCCGCGTAAGAGCATCAG
ACTGGGGATCCCCTTTTCGCCGGGAGAAGGAAGTGTCCATTTTTCTTCACCTCAGGAACTTGAATGAC
AACCAGCCTATGTTTGAAGAAGTCAACTGTACAGGGTCTATCTGCCAAGACTGGCCAGTAGGGAAATC
GATAATGACTATGTCAGCCATAGATGTGGATGAGCTTCAGAACCTAAAATACGAGATTGTATCAGGCA
ATGAACTAGAGTATTTTGATCTAAATCATTTCTCCGGAGTGATATCCCTCAAACGCCCTTTTATCAAT
CTTACTGCTGGTCAACCCACCACTTATTCCCTGAAGATTACAGCCTCAGATGGCAAAAACTATGCCTC
ACCCACAACTTTGAATATTACTGTGGTG

| | | | |
|---|---|---|---|
| NOV6g, 317871203 | | | |
| Protein Sequence | SEQ ID NO: 52 | 641 aa | MW at 72058.0kD |

EKPLEGILSSSAWHFTHSHYNATTYENSSPKTYVESFEKMGIYLAEPGWAVRYRIISGDVANVFK
TEEYVVGNFCFLRIRTKSSNTALLNREVRDSYTLIIQATEKTLELEALTRVVVHILDQNDLKPLFSPP
SYRVTISEDMPLKSPICKVTATDADLGQNAEFYYAFNTRSEMFAIHPTSGVVTVAGKLNVTWRGKHEL
QVLAVDRMRKISEGNGFGSLAALVVHVEPALRKPPAIASVVVTPPDSNDGTTYATVLVDANSSGAEVE
SVEVVGGDPGKHFKAIKSYARSNEFSLVSVKDINWMEYLHGFNLSLQARSGSCPYFYSQIRGFHLPPS
KLSSLKFEKAVYRVQLSEFSPPGSRVVMVRVTPAFPNLQYVLKPSSENVGFKLNARTGLITTTKLMDF
HDRAHYQLHIRTSPGQASTVVVIDIVDCNNHAPLFNRSSYDGTLDENIPPGTSVLAVTATDRDHGENG
YVTYSIAGPKMJPFSIDPYLGIISTSKPMDYELMKRIYTFRVRASDWGSPFRREKEVSIFLQLRNLND
NQPMFEEVNCTGSICQDWPVGKSIMTMSAIDVDELQNLKYEIVSGNELEYFDLNHFSGVISLKRPFIN
LTAGQPTSYSLKITASDGKNYASPTTLNITVV

| | | |
|---|---|---|
| NOV6h, 317871219 | SEQ ID NO: 53 | 1518 bp |
| DNA Sequence | ORF Start: at 1 | ORF Stop: end of sequence |

AGAGTCACCATCTCTGAGGACATGCCCCTGAAGAGCCCCATCTGCAAGGTGACTGCCAC
AGATGCTGATCTAGGCCACAATGCTGAGTTCTATTATGCCTTTAACACAAGGTCAGAGATGTTTGCCA
TCCATCCCACCAGCGGTGTGGTCACTGTGGCTGGGAAGCTTAACGTCACCTGGCGAGGAAAGCATGAG
CTCCAGGTGCTAGCTCTGGACCGCATGCGGAAAATCTCTGAGGGCAATGGGTTTGGCAGCCTGGCTGC
ACTTGTGGTTCATGTGGAGCCTGCCCTCAGGAAGCCCCCAGCCATTGCTTCAGTGGTGGTGACTCCAC
CAGACAGCAATGATGGTACCACCTATGCCACTGTACTGGTCGATGCAAATAGCTCAGGAGCTGAAGTG
CAGTCAGTGGAAGTTGTTGGTGGTGACCCTGGAAAGCACTTCAAAGCCATCAAGTCTTATGCCCGGAG
CAATGAGTTCAGTTTGGTGTCTGTCAAAGACATCAACTGGATGGAGTACCTTCATGGGTTCAACCTCA
GCCTCCAGGCCAGGAGTGGGAGCGGCCCTTATTTTTATTCCCAGATCAGGGGCTTTCACCTACCACCT
TCCAAACTGTCTTCCCTCAAATTCGAGAAGGCTGTTTACAGAGTGCAGCTTAGTGAGTTTTCCCCTCC
TGGCAGCCGCGTGGTGATGGTGAGAGTCACCCCAGCCTTCCCCAACCTGCAGTATGTTCTAAAGCCAT
CTTCAGAGAATGTAGGATTTAAACTTAATGCTCGAACTGGGTTGATCACCACCACAAAGCTCATGGAC
TTCCACGACAGAGCCCACTATCAGCTACACATCAGAACCTCACCGGGCCAGGCCTCCACCGTGGTGGT
CATTGACATTGTGGACTGCAACAACCATCCCCCCCTCTTCAACAGGTCTTCCTATGATGGTACCTTCG
ATCAGAACATCCCTCCAGGCACCAGTGTTTTGGCTGTGACTGCCACTGACCGGGATCATGGGGAAAAT
GGATATGTCACCTATTCCATTGCTGGACCAAAAGCTTTGCCATTTTCTATTGACCCTTACCTGGGGAT
CATCTCCACCTCCAAACCCATGGACTATGAACTCATGAAAAGAATTTATACCTTCCGGGTAAGAGCAT
CAGACTGGGGATCCCCTTTTCGCCGGGAGAAGGAAGTGTCCATTTTTCTTCAGCTCAGGAACTTGAAT
GACAACCAGCCTATGTTTGAAGAAGTCAACTGTACAGGTTCTATCTGCCAAGACTGGCCAGTAGGCAA
ATCGATAATGACTATGTCAGCCATAGATGTGGATGAGCTTCACAACCTAAAATACGAGATTGTATCAG
GCAATGAACTAGAGTATTTTGATCTAAATCATTTCTCCGGAGTGATATCCCTCAAACGCCCTTTTATC
AATCTTACTGCTGGTCAACCCACCAGTTATTCCCTGAAGATTACAGCCTCAGATGGCAAAAACTATGC
CTCACCCACAACTTTGAATATTACTGTGGTG

| | | | |
|---|---|---|---|
| NOV6h, 317871219 | | | |
| Protein Sequence | SEQ ID NO: 54 | 506 aa | MW at ˜56527kD |

RVTISEDMPLKSPICKVTATDADLGQNAEFYYAFNTRSEMFAIHPTSGVVTVAGKLNVTWRGKHE
LQVLAVDRMRKISECNGFGSLAALVVHVEPALRKPPAIASVVVTPPDSNDGTTYATVLVDANSSGAEV
ESVEVVGGDPGKHFKAIKSYARSNEFSLVSVKDINWMEYLHGFNLSLQARSGSGPYFYSQIRGFHLPP
SKLSSLKFEKAVYRVQLSEFSPPGSRVVMVRVTPAFPNLQYVLKPSSENVGFKLNARTGLITTTKLMD
FHDRAHYQLHIRTSPGQASTVVVIDIVDCNNHAPLFNRSSYDGTLDENIPPGTSVLAVTATDRDHGEN
GYVTYSIAGPKALPFSIDPYLGIISTSKPMDYELMKRIYTFRVRASDWGSPFRREKEVSIFLQLRNLN
DNQPMFEEVNCTGSICQDWPVGKSIMTMSAIDVDELQNLKYEIVSGNELEYFDLNHFSGVISLKRPFI
NLTAGQPTSYSLKITASDGKNYASPTTLNITVV

| | | |
|---|---|---|
| NOV6i, 317871243 | SEQ ID NO: 55 | 1518 bp |
| DNA Sequence | ORF Start at 1 | ORF Stop: end of sequence |

AGAGTCACCATCTCTGAGGACATGCCCCTGAAGAGCCCCATCTCCAAGGTGACTGCCAC
AGATGCTGATCTAGGCCAGAATGCTGAGTTCTATTATGCCTTTAACACAAGGTCACAGATGTTTGCCA
TCCATCCCACCAGCGGTGTGGTCACTGTGGCTCGGAAGCTTAACGTCACCTGGCGAGGAAAGCATGAG
CTCCAGGTGCTAGCTGTGGACCGCATGCGGAAAATCTCTGAGGGCAATGGGTTTGGCAGCCTGGCTGC
ACTTGTGGTTCATGTGGAGCCTGCCCTCAGCAAGCCCCCAGCCATTGCTTCGGTGGTGGTGACTCCAC
CAGACAGCAATGATGGTACCACCTATGCCACTGTACTGGTCGATGCAAATAGCTCAGGACCTGAAGTG
GAGTCAGTGGAAGTTGTTGGTGGTGACCCTGGAAAGCACTTCAAAGCCATCAAGTCTTATGCCCGGAG
CAATGAGTTCAGTTTGGTGTCTGTCAAACACATCAACTGGATGGAGTACCTTCATGGGTTCAACCTCA
GCCTCCAGGCCAGGAGTGGGAGCGGCCCTTATTTTTATTCCCAGATCAGGGGCTTTCACCTACCACCT
TCCAAACTGTCTTCCCTCAAATTCGAGAAGCCTGTTTACAGAGTGCAGCTTAGTGAGTTTTCCCCTCC
TGGCAGCCGCGTGGTGATGGTGAGAGTCACCCCAGCCTTCCCCAACCTGCAGTATGTTCTAAAGCCAT
CTTCAGAGAATGTAGGATTTAAACTTAATGCTCGAACTGGGTTGATCACCACCACAAAGCTCATGGAC
TTCCACCACAGAGCCCACTATCAGCTACACATCAGAACCTCACCGGGCCAGGCCTCCACCGTGGTGGT
CATTGACATTGTGGACTGCAACAACCATGCCCCCCTCTTCAACAGGTCTTCCTATGATGGTACCTTGG
ATGAGAACATCCCTCCAGCCACCAGTGTTTTGGCTGTGACTGCCACTGACCGGGATCATGGGGAAAAT
GGATATGTCACCTATTCCATTGCTGGACCAAAAGCTTTGCCATTTTCTATTGACCCTTACCTGGGGAT
CATCTCCACCTCCAAACCCATGGACTATGAACTCATGAAAAGAATTTATACCTTCCGGGTAAGAGCAT
CAGACTGGGGATCCCCTTTTCGCCGGGAGAAGGAAGTGTCCATTTTTCTTCAGCTCAGGAACTTGAAT
GACAACCAGCCTATGTTTGAAGAAGTCAACTGTACAGGGTCTATCCGCCAAGACTGGCCAGTAGGGAA
ATCGATAATGACTATGTCAGCCATAGATGTGGATGAGCTTCAGAACCTAAAATACGAGATTGTATCAG
GCAATGAACTAGAGTATTTTGATCTAAATCATTTCTCCGGAGTGATATCCCTCAAACGCCCTTTTATC
AATCTTACTGCTGGTCAACCCACCAGTTATTCCCTGAAGATTACAGCCTCAGATGGCAAAAACTATGC
CTCACCCACAACTTTGAATATTACTGTGGTG

| | | | |
|---|---|---|---|
| NOV6i, 317871243 | | | |
| Protein Sequence | SEQ ID NO: 56 | 506 aa | MW at ˜56580kD |

RVTISEDMPLKSPICKVTATDADLGQNAEFYYAFNTRSEMFAIHPTSGVVTVAGKLNVTWRGKHE
LQVLAVDRMRKISEGNGFGSLAALVVHVEPALRKPPAIASVVVTPPDSNDGTTYATVLVDANSSGAEV
ESVEVVGGDPGKHFKAIKSYARSNEFSLVSVKDINWMEYLHGFNLSLQARSGSGPYFYSQIRGFHLPP
SKLSSLKFEKAVYRVQLSEFSPPGSRVVMVRVTPAFPNLQYVLKPSSENVGFKLNARTGLITTTKLMD
FHDRAHYQLHIRTSPGQASTVVVIDIVDCNNHAPLFNRSSYDGTLDENIPPGTSVLAVTATDRDHGEN
CYVTYSIAGPKALPFSIDPYLGIISTSKPMDYELMKRIYTFRVRASDWGSPFRREKEVSIFLQLRNLN
DNQPMFEEVNCTGSIRQDWPVGKSIMTMSAIDVDELQNLKYEIVSGNELEYFDLNHFSGVISLKRPFI
NLTAGQPTSYSLKITASDGKNYASPTTLNITVV

| | | |
|---|---|---|
| NOV6j, 317871246 | SEQ ID NO: 57 | 1992 bp |
| DNA Sequence | ORF Start: at 1 | ORF Stop: end of sequence |

ACAGGGTCTATCCGCCAAGACTGCCCAGTAGGGA
AATCGATAATGACTATGTCAGCCATAGATGTGGATGAGCTTCAGAACCTAAAATACGAGATTGTATCA
GGCAATGAACTAGAGTATTTTGATCTAAATCATTTCTCCGGAGTGATATCCCTCAAACGCCCTTTTAT
CAATCTTACTGCTGGTCAACCCACCAGTTATTCCCTGAACATTACAGCCTCAGATGGCAAAAACTATG
CCTCACCCACAACTTTGAATATTACTGTGGTGAAGGACCCTCATTTTGAAGTTCCTGTAACATGTGAT
AAAACAGGGCTATTGACACAATTCACAAAGACTATCCTCCACTTTATTGGGCTTCAGAACCAGCAGTC
CAGTGATGAGGAATTCACTTCTTTAAGCACATATCAGATTAATCATTACACCCCACAGTTTGAGGACC
ACTTCCCCCAATCCATTGATGTCCTTGAGAGTGTCCCTATCAACACCCCCTTGGCCCGCCTAGCAGCC
ACTGACCCTGATGCTGGTTTTAATGGCAAACTGGTCTATGTGATTGCAGATGGCAATGAGGAGGGCTG
CTTTGACATAGAGCTGGAGACAGGGCTGCTCACTGTAGCTGCTCCCTTGGACTATGAAGCCACCAATT
TCTACATCCTCAATGTAACAGTATATGACCTGGGCACACCCCAGAAGTCCTCCTGGAAGCTGCTGACA
GTGAATGTGAAAGACTGGAATGACAACGCACCCAGATTTCCTCCCGGTGCGTACCAGTTAACCATCTC
GGAGGACACAGAAGTTGGAACCACAATTGCAGAGCTGACAACCAAAGATGCTGACTCGGAAGACAATG
CCAGGGTTCGCTACACCCTGCTAAGTCCCACAGAGAAGTTCTCCCTCCACCCTCTCACTGGGCAACTG
GTTGTTACAGGACACCTGGACCGCGAATCACAGCCTCGGTACATACTCAAGGTGGAGGCCAGGGATCA
GCCCACCAAAGGCCACCAGCTCTTCTCTGTCACTGACCTGATAATCACATTGGAGGATGTCAACGACA
ACTCTCCCCAGTGCATCACAGAACACAACAGGCTGAAGGTTCCAGAGGACCTGCCCCCCGGGACTGTC
TTGACATTTCTGGATGCCTCTGATCCTGACCTCGGCCCCGCAGGTGAAGTGCGATATGTTCTGATGGA
TGGCGCCCATGGGACCTTCCGGGTGGACCTGATGACAGGGGCGCTCATTCTGGAGAGAGACCTGGACT
TTGAGAGGCGAGCTGGGTACAATCTGAGCCTGTGGCCCAGTGATGGTGGGAGGCCCCTAGCCCGCAGG
ACTCTCTGCCATGTGGAGGTGATCGTCCTGGATGTGAATGAGAATCTCCACCCTCCCCACTTTGCCTC
CTTCGTGCACCAGGGCCAGGTGCAGGAGAACAGCCCCTCGGGAACTCAGGTGATTGTAGTGGCTGCCC
AGGACGATGACAGTGGCTTGGATGGGGAGCTCCAGTACTTCCTGCGTGCTGGCACTGGACTCGCAGCC
TTCAGCATCAACCAAGATACAGGAATGATTCAGACTCTGGCACCCCTGGACCGAGAATTTGTATCTTA
CTACTGGTTGACGGTATTAGCAGTGGACAGGGGTTCTGTGCCCCTCTCTTCTGTAACTGAAGTCTACA
TCGAGGTTACGGATGCCAATGACAACCCACCCCAGATGTCCCAAGCTGTGTTCTACCCCTCCATCCAG
GAGGATGCTCCCGTGGGCACCTCTGTGCTTCAACTGGATGCCTGGGACCCAGACTCCAGCTCCAAAGG
GAAGCTGACCTTCAACATCACCAGTGGGAACCACATGGGATTCTTTATGATTCACCCTGTTACAGGTC
TCCTATCTACAGCCCAGCAGCTGGACAGAGAGAACAAGGATGAACACATCCTGGAGGTGACTGTGCTC
GACAATGGGGAACCCTCACTGAAGTCCACCTCCAGGGTGGTGGTAGGCATCTTG

| | | | |
|---|---|---|---|
| NOV6j, 317871246 | | | |
| Protein Sequence | SEQ ID NO: 58 | 664 aa | MW at ˜74703kD |

TGSIRQDWPVGKSIMPMSAIDVDELQNLKYEIVSGNELEYFDLNHFSGVISLKRPFI
NLTAGQPTSYSLKITASDGKNYASPTTLNITVVKDPHFEVPVTCDKTGVLTQFTKTILHFIGLQNQES
SDEEFTSLSTYQINHYTPQFEDHFPQSIDVLESVPINTPLARLAATDPDAGFNGKLVYVIADGNEEGC
FDIELETGLLTVAAPLDYEATNFYILNVTVYDLGTPQKSSWKLLTVNVKDWNDNAPRFPPGGYQLTIS
EDTEVGTTIAELTTKDADSEDNGRVRYTLLSPTEKFSLHPLTGELVVTGHLDRESEPRYILKVEARDQ
PSKGHQLFSVTDLIITLEDVNDNSPQCITEHNRLKVPEDLPPGTVLTFLDASDPDLGPAGEVRYVLMD
GAHGTFRVDLMTGALILERELDFERPAGYNLSLWASDGGRPLARRTLCHVEVIVLDVNENLHPPHFAS
FVHQGQVQENSPSGTQVIVVAAQDDDSGLDGELQYFLRAGTGLAAFSINQDTGMIQTLAPLDREFVSY
YWLTVLAVDRGSVPLSSVTEVYIEVTDANDNPPQMSQAVFYPSIQEDAPVGTSVLQLDAWDPDSSSKG
KLTFNITSGNHMGFFMIHPVTGLLSTAQQLDRENKDEHILEVTVLDNGEPSLKSTSRVVVGIL

| | | |
|---|---|---|
| NOV6k, 317999764 | SEQ ID NO: 59 | 1773 bp |
| DNA Sequence | ORF Start: at 1 | ORF Stop: end of sequence |

TACCCCTCCATCCAGGAGGATGCTCCCGTGGGCACCTCTGTGCTTCAACTGGATGCCTG
GGACCCAGACTCCAGCTCCAAAGGGAAGCTGACCTTCAACATCACCAGTGGGAACCACATGGGATTCT
TTATCATTCACCCTGTTACAGGTCTCCTATCTACAGCCCAGCAGCTGGACAGAGAGAACAAGGATGAA
CACATCCTGGAGGTGACTGTGCTGGACAATGGGGAACCCTCACTGAAGTCCACCTCCAGGGTGCTGGT
AGGCATCTTGGACGTCAATGACAATCCACCTATATTCTCCCACAAGCTCTTCAATGTCCGCCTTCCAG
AGAGGCTGAGCCCTGTGTCCCCTGGGCCTGTGTACAGGCTGGTGGCTTCACACCTGGATGAGGGTCTT
AATGGCAGAGTCACCTACAGTATCGAGGACAGCGATGAGGAGCCCTTCACTATCGACCTGGTCACAGG
TGTGGTTTCATCCAGCAGCACTTTTACAGCTGGAGAGTACAACATCCTAACGATCAAGGCAACAGACA
CTGGGCAGCCACCACTCTCAGCCAGTGTCCGCCTACACATTGACTGGATCCCTTGGCCCCGGCCGTCC
TCCATCCCTCTGGCCTTTGATGAGACCTACTACAGCTTTACGGTCATGGAGACGGACCCTGTGAACCA
CATGGTGGGGGTCATCAGCGTAGAGGGCAGACCCGGACTCTTCTGGTTCAACATCTCAGGTGGGGATA
AGGACATGGACTTTGACATTGACAAGACCACAGGCAGCATCGTCATTGCCAGGCCTCTTGATACCAGG
AGAAGGTCGAACTATAACTTGACTGTTGAGGTGACAGATGGGTCCCGCACCATTGCCACACAGGTCCA
CATCTTCATCATTGCCAACATTAACCACCATCGGCCCCAGTTTCTGGAAACTCGTTATGAAGTCAGAG
TTCCCCAGGACACCGTGCCAGGCGTAGAGCTCCTGCGAGTCCAGGCCATAGATCAAGACAAGGGCAAA
AGCCTCATCTATACCATACATGGCAGCCAAGACCCAGGAAGTGCCAGCCTCTTCCAGCTGGACCCAAG
CAGTGGTGTCCTGGTAACGGTGGGAAAATTGGACCTCGGCTCGGGGCCCTCCCAGCACACACTGACAG
TCATGGTCCGAGACCAGGAAATACCTATCAAGAGGAACTTCGTGTGGGTGACCATTCATGTGGAGGAT
GGAAACCTCCACCCACCCCGCTTCACTCAGCTCCATTATGAGGCAAGTGTTCCTGACACCATAGCCCC
CGGCACAGAGCTGCTGCAGGTCCGAGCCATGGATGCTGACCGGGGAGTCAATGCTGAGGTCCACTACT
CCCTCCTGAAAGGGAACAGCGAAGGTTTCTTCAACATCAATGCCCTGCTAGGCATCATTACTCTAGCT
CAAAAGCTTGATCAGGCAAATCATGCCCCACATACTCTGACAGTGAAGGCAGAAGATCAAGGCTCCCC
ACAATGGCATGACCTGGCTACAGTGATCATTCATGTCTATCCCTCAGATAGGAGTGCCCCCATCTTTT
CAAAATCTGAGTACTTTGTAGAGATCCCTGAATCAATCCCTGTTGGTTCCCCAATCCTCCTTGTCTCT
GCTATGAGCCCCTCTGAAGTTACCTATGAGTTAAGAGAGGGAAATAAGGATGGAGTCTTCTCTATGAA
CTCATATTCTGGCCTTATTTCCACCCAGAAGAAATTGGACCATGAGAAAATCTCGTCTTACCAGCTGA
AAATCCGAGGCAGC

| | | | |
|---|---|---|---|
| NOV6k, 317999764 | | | |
| Protein Sequence | SEQ ID NO: 60 | 591 aa | MW at ˜65888kD |

YPSIQEDAPVGTSVLQLDAWDPDSSSKGKLTFNITSGNHMGFFMIHPVTGLLSTAQQLDRENKDE
HILEVTVLDNGEPSLKSTSRVVVGILDVNDIPPIFSHKLFNVRLPERLSPVSPGPVYRLVASDLDEGL
NGRVTYSIEDSDEEAFSIDLVTCVVSSSSTFTAGEYNILTIKATDSGQPPLSASVRLHIEWIPWPRPS
SIPLAFDETYYSFTVMETDPVNHMVGVISVEGRPGLFWFNISGGDKDMDFDIEKTTGSIVIARPLDTR
RRSNYNLTVEVTDGSRTIATQVHIFMIANINHHRPQFLETRYEVRVPQDTVPGVELLRVQAIDQDKGK
SLIYTIHGSQDPGSASLFQLDPSSGVLVTVGKLDLGSGPSQHTLTVMVRDQEIPIKRNFVWVTIHVED
GNLHPPRFTQLHYEASVPDTIAPGTELLQVRAMDADRGVNAEVHYSLLKGNSEGFFNINALLGIITLA
QKLDQANNAPHTLTVKAEDQGSPQWHDLATVIIHVYPSDRSAPIFSKSEYFVEIPESIPVGSPILLVS
AMSPSEVTYELREGNKDGVFSMNSYSGLISTQKKLDHEKISSYQLKIRGS

| | | |
|---|---|---|
| NOV6l, 318176301 | SEQ ID NO: 61 | 2019 bp |
| DNA Sequence | ORF Start: at 1 | ORF Stop: end of sequence |

GACCCAAGTGTTCCTGACACCATAGCCCCCGGCACAGAGCTGCTCCAGGTCCGAGCCAT
GGATGCTCACCGGGGAGTCAATGCTGAGGTCCACTACTCCCTCCTGAAAGGGAACAGCGAAGGTTTCT
TCAACATCAATGCCCTGCTAGGCATCATTACTCTACCTCAAAAGCTTGATCAGGCAAATCATGCCCCA
CATACTCTGACAGTGAAGGCAGAAGATCAAGGCTCCCCACAATGCCATGACCTGGCTACAGTGATCAT
TCATGTCTATCCCTCACATAGGAGTGCCCCCATCTTTTCAAAATCTGAGTACTTTGTAGAGATCCCTG
AATCAATCCCTGTTGGTTCCCCAATCCTCCTTGTCTCTGCTATGAGCCCCTCTGAAGTTACCTATGAG
TTAAGAGAGGGAAATAAGGATGGAGTCTTCTCTATGAACTCATATTCTGGCCTTATTTCCACCCAGAA
GAAATTGGACCATGAGAAAATCTCGTCTTACCAGCTGAAAATCCGAGGCAGCAATATGGCAGGTGCAT
TTACTGATGTCATGGTGGTGGTTGACATAATTGATGAAAATGACAATGCTCCTATGTTCTTAAAGTCA
ACTTTTGTGGGCCAAATTAGTGAAGCAGCTCCACTGTATAGCATGATCATGGATAAAAACAACAACCC
CTTTGTGATTCATGCCTCTGACAGTGACAAAGAAGCTAATTCCTTGTTGGTCTATAAAATTTTGGAGC
CGCAGGCCTTCAAGTTTTTCAAAATTGATCCCAGCATGGGAACCCTAACCATTGTATCAGAGATGGAT
TATCAGAGCATGCCCTCTTTCCAATTCTGTGTCTATGTCCATGACCAAGGAAGCCCTGTATTATTTGC
ACCCAGACCTGCCCAAGTCATCATTCATGTCAGAGATGTGAATGATTCCCCTCCCAGATTCTCAGAAC
AGATATATGAGGTAGCAATAGTCGGGCCTATCCATCCAGGCATGGAGCTTCTCATGGTGCGGGCCAGC
GATGAAGACTCAGAAGTCAATTATAGCATCAAAACTGGCAATGCTGATGAAGCTGTTACCATCCATCC
TGTCACTCGTAGCATATCTGTGCTGAATCCTGCTTTCCTGGGACTCTCTCGGAAGCTCACCATCAGGG
CTTCTGATGGCTTGTATCAAGACACTCCGCTGGTAAAAATTTCTTTGACCCAAGTGCTTGACAAAAGC
TTGCAGTTTGATCAGGATGTCTACTGGGCAGCTGTGAAGGAGAACTTGCAGGACAGAAAGGCACTGGT
GATTCTTGGTGCCCAGGGCAATCATTTGAATGACACCCTTTCCTACTTTCTCTTGAATGGCACAGATA
TGTTTCATATGGTCCAGTCAGCAGGTGTGTTGCAGACAAGAGGTGTGGCGTTTGACCGGGAGCAGCAG
GACACTCATGAGTTGGCAGTGGAAGTGAGGGACAATCGGACACCTCAGCGGGTCGCTCAGGGTTTGGT
CAGAGTCTCTATTGAGCATGTCAATGACAATCCCCCCAAATTTAAGCATCTCCCCTATTACACAATCA
TCCAAGATGGCACAGAGCCAGGGGATGTCCTCTTTCAGGTATCTGCCACTGATGAGGACTTGGGCACA
AATGGGGCTGTTACATATGAATTTGCAGAAGATTACACATATTTCCGAATTGACCCCTATCTTGGGGA
CATATCACTCAAGAAACCCTTTGATTATCAAGCTTTAAATAAATATCACCTCAAAGTCATTGCTCGGG
ATGGAGGAACGCCATCCCTCCAGAGTGAGGAAGAGGTACTTGTCACTGTGAGAAATAAATCCAACCCA
CTGTTTCAGAGTCCTTATTACAAAGTCAGAGTACCTGAAAATATCACCCTCTATACCCCAATTCTCCA
CACCCAGGCCCGGAGTCCAGAGGGACTCCGGCTCATCTACAACATTGTGGAGGAAGAACCCTTGATGC
TGTTCACCACTGACTTCAAGACTGGTGTCCTAACAGTAACAGGGCCTTTGGACTAT

| | | | |
|---|---|---|---|
| NOV6l, 318176301 | | | |
| Protein Sequence | SEQ ID NO: 62 | 673 aa | MW at ˜75571kD |

EASVPDTIAPGTELLQVRAMDADRGVNAEVHYSLLKGNSECFFNINALLGIITLAQKLDQANHAP
HTLTVKAEDQGSPQWHDLATVIIHVYPSDRSAPIFSKSEYFVEIPESIPVGSPILLVSAMSPSEVTYE
LREGNKDGVFSMNSYSGLISTQKKLDHEKISSYQLKIRGSNMAGAFTDVMVVVDIIDENDNAPMFLKS
TFVGQISEAAPLYSMIMDKNNNPFVIHASDSDKEANSLLVYKILEPEALKFFKIDPSMGTLTIVSEMD
YESMPSFQFCVYVHDQGSPVLFAPRPAQVIIHVRDVNDSPPRFSEQIYEVAIVGPIHPGMELLMVRAS
DEDSEVNYSIKTGNADEAVTIHPVTGSISVLNPAFLGLSRKLTIRASDGLYQDTALVKISLTQVLDKS
LQFDQDVYWAAVKENLQDRKALVILGAQGNHLNDTLSYFLLNGTDMFHMVQSAGVLQTRGVAFDREQQ
DTHELAVEVRDNRTPQRVAQGLVRVSIEDVNDNPPKFKHLPYYTIIQDGTEPGDVLFQVSATDEDLGT
NGAVTYEFAEDYTYFRIDPYLGDISLKKPFDYQALNKYHLKVIARDGGTPSLQSEEEVLVTVRNKSNP
LFQSPYYKVRVPENITLYTPILHTQARSPEGLRLIYNIVEEEPLMLFTTDFKTGVLTVTGPLDY

| | | |
|---|---|---|
| NOV6m, CG51923-02 | SEQ ID NO: 63 | 3666 bp |
| DNA Sequence | ORF Start: at 1 | ORF Stop: end of sequence |

TATAAGGCTGTCCTCACTGAGAATATGCCAGTGGGGACCTCAGTCATTCAAGTGACTGCCATTGACAA
GGACACTGGGAGAGATGGCCAGGTGAGCTACAGGCTGTCTGCAGACCCTGGTAGCAATGTCCATGAGC
TTTTTGCCATTGACAGTGAGAGTGGTTGCATCACCACACTCCAGGAACTTGACTGTGAGACCTGCCAG
ACTTATCATTTTCATGTGGTGGCCTATGACCACGGACAGACCATCCAGCTATCCTCTCAGGCCCTGGT
TCAGGTCTCCATTACAGATGAGAATGACAATGCTCCCCGATTTGCTTCTGAAGAGTACAGAGGATCTG
TGGTTGAGAACAGTGAGCCTGGCGAACTGGTGGCGACTCTAAAGACCCTGGATGCTGACATTTCTGAG
CAGAACAGGCAGGTCACCTGCTACATCACAGAGGGAGACCCCCTGGGCCAGTTTGGCATCAGCCAAGT
TGGAGATGAGTGGAGGATTTCCTCAAGGAAGACCCTGGACCGCGAGCATACAGCCAAGTACTTGCTCA
GAGTCACAGCATCTGATGGCAAGTTCCAGGCTTCGGTCACTGTGGAGATCTTTGTCCTGGACGTCAAT
GATAACAGCCCACAGTGTTCACAGCTTCTCTATACTGGCAAGGTTCATGAAGATGTATTTCCAGGACA
CTTCATTTTGAAGGCTTCTGCCACAGACTTGGACACTGATACCAATGCTCAGATCACATATTCTCTGC
ATGGCCCTGGGGCGCATGAATTCAAGCTGGATCCTCATACAGGGGAGCTGACCACACTCACAGCCCTA
GACCGAGAAAGGAAGGATGTGTTCAACCTTGTTGCCAAGGCGACGGATGGAGGTGGCCGATCGTGCCA
GGCAGACATCACCCTCCATGTGGAGGATGTGAATGACAATGCCCCGCGGTTCTTCCCCAGCCACTGTG
CTGTGGCTGTCTTCGACAACACCACAGTGAAGACCCCTGTGGCTGTAGTATTTGCCCGGGATCCCGAC
CAAGGCGCCAATGCCCAGGTGGTTTACTCTCTGCCGGATTCAGCCGAAGGCCACTTTTCCATCGACGC
CACCACGGGGGTGATCCGCCTGGAAAAGCCGCTGCAGGTCAGGCCCCAGGCACCACTGGAGCTCACGG
TCCGTGCCTCTGACCTGGGCACCCCAATACCGCTGTCCACGCTGGGCACCGTCACAGTCTCGGTGGTG
GGCCTAGAAGACTACCTGCCCGTGTTCCTGAACACCGAGCACAGCGTGCAGGTGCCCGAGGACGCCCC
ACCTGGCACGGAGGTGCTGCAGCTGGCCACCCTCACTCGCCCGGGCGCAGAGAAGACCGGCTACCGCG
TGGTCAGCGGGAACGAGCAAGGCAGGTTCCGCCTGGATGCTCGCACAGGGATCCTGTATGTCAACGCA
AGCCTGGACTTTGAGACAAGCCCCAAGTACTTCCTGTCCATTGAGTGCAGCCGGAAGAGCTCCTCTTC
CCTCAGTGACGTGACCACAGTCATCGTCAACATCACTGATGTCAATGAACACCGGCCCCAATTCCCCC
AAGATCCATATAGCACAAGGGTCTTAGAGAATGCCCTTGTGGGTGACGTCATCCTCACGGTATCAGCG
ACTGATGAAGATGGACCCCTAAATAGTGACATTACCTATAGCCTCATAGGAGGGAACCAGCTTGGGCA
CTTCACCATTCACCCCAAAAAGGGGGAGCTACAGGTGGCCAAGGCCCTGGACCGGGAACAGGCCTCTA
GTTATTCCCTGAAGCTCCGAGCCACAGACAGTGGGCAGCCTCCACTGCATGAGGACACAGACATCGCT
ATCCAAGTGGCTGATGTCAATGATAACCCACCGAGATTCTTCCAGCTCAACTACAGCACCACTGTCCA
GGAGAACTCCCCCATTGGCAGCAAAGTCCTGCAGCTGATCCTGAGTGACCCAGATTCTCCAGAGAATG
GCCCCCCCTACTCGTTTCGAATCACCAAGGGGAACAACGGCTCTGCCTTCCGAGTGACCCCGGATGGA
TGGCTGGTGACTGCTGACGGCCTAAGTAGGACGGCTCAGGAATGGTATCAGCTTCAGATCCAGGCGTC
AGACAGTGGCATCCCTCCCCTCTCGTCTTCGACGTCTGTCCGTGTCCATGTCACAGAGCAGAGCCACT
ATGCACCTTCTGCTCTCCCACTGGAGATCTTCATCACTGTTGGAGAGGATGAGTTCCAGGGTGGCATG
GTGGGTAAGATCCATGCCACAGACCGAGACCCCCAGGACACGCTGACCTATAGCCTGGCAGAAGAGGA
GACCCTGGGCAGGCACTTCTCAGTGGGTGCGCCTGATGGCAAGATTATCGCCGCCCAGGACCTGCCTC
GTGGCCACTACTCGTTCAACGTCACGGTCAGCGATGGGACCTTCACCACGACTGCTGGGGTCCATGTG
TATGTGTGGCATGTGGGGCAGGAGGCTCTGCAGCAGGCCATATGGATGGGCTTCTACCAGCTCACCCC
CGAGGAGCTGGTCAGTGACCACTGGCGGAACCTGCAGAGGTTCCTCAGCCATAAGCTGGACATCAAAC
GGGCTAACATTCACTTGGCCAGCCTCCAGCCTGCAGAGGCCGTGGCTGCTGTGGACGTGCTCCTGGTC
TTTGAGGGGCATTCTGGAACCTTCTACGAGTTTCAGGAGCTAGCATCCATCATCACTCACTCAGCCAA
GGAGATGGAGCATTCAGTGGGGGTTCAGATGCGGTCAGCTATGCCCATGGTGCCCTGCCAGGGGCCAA
CCTGCCAGGGTCAAATCTGCCATAACACAGTCCATCTGGACCCCAAGGTTGGGCCCACGTACAGCACC
GCCAGGCTCAGCATCCTAACCCCGCGGCACCACCTGCAGAGGAGCTGCTCCTGCAATGGTACTGCTAC
AAGGTTCAGTGGTCAGAGCTATGTGCGGTACAGGGCCCCAGCGGCTCGGAACTGGCACATCCATTTCT
ATCTGAAAACACTCCAGCCACAGGCCATTCTTCTATTCACCAATGAAACAGCGTCCGTCTCCCTGAAG
CTGGCCAGTGGAGTGCCCCAGCTGGAATACCACTGTCTGGGTGGTTTCTATGGAAACCTTTCCTCCCA
GCGCCATGTGAATGACCACGAGTGGCACTCCATCCTGGTGGAGGAGATGGACGCTTCCATTCGCCTGA
TGGTTGACAGCATGGGCAACACCTCCCTTGTGGTCCCAGAGAACTGCCGTGGTCTGAGGCCCGAAAGG
CACCTCTTGCTGGGCGGCCTCATTCTGTTGCATTCTTCCTCGAATGTCTCCCAGGGCTTTGAAGGCTG
CCTGGATGCTGTCGTGGTCAACGAAGAGGCTCTAGATCTGCTGGCCCCTGGCAAGACGGTGGCAGGCT
TGCTGGAGACACAAGCCCTCACCCAGTGCTGCCTCCACAGTGACTACTGCAGCCAGAACACATCCCTC
AATGGTGGGAAGTGCTCATGGACCCACGGGGCAGCCTATGTCTGCAAATGTCCCCCACAGTTCTCTGG
GAAGCACTGTGAACAACGAAGGGAGAACTGTACTTTTGCACCCTGCCTGGAAGGTGGAACTTCCATCC
TCTCCCCCAAAGGAGCTTCCTGTAACTGCCCTCATCCTTACACAGGAGACAGGTGTGAAATG

| | | | |
|---|---|---|---|
| NOV6m, CG51923-02 | | | |
| Protein Sequence | SEQ ID NO: 64 | 1222 aa | MW at 133578.0kD |

YKAVLTENMPVGTSVIQVTAIDKDTGRDGQVSYRLSADPGSNVHELFAIDSESGWITTLQELDCETCQ
TYHFHVVAYDHGQTIQLSSQALVQVSITDENDNAPRFASEEYRGSVVENSEPGELVATLKTLDADISE
QNRQVTCYITEGDPLGQFGISQVGDEWRISSRKTLDREHTAKYLLRVTASDGKFQASVTVEIFVLDVN
DNSPQCSQLLYTGKVHEDVFPGHFILKASATDLDTDTNAQITYSLHGPGAHEFKLDPHTGELTTLTAL
DRERKDVFNLVAKATDGCGRSCQADITLHVEDVNDNAPRFFPSHCAVAVFDNTTVKTPVAVVFARDPD
QGANAQVVYSLPDSAEGHFSIDATTGVIRLEKPLQVRPQAPLELTVRASDLGTPIPLSTLGTVTVSVV
GLEDYLPVFLNTEHSVQVPEDAPPGTEVLQLATLTRPCAEKTGYRVVSGNEQGRFRLDARTGILYVNA
SLDFETSPKYFLSIECSRKSSSSLSDVTTVMVNITDVNEHRPQFPQDPYSTRVLENALVGDVILTVSA
TDEDCPLNSDITYSLIGGNQLCHFTIHPKKCELQVAKALDREQASSYSLKLRATDSGQPPLHEDTDIA
IQVADVNDNPPRFFQLNYSTTVQENSPIGSKVLQLILSDPDSPENGPPYSFRITKGNNGSAFRVTPDG
WLVTAEGLSRRAQEWYQLQIQASDSGIPPLSSSTSVRVHVTEQSHYAPSALPLEIFITVGEDEFQGGM
VGKIHATDRDPQDTLTYSLAEEETLGRHFSVGAPDGKIIAAQDLPRGHYSFNVTVSDGTFTTTAGVHV
YVWHVGQEALQQATWMGFYQLTPEELVSDHWRNLQRFLSHKLDIKRANIHLASLQPAEAVAGVDVLLV
FEGHSGTFYEFQELASIITHSAKEMEHSVGVQMRSAMPMVPCQGPTCQGQICHNTVHLDPKVGPTYST
ARLSILTPRHHLQRSCSCNGTATRFSGQSYVRYRAPAARNWHIHFYLKTLQPQAILLFTNETASVSLK
LASGVPQLEYHCLGGFYGNLSSQRHVNDHEWHSILVEEMDASIRLMVDSMGNTSLVVPENCRGLRPER
HLLLGGLILLHSSSNVSQCFEGCLDAVVVNEEALDLLAPGKTVAGLLETQALTQCCLHSDYCSQNTCL
NGGKCSWTHGAGYVCKCPPQFSGKHCEQGRENCTFAPCLEGGTCILSPKGASCNCPHPYTGDRCEM

| | | |
|---|---|---|
| NOV6n, CG51923-03 | SEQ ID NO: 65 | 14279 bp |
| DNA Sequence | ORF Start: ATG at 14 | ORF Stop: TAG at 12806 |

GGAGTTTTCCACCATGACTATTGCCCTGCTGGGTTTTGCCATATTCTTGCTCCATTGTGCGACCTGTG
AGAAGCCTCTAGAAGGGATTCTCTCCTCCTCTGCTTGGCACTTCACACACTCCCATTACAATGCCACC
ATCTATGAAAATTCTTCTCCCAAGACCTATGTGGAGAGCTTCGAGAAAATGGGCATCTACCTCGCGGA
GCCACAGTGGGCAGTGAGGTACCGGATCATCTGTGGGGATGTGGCCAATGTATTTAAAACTGAGGAGT
ATGTGGTGGGCAACTTCTGCTTCCTAAGAATAAGGACAAAGAGCAGCAACACAGCTCTTCTGAACAGA
GAGGTGCGAGACAGCTACACCCTCATCATCCAAGCCACAGAGAAGACCTTGGAGTTGGAAGCTTTGAC
CCGTGTGGTGGTCCACATCCTGGACCAGAATGACCTGAAGCCTCTCTTCTCTCCACCTTCGTACAGAG
TCACCATCTCTGAGGACATGCCCCTGAAGAGCCCCATCTGCAAGGTGACTGCCACAGATGCTGATCTA
GGCCAGAATGCTGAGTTCTATTATGCCTTTAACACAAGGTCAGAGATGTTTGCCATCCATCCCACCAG
CGGTGTGGTCACTGTGGCTGGGAAGCTTAACGTCACCTGGCGAGGAAAGCATGAGGTCCAGGTGCTAG
CTGTGGACCGCATGCGGAAAATCTCTGAGGGCAATGGGTTTGGCAGCCTGGCTGCACTTGTGGTTCAT
GTGGAGCCTGCCCTCAGGAAGCCCCCAGCCATTGCTTCGGTCGTGGTGACTCCACCAGACAGCAATGA
TGGTACCACCTATGCCACTGTACTGGTCGATGCAAATAGCTCAGGAGCTGAAGTGGAGTCAGTGGAAG
TTGTTGGTGGTGACCCTGGAAAGCACTTCAAAGCCATCAAGTCTTATGCCCCGAGCAATGAGTTCAGT
TTGGTGTCTGTCAAAGACATCAACTGGATGGAGTACCTTCATGGGTTCAACCTCAGCCTCCAGGCCAG
GAGTGGGAGCGGCCCTTATTTTTATTCCCAGATCAGGGGCTTTCACCTACCACCTTCCAAACTGTCTT
CCCTCAAATTCGAGAAGGCTGTTTACAGAGTGCAGCTTAGTGAGTTTTCCCCTCCTGGCACCCGCGTG
GTGATGGTGAGAGTCACCCCAGCCTTCCCCAACCTGCAGTATGTTCTAAAGCCATCTTCAGAGAATGT
AGGATTTAAACTTAATGCTCGAACTGGGTTGATCACCACCACAAAGCTCATGGACTTCCACGACAGAG
CCCACTATCAGCTACACATCAGAACCTCACCGGGCCAGGCCTCCACCGTGGTGGTCATTGACATTGTG
GACTGCAACAACCATGCCCCCCTCTTCAACAGGTCTTCCTATGATGGTACCTTGGATGAGAACATCCC
TCCAGGCACCAGTGTTTTGGCTGTGACTGCCACTGACCGCGATCATGGGGAAAATGGATATGTCACCT
ATTCCATTGCTGGACCAAAAGCTTTGCCATTTTCTATTGACCCCTACCTGGGGATCATCTCCACCTCC
AAACCCATGGACTATGAACTCATGAAAAGAATTTATACCTTCCGGGTAAGAGCATCAGACTGGGGATC
CCCTTTTCGCCGGGAGAAGGAAGTGTCCATTTTTCTTCAGCTCAGGAACTTGAATGACAACCAGCCTA
TGTTTGAAGAAGTCAACTGTACAGGGTCTATCCGCCAAGACTGGCCAGTAGGGAAATCGATAATGACT
ATGTCAGCCATAGATGTGGATGAGCTTCAGAACCTAAAATACGAGATTGTATCAGGCAATGAACTAGA
GTATTTTGATCTAAATCATTTCTCCGGAGTGATATCCCTCAAACGCCCTTTTATCAATCTTACTGCTG
GTCAACCCACCAGTTATTCCCTGAAGATTACAGCCTCAGATGGCAAAAACTATGCCTCACCCACAACT
TTGAATATTACTGTGGTGAAGGACCCTCATTTTGAAGTTCCTGTAACATGTGATAAAACAGGGGTATT
GACACAATTCACAAAGACTATCCTCCACTTTATTGGGCTTCAGAACCAGGAGTCCAGTGATGAGGAAT
TCACTTCTTTAAGCACATATCAGATTAATCATTACACCCCACAGTTTGAGGACCACTTCCCCCAATCC
ATTGATGTCCTTGAGAGTGTCCCTATCAACACCCCCTTGGCCCGCCTAGCAGCCACTGACCCTGATGC
TGGTTTTAATGCCAAACTGGTCTATGTGATTGCAGATGGCAATGAGGAGGGCTGCTTTGACATAGAGC
TGGAGACAGGGCTGCTCACTGTAGCTGCTCCCTTGGACTATGAAGCCACCAATTTCTACATCCTCAAT
GTAACAGTATATGACCTGGGCACACCCCAGAAGTCCTCCTGGAAGCTGCTGACAGTCAATCTGAAAGA
CTGGAATGACAACGCACCCAGATTTCCTCCCGGTGGGTACCAGTTAACCATCTCGGAGGACACAGAAG
TTGGAACCACAATTGCAGAGCTGACAACCAAAGATGCTGACTCGGAAGACAATGGCAGGGTTCGCTAC
ACCCTGCTAAGTCCCACAGAGAAGTTCTCCCTCCACCCTCTCACTGGGGAACTGGTTGTTACAGGACA
CCTGGACCGCGAATCAGAGCCTCGGTACATACTCAAGGTGGAGGCCAGGGATCAGCCCAGCAAAGGCC
ACCAGCTCTTCTCTGTCACTGACCTCATAATCACATTGGAGGATGTCAACGACAACTCTCCCCAGTGC
ATCACAGAACACAACAGGCTGAAGGTTCCAGAGGACCTGCCCCCCGGCACTGTCTTGACATTTCTGGA
TGCCTCTGATCCTGACCTGGGCCCCGCAGGTGAAGTGCGATATGTTCTGATGGATGGCGCCCATGGGA
CCTTCCGGGTGGACCTGATGACAGGGGCGCTCATTCTGGAGAGAGAGCTGGACTTTGAGAGGCGAGCT
GGGTACAATCTGAGCCTGTGGGCCAGTGATGGTGGGAGGCCCCTAGCCCGCAGCACTCTCTGCCATGT
GGAGGTGATCGTCCTGGATCTGAATGAGAATCTCCACCCTCCCCACTTTGCCTCCTTCGTGCACCAGG
GCCAGGTGCAGGAGAACACCCCCTCGGGAACTCAGGTGATTGTAGTGGCTGCCCAGGACGATGACAGT
GGCTTGGATGGGGAGCTCCAGTACTTCCTGCGTGCTGGCACTGGACTCGCAGCCTTCAGCATCAACCA
AGATACAGGAATGATTCAGACTCTGGCACCCCTGGACCGAGAATTTGCATCTTACTACTGGTTGACGG
TATTAGCACTGGACAGGGGTTCTGTGCCCCTCTCTTCTGTAACTGAAGTCTACATCGAGGTTACCGAT
GCCAATCACAACCCACCCCAGATGTCCCAAGCTGTGTTCTACCCCTCCATCCAGGAGGATGCTCCCGT
GGGCACCTCTGTGCTTCAACTGGATGCCTGGGACCCAGACTCCAGCTCCAAAGGGAAGCTGACCTTCA
ACATCACCAGTGGGAACTACATGGGATTCTTTATGATTCACCCTGTTACAGGTCTCCTATCTACAGCC
CAGCAGCTGGACAGAGAGAACAAGGATGAACACATCCTGGAGGTGACTGTGCTGGACAATGGGGAACC
CTCACTGAAGTCCACCTCCAGGGTGGTGGTAGGCATCTTGGACGTCAATGACAATCCACCTATATTCT
CCCACAAGCTCTTCAATGTCCGCCTTCCAGAGAGGCTGAGCCCTGTGTCCCCTGGGCCTGTGTACAGG
CTCGTGGCTTCAGACCTGGATGAGGGTCTTAATGGCAGAGTCACCTACAGTATCGAGGACAGCTATGA
GGAGGCCTTCAGTATCGACCTGGTCACAGGTGTGGTTTCATCCAACAGCACTTTTACAGCTGGAGAGT
ACAACATCCTAACGATCAAGGCAACAGACAGTGGGCAGCCACCACTCTCAGCCAGTGTCCGGCTACAC
ATTGAGTGGATCCCTTGGCCCCGGCCGTCCTCCATCCCTCTGGCCTTTGATGAGACCTACTACAGCTT
TACGGTCATGGAGACGGACCCTGTGAACCACATGGTGGGGGTCATCAGCGTAGAGGGCAGACCCGGAC
TCTTCTCGTTCAACATCTCAGGTGGGGATAAGGACATGGACTTTGACATTGAGAAGACCACAGCCAGC
ATCGTCATTGCCAGGCCTCTTGATACCAGGAGAAGGTCGAACTATAACTTGACTGTTGAGGTGACAGA
TGGGTCCCGCACCATTGCCACACAGGTCCACATCTTCATGATTGCCAACATTAACCACCATCGGCCCC
AGTTTCTGGAAACTCGTTATGAAGTCAGAGTTCCCCAGGACACCGTGCCAGGGGTAGAGCTCCTGCGA
GTCCAGGCCATAGATCAAGACAAGGCCAAAACCCTCATCTATACCATACATGGCAGCCAAGACCCAGG
AAGTGCCAGCCTCTTCCAGCTGGACCCAAGCAGTGGTGTCCTGGTAACGGTGGGAAAATTGGACCTCG
GCTCGGGGCCCTCCCAGCACACACTGACAGTCATGGTCCGAGACCAGGAAATACCTATCAAGAGGAAC
TTCGTGTGGGTGACCATTCATGTGGACGATGGAAACCTCCACCCACCCCGCTTCACTCAGCTCCATTA
TGAGCCAAGTGTTCCTGACACCATAGCCCCCGGCACAGAGCTGCTGCAGGTCCGAGCCATGGATGCTG
ACCGGGGAGTCAATGCTGAGGTCCACTACTCCCTCCTGAAAGGGAACAGCGAAGGTTTCTTCAACATC
AATGCCCTGCTAGGCATCATTACTCTAGCTCAAAAGCTTGATCAGGCAAATCATGCCCCACATACTCT
GACAGTGAAGGCAGAAGATCAAGGCTCCCCACAATGGCATGACCTGGCTACAGTGATCATTCATGTCT
ATCCCTCAGATAGGAGTGCCCCCATCTTTTCAAAATCTGAGTACTTTCTACAGATCCCTCAATCAATC
CCTGTTGGTTCCCCAATCCTCCTTGTCTCTGCTATGAGCCCCTCTGAAGTTACCTATGAGTTAAGAGA
GGGAAATAAGGATGGAGTCTTCTCTATGAACTCATATTCTGGCCTTATTTCCACCCACAAGAAATTGG
ACCATGAGAAAATCTCGTCTTACCAGCTGAAAATCCQAGGCAGCAATATGGCAGCTGCATTTACTGAT
GTCATGGTGGTGGTTGACATAATTGATGAAAATGACAATGCTCCTATGTTCTTAAAGTCAACTTTTGT
GCCCCAAATTAGTGAAGCACCTCCACTGTATAGCATGATCATGGATAAAAACAACAACCCCTTTGTGA
TTCATGCCTCTGACACTGACAAACAAGCTAATTCCTTGTTGGTCTATAAAATTTTGGAGCCGGACGCC
TTGAAGTTTTTCAAAATTGATCCCAGCATGGGAACCCTAACCATTGTATCAGAGATGGATTATGAGAG
CATGCCCTCTTTCCAATTCTGTGTCTATGTCCATGACCAAGGAAGCCCTGTATTATTTGCACCCAGAC
CTGCCCAAGTCATCATTCATGTCAGAGATGTGAATGATTCCCCTCCCAGATTCTCAGAACAGATATAT
GAGGTAGCAATAGTCGGGCCTATCCATCCAGGCATGGAGCTTCTCATGGTGCGGGCCAGCGATGAAGA
CTCAGAAGTCAATTATAGCATCAAAACTGGCAATGCTGATGAAGCTGTTACCATCCATCCTGTCACTG
GTAGCATATCTGTGCTGAATCCTGCTTTCCTGGGACTCTCTCGGAAGCTCACCATCAGGGCTTCTGAT
GGCTTGTATCAAGACACTGCGCTCGTAAAAATTTCTTTGACCCAAGTGCTTGACAAAAGCTTGCAGTT
TGATCAGGATGTCTACTGGGCAGCTGTGAAGGAGAACTTGCAGGACAGAAAGGCACTGGTGATTCTTG
GTGCCCAGGGCAATCATTTGAATGACACCCTTTCCTACTTTCTCTTGAATGGCACAGATATGTTTCAT
ATGGTCCAGTCAGCAGGTGTGTTGCAGACAAGAGGTGTGGCGTTTGACCGGGAGCAGCAGGACACTCA
TGAGTTGGCAGTGGAAGTGAGGGACAATCGGACACCTCAGCCGGTGGCTCAGGGTTTGGTCAGAGTCT
CTATTGAGGATGTCAATGACAATCCCCCCAAATTTAAGCATCTGCCCTATTACACAATCATCCAAGAT
GGCACAGAGCCAGGGGATGTCCTCTTTCAGGTATCTGCCACTGATCACGACTTGGGGACAAATGGGGC
TGTTACATATGAATTTGCAGAAGATTACACATATTTCCGAATTGACCCCTATCTTGGGGACATATCAC
TCAAGAAACCCTTTGATTATCAAGCTTTAAATAAATATCACCTCAAAGTCATTGCTCGGGATGGAGGA
ACGCCATCCCTCCAGAGTGAGGAAGAGGTACTTGTCACTGTGAGAAATAAATCCAACCCACTGTTTCA
GAGTCCTTATTACAAAGTCAGAGTACCTGAAAATATCACCCTCTATACCCCAATTCTCCACACCCAGC
CCCCGAGTCCAGAGGGACTCCGGCTCATCTACAACATTCTGGAGGAAGAACCCTTGATGCTGTTCACC
ACTGACTTCAAGACTGGTGTCCTAACAGTAACAGGGCCTTTGGACTATGAGTCCAAGACCAAACATGT
GTTCACAGTCAGAGCCACGGATACAGCTCTGGGGTCATTTTCTGAAGCCACAGTGGAAGTCCTAGTGG
AGCATGTCAATGATAACCCTCCCACTTTTTCCCAATTGGTCTATACCACTTCCATCTCAGAAGGCTTG
CCTGCTCAGACCCCTGTGATCCAACTGTTGGCTTCTGACCAGGACTCAGGGCGGAACCGTGACGTCTC
TTATCAGATTGTGGAGGATGGCTCAGATGTTTCCAACTTCTTCCAGATCAATGGGAGCACAGGGGAGA
TGTCCACAGTTCAAGAACTGGATTATGAAGCCCAACAACACTTTCATGTGAAAGTCAGGGCCATGGAT
AAAGGAGATCCCCCACTCACTGGTGAAACCCTTGTGGTTGTCAATGTGTCTGATATCAATGACAACCC
CCCAGAGTTCAGACAACCTCAATATCAAGCCAATGTCACTGAACTGGCAACCTGTGGACACCTGGTTC
TTAAAGTCCAGGCTATTGACCCTGACAGCAGAGACACCTCCCGCCTGGAGTACCTGATTCTTTCTGGC
AATCAGGACAGGCACTTCTTCATTAACAGCTCATCGCGAATAATTTCTATGTTCAACCTTTCCAAAAA
GCACCTGGACTCTTCTTACAATTTGAGGQTAGGTGCTTCTGATGGACTCTTCCGAGCAACTGTGCCTG
TGTACATCAACACTACAAATGCCAACAACTACAGCCCAGAGTTCCAGCAGCACCTTTATGAGGCAGAA
TTAGCAGAGAATGCAATGGTTGGAACCAAGGTCATTGATTTGCTAGCCATAGACAAAGATAGTGGTCC
CTATCGCACTATAGATTATACTATCATCAATAAACTAGCAAGTGAGAAGTTCTCCATAAACCCCAATG
GCCAGATTGCCACTCTGCAGAAACTGGATCGGGAAAATTCAACAGAGAGAGTCATTGCTATTAAGGTC
ATGGCTCGGGATGGAGGAGGAAGAGTAGCCTTCTGCACGGTGAAGATCATCCTCACAGATGAAAATGA
CAACCCCCCACAGTTCAAACCATCTGAGTACACAGTATCCATTCAATCCAATGTCAGTAAAGACTCTC
CGGTTATCCAGGTGTTGGCCTATGATGCAGATGAAGGTCAGAACGCAGATGTCACCTACTCAGTGAAC
CCAGAGGACCTACTTAAAGATGTCATTGAAATTAACCCAGTCACTGCTGTCGTCAAGGTGAAAGACAG
CCTGGTGGGATTCGAAAATCAGACCCTTGACTTCTTCATCAAAGCCCAAGATGGAGGCCCTCCTCACT
GGAACTCTCTGGTGCCAGTACGACTTCACGTGGTTCCTAAAAAAGTATCCTTACCGAAATTTTCTGAA
CCTTTGTATACTTTCTCTGCACCTGAAGACCTTCCAGAGGGGTCTGAAATTGGGATTGTTAAAGCAGT
GGCAGCTCAAGATCCAGTCATCTACAGTCTAGTGCGGGGCACTACACCTGACAGCAACAAGGATGGTG
TCTTCTCCCTAGACCCAGACACAGGGGTCATAAAGGTGAGGAAGCCCATGGACCACGAATCCACCAAA
TTGTACCAGATTGATGTGATGGCACATTGCCTTCAGAACACTGATGTGGTGTCCTTGGTCTCTGTCAA
CATCCAAGTGGGAGACGTCAATGACAATAGGCCTGTATTTGACGCTGATCCATATAAGGCTGTCCTCA
CTGAGAATATGCCAGTGGGGACCTCACTCATTCAAGTGACTGCCATTGACAAGGACACTGGGAGAGAT
GGCCAGGTGAGCTACAGGCTGTCTGCAGACCCTGGTAGCAATGTCCATGAGCTCTTTGCCATTGACAG
TGAGAGTCGTTGGATCACCACACTCCAGGAACTTGACTGTGAGACCTGCCAGACTTATCATTTTCATG
TGGTGGCCTATGACCACGGACAGACCATCCAGCTATCCTCTCAGGCCCTCGTTCACGTCTCCATTACA
GATGAGAATGACAATGCTCCCCGATTTGCTTCTGAAGAGTACAGAGGATCTGTGGTTGAGAACAGTGA
GCCTGGCGAACTGGTGGCCACTCTAAAGACCCTGGATGCTGACATTTCTGAGCAGAACAGGCAGGTCA
CCTGCTACATCACAGACGGAGACCCCCTGGGCCAGTTTGGCATCAGCCAAGTTGGAGATGAGTGGAGG
ATTTCCTCAAGGAAGACCCTGGACCCCGAGCATACAGCCAAGTACTTGCTCAGAGTCACAGCATCTGA
TGGCAAGTTCCAGGCTTCGGTCACTGTGGAGATCTTTGTCCTGGACGTCAATGATAACAGCCCACAGT
GTTCACAGCTTCTCTATACTGGCAAGGTTCATGAAGATGTATTTCCAGGACACTTCATTTTGAAGCTT
TCTGCCACAGACTTGGACACTGATACCAATCCTCAGATCACATATTCTCTGCATGGCCCTGGGGCGCA
TGAATTCAAGCTGGATCCTCATACAGGGGAGCTGACCACACTCACTGCCCTAGACCGAGAAAGGAAGG
ATGTGTTCAACCTTGTTGCCAAGGCGACCGATGGAGGTGGCCGATCGTGCCAGGCAGACATCACCCTC
CATGTGGAGGATGTGAATGACAATGCCCCGCGGTTCTTCCCCAGCCACTGTGCTGTGGCTGTCTTCGA
CAACACCACAGTGAAGACCCCTGTGGCTGTAGTATTTGCCCGGGATCCCGACCAAGGCGCCAATGCCC
AGGTGGTTTACTCTCTGCCGGATTCAGCCGAAGGCCACTTTTCCATCGACGCCACCACGGGCGTGATC
CGCCTGGAAAAGCCGCTGCAGGTCAGGCCCCAGGCACCACTGGAGCTCACGGTCCGTGCCTCTGACCT
GGGCACCCCAATACCGCTGTCCACGCTGGGCACCGTCACAGTCTCGGTGGTGGGCCTAGAAGACTACC
TGCCCGTGTTCCTGAACACCGAGCACACCGTGCAGGTGCCCGAGGACGCCCCACCTGGCACGGAGGTG
CTGCAGCTGGCCACCCTCACTCCCCCGGCCGCAGAGAAGACCGGCTACCGCGTGGTCAGCGGGAACGA
GCAAGGCAGGTTCCGCCTGGATGCTCGCACAGGGATCCTGTATGTCAACGCAAGCCTGGACTTTGAGA
CAAGCCCCAAGTACTTCCTGTCCATTGAGTGCAGCCGGAAGAGCTCCTCTTCCCTCAGTGACGTGACC
ACAGTCATGGTCAACATCACTGATGTCAATCAACACCGGCCCCAATTCCCCCAAGATCCATATAGCAC
AAGGGTCTTAGAGAATGCCCTTGTGGGTGACGTCATCCTCACGGTATCAGCGACTGATGAAGATGGAC
CCCTAAATAGTGACATTACCTATAGCCTCATAGGAGCGAACCAGCTTGGGCACTTCACCATTCACCCC
AAAAAGGGGGAGCTACAGGTGGCCAAGGCCCTCGACCGGGAACAGGCCTCTAGTTATTCCCTGAACCT
CCGAGCCACAGACAGTGGGCAGCCTCCACTGCATGAGGACACAGACATCGCTATCCAAGTGGCTGATG
TCAATGATAACCCACCGAGATTCTTCCAGCTCAACTACAGCACCACTGTCCAGGAGAACTCCCCCATT
GGCAGCAAAGTCCTGCAGCTGATCCTGAGTGACCCAGATTCTCCAGAGAATGGCCCCCCCTACTCGTT
TCGAATCACCAAGGCGAACAACGGCTCTGCCTTCCGAGTGACCCCGGATGGATGGCTGGTGACTGCTG
AGGGCCTAAGCAGGAGGGCTCAGGAATGGTATCAGCTTCAGATCCAGGCGTCAGACAGTGGCATCCCT
CCCCTCTCGTCTTTGACGTCTGTCCGTGTCCATGTCACAGAGCAGAGCCACTATGCACCTTCTGCTCT
CCCACTGGAGATCTTCATCACTGTTGGAGAGGATGAGTTCCAGGGTGGCATGGTGGGTAAGATCCATG
CCACAGACCGAGACCCCCAGGACACGCTGACCTATAGCCTGGCAGAAGAGGAGACCCTGGGCAGGCAC
TTCTCAGTCGGTGCGCCTGATGGCAAGATTATCGCCGCCCAGGGCCTGCCTCGTGGCCACTACTCGTT
CAACGTCACGGTCAGCGATGGGACCTTCACCACGACTGCTGGGGTCCATGTGTACGTGTGGCATGTGG
GGCAGGAGGCTCTGCAGCAGGCCATGTGGATGGGCTTCTACCAGCTCACCCCCGAGGAGCTGGTGAGT
GACCACTGGCGGAACCTGCAGAGGTTCCTCAGCCATAAGCTGGACATCAAACGGGCTAACATTCACTT
GGCCAGCCTCCAGCCTGCAGAGGCCGTGGCTGGTGTGGATGTGCTCCTGGTCTTTGAGGGGCATTCTG
GAACCTTCTACGAGTTTCAGGAGCTAGCATCCATCATCACTCACTCAGCCAAGGAGATGGAGCATTCA
GTGGGGGTTCAGATGCGGTCAGCTATGCCCATGGTGCCCTGCCAGGGGCCAACCTGCCAGGGTCAAAT
CTGCCATAACACAGTGCATCTGGACCCCAAGGTTGGGCCCACGTACAGCACCGGCCAGGCNTTAACAT
CCCTAACCCCGCGGCACCACCTGCAGAGGAGCTGCTCCTGCAATGGTACTGCTACAAGGTTCAGTGGT
CAGAGCTATGTGCGGTACAGGGTCCCAGCGGCTCGGAACTGGCACATCCATTTCTATCTGAAAACACT
CCAGCCACAGGCCATTCTTCTATTCACCAATGAAACAGCGTCCGTCTCCCTGAAGGGCTTTGAAGGCT
GCCTGGATGCTGTCGTGGTCAACGAAGAGGCTCTAGATCTGCTCGCCCCTGGCAAGACGGTGGCAGGC
TTGCTGGAGACACAACCCCTCACCCAGTGCTGCCTCCACAGTGACTACTGCAGCCAGAACACATGCCT
CAATGGTGGGAAGTGCTCATGGACCCACGGGGCAGGCTATGTCTGCAAATGTCCCCCACAGTTCTCTG
GGAAGCACTGTGAACAAGGAAGGGAGAACTGTACTTTTGCACCCTGCCTGGAAGGTGGAACTTGCATC
CTCTCCCCCAAAGGAGCTTCCTGTAACTGCCCTCATCCTTACACAGGAGACAGGTGTGAAATGGAGGC
GAGGGGTTGTTCAGAAGGACACTGCCTAGTCACTCCCGAGATCCAAAGGGGGGACTGGGGGCAGCAGC
AGTTACTGATCATCACAGTGGCCGTGGCGTTCATTATCATAAGCACTGTCGGGCTTCTCTTCTACTGC
CGCCGTTGCAAGTCTCACAAGCCTGTGGCCATGGAGGACCCAGACCTCCTGGCCAGGAGTGTTGGTGT
TGACACCCAAGCCATGCCTGCCATCGAGCTCAACCCATTGAGTGCCAGCTCCTGCAACAACCTCAACC
AACTGGAACCCAGCAAGGCCTCTGTTCCAAATGAACTCGTCACATTTGGACCCAATTCTAACCAACGG
CCAGTGGTCTGCAGTGTGCCCCCCAGACTCCCGCCAGCTGCGGTCCCTTCCCACTCTGACAATGGGCC
TGTCATTAAGAGAACCTGGTCCAGTGAGGAGATGGTGTACCCTGGCGGAGCCATGGTCTGCCCCCCTA
CTTACTCCAGGAACGAACGCTGGGAATACCCCCACTCCGAAGTGACTCAGGGCCCTCTGCCGCCCTCG
GCTCACCGCCACTCAACCCCAGTCGTGATGCCAGAGCCTAATGGCCTCTATGGGGGCTTCCCCTTCCC
CCTGGAGATGGAAAACAAGCGGGCACCTCTCCCACCCCGTTACAGCAACCAGAACCTGGAAGATCTGA
TGCCCTCTCGGCCCCCTAGTCCCCGGGAGCGCCTGGTTGCCCCCTGTCTCAATGAGTACACGGCCATC
AGCTACTACCACTCGCAGTTCCGGCACGGAGGGGGAGGGCCCTGCCTGGCAGACGGGGGCTACAAGGG
GGTGGGTATGCGCCTCAGCCGAGCTGGCCCCTCTTATGCTGTCTGTGAGGTGGACGGGGCACCTCTTG
CAGGCCAGGGCCACCCCCCCGTGCCCCCCAACTATGAGGGCTCTGACATGGTGGAGAGTGATTATGGC
AGCTGTGAGGAGGTCATGTTCTAGCTTCCCATTCCCAGAGCAAGGCAGGCGGGAGGCCAAGGACTGGA
CTTGGCTTATTTCTTCCTGTCTCGTACQGGCTGAGTTCAGTGTGGCTGGGAGAGTGCGAGGGAAGCCC
TCAGCCCAGGCTGTTGTCCCTTCAAATGTGCTCTTCCAATCCCCCACCTAGTCCCTGAGGGTGGAGGC
AAGCTGAGGATAGAGCTCCAGAAACAGCACTAGGGTCCCAGGAGAGGGGCATTTCTAGAGCAGTGACC
CTCGAAAACCAGGAACAATTGACTCCCGCGGTGGGCGAGAGACAGGAGGGCTCCCTCATCTGCCGGCT
CTCAGTCCCCGGGGCAGAGCCTGATTGACTGTGCTCGCTCAACTTCACCAACATGCATTCTCATACCT
GCCCACAGCTCCATTTTGCAGGCAGGCAGGTTGGTGCCTCACAGACAACCACTACGCGGGCCGTACAG
AGGAGCTCTAGAGGGCTGCGTGGCATCCTCCTAGGGGCTGAGAGGTGAGCAGCAGGGGAGCGGGCACA
GTCCCCTCTGCCCCTGCCTCAGTCGAGCACTCACTGTGTCTTTGTCAAGTGTCTGCTCCACGTCACGC
ACTCTGCTTTGCACCGGGGAGAAAATGGTGATGCACGGCAACAAGCACTCCGAGGAGCACCACCAGGC
CTCGGGCCCCAGAGGTCCCACTCCTCAGCCTACACGCAGAGGAACGGGCCCACCTCAGAGTCACACCA
CTGGCTGCCAGTCAGGGCCTGCCACGAGTCTACACAGCTCTGAACCTTCTTTGTTAAAGAATTCAGAC
CTCATGGAACTCTGGGTTCTTCATCCCAAGTTTCCCAGGCACTTTTGGCCAAAGGAAGGAAGGAACTA
ATTCTTCATTTTAAAAATTCTTAGGCACTTTTTGACCTTGCTGTCTGGATGAGTTTCCTCAATGGGAT
TTTTCTTCCCTAGACACAAGGAAGTCTGAACTCCTATTTAGGGCCGGTTGGAAGCAGGGAGCTGGACC
GCAGTGTCCAGGCTGGACACCTGCCATTGCCTCCTCTCCATTGCAGACGCCTGCCCATCAAGTATTAC
TCCGGCGACTCAACCCTATGCATGGAGGGTCAATGTGGGCACATGTCTACACATGTGGGTGCCCATGG
ATAGTACGTGTGTACACATGTGTAGACTGTATGTAGCCAGGAGTGGTGGGGACCAGAAGCCTCTGTGG
CCTTTGGTGACCTCACCACTCCCTCCCACCCAGTCCCTCCCTCTGGTCCACTGCCTTTTCATATGTGT
TGTTTCTGGAGACAGAAGTCAAAAGGAAGAGCAGTCCAGCCTTGCCCACAGGGCTGCTGCTTCATCCG
AGAGGGAGATGTGTGGGCGACAGCCAATTTGTGTCAGTGGTTTGTGGCTGTGTGTGTCACTGTGAGTG
TGAGTGACAGATACATAGTTTCATTGGTCATTTTTTTTTTAACAATAAAGTATCTTTTTTTACTGTT

| | | | |
|---|---|---|---|
| NOV6n, CG51923-03 | | | |
| Protein Sequence | SEQ ID NO: 66 | 4264 aa | MW at 469871.7kD |

MTIALLGFAIFLLHCATCEKPLEGILSSSAWHFTHSHYNATIYENSSPKTYVESFEKMGIYLAEPQWA
VRYRIISGDVANVFKTEEYVVGNFCFLRIRTKSSNTALLNREVRDSYTLIIQATEKTLELEALTRVVV
HILDQNDLKPLFSPPSYRVTISEDMPLKSPICKVTATDADLGQNAEFYYAFNTRSEMFAIHPTSGVVT
VAGKLNVTWRGKHELQVLAVDRMRKISEGNGFGSLAALVVHVEPALRKPPAIASVVVTPPDSNDGTTY
ATVLVDANSSGAEVESVEVVGGDPGKHFKAIKSYARSNEFSLVSVKDINWMEYLHGFNLSLQARSGSG
PYFYSQIRCFHLPPSKLSSLKFEKAVYRVQLSEFSPPGSRVVMVRVTPAFPNLQYVLKPSSENVGFKL
NARTGLITTTKLMDFHDRAHYQLHIRTSPGQASTVVVIDIVDCNNHAPLFNRSSYDGTLDENIPPGTS
VLAVTATDRDHGENGYVTYSIAGPKALPFSIDPYLGIISTSKPMDYELMKRIYTFRVRASDWGSPFRR
EKEVSIFLQLRNLNDNQPMFEEVNCTGSIRQDWPVGKSIMTMSAIDVDELQNLKYEIVSGNELEYFDL
NHFSGVISLKRPFINLTAGQPTSYSLKITASDGKNYASPTTLNITVVKDPHFEVPVTCDKTGVLTQFT
KTILHFIGLQNQESSDEEFTSLSTYQINHYTPQFEDHFPQSIDVLESVPINTPLARLAATDPDAGFNG
KLVYVIADGNEEGCFDIELETGLLTVAAPLDYEATNFYILNVTVYDLGTPQKSSWKLLTVNVKDWNDN
APRFPPCGYQLTISEDTEVGTTIAELTTKDADSEDNGRVRYTLLSPTEKFSLHPLTGELVVTGHLDRE
SEPRYILKVEARDQPSKGHQLFSVTDLIITLEDVNDNSPQCITEHNRLKVPEDLPPGTVLTFLDASDP
DLGPAGEVRYVLMDGAHGTFRVDLMTGALILERELDFERRAGYNLSLWASDGGRPLARRTLCHVEVIV
LDVNENLHPPHFASFVHQGQVQENSPSGTQVIVVAAQDDDSGLDGELQYFLRAGTGTAAFSINQDTGM
IQTLAPLDREFASYYWLTVLAVDRGSVPLSSVTEVYIEVTDANDNPPQMSQAVFYPSIQEDAPVGTSV
LQLDAWDPDSSSKGKLTFNITSGNYMGFFMIHPVTGLLSTAQQLDRENKDEHILEVTVLDNGEPSLKS
TSRVVVGILDVNDNPPIFSHKLFNVRLPERLSPVSPGPVYRLVASDLDEGLNGRVTYSIEDSYEEAFS
IDLVTGVVSSNSTFTAGEYNILTIKATDSGQPPLSASvRLHIEWIPWPRPSSIPLAFDETYYSFTVME
TDPVNHMVGVISVEGRPGLFWFNISGGDKDMDFDIEKTTGSIVIARPLDTRRRSNYNLTVEVTDGSRT
IATQVHIFMIANINHHRPQFLETRYEVRVPQDTVPCVELLRVQAIDQDKGKSLIYTIHGSQDPGSASL
FQLDPSSGVLVTVGKLDLGSGPSQHTLTVMVRDQEIPIKRNFVWVTIHVEDGNLHPPRFTQLHYEASV
PDTIAPGTELLQVRAMDADRCVNAEVHYSLLKGNSEGFFNINALLCIITLAQKLDQANHAPHTLTVKA
EDQGSPQWHDLATVIIHVYPSDRSAPIFSKSEYFVEIPESIPVGSPILLVSAMSPSEVTYELREGNKD
GVFSMNSYSGLISTQKKLDHEKISSYQLKIRGSNMAGAFTDVMVVVDIIDENDNAPMFLKSTFVGQIS
EAAPLYSMIMDKNNNPFVIHASDSDKEANSLLVYKILEPEALKFFKIDPSMGTLTIVSEMDYESMPSF
QFCVYVHDQGSPVLFAPRPAQVIIHVRDVNDSPPRFSEQIYEVAIVGPIHPGMELLMVRASDEDSEVN
YSIKTGNADEAVTIHPVTGSISVLNPAFLGLSRKLTIRASDGLYGDTALVKISLTGVLDKSLGFDGDV
YWAAVKENLQDRKALVILGAQGNHLNDTLSYFLLNGTDMFHMVQSAGVLQTRGVAFDREQQDTHELAV
EVRDNRTPQRVAQGLVRVSIEDVNDNPPKFKHLPYYTIIQDGTEPGDVLFQVSATDEDLGTNGAVTYE
FAEDYTYFRIDPYLGDISLKKPFDYQALNKYHLKVIARDGGTPSLQSEEEVLVTVRNKSNPLFQSPYY
KVRVPENITLYTPILHTQARSPEGLRLIYNIVEEEPLMLFTTDFKTGVLTVTGPLDYESKTKIWFTVR
ATDTALGSFSEATVEVLVEDVNDNPPTFSQLVYTTSISEGLPAQTPVIQLLASDQDSGRNRDVSYQIV
EDCSDVSKFFQINGSTGEMSTVQELDYEAQQHFHVKVRAMDKGDPPLTGETLVVVNVSDINDNPPEFR
QPQYEANVSELATCGHLVLKVQAIDPDSRDTSRLEYLILSGNQDRHFFINSSSGIISMFNLCKKHLDS
SYNLRVGASDGVFRATVPVYINTTNANKYSPEFQQHLYEAELAENAMVGTKVIDLLAIDKDSGPYGTI
DYTIINKLASEKFSINPNGQIATLQKLDRENSTERVIAIKVMARDCGGRVAFCTVKIILTDENDNPPQ
FKASEYTVSIQSNVSKDSPVIQVLAYDADEGQNADVTYSVNPEDLVKDVIEINPVTGVVKVKDSLVGL
ENQTLDFFIKAQDGGPPHWNSLVPVRLQVVPKKVSLPKFSEPLYTFSAPEDLPEGSEIGIVKAVAAQD
PVIYSLVRGTTPESNKDGVFSLDPDTGVIKVRKPMDHESTKLYQIDVMAHCLQNTDVVSLVSVNIQVG
DVNDNRPVFEADPYKAVLTENMPVGTSVIQVTAIDKDTGRDGQVSYRLSADPGSNVHELFAIDSESGW
ITTLQELDCETCQTYHFNVVAYDHGQTIQLSSQALVQVSITDENDNAPRFASEEYRGSVVENSEPGEL
VATLKTLDADISEQNRQVTCYITEGDPLGQFGISQVGDEWRISSRKTLDREHTAKYLLRVTASDGKFQ
ASVTVEIFVLDVNDNSPQCSQLLYTGKVHEDVFPGHFILKVSATDLDTDTNAQITYSLHGPGAHEFKL
DPHTGELTTLTALDRERKDVFNLVAKATDGGGRSCQADITLHVEDVNDNAPRFFPSHCACAVFDNTTV
KTPVAVVFARDPDQGANAQVVYSLPDSAEGHFSIDATTGVIRLEKPLQVRPQAPLELTVRASDLGTPI
PLSTLGTVTVSVVGLEDYLPVFLNTEHSVQVPEDAPPGTEVLQLATLTRPGAEKTGYRVVSGNEQGRF
RLDARTGILYVNASLDFETSPKYFLSIECSRKSSSSLSDVTTVMVNITDVNEHRPQFPQDPYSTRVLE
NALVGDVILTVSATDEDGPLNSDITYSLIGGNQLGHFTIHPKKGELQVAKALDREQASSYSLKLRATD
SGQPPLHEDTDIAIQVADVNDNPPRFFQLNYSTTVQENSPIGSKVLQLILSDPDSPENGPPYSFRITK
GNNGSAFRVTPDGWLVTAEGLSRRAQEWYQLQIQASDSGIPPLSSLTSVRVHVTEQSHYAPSALPLEI
FITVGEDEFQGGMVGKIHATDRDPQDTLTYSLAEEETLGRHFSVGAPDGKIIAAQGLPRGHYSFNVTV
SDGTFTTTAGVHVYVWHVGQEALQQAMWMGFYQLTPEELVSDHWRNLQRFLSHKLDIKRANIHLASLQ
PAEAVAGVDVLLVFEGHSGTFYEFQELASIITHSAKEMEHSVGVQMRSAMPMVPCQGPTCQGQICHNT
VHLDPKVGPTYSTGQALTSLTPRHHLQRSCSCNGTATRFSGQSYVRYRVPAARNWHIHFYLKTLQPQA
ILLFTNETASVSLKGFEGCLDAVVVNEEALDLLAPGKTVACLLETQALTQCCLHSDYCSQNTCLNGGK
CSWTHGAGYVCKCPPQFSGKHCEQGRENCTFAPCLEGGTCILSPKGASCNCPHPYTGDRCEMEARGCS
EGHCLVTPEIQRGDWGQQELLIITVAVAFIIISTVGLLFYCRRCKSHKPVAMEDPDLLARSVGVDTQA
MPAIELNPLSASSCNNLNQLEPSKASVPNELVTFGPNSKQRPVVCSVPPRLPPAAVPSHSDNGPVIKR
TWSSEEMVYPGGAMVWPPTYSRNERWEYPHSEVTQGPLPPSAHRHSTPVVMPEPUGLYGGFPFPLEME
NKRAPLPPRYSNQNLEDLMPSRPPSPRERLVAPCLNEYTAISYYHSQFRQGGGGPCLADGGYKGVGMR
LSRAGPSYAVCEVEGAPLAGQGQPRVPPNYEGSDMVESDYGSCEEVMF

A ClustalW comparison of the above protein sequences yields the following relationships between the NOV6 sequences. In comparison to NOV6a, CG51923-01, NOV6n is 4264 amino acid residues having the following sequence changes: amino acids 3754 to 3759, -ARLSI becomes GQALTS; A3789V; amino acids 3900 to 3907, HSSSNVSQ are deleted; P4117L; E4160G. NOV6m corresponds to amino acid residues 2802 to 4023 of NOV6a with the following sequence changes: V3033A; L3514S; G3591D; M3631I. NOV6l corresponds to amino acid residues 1561 to 2233 of NOV6a. NOV6k corresponds to amino acids 1143 to 1733 of NOV6a with the following sequence changes: Y1181H; Y1287D; N1303S. Both NOV6b and NOV6c correspond to amino acids 2561 to 3233 of NOV6a with NOV6b having an amino acid change Q2991H. NOV6e and NOV6f correspond to amino acids 1 to 659 of NOV6a and NOV6e has-an amino acid change R574C. NOV6g corresponds to amino acid residues 19-659 of NOV6a with an amino acid change of R574C. NOV6h and NOV6i correspond to amino acids 154-659 of NOV6a and NOV6h has an amino acid change R574C. NOV6d corresponds to amino acids 3559 to 4043 of NOV6a with an amino acid change M3631I. NOV6j corresponds to NOV6a amino acids 570 to 1233 with A1100V and Y1181H amino acid changes.

Further analysis of the NOV6a protein yielded the following properties shown in Table 6B.

TABLE 6B
Protein Sequence Properties NOV6a

| SignalP | Cleavage site between residues 19 and 20 | | | | |
|---|---|---|---|---|---|
| analysis: | PSG: a new signal peptide prediction method | | | | |
| PSORT II | N-region: length 0; pos. chg 0; neg. chg 0 | | | | |
| analysis: | H-region: length 18; peak value 11.25 | | | | |
| | PSG score: 6.85 | | | | |
| | GvH: von Heijne's method for signal seq. recognition | | | | |
| | GvH score (threshold: −2.1): −0.05 | | | | |
| | possible cleavage site: between 18 and 19 | | | | |
| | >>> Seems to have a cleavable signal peptide (1 to 18) | | | | |
| | ALOM: Klein et al's method for TM region allocation | | | | |
| | Init position for calculation: 19 | | | | |
| | Tentative number of TMS(s) for the threshold 0.5: 1 | | | | |
| | Number of TMS(s) for threshold 0.5: 1 | | | | |
| | INTEGRAL | Likelihood = | −10.40 | Transmembrane | 4049-4065 |

| PERIPHERAL | Likelihood = | 1.01 (at 3195) | |
|---|---|---|---|
| ALOM score: −10.40 (number of TMSs: 1) | | | |
| MTOP: Prediction of membrane topology (Hartmann et al.) | | | |
| Center position for calculation: 9 | | | |
| Charge difference: −1.5 C(−0.5)-N(1.0) | | | |
| N >= C: N-terminal side will be inside | | | |
| >>> membrane topology: type 1a (cytoplasmic tail 4066 to 4349) | | | |
| MITDISC: discrimination of mitochondrial targeting seq | | | |
| R content: | 0 | Hyd Moment(75): | 0.99 |
| Hyd Moment(95): | 1.82 | G content: | 1 |
| D/E content: | 1 | S/T content: | 2 |

Score: −6.08
Gavel: prediction of cleavage sites for mitochondrial preseq
cleavage site motif not found
NUCDISC: discrimination of nuclear localization signals
pat4: none
pat7: PFRREKE (4) at 541
pat7: PLDTRRR (3) at 1407
bipartite: none
content of basic residues: 8.0%
NLS Score: 0.13
KDEL: ER retention motif in the C-terminus: none
ER Membrane Retention Signals: none
SKL: peroxisomal targeting signal in the C-terminus: none
PTS2: 2nd peroxisomal targeting signal: found
KLASGVPQL at 3821
VAC: possible vacuolar targeting motif: none
KNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 4066
LL at 4086
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
44.4%: endoplasmic reticulum
22.2%: Golgi
22.2%: extracellular, including cell wall
11.1%: plasma membrane
>> prediction for CG51923-01 is end (k = 9)

A search of the NOV6a protein against the Geneseq database, a proprietary database that contains sequences published in patents and patent publication, yielded several homologous proteins shown in Table 6C.

TABLE 6C
Geneseq Results for NOV6a

| | | NOV6a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAO26792 | Human cadherin (CAD) protein, | 1 . . . 4349 | 4349/4349 (100%) | 0.0 |
| | SEQ ID No 15 - Homo sapiens, | 1 . . . 4349 | 4349/4349 (100%) | |
| | 4349 aa. [WO200299042-A2, 12 | | | |
| | DEC. 2002] | | | |
| AAU79940 | Human protocadherin Fat 2 | 1 . . . 4349 | 4349/4349 (100%) | 0.0 |
| | (FAT2) protein NOV2 - Homo | 1 . . . 4349 | 4349/4349 (100%) | |
| | sapiens, 4349 aa. | | | |
| | [WO200229038-A2, 11 APR. 2002] | | | |
| ABB97540 | Novel human protein SEQ ID | 1 . . . 4349 | 4346/4349 (99%) | 0.0 |
| | NO: 808 - Homo sapiens, 4349 | 1 . . . 4349 | 4347/4349 (99%) | |
| | aa. [WO200222660-A2, 21 MAR. 2002] | | | |
| ABB97541 | Novel human protein SEQ ID | 1 . . . 3821 | 3819/3821 (99%) | 0.0 |
| | NO: 809 - Homo sapiens, 4263 | 1 . . . 3821 | 3819/3821 (99%) | |
| | aa. [WO200222660-A2, 21 MAR. 2002] | | | |
| AAO26791 | Human cadherin (CAD) protein, | 26 . . . 4033 | 1844/4089 (45%) | 0.0 |
| | SEQ ID No 14 - Homo sapiens, | 27 . . . 4100 | 2647/4089 (64%) | |
| | 4590 aa. [WO200299042-A2, 12 | | | |
| | DEC. 2002] | | | |

In a BLAST search of public sequence databases, the NOV6a protein was found to have homology to the proteins shown in the BLASTP data in Table 6D.

TABLE 6D
Public BLASTP Results for NOV6a

| | | NOV6a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9NYQ8 | Protocadherin Fat 2 precursor | 1 . . . 4349 | 4349/4349 (100%) | 0.0 |
| | (hFat2) (Multiple epidermal | 1 . . . 4349 | 4349/4349 (100%) | |
| | growth factor-like domains 1) - | | | |
| | Homo sapiens (Human), 4349 aa. | | | |
| CAD35056 | Sequence 364 from Patent | 1 . . . 4349 | 4346/4349 (99%) | 0.0 |
| | WO0222660 - Homo sapiens | 1 . . . 4349 | 4347/4349 (99%) | |
| | (Human), 4349 aa. | | | |
| CAD35057 | Sequence 365 from Patent | 1 . . . 3821 | 3819/3821 (99%) | 0.0 |
| | WO0222660 - Homo sapiens | 1 . . . 3821 | 3819/3821 (99%) | |
| | (Human), 4263 aa. | | | |
| O88277 | Protocadherin Fat 2 precursor | 1 . . . 4349 | 3557/4351 (81%) | 0.0 |
| | (Multiple epidermal growth | 1 . . . 4351 | 3915/4351 (89%) | |
| | factor-like domains 1) - Rattus | | | |
| | norvegicus (Rat), 4351 aa. | | | |
| Q9QXA3 | Mouse fat 1 cadherin - Mus | 33 . . . 4167 | 1890/4317 (43%) | 0.0 |
| | musculus (Mouse), 4587 aa (fragment). | 35 . . . 4315 | 2701/4317 (61%) | |

PFam analysis predicts that the NOV6a protein contains the domains shown in the Table 6E.

TABLE 6E
Domain Analysis of NOV6a

| | | Identities/ | |
|---|---|---|---|
| | NOV6a Match Region | Similarities | |
| Pfam | Amino Acid residues | for the | Expect |
| Domain | of SEQ ID NO: 40 | Matched Region | Value |
| cadherin | 38 . . . 139 | 25/113 (22%) | 0.05 |
| | | 67/113 (59%) | |
| cadherin | 153 . . . 247 | 28/109 (26%) | 2.1e−08 |
| | | 69/109 (63%) | |
| cadherin | 367 . . . 449 | 21/107 (20%) | 0.94 |
| | | 54/107 (50%) | |
| cadherin | 463 . . . 553 | 40/107 (37%) | 8.9e−20 |
| | | 69/107 (64%) | |
| cadherin | 569 . . . 659 | 27/110 (25%) | 2.9e−06 |
| | | 64/110 (58%) | |
| cadherin | 720 . . . 811 | 35/107 (33%) | 4.4e−23 |
| | | 70/107 (65%) | |
| cadherin | 825 . . . 916 | 35/107 (33%) | 8.6e−24 |
| | | 75/107 (70%) | |
| cadherin | 930 . . . 1019 | 33/107 (31%) | 2.8e−12 |
| | | 62/107 (58%) | |
| cadherin | 1037 . . . 1128 | 41/107 (38%) | 7.6e−21 |
| | | 70/107 (65%) | |
| cadherin | 1142 . . . 1233 | 39/107 (36%) | 3.7e−20 |
| | | 69/107 (64%) | |
| cadherin | 1247 . . . 1337 | 33/110 (30%) | 2.2e−08 |
| | | 66/110 (60%) | |
| cadherin | 1354 . . . 1438 | 29/107 (27%) | 1.4e−05 |
| | | 63/107 (59%) | |
| cadherin | 1453 . . . 1546 | 29/107 (27%) | 4.1e−08 |
| | | 67/107 (63%) | |
| cadherin | 1560 . . . 1651 | 40/107 (37%) | 1.8e−21 |
| | | 69/107 (64%) | |
| cadherin | 1665 . . . 1749 | 26/107 (24%) | 6e−13 |
| | | 63/107 (59%) | |
| cadherin | 1763 . . . 1861 | 23/116 (20%) | 8.6e−09 |
| | | 73/116 (63%) | |
| cadherin | 1877 . . . 1959 | 30/111 (27%) | 0.27 |
| | | 53/111 (48%) | |
| cadherin | 1973 . . . 2061 | 23/108 (21%) | 7.1e−06 |
| | | 60/108 (56%) | |
| cadherin | 2075 . . . 2164 | 36/107 (34%) | 4.6e−17 |
| | | 64/107 (60%) | |
| cadherin | 2176 . . . 2263 | 32/107 (30%) | 1.5e−09 |
| | | 64/107 (60%) | |
| cadherin | 2277 . . . 2370 | 31/109 (28%) | 9.3e−27 |
| | | 73/109 (67%) | |
| cadherin | 2384 . . . 2472 | 34/111 (31%) | 3e−09 |
| | | 65/111 (59%) | |
| cadherin | 2486 . . . 2576 | 34/107 (32%) | 3.7e−15 |
| | | 66/107 (62%) | |
| cadherin | 2590 . . . 2682 | 25/111 (23%) | 1.9e−05 |
| | | 66/111 (59%) | |
| cadherin | 2696 . . . 2786 | 31/112 (28%) | 9.8e−07 |
| | | 68/112 (61%) | |
| cadherin | 2802 . . . 2897 | 36/110 (33%) | 2.5e−22 |
| | | 76/110 (69%) | |
| cadherin | 2911 . . . 3002 | 37/107 (35%) | 4.7e−12 |
| | | 63/107 (59%) | |
| cadherin | 3016 . . . 3104 | 33/107 (31%) | 6e−21 |
| | | 69/107 (64%) | |
| cadherin | 3119 . . . 3209 | 35/107 (33%) | 1.3e−13 |
| | | 66/107 (62%) | |
| cadherin | 3223 . . . 3312 | 30/107 (28%) | 1e−12 |
| | | 69/107 (64%) | |
| cadherin | 3326 . . . 3417 | 43/107 (40%) | 7e−27 |
| | | 69/107 (64%) | |
| cadherin | 3431 . . . 3522 | 36/108 (33%) | 6.9e−20 |
| | | 74/108 (69%) | |
| cadherin | 3536 . . . 3620 | 25/108 (23%) | 0.0012 |
| | | 56/108 (52%) | |
| laminin_G | 3800 . . . 3924 | 33/156 (21%) | 0.0028 |
| | | 76/156 (49%) | |
| EGF | 3951 . . . 3983 | 18/47 (38%) | 3.6e−06 |
| | | 27/47 (57%) | |
| EGF | 3990 . . . 4021 | 17/47 (36%) | 0.00016 |
| | | 26/47 (55%) | |

Various open reading frames of CG51923-01 were cloned as follows: assemblies 317868343 and 317868367, residues 1 to 659; assembly 317871203, residues 19 to 659; assemblies 317871219 and 317871243, residues 154 to 659; assembly 317871246, residues 570 to 1233; assembly 317999764, residues 1143 to 1733; assembly 318176301, residues 1561 to 2233; assemblies 305869563 and 305869567 residues 2560 to 3233. The cloned inserts differ from the original sequence as follows: assembly 317868343 has three silent SNPs and one R574C amino acid change; assembly 317868367 has one silent SNP; assembly 317871203 has two silent SNPs and one R574C amino acid change; assembly 317871219 has three silent SNPs and one R574C amino acid change; assembly 317871243 has one silent SNP; assembly 317871246 has two amino acid changes: A1100V and Y1181H; assembly 317999764 has three amino acid changes: Y1181H, Y1287D and N1303S; assembly 318176301 has no changes; assembly 305869563 differs from the original sequence by a single amino acid change: Q2992H while the cloned insert of assembly 305869567 is 100% identical to the original sequence.

### Example 7

### NOV7, CG52919, SEZ-6

The NOV7 clone was analyzed, and the nucleotide and encoded polypeptide sequences are shown in Table 7A.

TABLE 7A
NOV7 Sequence Analysis

| | | |
|---|---|---|
| NOV7a, CG52919-06 | SEQ ID NO: 67 | 1694 bp |
| DNA Sequence | ORF Start: ATG at 25 | ORF Stop: TGA at 1654 |

CAGGGCGCGGCCGCAACCAGCACCATGCGCCCGGTAGCCCTGCTGCTCCTGCCCTCGCTGCTGGCGCT
CCTGGCTCACGGACTCTCTTTAGAGGCCCCAACCGTGGGGAAAGGACAAGCCCCAGGCATCGAGGAGA
CAGATGGCGAGCTGACAGCAGCCCCCACACCTGAGCAGCCAGAACGAGGCGTCCACTTTGTCACAACA
GCCCCCACCTTGAAGCTGCTCAACCACCACCCGCTGCTTGAGGAATTCCTACAAGAGCGGCTCGAAAA
GGGAGATGACGAGCTGAGGCCAGCACTGCCCTTCCAGCCTGACCCACCTGCACCCTTCACCCCAAGTC
CCCTTCCCCGCCTGGCCAACCAGGACAGCCGCCCTGTCTTTACCAGCCCCACTCCAGCCATGGCTGCG
GTACCCACTCAGCCCCAGTCCAAGGAGGGACCCTGGAGTCCCGAGTCAGAGTCCCCTATGCTTCGAAT
CACAGCTCCCCTACCTCCACGGCCCAGCATGGCAGTGCCCACCCTACGCCCAGGGGAGATAGCCAGCA
CTACACCCCCCAGCAGAGCCTGCACACCAACCCAAGAGGCTCCTGGAGACATGGGAAGGCCGTGGGTT
GCAGAGCTTGTGTCCCAGGGCGCAGGGATCGGGATCCAGGGGACCATCACCTCCTCCACAGCTTCAGG
AGATGATGAGGAGACCACCACTACCACCACCATCATCACCACCACCATCACCACAGTCCAGACACCAG
GCCCTTGTAGCTGGAATTTCTCAGGCCCAGAGGGCTCTCTGCACTCCCCTACAGACCTCAGCTCCCCC
ACTGATGTTGGCCTGGACTGCTTCTTCTACATCTCTGTCTACCCTGGCTATGGCGTGGAAATCAAGGT
CCAGAATATCAGCCTCCGGCAAGGGGAGACAGTGACTGTGGAACGCCTGGGGGGGCCTGACCCACTCC
CCCTGGCCAACCAGTCTTTCCTGCTGCGGGGCCAAGTCATCCGCAGCCCCACCCACCAAGCGGCCCTG
AGGTTCCAGAGCCTCCCGCCACCGGCTGGCCCTGGCACCTTCCATTTCCATTACCAAGCCTATCTCCT
GAGCTGCCACTTTCCCCGTCGTCCAGCTTATCGAGATGTGACTGTCACCAGCCTCCACCCAGGGGGTA
GTGCCCGCTTCCATTGTGCCACTGGCTACCAGCTGAAGGGCGCCAGGCATCTCACCTGTCTCAATGTC
ACCCAGCCCTTCTGGGATTCAAAGGAGCCCGTCTGCATCGCTGCTTGCGGCGGAGTGATCCGCAATGC
CACCACCGGCCCCATCGTCTCTCCAGGCTTCCCGGGCAACTACAGCAACAACCTCACCTGTCACTGGC
TGCTTGAGGCTCCTGAGGGCCAGCGGCTACACCTGCACTTTGAGAAGGTTTCCCTGGCAGAGGATGAT
GACAGGCTCATCATTCCCAATGGGGACAACGTGGAGGCCCCACCAGTGTATGATTCCTATGAGGTGGA
ATACCTGCCCATTGAGGGCCTCCTCAGCTCTGGCAAACACTTCTTTGTTCAGCCCCGCCCCCGCCCCC
GCCCCTACAACCGCATTACCATAGAGTCAGCCTTTGACAATCCAACTTACGAGACTGGATCTCTTTCC
CTTGCAGGAGACGAGAGAATATGAAGTCTCCATCTAGGTGGGGGCAGTCTAGGGAAGTCAAC

| | | | |
|---|---|---|---|
| NOV7a, CG52919-06 | | | |
| Protein Sequence | SEQ ID NO: 68 | 543 aa | MW at 58351.0kD |

| | |
|---|---|
| MRPVALLLLPSLLALLAHGLSLEAPTVGKGQAPGIEETDGELTAAPTPEQPERGVHFVTTAPTLKLLN | / |

HHPLLEEFLQEGLEKGDEELRPALPFQPDPPAPFTPSPLPRLANQDSRPVFTSPTPAMAAVPTQPQSK
EGPWSPESESPMLRITAPLPPGPSMAVPTLGPGEIASTTPPSRAWTPTQEGPGDMGRPWVAEVVSQGA
GIGIQGTITSSTASGDDEETTTTTTIITTTITTVQTPGPCSWNFSGPEGSLDSPTDLSSPTDVGLDCF
FYISVYPGYGVEIKVQNISLREGETVTVEGLGGPDPLPLANQSFLLRGQVIRSPTHQAALRFQSLPPP
AGPGTFHFHYQAYLLSCHFPRRPAYGDVTVTSLHPGGSARFHCATGYQLKGARHLTCLNVTQPFWDSK
EPVCIAACGGVIRNATTGRIVSPGFPGNYSNNLTCHWLLEAPEGQRLHLHFEKVSLAEDDDRLIIRNG
DNVEAPPVYDSYEVEYLPIEGLLSSGKHFFVEPRPRPRPYNRITIESAFDNPTYETGSLSLAGDERI

| | | |
|---|---|---|
| NOV7b, 298521010 | SEQ ID NO: 69 | 444 bp |
| DNA Sequence | ORF Start: at 1 | ORF Stop: end of sequence |

TGCCACTTTCCCCGTCGTCCAGCTTATGGAGATGTGACTGTCACCAGCCTCCACCCAG
GGGGTAGTGCCCGCTTCCATTGTCCCACTGGCTACCAGCTGAAGGGCGCCAGGCATCTCACCTGTCTC
AATGTCACCCAGCCCTTCTGGGATTCAAAGGAGCCCGTCTGCATCGCTGCTTGCGGCGGAGTGATCCG
CAATGCCACCACCCCCCGCATCGTCTCTCCAGGCTTCCCGGGCAACTACAGCAACAACCTCACCTGTC
ACTGGCTGCTTGAGGCTCCTGAGGGCCAGCGGCTACACCTGCACTTTGAGAAGGTTTCCCTGGCAGAG
GATGATGACAGGCTCATCATTCGCAATGGGGACAACGTGGAGGCCCCACCAGTGTATGATTCCTATCA
GGTGGAATACCTCCCCATTGAGGGCCTGCTCAGCTCTGGCAAACAC

| | | | |
|---|---|---|---|
| NOV7b, 298521010 | | | |
| Protein Sequence | SEQ ID NO: 70 | 1148 aa | MW at ˜16958kD |

CHFPRRPAYGDVTVTSLHPGGSARFHCATGYQLKGARHLTCLNVTQPFWDSKEPVCIAACGGVIR
NATTGRIVSPGFPGNYSNNLTCHWLLEAPEGQRLHLHFEKVSLAEDDDRLIIRNGDNVEAPPVYDSYE
VEYLPIEGLLSSGKH

| | | |
|---|---|---|
| NOV7c, CG52919-09 | SEQ ID NO: 71 | 1572 bp |
| DNA Sequence | ORF Start: at 1 | ORF Stop: at 1583 |

CTCTCTTTAGAGGCCCCAACCGTGGGGAAAGGACAAGCCCCAGGCATCGAGGAGACAG
ATGGCGAGCTGACAGCAGCCCCCACACCTGAGCAGCCAGAACGAGGCGTCCACTTTGTCACAACAGCC
CCCACCTTGAAGCTGCTCAACCACCACCCGCTGCTTGAGGAATTCCTACAAGAGGGGCTGGAAAAGGG
AGATGAGGAGCTGAGGCCAGCACTGCCCTTCCAGCCTGACCCACCTGCACCCTTCACCCCAAGTCCCC
TTCCCCGCCTGGCCAACCAGGACAGCCCCCCTGTCTTTACCAGCCCCACTCCAGCCATGGCTGCGGTA
CCCACTCAGCCCCAGTCCAAGGAGGGACCCTGGAGTCCGGAGTCAGAGTCCCCTATGCTTCCAATCAC
AGCTCCCCTACCTCCAGGGCCCAGCATGGCAGTGCCCACCCTAGGCCCAGGGGACATAGCCACCACTA
CACCCCCCAGCAGAGCCTGGACACCAACCCAAGAGGGTCCTGGAGACATGGGAAGGCCGTGGGTTGCA
GAGGTTGTGTCCCAGGGCGCAGGGATCGGGATCCAGGGGACCATCACCTCCTCCACAGCTTCAGGAGA
TGATGAGCAGACCACCACTACCACCACCATCATCACCACCACCATCACCACAGTCCAGACACCAGGCC
CTTGTAGCTCGAATTTCTCAGGCCCAGAGGGTTCTCTGGACTCCCCTACAGACCTCAGCTCCCCCACT
GATGTTGGCCTGGACTGCTTCTTCTACATCTCTGTCTACCCTGGCTATGGCGTGCAAATCAAGGTCCA
GAATATCAGCCTCCGGGAAGGGGAGACAGTGACTGTCGAAGGCCTGGGGGGGCCTGACCCACTGCCCC
TGGCCAACCAGTCTTTCCTGCTGCGGGGCCAAGTCATCCGCAGCCCCACCCACCAAGCGGCCCTGAGG
TTCCACAGCCTCCCGCCACCGGCTGGCCCTGGCACCTTCCATTTCCATTACCAAGCCTATCTCCTGAG
CTGCCACTTTCCCCGTCGTCCAGCTTATGGAGATGTGACTGTCACCAGCCTCCACCCAGGGGGTAGTG
CCCGCTTCCATTGTGCCACTGCCTACCAGCTGAAGGGCGCCAGGCATCTCACCTGTCTCAATGTCACC
CAGCCCTTCTGGGATTCAAAGGAGCCCGTCTGCATCCCTGCTTGCCGCGGAGTGATCCGCAATGCCAC
CACCGGCCGCATCGTCTCTCCAGGCTTCCCGGGCAACTACAGCAACAACCTCACCTGTCACTCGCTGC
TTGAGGCTCCTGAGGGCCAGCGGCTACACCTGCACTTTGAGAAGGTTTCCCTGGCAGAGGATGATGAC
AGGCTCATCATTCGCAATGGGGACAACGTGCAGGCCCCACCAGTGTATGATTCCTATCAGGTGGAATA
CCTGCCCATTGAGGGCCTGCTCAGCTCTGGCAAACACTTCTTTGTTGAGCCCCGCCCCCGCCCCCGCC
CCTACAACCGCATTACCATAGAGTCAGCGTTTGACAATCCAACTTACGAGACTGGATCTCTTTCCCTT
GCAGGAGACGAGAGAATA

| | | | |
|---|---|---|---|
| NOV7c, CG52919-09 | | | |
| Protein Sequence | SEQ ID NO: 72 | 527 aa | MW at 56714.8kD |

LSLEAPTVGKGQAPGIEETDGELTAAPTPEQPERGVHFVTTAPTLKLLNHHPLLEEFLQEGLEKG
DEELRPALPFQPDPPAPFTPSPLPRLANQDSRPVFTSPTPAMAAVPTQPQSKEGPWSPESESPMLRIT
APLPPGPSMAVPTLGPGEIASTTPPSRAWTPTQEGPGDMGRPWVAEVVSQGAGIGIQGTITSSTASGD
DEETTTTTTIITTTITTVQTPGPCSWNFSGPEGSLDSPTDLSSPTDVGLDCFFYISVYPGYGVEIKVQ
NISLREGETVTVEGLGGPDPLPLANQSFLLRGQVIRSPTHQAALRFQSLPPPAGPGTFHFHYQAYLLS
CHFPRRPAYGDVTVTSLHPGGSARFHCATGYQLKGARHLTCLNVTQPFWDSKEPVCIAACGGVIRNAT
TGRIVSPGFPGNYSNNLTCHWLLEAPEGQRLHLHFEKVSLAEDDDRLIIRNGDNVEAPPVYDSYEVEY
LPIEGLLSSGKHFFVEPRPRPRPYNRITIESAFDNPTYETGSLSLAGDERI

A ClustalW comparison of the above protein sequences yields the following comparison. NOV7a is a 543 amino acid long protein sequence. NOV7b is the mature protein sequence corresponding to amino acid residues 20 to 543 of NOV7a. NOV7c corresponds to amino acid residues 356 to 504 of NOV7a, which includes the sushi and CUB domain as predicted by pfam, see below.

Further analysis of the NOV7a protein yielded the following properties shown in Table 7B.

TABLE 7B
Protein Sequence Properties NOV7a

| SignalP | Cleavage site between residues 20 and 21 | | | |
|---|---|---|---|---|
| analysis: | PSG: a new signal peptide prediction method | | | |
| PSORT II | N-region: length 2; pos. chg 1; neg. chg 0 | | | |
| analysis: | H-region: length 20; peak value 8.99 | | | |
| | PSG score: 4.59 | | | |
| | GvH: von Heijne's method for signal seq. recognition | | | |
| | GvH score (threshold: −2.1): 2.15 | | | |
| | possible cleavage site: between 17 and 18 | | | |
| | >>> Seems to have a cleavable signal peptide (1 to 17) | | | |
| | ALOM: Klein et al's method for TM region allocation | | | |
| | Init position for calculation: 18 | | | |
| | Tentative number of TMS(s) for the threshold 0.5: 0 | | | |
| | number of TMS(s) . . . fixed | | | |
| | PERIPHERAL | Likelihood = | 4.72 (at 267) | |
| | ALOM score: 4.72 (number of TMSs: 0) | | | |
| | MTOP: Prediction of membrane topology (Hartmann et al.) | | | |
| | Center position for calculation: 8 | | | |
| | Charge difference: −1.5 C(0.5)-N(2.0) | | | |
| | N >= C: N-terminal side will be inside | | | |
| | MITDISC: discrimination of mitochondrial targeting seq | | | |
| | R content: | 1 | Hyd Moment(75): | 5.75 |
| | Hyd Moment(95): | 8.42 | G content: | 1 |
| | D/E content: | 1 | S/T content: | 2 |

Score: −3.74
Gavel: prediction of cleavage sites for mitochondrial preseq
R-2 motif at 12 MRP|VA
NUCDISC: discrimination of nuclear localization signals
pat4: none
pat7: none
bipartite: none
content of basic residues: 6.4%
NLS Score: −0.47
KDEL: ER retention motif in the C-terminus: none
ER Membrane Retention Signals:
XXRR-like motif in the N-terminus: RPVA none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 55.5
COIL: Lupas's algorithm to detect coiled-coil regions
total: 0 residues
Final Results (k = 9/23):
55.6%: extracellular, including cell wall
33.3%: mitochondrial
11.1%: vacuolar
>> prediction for CG52919-06 is exc (k = 9)

A search of the NOV7a protein against the Geneseq database, a proprietary database that contains sequences published in patents and patent publication, yielded several homologous proteins shown in Table 7C.

TABLE 7C
Geneseq Results for NOV7a

| | | NOV7a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB70542 | Human PRO12 protein sequence | 1 . . . 543 | 518/543 (95%) | 0.0 |
| | SEQ ID NO: 24 - Homo sapiens, | 1 . . . 526 | 518/543 (95%) | |
| | 526 aa. [WO200110902-A2, 15 FEB. 2001] | | | |
| AAB70541 | Human PRO11 protein sequence | 1 . . . 533 | 513/533 (96%) | 0.0 |
| | SEQ ID NO: 22 - Homo sapiens, | 1 . . . 516 | 513/533 (96%) | |
| | 525 aa. [WO200110902-A2, 15 FEB. 2001] | | | |
| AAB70540 | Human PRO10 protein sequence | 1 . . . 533 | 513/533 (96%) | 0.0 |
| | SEQ ID NO: 20 - Homo sapiens, | 1 . . . 516 | 513/533 (96%) | |
| | 525 aa. [WO200110902-A2, 15 FEB. 2001] | | | |
| AAU81976 | Human secreted protein SECP2 - | 1 . . . 508 | 507/508 (99%) | 0.0 |
| | Homo sapiens, 994 aa. | 1 . . . 508 | 507/508 (99%) | |
| | [WO200198353-A2, 27 DEC. 2001] | | | |
| ABP69306 | Human polypeptide SEQ ID NO | 1 . . . 508 | 507/508 (99%) | 0.0 |
| | 1353 - Homo sapiens, 544 aa. | 1 . . . 508 | 507/508 (99%) | |
| | [WO200270539-A2, 12 SEP. 2002] | | | |

In a BLAST search of public sequence databases, the NOV7a protein was found to have homology to the proteins shown in the BLASTP data in Table 7D.

TABLE 7D
Public BLASTP Results for NOV7a

| | | NOV7a | Identities/ | |
|---|---|---|---|---|
| Protein | | Residues/ | Similarities | |
| Accession | | Match | for the | Expect |
| Number | Protein/Organism/Length | Residues | Matched Portion | Value |
| CAC33420 | Sequence 23 from Patent | 1 . . . 543 | 518/543 (95%) | 0.0 |
| | WO0110902 - Homo sapiens | 1 . . . 526 | 518/543 (95%) | |
| | (Human), 526 aa. | | | |
| CAC33418 | Sequence 19 from Patent | 1 . . . 533 | 513/533 (96%) | 0.0 |
| | WO0110902 - Homo sapiens | 1 . . . 516 | 513/533 (96%) | |
| | (Human), 525 aa. | | | |
| CAC33417 | Sequence 17 from Patent | 1 . . . 533 | 509/533 (95%) | 0.0 |
| | WO0110902 - Homo sapiens | 1 . . . 516 | 509/533 (95%) | |
| | (Human), 525 aa. | | | |
| CAC33416 | Sequence 15 from Patent | 1 . . . 508 | 503/508 (99%) | 0.0 |
| | WO0110902 - Homo sapiens | 1 . . . 508 | 504/508 (99%) | |
| | (Human), 994 aa. | | | |
| CAC33415 | Sequence 13 from Patent | 1 . . . 508 | 503/508 (99%) | 0.0 |
| | WO0110902 - Homo sapiens | 1 . . . 508 | 504/508 (99%) | |
| | (Human), 993 aa. | | | |

PFam analysis predicts that the NOV7a protein contains the domains shown in the Table 7E.

TABLE 7E
Domain Analysis of NOV7a

| | | Identities/ | |
|---|---|---|---|
| | | Similarities | |
| Pfam | NOV7a | for the | Expect |
| Domain | Match Region | Matched Region | Value |
| sushi | 357 . . . 412 | 15/65 (23%) | 1.9e−05 |
| | | 41/65 (63%) | |
| CUB | 416 . . . 504 | 28/116 (24%) | 1.3e−05 |
| | | 65/116 (56%) | |

### Example 8

### NOV8, CG94946, Agrin Precursor

The NOV8 clone was analyzed, and the nucleotide and encoded polypeptide sequences are shown in Table 8A.

TABLE 8A
NOV8 Sequence Analysis

| | | |
|---|---|---|
| NOV8a, CG94946-01 | SEQ ID NO: 73 | 6224 bp |
| DNA Sequence | ORF Start: ATG at 37 | ORF Stop: TGA at 6196 |

CCGGCGCGGCCCGCGCGCTCTTCCGCCGCCTCTCGCATGCGCCATGGCCGGCCGGTCCCACCCGGGCC
CGCTGCGGGGCCGCCGCTGCTGCCTCTCCTTGTGGTGGCCGCGTGCGTCCTGCCCGGAGCCGGCGGGA
CATGCCCGGAGCGCGCGCTGGAGCGGCGCGAGGAGGAGGCGAACGTGGTGCTCACCGGGACGGTGGAG
GAGATCCTCAACGTGGACCCGGTGCAGCACACGTACTCCTGCAAGGTTCGGGTCTGGCGGTACTTCAA
GGGCAAAGACCTGGTGGCCCGGGAGAGCCTGCTGGACGGCGGCAACAAGGTGGTGATCAGCGGCTTTG
GAGACCCCCTCATCTGTGACAACCAGGTGTCCACTGGGGACACCAGGATCTTCTTTGTGAACCCTGCA
CCCCCATACCTGTGGCCAGCCCACAAGAACGAGCTGATGCTCAACTCCAGCCTCATGCGCATCACCCT
GCGGAACCTGGAGGAGGTGGAGTTCTGTGTGGAAGATAAACCCGGGACCCACTTCACTCCAGTGCCTC
CGACGCCTCCTGATGCGTGCCGGGGAATGCTGTGCGGCTTCGGCGCCGTGTGCGAGCCCAACGCGGAG
GGGCCGGGCCGGGCGTCCTGCGTCTGCAAGAAGAGCCCGTGCCCCAGCGTGGTGGCGCCTGTGTGTGG
GTCGGACGCCTCCACCTACAGCAACGAATGCGAGCTGCAGCGGGCGCAGTGCAGCCAGCAGCGCCGCA
TCCGCCTGCTCAGCCGCGGGCCGTGCGGCTCGCGGGACCCCTGCTCCAACGTGACCTGCAGCTTCGGC
AGCACCTGTGCGCCCTCCGCCGACGGGCTGACGGCCTCGTGCCTGTGCCCCGCGACCTGCCGTGGCGC
CCCCGAGGGGACCGTCTGCGGCAGCGACGGCGCCCACTACCCCGGCGAGTGCCAGCTCCTGCCCCGCG
CCTGCGCCCGCCAGGAGAATGTCTTCAAGAAGTTCGACGGCCCTTGTGACCCCTGTCAGGGCGCCCTC
CCTCACCCGAGCCGCAGCTGCCGTGTGAACCCGCCCACGCGGCGCCCTGAGATGCTCCTACGGCCCGA
GAGCTGCCCTGCCCGGCAGGCGCCAGTGTGTGGGGACGACGGAGTCACCTACGAAAACGACTGTCTCA
TGGGCCGATCGGGGGCCGCCCGGGGTCTCCTCCTGCAGAAAGTGCGCTCCGGCCAGTGCCAGGGTCGA
GACCAGTGCCCGGAGCCCTGCCGGTTCAATGCCGTGTGCCTGTCCCGCCGTGGCCGTCCCCGCTGCTC
CTGCGACCGCGTCACCTGTGACGGGGCCTACAGGCCCGTGTGTGCCCAGGACGGGCGCACGTATGACA
GTGATTGCTGGCGGCAGCAGGCTGAGTGCCGCCACCAGCGTGCCATCCCCAGCAAGCACCAGGGCCCG
TGTGACCAGGCCCCGTCCCCATGCCTCGGGGTGCAGTGTGCATTTGGGGCGACGTGTGCTGTGAAGAA
CCGGCAGGCAGCGTGTGAATGCCTGCAGGCGTGCTCGAGCCTCTACGATCCTGTGTGCGGCAGCGACG
GCGTCACATACGGCAGCGCGTGCGAGCTGGAGGCCACGGCCTGTACCCTCGGGCGGGAGATCCAGGTG
GCGCGCAAAGGACCCTGTGACCGCTGCGGGCAGTGCCGCTTTGGAGCCCTGTGCGAGGCCGAGACCGG
GCGCTGCGTGTGCCCCTCTGAATGCGTGGCTTTGGCCCAGCCCGTGTGTCGCTCCGACGCGCACACGT
ACCCCAGCGACTGCATGCTGCACGTGCACGCCTGCACACACCAGATCACCCTGCACGTGGCCTCAGCT
GGACCCTGTGAGACCTGTGGAGATGCCGTGTGTGCTTTTGGGGCTGTGTGCTCCGCAGGGCAGTGTGT
GTGTCCCCGGTGTCAGCACCCCCCGCCCGGCCCCGTGTGTGGCAGCGACGGTGTCACCTACGGCAGTG
CCTGCGAGCTACCGOAAGCCGCCTGCCTCCAGCAGACACAGATCGAGGAGGCCCCGGCAGGGCCGTGC
GAGCAGGCCGAGTGCGGTTCCGGAGGCTCTGGCTCTGGGGACGACGGTGACTGTCAGCAGGAGCTGTG
CCGGCAGCGCGGTGGCATCTGGGACGAGGACTCGGAGGACGGGCCGTGTGTCTGTGACTTCAGCTGCC
AGAGTGTCCCAGGCAGCCCGGTGTGCGGCTCAGATGCGGTCACCTACACCACCGAGTGTGAGCTGAAG
AAGGCCAGGTGTGAGTCACAGCGAGGGCTCTACGTAGCGGCCCAGGGAGCCTGCCGAGGCCCCGCCTT
CGCCCCGCTGCCGCCTGTGGCCCCCTTACACTGTGCCCAGACGCCCTACGGCTGCTGCCAGGACAATA
TCACCGCAGCCCGGGGCGTGGGCCTGGCTGGCTGCCCCAGTGCCTGCCAGTGCAACCCCCATGGCTCT
TACGGCGGCACCTGTGACCCACCCACAGGCCAGTGCTCCTGCCGCCCAGGTGTGGCGCGCCTCAGGTG
TGACCGCTGTGAGCCTGGCTTCTGGAACTTTCGAGGCATCGTCACCGATGGCCGGAGTGGCTGTACAC
CCTGCAGCTGTGATCCCCAAGGCGCCGTGCCGGATGACTGTGAGCAGATGACGGGGCTGTGCTCGTGT
AAGCCCGGGGTGGCTGGACCCAAGTGTGGGCAGTGTCCAGACGGCCGTGCCCTGGGCCCCGCGGGCTG
TGAACCTCACGCTTCTGCGCCTGCGACCTGTCCGGAGATGCGCTGTGAGTTCGGTGCGCGGTGCGTGG
AGGAGTCTGGCTCAGCCCACTCTGTCTGCCCGATGCTCACCTGTCCAGAGGCCAACGCTACCAAGGTC
TGTGGGTCAGATGGAGTCACATACGGCAACGAGTGTCAGCTGAAGACCATCGCCTGCCGCCAGGGCCT
GCAAATCTCTATCCAGAGCCTGGGCCCGTGCCAGGAGGCTGTTGCTCCCAGCACTCACCCGACATCTG
CCTCCGTGACTGTGACCACCCCAGGGCTCCTCCTGAGCCAGGCACTGCCGGCCCCCCCCGCCGCCCTC
CCCCTGGCTCCCAGCAGTACCCCACACAGCCAGACCACCCCTCCGCCCTCATCGCGACCTCGGACCAC
TGCCAGCGTCCCCAGGACCACCGTGTGGCCCGTGCTGACGGTGCCCCCCACGGCACCCTCCCCTGCAC
CCAGCCTGCTGGCGTCCGCCTTTGGTGAATCTGGCAGCACTGATGGAAGCAGCGATGAGGAACTGACC
GGGGACCAGGAGGCCAGTGGGGGTGGCTCTGGGGGGCTCGACCCCTTGGAGGGCAGCAGCGTGGCCAC
CCCTGGGCCACCTGTCGAGAGGGCTTCCTGCTACAACTCCGCGTTGGGCTGCTGCTCTGATGGGAAGA
CGCCCTCCCTGGACGCAGAGGGCTCCAACTGCCCCGCCACCAAGGTGTTCCAGGGCGTCCTGGAGCTG
GAGGGCGTCGAGGGCCAGGAGCTGTTCTACACGCCCGAGATGGCTGACCCCAAGTCAGAACTGTTCGG
GGAGACAGCCAGGAGCATTGACAGCACCCTGGACGACCTCTTCCGGAATTCAGACGTCAAGAAGGATT
TCCGGAGTGTCCGCTTGCGGGACCTGGGGCCCGGCAAATCCGTCCGCGCCATTGTGGATGTGCACTTT
GACCCCACCACACCCTTCAGGGCACCCGACGTGGCCCGGGCCCTGCTCCGGCAGATCCAGGTGTCCAG
GCGCCGGTCCTTGGGGCTGAGGCGGCCGCTGCAGGAGCACGTGCGATTTATGGACTTTGACTGGTTTC
CTGCGTTTATCACGGGGGCCACGTCAGGAGCCATTGCTCCGGGAGCCACGGCCAGAGCCACCACTGCA
TCGCGCCTGCCGTCCTCTGCTGTGACCCCTCGGGCCCCGCACCCCAGTCACACAAGCCAGCCCGTTGC
CAAGACCACGGCAGCCCCCACCACACGTCGGCCCCCCACCACTGCCCCCAGCCGTGTGCCCGCACGTC
GGCCCCCGGCCCCCCAGCAGCCTCCAAAGCCCTGTGACTCACAGCCCTGCTTCCACGGGGGGACCTCC
CAGGACTGGGCATTGGGCGGGGGCTTCACCTGCAGCTGCCCGGCAGGCAGGGGAGGCGCCGTCTGTGA
GAAGGTGCTTGGCGCCCCTGTGCCGGCCTTCGAGGGCCGCTCCTTCCTGGCCTTCCCCACCCTCCGCG
CCTACCACACGCTGCGCCTGGCACTGGAATTCCGCGCGCTGGAGCCTCAGGGGCTGCTGCTGTACAAT
GGCAACGCCCGGGGCAAGGACTTCCTGGCATTGGCGCTGCTAGATGGCCGCGTGCAGCTCAGGTTTGA
CACAGGTTCGGGGCCGGCGGTGCTGACCAGTGCCGTGCCGGTAGAGCCGGGCCAGTCGCACCGCCTGG
AGCTGTCCCGGCACTCGCGCCGGGGCACCCTCTCGGTGGATGGTGAGACCCCTGTTCTGGGCGAGAGT
CCCACTGGCACCGACGGCCTCAACCTGGACACAGACCTCTTTGTGGGCGGCGTACCCGACGACCAGGC
TCCCGTGGCGCTGGAGCGCACCTTCGTGGGCGCCGGCCTGAGGGGGTGCATCCGTTTGCTGGACGTCA
ACAACCAGCGCCTGGAGCTTGGCATTGGGCCGGGGGCTGCCACCCGAGGCTCTGGCGTGGGCGAGTGC
GGGGACCACCCCTGCCTGCCCAACCCCTGCCATGGCGGGGCCCCATGCCAGAACCTGGACGCTGGAAG
GTTCCATTGCCAGTGCCCGCCCGGCCGCGTCGGACCAACCTGTGCCGATGAGAAGACCCCCTGCCAGC
CCAACCCCTGCCATGGGGCGGCGCCCTGCCGTGTGCTGCCCGAGGGTGGTGCTCAGTGCCAGTCCCCC
CTGCGGCGTGAGGGCACCTTCTGCCAGACAGCCTCGGGGCAGGACGGCTCTGGGCCCTTCCTGGCTGA
CTTCAACGGCTTCTCCCACCTGGAGCTGAGAGGCCTGCACACCATTGCACGGGACCTGGGGGAGAAGA
TGGCGCTGGAGGCCGTGTTCCTGGCACGAGGCCCCAGCGGCCTCCTGCTCTACAACGGGCAGAAGACG
GACGGCAAGGGGGACTTCGTGTCGCTGGCACTGCGGGACCGCCGCCTGGAGTTCCGCTACGACCTCGG
CAAGGGGGCAGCGGTCATCAGGACCAGGGAGCCAGTCACCCTGGGAGCCTGGACCAGGGTCTCACTGG
AGCGAAACGGCCGCAAGGGTGCCCTGCGTGTGGGCGACGGCCCCCGTCTGTTGGGGGAGTCCCCGAAA
TCCCGCAAGGTTCCGCACACCGTCCTCAACCTGAAGGAGCCGCTCTACGTAGGGGGCGCTCCCGACTT
CAGCAAGCTGGCCCGTGCTGCTGCCGTGTCCTCTGGCTTCGACGGTGCCATCCAGCTGGTCTCCCTCC
GAGGCCGCCAGCTGCTGACCCCGGAGCACGTGCTGCGGCAGGTGCACGTCACGTCCTTTGCAGGTCAC
CCCTGCACCCGGGCCTCACGCCACCCCTGCCTCAATGGGGCCTCCTGCGTCCCGAGGGACGCTGCCTA
TGTGTGCCTGTGTCCCGGGGGATTCTCAGGACCGCACTGCGAGAACGGGCTGGTGGAGAAGTCAGCGG
GGGACCTGGATACCTTGGCCTTTGACGGGCGGACCTTTGTCGAGTACCTCAACGCTGTGACCGAGAGC
GAGAAGGCACTCCAGAGCAACCACTTTGAACTGAGCCTGCGCACTGAGGCCACGCAGGGGCTGGTGCT
CTGGAGTGGCAAGGCCACGGAGCGGGCAGACTATGTGGCACTGGCCATTGTGGACGGGCACCTGCAAC
TGAGCTACAACCTGGGCTCCCAGCCCGTGGTGCTGCGTTCCACCCTGCCCGTCAACACCAACCGCTGG
TTGCGGGTCGTGGCACATAGGGAGCAGAGGGAAGGTTCCCTGCAGGTGGGCAATGAGGCCCCTGTGAC
CGGCTCCTCCCCGCTGGGCGCCACGCAGCTGGACACTGATGGAGCCCTGTGGCTTGGGGGCCTGCCGG
AGCTGCCCGTGGGCCCAGCACTGCCCAACGCCTACGGCACAGGCTTTGTGGGCTGCTTGCGGGATGTC
GTGGTGGGCCGGCACCCGCTGCACCTGCTGGAGGACGCCGTCACCAAGCCAGAGCTCCGGCCCTGCCC
CACCCCATGAGCTGGCACCACAGCCCCGCGCCCGCT

| | | | |
|---|---|---|---|
| NOV8a, CG94946-01 | | | |
| Protein Sequence | SEQ ID NO: 74 | 2053 aa | Mw at 215628.0kD |

MRHGRPVPPGPAAGRPLLPLLVVAACVLPGAGGTCPERALERREEEANVVLTGTVEEILNVDPVQHTY
SCKVRVWRYLKGKDLVARESLLDGGNKVVISGFGDPLICDNQVSTGDTRIFFVNPAPPYLWPAHKNEL
MLNSSLMRITLRNLEEVEFCVEDKPGTHFTPVPPTPPDACRGMLCGFGAVCEPNAEGPGRASCVCKKS
PCPSVVAPVCGSDASTYSNECELQRAQCSQQRRIRLLSRGPCGSRDPCSNVTCSFGSTCARSADGLTA
SCLCPATCRGAPEGTVCGSDGADYPGECQLLRRACARQENVFKKFDGPCDPCQGALPDPSRSCRVNPR
TRRPEMLLRPESCPARQAPVCGDDGVTYENDCVMGRSGAARGLLLQKVRSGQCQGRDQCPEPCRFNAV
CLSRRGRPRCSCDRVTCDGAYRPVCAQDGRTYDSDCWRQQAECRQQRAIPSKHQGPCDQAPSPCLGVQ
CAFGATCAVKNGQAACECLQACSSLYDPVCGSDGVTYGSACELEATACTLGREIQVARKGPCDRCGQC
RFGALCEAETGRCVCPSECVALAQPVCGSDGHTYPSECMLHVHACTHQISLHVASAGPCETCGDAVCA
FGAVCSAGQCVCPRCEHPPPGPVCGSDGVTYGSACELREAACLQQTQIEEARAGPCEQAECGSGGSGS
GEDGDCEQELCRQRGGIWDEDSEDGPCVCDFSCQSVPGSPVCGSDGVTYSTECELKKARCESQRGLYV
AAQGACRGPAFAPLPPVAPLHCAQTPYGCCQDNITAARGVGLAGCPSACQCNPHGSYGGTCDPATGQC
SCRPGVGGLRCDRCEPGFWNFRGIVTDGRSGCTPCSCDPQGAVRDDCEQMTGLCSCKPGVAGPKCGQC
PDGRALGPAGCEADASAPATCAEMRCEFGARCVEESGSAHCVCPMLTCPEANATKVCGSDGVTYGNEC
QLKTIACRQGLQISIQSLGPCQFAVAPSTHPTSASVTVTTPGLLLSQALPAPPGALPLAPSSTAHSQT
TPPPSSRPRTTASVPRTTVWPVLTVPPTAPSPAPSLVASAFGESGSTDGSSDEELSGDQEASCGGSGG
LEPLEGSSVATPGPPVERASCYNSALGCCSDGKTPSLDAEGSNCPATKVFQGVLELEGVEGQELFYTP
EMADPKSELFGETARSIESTLDDLFRNSDVKKDFRSVRLRDLGPGKSVRAIVDVHFDPTTAFRAPDVA
RALLRQIQVSRRRSLGVRRPLQEHVRFMDFDWFPAFITGATSGAIAAGATARATTASRLPSSAVTPRA
PHPSHTSQPVAKTTAAPTTRRPPTTAPSRVPGRRPPAPQQPPKPCDSQPCFHGGTCQDWALGGGFTCS
CPAGRGGAVCEKVLGAPVPAFEGRSFLAFPTLRAYHTLRLALEFRALEPQGLLLYNGNARGKDFLALA
LLDGRVQLRFDTGSGPAVLTSAVPVEPGQWHRLELSRHWRRGTLSVDGETPVLGESPSCTDGLNLDTD
LFVGGVPEDQAAVALERTFVGAGLRGCIRLLDVNNQRLELGIGPGAATRGSCVCECGDHPCLPNPCHG
GAPCQNLEAGRFHCQCPPGRVGPTCADEKSPCQPNPCHGAAPCRVLPEGGAQCECPLGREGTFCQTAS
GQDGSGPFLADFNGFSHLELRGLHTIARDLGEKMALEAVFLARGPSGLLLYNGQKTDGKGDFVSLALR
DRRLEFRYDLGKGAAVIRSREPVTLGAWTRVSLERNGRKGALRVGDGPRVLGESPKSRKVPHTVLNLK
EPLYVGGAPDFSKLARAAAVSSGFDGAIQLVSLGGRQLLTPEHVLRQVDVTSFAGHPCTRASGHPCLN
GASCVPREAAYVCLCPGGFSGPHCEKGLVEKSAGDVDTLAFDGRTFVEYLNAVTESEKALQSNHFELS
LRTEATQGLVLWSGKATERADYVALAIVDGHLQLSYNLGSQPVVLRSTVPVNTNRWLRVVAHREQREG
SLQVGNEAPVTGSSPLGATQLDTDGALWLGGLPELPVGPALPKAYGTGFVGCLRDVVVGRHPLHLLED
AVTKPELRPCPTP

| | | |
|---|---|---|
| NOV8b, 308909220 | SEQ ID NO: 75 | 1935 bp |
| DNA Sequence | ORF Start: at 1 | ORF Stop: end of sequence |

TTCCCGGCGCTGGAGCCTCAGGGGCTGCTGCTGTACAATGGCAACGCCCGGGGCAAGG
ACTTCCTGGCATTGCCGCTGCTACATGGCCGCGTGCAGCTCAGGTTTGACACACGTTCGGCGCCGGCG
GTGCTGACCAGTGCCGTGCCGGTAGAGCCGGGCCAGTGGCACCGCCTGGAGCTGTCCCGGCACTGGCG
CCGGGGCACCCTCTCGGTGGATGGTGAGACCCCTGTTCTGGGCGAGAGTCCCAGTGGCACCGACGGCC
TCAACCTGGACACACACCTCTTTGTGGGCGGCGTACCCGAGGACCAGGCTGCCGTGGCGCTGGAGCGG
ACCTTCGTGGGCGCCGGCCTGAGGGGGTGCATCCGTTTGCTCGACGTCAACAACCAGCGCCTGGAGCT
TGGCATTGGGCCGGGGGCTGCCACCCGAGGCTCTGGCGTGGGCGAGTGCGGGGACCACCCCTGCCTGC
CCAACCCCTGCCATCGCGGGGCCCCATGCCAGAACCTGGAGGCTGGAAGGTTCCATTGCCAGTGCCCG
CCCGGCCGCGTCGGACCAACCTGTGCCGATGAGAAGAGCCCCTGTCAGCCCAACCCCTGCCATGGGGC
GGCGCCCTCCCGTGTGCTGCCCGAGGGTGGTGCTCAGTGCGAGTCCCCCCTGGGGCGTGAGGGCACCT
TCTGCCAGACAGCCTCGGGGCAGGACGGCTCTGGGCCCTTCCTGGCTGACTTCAACGGCTTCTCCCAC
CTGGAGCTGAGAGGCCTGCACACCTTTGCACGGGACCTGGGGGAGAAGATGGCGCTGGAGGTCGTGTT
CCTGCCACGAGGCCCCAGCGGCCTCCTGCTCTACAACGGGCAGAAGACGGACGGCAAGGGGGACTTCG
TGTCGCTGGCACTGCGGGACCGCCGCCTGGAGTTCCGCTACGACCTGGGCAAGCGGGCACCGGTCATC
AGGAGCAGGGAGCCAGTCACCCTGGGAGCCTGGACCAGGGTCTCACTGGAGCCAAACGGCCGCAAGGG
TGCCCTGCGTGTCGGCGACGGCCCCCGTGTGTTGGGGGAGTCCCCGGTTCCGCACACCGTCCTCAACC
TGAAGGAGCCGCTCTACGTAGGGGGCGCTCCCGACTTCAGCAAGCTGCCCCGTGCTGCTGCCCTGTCC
TCTGGCTTCGACGGTGCCATCCAGCTGGTCTCCCTCGGAGGCCGCCAGCTGCTGACCCCGGAGCACGT
GCTGCGCCAGGTGGACGTCACGTCCTTTGCAGGTCACCCCTGCACCCGGGCCTCAGCCCACCCCTGCC
TCAATGGGGCCTCCTGCGTCCCGAGGGAGGCTGCCTATGTGTGCCTGTGTCCCGGGGGATTCTCAGGA
CCGCACTGCGAGAAGGGGCTGGTGGAGAAGTCAGCGGGGGACGTGCATACCTTGGCCTTTGACGGGCG
GACCTTTGTCGAGTACCTCAACGCTGTGACCGAGAGCGAGAAGGCACTGCAGAGCAACCACTTTGAAC
TGAGCCTGCGCACTGAGGCCACGCAGGGGCTGGTGCTCTGGAGTGGCAAGGCCACGGAGCGGGCAGAC
TATGTGGCACTGGCCATTGTGGACGGGCACCTGCAACTGAGCTACAACCTGGGCTCCCAGCCCGTGGT
CCTGCGTTCCACCGTGCCCGTCAACACCAACCGCTGGTTGCGGGTCGTGGCACATAGGGAGCAGAGGG
AAGGTTCCCTGCAGGTGGGCAATGAGGCCCCTGTGACCGGCTCCTCCCCGCTGGGCGCCACGCAGCTG
GACACTGATGGACCCCTGTGGCTTGGGGGCCTGCCGGAGCTGCCCGTGGGCCCAGCACTGCCCAAGGC
CTACGGCACAGGCTTTGTGGGCTGCTTGCGGGACGTGGTGGTGGGCCCGCACCCGCTGCACCTGCTGG
AGGACCCCCTCACCAAGCCAGAGCTGCGGCCCTGCCCCACC

| | | | |
|---|---|---|---|
| NOV8b, 308909220 | | | |
| Protein Sequence | SEQ ID NO: 76 | 645 aa | MW at ˜68813kD |

FRALEPQGLLLYNGNARGKDFLALALLDGRVQLRFDTGSGPAVLTSAVPVEPGQWHRLELSRHWR
RGTLSVDGETPVLGESPSGTDGLNLDTDLFVGGVPEDQAAVALERTFVGAGLRGCIRLLDVNNQRLEL
GIGPGAATRGSGVGECGDHPCLPNPCHGGAPCQNLEAGRFHCQCPPGRVGPTCADEKSPCQPNPCHGA
APCRVLPEGGAQCECPLGREGTFCQTASGQDGSGPFLADFNGFSHLELRGLHTFARDLGEKMALEVVF
LARGPSGLLLYNGQKTDGKGDFVSLALRDRRLEFRYDLGKGAAVIRSREPVTLGAWTRVSLERNGRKG
ALRVGDGPRVLGESPVPHTVLNLKEPLYVGGAPDFSKLARAAAVSSGFDGAIQLVSLGGRQLLTPEHV
LRQVDVTSFAGHPCTRASGHPCLNGASCVPREAAYVCLCPGGFSGPHCEKGLVEKSAGDVDTLAFDGR
TFVEYLNAVTESEKALQSNHFELSLRTEATQGLVLWSGKATERADYVALAIVDGHLQLSYNLGSQPVV
LRSTVPVNTNRWLRVVAHREQREGSLQVGNEAPVTGSSPLGATQLDTDGALWLGGLPELPVGPALPKA
YGTGFVGCLRDVVVGRHPLHLLEDAVTKPELRPCPT

A ClustalW comparison of the above protein sequences yields the following sequence comparisons. NOV8b corresponds to NOV8a protein sequence amino acid residues 1404-2052 with the following changes: 11658F; A1670V; and deletion of 1756-1759 of the NOV8a sequence and furthermore includes several laminin G and-EGF domains as predicted by pfam, below.

Further analysis of the NOV8a protein yielded the following properties shown in Table 8B.

TABLE 8B
Protein Sequence Properties NOV8a

| SignalP | Cleavage site between residues 34 and 35 | | | | |
|---|---|---|---|---|---|
| analysis: | PSG: a new signal peptide prediction method | | | | |
| PSORT II | N-region: length 5; pos. chg 2; neg. chg 0 | | | | |
| analysis: | H-region: length 9; peak value 3.79 | | | | |
| | PSG score: −0.61 | | | | |
| | GvH: von Heijne's method for signal seq. recognition | | | | |
| | GvH score (threshold: −2.1): 0.49 | | | | |
| | possible cleavage site: between 33 and 34 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 1 | | | | |
| | Number of TMS(s) for threshold 0.5: 1 | | | | |
| | INTEGRAL | Likelihood = | −4.35 | Transmembrane | 17-33 |

| PERIPHERAL | Likelihood = | 0.53 (at 609) | |
|---|---|---|---|
| ALOM score: −4.35 (number of TMSs: 1) | | | |
| MTOP: Prediction of membrane topology (Hartmann et al.) | | | |
| Center position for calculation: 24 | | | |
| Charge difference: −6.5 C(−2.0)-N(4.5) | | | |
| N >= C: N-terminal side will be inside | | | |
| >>> membrane topology: type 2 (cytoplasmic tail 1 to 17) | | | |
| MITDISC: discrimination of mitochondrial targeting seq | | | |
| R content: | 3 | Hyd Moment(75): | 2.17 |
| Hyd Moment(95): | 10.07 | G content: | 6 |
| D/E content: | 1 | S/T content: | 1 |

```
Score: −4.90
Gavel: prediction of cleavage sites for mitochondrial preseq
R-2 motif at 25 GRP|LL
NUCDISC: discrimination of nuclear localization signals
pat4: none
pat7: PRTRRPE (5) at 339
pat7: PKSRKVP (5) at 1755
bipartite: none
content of basic residues: 9.5%
NLS Score: 0.39
KDEL: ER retention motif in the C-terminus: none
ER Membrane Retention Signals:
XXRR-like motif in the N-terminus: RHGR none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 76.7
COIL: Lupas's algorithm to detect coiled-coil regions
total: 0 residues
Final Results (k = 9/23):
34.8%: mitochondrial
34.8%: nuclear
13.0%: cytoplasmic
  4.3%: extracellular, including cell wall
  4.3%: vacuolar
  4.3%: Golgi
  4.3%: peroxisomal
>> prediction for CG94946-01 is mit (k = 23)
```

A search of the NOV8a protein against the Geneseq database, a proprietary database that contains sequences published in patents and patent publication, yielded several homologous proteins shown in Table 8C.

TABLE 8C
Geneseq Results for NOV8a

| | | NOV8a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABU52400 | Human GPCR related protein | 160 . . . 2053 | 1841/1894 (97%) | 0.0 |
| | NOV40a - Homo sapiens, 1931 | 51 . . . 1931 | 1853/1894 (97%) | |
| | aa. [WO200279398-A2, 10 OCT. 2002] | | | |
| ABP43859 | Human mRNA precursor - Homo | 137 . . . 1669 | 1530/1533 (99%) | 0.0 |
| | sapiens, 1741 aa. | 1 . . . 1533 | 1530/1533 (99%) | |
| | [WO200231111-A2, 18 APR. 2002] | | | |
| AAW26609 | Human agrin - Homo sapiens, | 1591 . . . 2053 | 460/471 (97%) | 0.0 |
| | 492 aa. [WO9721811-A2, 19 JUN. 1997] | 22 . . . 492 | 461/471 (97%) | |
| AAB93754 | Human protein sequence SEQ ID | 583 . . . 968 | 381/386 (98%) | 0.0 |
| | NO: 13424 - Homo sapiens, 413 | 1 . . . 386 | 384/386 (98%) | |
| | aa. [EP1074617-A2, 07 FEB. 2001] | | | |
| AAY73993 | Human prostate tumor EST | 1634 . . . 2053 | 414/420 (98%) | 0.0 |
| | fragment derived protein #180 - | 1 . . . 416 | 414/420 (98%) | |
| | Homo sapiens, 416 aa. | | | |
| | [DE19820190-A1, 04 NOV. 1999] | | | |

In a BLAST search of public sequence databases, the NOV8a protein was found to have homology to the proteins shown in the BLASTP data in Table 8D.

TABLE 8D
Public BLASTP Results for NOV8a

| | | NOV8a | Identities/ | |
|---|---|---|---|---|
| Protein | | Residues/ | Similarities | |
| Accession | | Match | for the | Expect |
| Number | Protein/Organism/Length | Residues | Matched Portion | Value |
| O00468 | AGRIN precursor - Homo | 24 . . . 2053 | 2022/2030 (99%) | 0.0 |
| | sapiens (Human), 2026 aa | 1 . . . 2026 | 2022/2030 (99%) | |
| | (fragment). | | | |
| P25304 | Agrin precursor - Rattus | 160 . . . 2053 | 1558/1914 (81%) | 0.0 |
| | norvegicus (Rat), 1959 aa. | 51 . . . 1959 | 1663/1914 (86%) | |
| P31696 | Agrin precursor - Gallus gallus | 128 . . . 2050 | 1234/1970 (62%) | 0.0 |
| | (Chicken), 1955 aa. | 1 . . . 1952 | 1479/1970 (74%) | |
| Q90404 | Agrin - Discopyge ommata | 716 . . . 2051 | 733/1353 (54%) | 0.0 |
| | (Electric ray), 1328 aa | 1 . . . 1325 | 932/1353 (68%) | |
| | (fragment). | | | |
| Q96IC1 | Hypothetical protein - Homo | 1562 . . . 2053 | 486/492 (98%) | 0.0 |
| | sapiens (Human), 488 aa | 1 . . . 488 | 486/492 (98%) | |
| | (fragment). | | | |

PFam analysis predicts that the NOV8a protein contains the domains shown in the Table 8E.

TABLE 8E
Domain Analysis of NOV8a

| | | Identities/ | |
|---|---|---|---|
| | NOV8a Match Region | Similarities | |
| Pfam | Amino Acid residues | for the | Expect |
| Domain | of SEQ ID NO: 74 | Matched Region | Value |
| NtA | 34 . . . 161 | 125/135 (93%) | 1.1e−111 |
| | | 128/135 (95%) | |
| kazal | 201 . . . 246 | 25/61 (41%) | 7.2e−18 |
| | | 36/61 (59%) | |
| kazal | 276 . . . 321 | 21/62 (34%) | 5.1e−13 |
| | | 33/62 (53%) | |
| kazal | 346 . . . 393 | 18/61 (30%) | 7.9e−12 |
| | | 33/61 (54%) | |
| kazal | 420 . . . 465 | 21/61 (34%) | 4.1e−16 |
| | | 38/61 (62%) | |
| kazal | 494 . . . 538 | 24/61 (39%) | 3.6e−19 |
| | | 38/61 (62%) | |
| kazal | 559 . . . 603 | 19/61 (31%) | 1.4e−18 |
| | | 38/61 (62%) | |
| kazal | 624 . . . 668 | 26/62 (42%) | 1.5e−17 |
| | | 37/62 (60%) | |
| kazal | 709 . . . 754 | 24/62 (39%) | 1.2e−16 |
| | | 40/62 (65%) | |
| laminin_EGF | 797 . . . 848 | 28/61 (46%) | 1.1e−20 |
| | | 46/61 (75%) | |
| laminin_EGF | 851 . . . 895 | 21/59 (36%) | 3.6e−11 |
| | | 37/59 (63%) | |
| kazal | 927 . . . 973 | 25/62 (40%) | 5.2e−18 |
| | | 41/62 (66%) | |
| SEA | 1134 . . . 1256 | 38/132 (29%) | 4.6e−37 |
| | | 111/132 (84%) | |
| EGF | 1337 . . . 1370 | 16/47 (34%) | 0.00055 |
| | | 24/47 (51%) | |
| laminin_G | 1404 . . . 1535 | 70/154 (45%) | 4.6e−55 |
| | | 120/154 (78%) | |
| EGF | 1557 . . . 1589 | 16/47 (34%) | 5.2e−06 |
| | | 27/47 (57%) | |
| EGF | 1596 . . . 1628 | 16/47 (34%) | 0.00021 |
| | | 25/47 (53%) | |
| laminin_G | 1672 . . . 1807 | 71/154 (46%) | 5.8e−52 |
| | | 122/154 (79%) | |
| EGF | 1826 . . . 1860 | 14/47 (30%) | 4.4e−07 |
| | | 25/47 (53%) | |
| laminin_G | 1905 . . . 2036 | 58/154 (38%) | 1.2e−50 |
| | | 125/154 (81%) | |

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various NOV genes was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s:18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 μl or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 59° C. primer optimal Tm; 2° C. maximum primer difference (if probe does not have 5′ G, probe Tm must be 10° C. greater than primer Tm; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers-sets. PCR reactions were done using TaqMan® One-Step RT-PCR Master Mix (Applied Biosystems, Catalog No. 4313803) following manufacturer's instructions. Reverse transcription was performed at 480 C for 30 minutes followed by amplification/PCR cycles: 95° C. 10 min, then 40 cycles at 95° C. for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1× TaqMan® Universal Master mix (Applied Biosystems; catalog No. 4324020), following the manufacturer's instructions. PCR amplification and analysis were done as described above.

Panels 1, 1.1, 1.2, and 1.3D

Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone-marrow, lymph node, pancreas, salivary gland, pituitary gland, adrenal gland, spinal cord, thymus, stomach, small intestine, colon, bladder, trachea, breast, ovary, uterus, placenta,-prostate, testis and adipose. The following abbreviations are used in reporting the results: metastasis (met); pleural effusion (pl. eff or pl effusion) and * indicates established from metastasis.

General_screening_panel v1.4, v1.5, v1.6 and v1.7

Panels 1.4, 1.5, 1.6 and 1.7 were as described for Panels 1, 1.1, 1.2 and 1.3D, above except that normal tissue samples were pooled from 2 to 5 different adults or fetuses.

Panels 2D, 2.2, 2.3 and 2.4

Panels 2D, 2.2, 2.3 and 2.4 included 2 control wells and 94 wells containing RNA or cDNA from human surgical specimens procured through the National Cancer Institute's Cooperative Human Tissue Network (CHTN) or the National Disease Research Initiative (NDRI), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

HASS Panel v 1.0

The HASS Panel v1.0 included 93 cDNA samples and two controls including: 81 samples of cultured human cancer cell lines subjected to serum starvation, acidosis and anoxia according to established procedures for various lengths of time; 3 human primary cells; 9 malignant brain cancers (4 medulloblastomas and 5 glioblastomas); and 2 controls. Cancer cell lines (ATCC) were cultured using recommended conditions and included: breast, prostate, bladder, pancreatic and CNS. Primary human cells were obtained from Clonetics (Walkersville, Md.). Malignant brain samples were gifts from the Henry Ford Cancer Center.

ARDAIS Panel v1.0 and v1.1

The ARDAIS Panel v1.0 and v1.1 included 2 controls and 22 test samples including: human lung adenocarcinomas, lung squamous cell carcinomas, and in some cases matched adjacent normal tissues (NAT) obtained from Ardais (Lexington, Mass.). Unmatched malignant and non-malignant RNA samples from lungs with gross histopathological assessment of tumor differentiation grade and stage and clinical state of the patient were obtained from Ardais.

ARDAIS Prostate v1.0

ARDAIS Prostate v1.0 panel included 2 controls and 68 test samples of human prostate malignancies and in some cases matched adjacent normal tissues (NAT) obtained from Ardais (Lexington, Mass.). RNA from unmatched malignant and non-malignant prostate samples with gross histopathological assessment of tumor differentiation grade and stage and clinical state of the patient were also obtained from Ardais.

ARDAIS Kidney v1.0

ARDAIS Kidney v1.0 panel included 2 control wells and 44 test samples of human renal cell carcinoma and in some cases matched adjacent normal tissue (NAT) obtained from Ardais (Lexington, Mass.). RNA from unmatched renal cell carcinoma and normal tissue with gross histopathological assessment of tumor differentiation grade and stage and clinical state of the patient were also obtained from Ardais.

ARDAIS Breast v1.0

ARDAIS Breast v1.0 panel included 2 control wells and 71 test samples of human breast malignancies and in some cases matched adjacent normal tissue (NAT) obtained from Ardais (Lexington, Mass.). RNA from unmatched malignant and non-malignant breast samples with gross histopathological assessment of tumor differentiation grade and stage and clinical state of the patient were also obtained from Ardais.

Panel 3D, 3.1 and 3.2

Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

Panels 4D, 4R, and 4.1D

Panels 4D, 4R, and 4.1D included 2 control wells and 94 test samples of RNA (Panel 4R) or cDNA (Panels 4D and 4.1D) from human cell lines or tissues related to inflammatory conditions. Controls included total RNA from normal tissues such as colon, lung (Stratagene, La Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDRI, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary cell types were activated by incubating with various cytokines (IL-1 beta ˜1-5 ng/ml, TNF alpha ˜5-10 ng/ml, IFN gamma ˜20-50 ng/ml, IL-4˜5-10 ng/ml, IL-9˜5-10 ng/ml, IL-13 5-10 ng/ml) or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media [DMEM, 5% FCS (Hyclone, Logan, Utah), 100 mM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco)] and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with ˜5 mg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of ˜2×10⁶ cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/ml IL-4 for 5-7 days. Macrophages were prepared by culturing monocytes for 5-7 days in culture media with ˜50 ng/ml 10% type AB Human Serum (Life technologies, Rockville, Md.) or MCSF (Macrophage colony stimulating factor; R&D, Minneapolis, Minn.). Monocytes, macrophages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calf.) for 6 or 12-14 hours.

CD4+ lymphocytes, CD8+ lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+RA and CD45+RO CD4+ lymphocytes were isolated by depleting mononuclear cells of CD8+, CD56+, CD14+and CD19+ cells using CD8, CD56, CD14 and CD19 Miltenyi beads and positive selection. CD45RO Miltenyi beads were then used to separate the CD45+RO CD4+lymphocytes from CD45+RA CD4+ lymphocytes. CD45+RA CD4+, CD45+RO CD4+ and CD8+ lymphocytes were cultured in culture media at 10⁶ cells/ml in culture plates precoated overnight with 0.5 mg/ml anti-CD28 (Pharmingen, San Diego, Calif.) and 3 μg/ml anti-CD3 (OKT3, ATCC) in PBS. After 6 and 24 hours, the cells were harvested for RNA preparation. To prepare chronically activated CD8+ lymphocytes, isolated CD8+ lymphocytes were activated for 4 days on anti-CD28, anti-CD3 coated plates and then harvested and expanded in culture media with IL-2 (1 ng/ml). These CD8+ cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 4-6 days before RNA was prepared.

B cells were prepared from minced and sieved tonsil tissue (NDRI). Tonsil cells were pelleted and resupended at 10⁶ cells/ml in culture media. Cells were activated using 5 μg/ml PWM (Sigma-Aldrich Corp., St. Louis, Mo.) or ˜10 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+ lymphocytes (Poietic Systems, German Town, Md.) were cultured at 10⁵-10⁶cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (1 ng/ml). Activated Th1, Th2 and Tr1 lymphocytes were re-stimulated for 5 days with anti-CD28/CD3 and cytokines as described above with the addition of anti-CD95L (1 /μg/ml) to prevent apoptosis. After 4-5 days, the Th1, Th2 and Tr2 lymphocytes were washed and expanded in culture media with IL-2 for 4-7 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×10⁵ cells/ml with 0.1 mM dbcAMP for 8 days, changing the media every 3 days and adjusting the cell concentration to 5×10⁵ cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with ˜5 ng/ml TNF alpha and 1 ng/ml IL-1 beta. RNA was prepared from resting NCI-H292, airway epithelial tumor cell line (ATCC) or from cells activated for 6 and 14 hours in culture media with 5 ng/ml IL-4, 5 ng/ml IL-9, 5 ng/ml IL-13, and 25 ng/ml IFN gamma.

RNA was prepared by lysing approximately 10⁷ cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −20° C. overnight. The precipitated RNA was spun down at 9,000 rpm for 15 min and washed in 70% ethanol. The pellet was redissolved in 300 μl of RNAse-free water with 35 ml buffer (Promega, Madison, Wis.) 5 μl DTT, 7 μl RNAsin and 8 μl DNAse and incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

AI_Comprehensive Panel_v1.0

Autoimmunity (AI) comprehensive panel v1.0 included two controls and 89.cDNA test samples isolated from male (M) and female (F) surgical and postmortem human tissues that were obtained from the Backus Hospital and Clinomics (Frederick, Md.). Tissue samples included: normal, adjacent (Adj); matched normal adjacent (match control); joint tissues (synovial (Syn) fluid, synovium, bone and cartilage, osteoarthritis (OA), rheumatoid arthritis (RA)); psoriatic; ulcerative colitis colon; Crohns disease colon; and emphysmatic, asthmatic, allergic and chronic obstructive pulmonary disease (COPD) lung.

Pulmonary and General Inflammation (PGI) Panel v1.0

Pulmonary and General inflammation (PGI) panel v1.0 included two controls and 39 test samples isolated as surgical or postmortem samples. Tissue samples include: five normal lung samples obtained from Maryland Brain and Tissue Bank, University of Maryland (Baltimore, Md.), International Bioresource systems, IBS (Tuscon, Ariz.), and Asterand (Detroit, Mich.), five normal adjacent intestine tissues (NAT) from Ardais (Lexington, Mass.), ulcerative colitis samples (UC) from Ardais (Lexington, Mass.); Crohns disease colon from NDRI, National Disease Research Interchange (Philadelphia, Pa.); emphysematous tissue samples from Ardais (Lexington, Mass.) and Genomic Collaborative Inc. (Cambridge, Mass.), asthmatic tissue from Maryland Brain and Tissue Bank, University of Maryland (Baltimore, Md.) and Genomic Collaborative Inc (Cambridge, Mass.) and fibrotic tissue from Ardais (Lexinton, Mass.) and Genomic Collaborative (Cambridge, Mass.).

Cellular OA/RA Panel

Cellular OA.RA panel includes 2 control wells and 35 test samples comprised of cDNA generated from total RNA isolated from human cell lines or primary cells representative of the human joint and its inflammatory condition. Cell types included normal human osteoblasts (Nhost) from Clonetics (Cambrex, East Rutherford, N.J.), human chondrosarcoma SW1353 cells from ATCC (Manossas, Va.)), human fibroblast-like synoviocytes from Cell Applications, Inc. (San Diego, Calif.) and MH7A cell line (a rheumatoid fibroblast-like synoviocytes transformed with SV40 T antigen) from Riken Cell bank (Tsukuba Science City, Japan). These cell types were activated by incubating with various cytokines (IL-1 beta ˜1-10 ng/ml, TNF alpha ˜5-50 ng/ml, or prostaglandin E2 for Nhost cells) for 1, 6, 18 or 24 h. All these cells were starved for at least 5 h and cultured in their corresponding basal medium with ˜0.1 to 1% FBS.

Minitissue OA/RA Panel

The OA/RA mini panel includes two control wells and 31 test samples comprised of cDNA generated from total RNA isolated from surgical and postmortem human tissues obtained from the University of Calgary (Alberta, Canada), NDRI (Philadelphia, Pa.), and Ardais Corporation (Lexington, Mass.). Joint tissue samples include synovium, bone and cartilage from osteoarthritic and rheumatoid arthritis patients undergoing reconstructive knee surgery, as well as, normal synovium samples (RNA and tissue). Visceral normal tissues were pooled from 2-5 different adults and included adrenal gland, heart, kidney, brain, colon, lung, stomach, small intestine, skeletal muscle, and ovary.

AI.05 Chondrosarcoma

AI.05 chondrosarcoma plates included SW1353 cells (ATCC) subjected to serum starvation and treated for 6 and 18 h with cytokines that are known to induce MMP (1, 3 and 13) synthesis (e.g. IL1beta). These treatments included: IL-1beta (10 ng/ml), IL-1beta+TNF-alpha (50 ng/ml), IL-1beta+Oncostatin (50 ng/ml) and PMA (100 ng/ml). Supernatants were collected and analyzed for MMP 1, 3 and 13 production. RNA was prepared from these samples using standard procedures.

Panels 5D and 5I

Panel 5D and 5I included two controls and cDNAs isolated from human tissues, human pancreatic islets cells, cell lines, metabolic tissues obtained from patients enrolled in the Gestational Diabetes study (described below), and cells from different stages of adipocyte differentiation, including differentiated (AD), midway differentiated (AM), and undifferentiated (U; human mesenchymal stem cells).

Gestational Diabetes study subjects were young (18-40 years), otherwise healthy women with and without gestational, diabetes undergoing routine (elective) Caesarean section. Uterine wall smooth muscle (UT), visceral (Vis) adipose, skeletal muscle (SK), placenta (PI) greater omentum adipose (GO Adipose) and subcutaneous (SubQ) adipose samples (less than 1 cc) were collected, rinsed in sterile saline, blotted and flash frozen in liquid nitrogen. Patients included: Patient 2, an overweight diabetic Hispanic not on insulin; Patient 7-9, obese non-diabetic Caucasians with body mass index (BMI) greater than 30; Patient 10, an overweight diabetic Hispanic, on insulin; Patient 11, an overweight nondiabetic African American; and Patient 12, a diabetic Hispanic on insulin.

Differentiated adipocytes were obtained from induced donor progenitor cells (Clonetics, Walkersville, Md.). Differentiated human mesenchymal stem cells (HuMSCs) were prepared as described in Mark F. Pittenger, et al., Multilineage Potential of Adult Human Mesenchymal Stem Cells Science Apr. 2, 1999: 143-147. mRNA was isolated and sscDNA was produced from Trizol lysates or frozen pellets. Human cell lines (ATCC, NCI or German tumor cell bank) included: kidney proximal convoluted tubule, uterine smooth muscle cells, small intestine, liver HepG2 cancer cells, heart primary stromal cells and adrenal cortical adenoma cells. Cells were cultured, RNA extracted and sscDNA was produced using standard procedures.

Panel 5I also contains pancreatic islets (Diabetes Research Institute at the University of Miami School of Medicine).

Human Metabolic RTQ-PCR Panel

Human Metabolic RTQ-PCR Panel included two controls (genomic DNA control and chemistry control) and 211 cDNAs isolated from human tissues and cell lines relevant to metabolic diseases. This panel identifies genes that play a role in the etiology and pathogenesis of obesity and/or diabetes. Metabolic tissues including placenta (Pl), uterine wall smooth muscle (Ut), visceral adipose, skeletal muscle (Sk) and subcutaneous (SubQ) adipose were obtained from the Gestational Diabetes study (described above). Included in the panel are: Patients 7 and 8, obese non-diabetic Caucasians; Patient 12 a diabetic Caucasian with unknown BMI, on insulin (treated); Patient 13, an overweight diabetic Caucasian, not on insulin (untreated); Patient 15, an obese, untreated, diabetic Caucasian; Patient 17 and 25, untreated diabetic Caucasians of normal weight; Patient 18, an obese, untreated, diabetic Hispanic; Patient 19, a non-diabetic Caucasian of normal weight; Patient 20, an overweight, treated diabetic Caucasian; Patient 21 and 23, overweight non-diabetic Caucasians; Patient 22, a treated diabetic Caucasian of normal weight; Patient 23, an overweight non-diabetic Caucasian; and Patients 26 and 27, obese, treated, diabetic Caucasians.

Total RNA was isolated from metabolic tissues including: hypothalamus, liver, pancreas, pancreatic islets, small intestine, psoas muscle, diaphragm muscle, visceral (Vis) adipose, subcutaneous (SubQ) adipose and greater omentum (Go) from 12 Type II diabetic (Diab) patients and 12 non diabetic (Norm) at autopsy. Control diabetic and non-diabetic subjects were matched where possible for: age; sex, male (M); female (F); ethnicity, Caucasian (CC); Hispanic (HI); African American (AA); Asian (AS); and BMI, 20-25 (Low BM), 26-30 (Med BM) or overweight (Overwt), BMI greater than 30 (Hi BMI) (obese).

RNA was extracted and ss cDNA was produced from cell lines (ATCC) by standard methods.

CNS Panels

CNS Panels CNSD.01, CNS Neurodegeneration V1.0 and CNS Neurodegeneration V2.0 included two controls and 46 to 94 test cDNA samples isolated from postmortem human brain tissue obtained from the Harvard Brain Tissue Resource Center (McLean Hospital). Brains were removed from calvaria of donors between 4 and 24 hours after death, and frozen at −80° C. in liquid nitrogen vapor.

Panel CNSD.01

Panel CNSD.01 included two specimens each from: Alzheimer's disease, Parkinson's disease, Huntington's disease, Progressive Supernuclear Palsy (PSP), Depression, and normal controls. Collected tissues included: cingulate gyrus (Cing Gyr), temporal pole (Temp Pole), globus palladus (Glob palladus), substantia nigra (Sub Nigra), primary motor strip (Brodman Area 4), parietal cortex (Brodman Area 7), prefrontal cortex (Brodman Area 9), and occipital cortex (Brodman area 17). Not all brain regions are represented in all cases.

Panel-CNS Neurodegeneration V1.0

The CNS Neurodegeneration V1.0 panel included: six Alzheimer's disease (AD) brains and eight normals which included no dementia and no Alzheimer's like pathology (control) or no dementia but evidence of severe Alzheimer's like pathology (Control Path), specifically senile plaque load rated as level 3 on a scale of 0-3; 0 no evidence of plaques, 3 severe AD senile plaque load. Tissues collected included: hippocampus, temporal cortex (Brodman Area 21), parietal cortex (Brodman area 7), occipital cortex (Brodman area 17) superior temporal cortex (Sup Temporal Ctx) and inferior temporal cortex (Inf Temproal Ctx).

Gene expression was analyzed after normalization using a scaling factor calculated by subtracting the Well mean (CT average for the specific tissue) from the Grand mean (average CT value-for all wells across all runs). The scaled CT value is the result of the raw CT value plus the scaling factor.

Panel CNS Neurodegeneration V2.0

The CNS Neurodegeneration V2.0 panel included sixteen cases of Alzheimer's disease (AD) and twenty-nine normal controls (no evidence of dementia prior to death) including fourteen controls (Control) with no dementia and no Alzheimer's like pathology and fifteen controls with no dementia but evidence of severe Alzheimer's like pathology (AH3), specifically senile plaque load rated as level 3 on a scale of 0-3; 0 no evidence of plaques, 3 severe AD senile plaque load. Tissues from the temporal cortex (Brodman Area 21) included the inferior and superior temporal cortex that was pooled from a given individual (Inf & Sup Temp Ctx Pool).

A. NOV1 CG121992: Chordin

Expression of NOV1a gene CG121992-03 was assessed using the primer-probe set Ag8269, described in Table AA. Results of the RTQ-PCR runs are shown in Table AB.

TABLE AA
Probe Name Ag8269

| | | | Start | SEQ ID |
|---|---|---|---|---|
| Primers | Sequences | Length | Position | No |
| Forward | 5′-gagaaggttagggagagc | 22 | 1255 | 85 |
| | acct-3′ | | | |
| Probe | TET-5′-ccttgcaggactaa | 23 | 1302 | 86 |
| | cccaggttc-3′-TAMRA | | | |
| Reverse | 5′-gtgtagaatctggagcct | 22 | 1326 | 87 |
| | caag-3′ | | | |

TABLE AB
Probe Name Ag7203

| | | | Start | SEQ ID |
|---|---|---|---|---|
| Primers | Sequences | Length | Position | No |
| Forward | 5′-aggagagggggggcact- | 17 | 2379 | 88 |
| | 3′ | | | |
| Probe | FAM-5′-acactgcaccttct | 27 | 2399 | 89 |
| | cacagtgcacctc-3′- | | | |
| | TAMRA | | | |
| Reverse | 5′-actgtttgcagcagtcgg | 19 | 2465 | 90 |
| | t-3′ | | | |

TABLE AC
General_screening_panel_v1.7

| Tissue Name | A |
|---|---|
| Adipose | 10.2 |
| HUVEC | 0.2 |
| Melanoma* Hs688(A).T | 0.0 |
| Melanoma* Hs688(B).T | 0.1 |
| Melanoma (met) SK-MEL-5 | 0.3 |
| Testis | 0.5 |
| Prostate ca. (bone met) PC-3 | 0.0 |
| Prostate ca. DU145 | 0.8 |
| Prostate pool | 1.2 |
| Uterus pool | 1.9 |
| Ovarian ca. OVCAR-3 | 0.0 |
| Ovarian ca. (ascites) SK-OV-3 | 0.0 |
| Ovarian ca. OVCAR-4 | 0.2 |
| Ovarian ca. OYCAR-5 | 1.5 |
| Ovarian ca. IGROV-1 | 3.8 |
| Ovarian ca. OVCAR-8 | 0.9 |
| Ovary | 2.6 |
| Breast ca. MCF-7 | 0.6 |
| Breast ca. MDA-MB-231 | 0.1 |
| Breast ca. BT 549 | 0.0 |
| Breast ca. T47D | 1.8 |
| 113452 mammary gland | 0.0 |
| Trachea | 3.7 |
| Lung | 2.5 |
| Fetal Lung | 6.7 |
| Lung ca. NCI-N417 | 0.0 |
| Lung ca. LX-1 | 0.0 |
| Lung ca. NCI-H146 | 6.8 |
| Lung ca. SHP-77 | 5.7 |
| Lung ca. NCI-H23 | 0.9 |
| Lung ca. NCI-H460 | 0.0 |
| Lung ca. HOP-62 | 0.0 |
| Lung ca. NCI-H522 | 1.6 |
| Lung ca. DMS-114 | 1.2 |
| Liver | 0.6 |
| Fetal Liver | 7.2 |
| Kidney pool | 7.2 |
| Fetal Kidney | 0.8 |
| Renal ca. 786-0 | 4.7 |
| Renal ca. A498 | 0.0 |
| Renal ca. ACHN | 0.2 |
| Renal ca. UO-31 | 0.0 |
| Renal ca. TK-10 | 0.1 |
| Bladder | 1.5 |
| Gastric ca. (liver met.) NCI-N87 | 0.0 |
| Stomach | 0.5 |
| Colon ca. SW-948 | 0.0 |
| Colon ca. SW480 | 0.0 |
| Colon ca. (SW480 met) SW620 | 0.0 |
| Colon ca. HT29 | 3.3 |
| Colon ca. HCT-116 | 0.0 |
| Colon cancer tissue | 0.0 |
| Colon ca. SW1116 | 0.8 |
| Colon ca. Colo-205 | 0.0 |
| Colon ca. SW-48 | 0.0 |
| Colon | 1.0 |
| Small Intestine | 3.0 |
| Fetal Heart | 0.3 |
| Heart | 1.5 |
| Lymph Node Pool | 0.0 |
| Lymph Node pool 2 | 6.6 |
| Fetal Skeletal Muscle | 1.3 |
| Skeletal Muscle pool | 0.4 |
| Skeletal Muscle | 1.6 |
| Spleen | 1.7 |
| Thymus | 0.9 |
| CNS cancer (glio/astro) SF-268 | 0.1 |
| CNS cancer (glio/astro) T98G | 0.0 |
| CNS cancer (neuro; met) SK-N-AS | 0.0 |
| CNS cancer (astro) SF-539 | 0.7 |
| CNS cancer (astro) SNB-75 | 0.0 |
| CNS cancer (glio) SNB-19 | 0.7 |
| CNS cancer (glio) SF-295 | 0.1 |
| Brain (Amygdala) | 4.7 |
| Brain (Cerebellum) | 100.0 |
| Brain (Fetal) | 16.4 |
| Brain (Hippocampus) | 3.5 |
| Cerebral Cortex pool | 3.8 |
| Brain (Substantia nigra) | 2.8 |
| Brain (Thalamus) | 4.9 |
| Brain (Whole) | 84.7 |
| Spinal Cord | 1.2 |
| Adrenal Gland | 5.2 |
| Pituitary Gland | 0.9 |
| Salivary Gland | 0.4 |
| Thyroid | 2.2 |
| Pancreatic ca. PANC-1 | 0.1 |
| Pancreas pool | 1.2 |

Column A - Rel. Exp. (%) Ag8269, Run 325595059

General_screening_panel_v1.7 Summary: Results using probe-primer sets Ag8269 and Ag7203 showed similar expression profile. This gene was highly expressed in brain (CT=25.27) and adipose (CT=28.57). This gene encodes a human chordin polypeptide. The chordin polypeptides have homology to Xenopus chordin, a secreted molecule that functions as a dorsalizing factor in early embryo development. Chordin binds and antagonizes BMP4, a member of the transforming growth factor (TGF)-beta superfamily. Therapeutic modulation of the activity of this gene is useful in the treatment of endocrine/metabolically related diseases, such as obesity and diabetes, and central nervous system disorders such as Alzheimer's disease, Parkinson's disease, epilepsy, multiple sclerosis, schizophrenia and depression.

B. NOV2 CG186275: Adam 22

Expression of gene CG186275-03 was assessed using the primer-probe sets Ag7761 and Ag8175, described in Tables BA and BB. Results of the RTQ-PCR runs are shown in Table BC.

TABLE BA
Probe Name Ag7761

| | | | Start | SEQ ID |
|---|---|---|---|---|
| Primers | Sequences | Length | Position | No |
| Forward | 5′-atataccagatacagttg | 28 | 329 | 91 |
| | actcatgttg-3′ | | | |
| Probe | TET-5′-accaagcaagcttc | 25 | 357 | 92 |
| | caggttgatgc-3′-TAMRA | | | |
| Reverse | 5′-gagaatgaatgacgttcc | 22 | 382 | 93 |
| | aaag-3′ | | | |

TABLE BB
Probe Name Ag8175

| | | | Start | SEQ ID |
|---|---|---|---|---|
| Primers | Sequences | Length | Position | No |
| Forward | 5′-cattctcgatgtcgtgct | 22 | 397 | 94 |
| | aaat-3′ | | | |
| Probe | TET-5′-catgatttgctgtc | 29 | 419 | 95 |
| | ctctgaatacataga-3′- | | | |
| | TAMRA | | | |
| Reverse | 5′-cttgcctccatgttcaat | 20 | 453 | 96 |
| | gt-3′ | | | |

TABLE BC
General_screening_panel_v1.7

| Tissue Name | A |
|---|---|
| Adipose | 3.8 |
| HUVEC | 0.7 |
| Melanoma* Hs688(A).T | 0.0 |
| Melanoma* Hs688(B).T | 1.1 |
| Melanoma (met) SK-MEL-5 | 2.2 |
| Testis | 1.1 |
| Prostate ca. (bone met) PC-3 | 0.0 |
| Prostate ca. DU145 | 4.9 |
| Prostate pool | 1.0 |
| Uterus pool | 0.4 |
| Ovarian ca. OVCAR-3 | 1.3 |
| Ovarian ca. (ascites) SK-OV-3 | 0.2 |
| Ovarian ca. OVCAR-4 | 0.3 |
| Ovarian ca. OVCAR-5 | 2.0 |
| Ovarian ca. IGROV-1 | 4.6 |
| Ovarian ca. OVCAR-8 | 1.6 |
| Ovary | 2.6 |
| Breast ca. MCF-7 | 1.1 |
| Breast ca. MDA-MB-231 | 3.1 |
| Breast ca. BT 549 | |
| Breast ca. T47D | 0.8 |
| 113452 mammary gland | 0.0 |
| Trachea | 1.2 |
| Lung | 1.2 |
| Fetal Lung | 3.4 |
| Lung ca. NCI-N417 | 2.9 |
| Lung ca. LX-1 | 0.7 |
| Lung ca. NCI-H146 | 9.7 |
| Lung ca. SHP-77 | 35.8 |
| Lung ca. NCI-H23 | 2.9 |
| Lung ca. NCI-H460 | 2.6 |
| Lung ca. HOP-62 | 0.9 |
| Lung ca. NCI-H522 | 6.9 |
| Lung ca. DMS-114 | 1.2 |
| Liver | 0.0 |
| Fetal Liver | |
| Kidney pool | 7.5 |
| Fetal Kidney | 2.7 |
| Renal ca. 786-0 | 1.5 |
| Renal ca. A498 | 1.0 |
| Renal ca. ACHN | |
| Renal ca. UO-31 | 0.4 |
| Renal ca. TK-10 | 10.9 |
| Bladder | 0.9 |
| Gastric ca. (liver met.) NCI-N87 | 0.0 |
| Stomach | |
| Colon ca. SW-948 | 2.6 |
| Colon ca. SW480 | 0.2 |
| Colon ca. (SW480 met) SW620 | 14.5 |
| Colon ca. HT29 | 8.8 |
| Colon ca. HCT-116 | 9.7 |
| Colon cancer tissue | |
| Colon ca. SW1116 | 2.6 |
| Colon ca. Colo-205 | 0.8 |
| Colon ca. SW-48 | 0.2 |
| Colon | 0.9 |
| Small Intestine | 0.5 |
| Fetal Heart | 0.5 |
| Heart | 0.4 |
| Lymph Node Pool | 0.6 |
| Lymph Node pool 2 | 2.9 |
| Fetal Skeletal Muscle | 0.9 |
| Skeletal Muscle pool | |
| Skeletal Muscle | 0.4 |
| Spleen | 1.5 |
| Thymus | 1.1 |
| CNS cancer (glio/astro) SF-268 | |
| CNS cancer (glio/astro) T98G | 1.4 |
| CNS cancer (neuro; met) SK-N-AS | 0.6 |
| CNS cancer (astro) SF-539 | 1.0 |
| CNS cancer (astro) SNB-75 | 1.0 |
| CNS cancer (glio) SNB-19 | 1.2 |
| CNS cancer (glio) SF-295 | 0.3 |
| Brain (Amygdala) | 13.8 |
| Brain (Cerebellum) | 100.0 |
| Brain (Fetal) | 21.5 |
| Brain (Hippocampus) | 8.1 |
| Cerebral Cortex pool | 5.7 |
| Brain (Substantia nigra) | 3.5 |
| Brain (Thalamus) | 2.0 |
| Brain (Whole) | 82.4 |
| Spinal Cord | 2.8 |
| Adrenal Gland | 5.5 |
| Pituitary Gland | 2.4 |
| Salivary Gland | 0.3 |
| Thyroid | 4.5 |
| Pancreatic ca. PANC-1 | 0.2 |
| Pancreas pool | |

Column A - Rel. Exp. (%) Ag7761, Run 318349329

General_screening_panel_v1.7 Summary: Ag7761 The highest expression of this gene was detected in cerebellum (CT=24). Generally, this gene is ubiquitously expressed at moderate to low levels. It was upregulated in some colon and lung cancers and therefore is useful as a marker for these cancers and to differentiate between cancerous and normal tissue of these organs. Therapeutic modulation of this gene, expressed protein and/or use of antibodies or small molecule drugs targeting the gene or gene product is useful in the treatment of colon and lung cancer.

C. NOV3, CG50586: Beta-Secretase

Expression of gene CG50586-03 and NOV3b, 260368280, were assessed using the primer-probe set Ag43, described in Table CA. Results of the RTQ-PCR runs are shown in Table CB.

TABLE CA
Probe Name Ag43

| | | | Start | SEQ ID |
|---|---|---|---|---|
| Primers | Sequences | Length | Position | No |
| Forward | 5′-aaatcgcaagacattcac | 23 | 558 | 97 |
| | tgtca-3′ | | | |
| Probe | TET-5′-cagcacactggact | 26 | 582 | 98 |
| | tccgagtggacc-3′-TAMRA | | | |
| Reverse | 5′-ccgccactccatcatcac | 19 | 610 | 99 |
| | t-3′ | | | |

TABLE CB
Panel 1

| Tissue Name | A |
|---|---|
| Endothelial cells | 0.0 |
| Endothelial cells (treated) | 0.0 |
| Pancreas | 0.2 |
| Pancreatic ca. CAPAN 2 | 0.0 |
| Adrenal gland | 0.6 |
| Thyroid | 0.2 |
| Salivary gland | 0.3 |
| Pituitary gland | 0.1 |
| Brain (fetal) | 11.5 |
| Brain (whole) | 0.0 |
| Brain (amygdala) | 18.9 |
| Brain (cerebellum) | 100.0 |
| Brain (hippocampus) | 57.0 |
| Brain (substantia nigra) | 23.2 |
| Brain (thalamus) | 12.6 |
| Brain (hypothalamus) | 4.3 |
| Spinal cord | 2.8 |
| glio/astro U87-MG | 0.0 |
| glio/astro U-118-MG | 0.0 |
| astrocytoma SW1783 | 0.0 |
| neuro*; met SK-N-AS | 2.0 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.0 |
| Heart | 0.4 |
| Skeletal muscle | 1.1 |
| Bone marrow | 0.1 |
| Thymus | 0.3 |
| Spleen | 0.1 |
| Lymph node | 0.1 |
| Colon (ascending) | 1.1 |
| Stomach | 2.1 |
| Small intestine | 0.8 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| Colon ca. HCT-15 | 0.0 |
| Colon ca. HCC-2998 | 0.1 |
| Gastric ca. (liver met) NCI-N87 | 0.0 |
| Bladder | 1.3 |
| Trachea | 0.7 |
| Kidney | 0.1 |
| Kidney (fetal) | 0.2 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.1 |
| Liver (fetal) | 0.0 |
| Liver ca. (hepatoblast) HepG2 | 0.0 |
| Lung | 0.0 |
| Lung (fetal) | 0.1 |
| Lung ca. (small cell) LX-1 | 0.0 |
| Lung ca. (small cell) NCI-H69 | 1.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 1.0 |
| Lung ca. (non-s. cell) HOP-62 | 0.1 |
| Lung ca. (non-s. cl) NCI-H522 | 0.3 |
| Lung ca: (squam.) SW 900 | 0.8 |
| Lung ca. (squam.) NCI-H596 | 1.2 |
| Mammary gland | 1.0 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.1 |
| Ovarian ca. OVCAR-3 | 0.5 |
| Ovarian ca. OVCAR-4 | 0.0 |
| Ovarian ca. OVCAR-5 | 0.1 |
| Ovarian ca. OVCAR-8 | 0.1 |
| Ovarian ca. IGROV-1 | 0.0 |
| Ovarian ca. (ascites) SK-OV-3 | 0.0 |
| Uterus | 0.1 |
| Placenta | 0.2 |
| Prostate | 0.7 |
| Prostate ca.* (bone met) PC-3 | 0.0 |
| Testis | 1.8 |
| Melanoma Hs688(A).T | 0.0 |
| Melanoma* (met) Hs688(B).T | 0.0 |
| Melanoma UACC-62 | 0.0 |
| Melanoma M14 | 0.1 |
| Melanoma LOX IMVI | 0.0 |
| Melanoma* (met) SK-MEL-5 | 0.2 |
| Melanoma SK-MEL-28 | 0.0 |

Column A - Rel. Exp. (%) Ag43, Run 87354406

Panel 1 Summary: Ag43 Highest expression of this gene was detected in brain tissues (CT=21.7) and spinal cord (CT=26.68). This gene encodes the human beta-secretase enzyme polynucleotide. Beta-secretase is capable of cleaving the beta-amyloid precursor protein (APP) (AAY33742;swedish mutant APP). This enzyme is useful in detecting human beta-secretase cleavage of polypeptides and for identifying beta-secretase inhibitors. Therapeutic modulation of the activity of this gene is useful in the treatment of central nervous system disorders such as Alzheimer's disease, Parkinson's disease, epilepsy, multiple sclerosis, schizophrenia and depression.

D. NOV4 CG50637: T-Cell Surface Glycoprotein CD1B Precursor

Expression of gene NOV4a, CG50637-01; NOV4b 277577082; and NOV4c 277577094 were assessed using the primer-probe set Ag2828, described in Table DA. Results of the RTQ-PCR runs are shown in Tables DB, DC and DD.

TABLE DA
Probe Name Ag2828

| | | | Start | SEQ ID |
|---|---|---|---|---|
| Primers | Sequences | Length | Position | No |
| Forward | 5′-ctgaggctgtctccttga | 20 | 1401 | 100 |
| | tg-3′ | | | |
| Probe | TET-5′-ctggaaggcttctc | 24 | 1376 | 101 |
| | tacaccccgg-3′-TAMRA | | | |
| Reverse | 5′-gtggtcctggtcctcata | 22 | 1326 | 102 |
| | tacc-3′ | | | |

TABLE DB
General_screening_panel_v1.5

| Tissue Name | A |
|---|---|
| Adipose | 2.0 |
| Melanoma* Hs688(A).T | 1.9 |
| Melanoma* Hs688(B).T | 1.3 |
| Melanoma* M14 | 4.5 |
| Melanoma* LOXIMVI | 1.3 |
| Melanoma* SK-MEL-5 | 1.8 |
| Squamous cell carcinoma SCC-4 | 0.6 |
| Testis Pool | 3.7 |
| Prostate ca.* (bone met) PC-3 | 5.2 |
| Prostate Pool | 6.9 |
| Placenta | 3.3 |
| Uterus Pool | 3.2 |
| Ovarian ca. OVCAR-3 | 6.3 |
| Ovarian ca. SK-OV-3 | 15.6 |
| Ovarian ca. OVCAR-4 | 1.0 |
| Ovarian ca. OVCAR-5 | 6.0 |
| Ovarian ca. IGROV-1 | 4.9 |
| Ovarian ca. OVCAR-8 | 7.4 |
| Ovary | 4.7 |
| Breast ca. MCF-7 | 18.4 |
| Breast ca. MDA-MB-231 | 6.9 |
| Breast ca. BT 549 | 7.3 |
| Breast ca. T47D | 1.9 |
| Breast ca. MDA-N | 1.3 |
| Breast Pool | 6.0 |
| Trachea | 4.6 |
| Lung | 1.7 |
| Fetal Lung | 3.1 |
| Lung ca. NCI-N417 | 2.6 |
| Lung ca. LX-1 | 0.5 |
| Lung ca. NCI-H146 | 1.3 |
| Lung ca. SHP-77 | 5.5 |
| Lung ca. A549 | 1.3 |
| Lung ca. NCI-H526 | 0.9 |
| Lung ca. NCI-H23 | 14.2 |
| Lung ca. NCI-H460 | 3.5 |
| Lung ca. HOP-62 | 0.9 |
| Lung ca. NCI-H522 | 9.8 |
| Liver | 0.8 |
| Fetal Liver | 1.8 |
| Liver ca. HepG2 | 1.2 |
| Kidney Pool | 12.1 |
| Fetal Kidney | 2.1 |
| Renal ca. 786-0 | 0.2 |
| Renal ca. A498 | 0.9 |
| Renal ca. ACHN | 1.1 |
| Renal ca. UO-31 | 2.8 |
| Renal ca. TK-10 | 2.2 |
| Bladder | 2.7 |
| Gastric ca. (liver met.) NCI-N87 | 14.4 |
| Gastric ca. KATO III | 11.4 |
| Colon ca. SW-948 | 1.7 |
| Colon ca. SW480 | 4.7 |
| Colon ca.* (SW480 met) SW620 | 1.5 |
| Colon ca. HT29 | 0.7 |
| Colon ca. HCT-116 | 0.1 |
| Colon ca. CaCo-2 | 1.3 |
| Colon cancer tissue | 2.6 |
| Colon ca. SW1116 | 1.3 |
| Colon ca. Colo-205 | 3.1 |
| Colon ca. SW-48 | 2.7 |
| Colon Pool | 6.4 |
| Small Intestine Pool | 4.9 |
| Stomach Pool | 2.5 |
| Bone Marrow Pool | 2.9 |
| Fetal Heart | 2.7 |
| Heart Pool | 2.9 |
| Lymph Node Pool | 6.1 |
| Fetal Skeletal Muscle | 5.2 |
| Skeletal Muscle Pool | 18.0 |
| Spleen Pool | 3.9 |
| Thymus Pool | 4.5 |
| CNS cancer (glio/astro) U87-MG | 1.8 |
| CNS cancer (glio/astro) U-118-MG | 0.4 |
| CNS cancer (neuro; met) SK-N-AS | 3.5 |
| CNS cancer (astro) SF-539 | 0.1 |
| CNS cancer (astro) SNB-75 | 1.6 |
| CNS cancer (glio) SNB-19 | 7.7 |
| CNS cancer (glio) SF-295 | 2.7 |
| Brain (Amygdala) Pool | 15.5 |
| Brain (cerebellum) | 100.0 |
| Brain (fetal) | 19.2 |
| Brain (Hippocampus) Pool | 11.7 |
| Cerebral Cortex Pool | 20.0 |
| Brain (Substantia nigra) Pool | 20.7 |
| Brain (Thalamus) Pool | 26.1 |
| Brain (whole) | 33.0 |
| Spinal Cord Pool | 6.2 |
| Adrenal Gland | 23.0 |
| Pituitary gland Pool | 3.0 |
| Salivary Gland | 3.1 |
| Thyroid (female) | 5.8 |
| Pancreatic ca. CAPAN2 | 0.2 |
| Pancreas Pool | 3.5 |

Column A - Rel. Exp. (%) Ag2828, Run 254396109

TABLE DC
Panel 4D

| Tissue Name | A |
|---|---|
| Secondary Th1 act | 0.0 |
| Secondary Th2 act | 2.9 |
| Secondary Tr1 act | 2.8 |
| Secondary Th1 rest | 9.5 |
| Secondary Th2 rest | 11.1 |
| Secondary Tr1 rest | 11.9 |
| Primary Th1 act | 10.8 |
| Primary Th2 act | 9.3 |
| Primary Tr1 act | 4.5 |
| Primary Th1 rest | 100.0 |
| Primary Th2 rest | 75.8 |
| Primary Tr1 rest | 47.3 |
| CD45RA CD4 lymphocyte act | 6.2 |
| CD45RO CD4 lymphocyte act | 11.9 |
| CD8 lymphocyte act | 13.9 |
| Secondary CD8 lymphocyte rest | 22.5 |
| Secondary CD8 lymphocyte act | 0.4 |
| CD4 lymphocyte none | 47.0 |
| 2ry Th1/Th2/Tr1 anti-CD95 CH11 | 27.5 |
| LAK cells rest | 8.9 |
| LAK cells IL-2 | 27.4 |
| LAK cells IL-2 + IL-12 | 14.9 |
| LAK cells IL-2 + IFN gamma | 28.7 |
| LAK cells IL-2 + IL-18 | 16.5 |
| LAK cells PMA/ionomycin | 2.6 |
| NK Cells IL-2 rest | 27.2 |
| Two Way MLR 3 day | 28.9 |
| Two Way MLR 5 day | 11.2 |
| Two Way MLR 7 day | 5.7 |
| PBMC rest | 42.6 |
| PBMC PWM | 27.9 |
| PBMC PHA-L | 21.6 |
| Ramos (B cell) none | 0.0 |
| Ramos (B cell) ionomycin | 0.0 |
| B lymphocytes PWM | 9.2 |
| B lymphocytes CD40L and IL-4 | 5.6 |
| EOL-1 dbcAMP | 1.5 |
| EOL-1 dbcAMP PMA/ionomycin | 2.8 |
| Dendritic cells none | 3.5 |
| Dendritic cells LPS | 1.1 |
| Dendritic cells anti-CD40 | 6.0 |
| Monocytes rest | 1.9 |
| Monocytes LPS | 0.0 |
| Macrophages rest | 20.9 |
| Macrophages LPS | 1.7 |
| HUVEC none | 5.6 |
| HUVEC starved | 4.5 |
| HUVEC IL-1beta | 0.0 |
| HUVEC IFN gamma | 2.0 |
| HUVEC TNF alpha + IFN gamma | 0.0 |
| HUVEC TNF alpha + IL4 | 0.8 |
| HUVEC IL-11 | 0.0 |
| Lung Microvascular EC none | 2.3 |
| Lung Microvascular EC TNFalpha + IL-1beta | 0.0 |
| Microvascular Dermal EC none | 12.2 |
| Microsvasular Dermal EC TNFalpha + IL-1beta | 3.5 |
| Bronchial epithelium TNFalpha + IL1beta | 0.2 |
| Small airway epithelium none | 2.3 |
| Small airway epithelium TNFalpha + IL-1beta | 7.9 |
| Coronery artery SMC rest | 2.5 |
| Coronery artery SMC TNFalpha + IL-1beta | 1.0 |
| Astrocytes rest | 11.5 |
| Astrocytes TNFalpha + IL-1beta | 17.8 |
| KU-812 (Basophil) rest | 3.5 |
| KU-812 (Basophil) PMA/ionomycin | 5.8 |
| CCD1106 (Keratinocytes) none | 11.5 |
| CCD1106 (Keratinocytes) TNFalpha + IL-1beta | 6.2 |
| Liver cirrhosis | 6.0 |
| Lupus kidney | 3.2 |
| NCI-H292 none | 46.7 |
| NCI-H292 IL-4 | 81.8 |
| NCI-H292 IL-9 | 69.7 |
| NCI-H292 IL-13 | 37.6 |
| NCI-H292 IFN gamma | 44.8 |
| HPAEC none | 7.8 |
| HPAEC TNF alpha + IL-1 beta | 0.0 |
| Lung fibroblast none | 14.6 |
| Lung fibroblast TNF alpha + IL-1 beta | 6.5 |
| Lung fibroblast IL-4 | 5.3 |
| Lung fibroblast IL-9 | 8.2 |
| Lung fibroblast IL-13 | 4.8 |
| Lung fibroblast IFN gamma | 6.1 |
| Dermal fibroblast CCD1070 rest | 14.7 |
| Dermal fibroblast CCD1070 TNF alpha | 17.3 |
| Dermal fibroblast CCD1070 IL-1 beta | 2.7 |
| Dermal fibroblast IFN gamma | 3.3 |
| Dermal fibroblast IL-4 | 25.7 |
| IBD Colitis 2 | 2.4 |
| IBD Crohn's | 3.7 |
| Colon | 54.3 |
| Lung | 27.7 |
| Thymus | 75.8 |
| Kidney | 77.9 |

Column A - Rel. Exp. (%) Ag2828, Run 162350533

TABLE DD
Panel 5 islet

| Tissue Name | A | B |
|---|---|---|
| 97457 Patient-02go adipose | 22.1 | 27.7 |
| 97476 Patient-07sk skeletal muscle | 18.3 | 20.6 |
| 97477 Patient-07ut uterus | 17.6 | 39.2 |
| 97478 Patient-07pl placenta | 24.1 | 49.7 |
| 99167 Bayer Patient 1 | 68.8 | 59.0 |
| 97482 Patient-08ut uterus | 12.1 | 24.7 |
| 97483 Patient-08pl placenta | 7.1 | 5.1 |
| 97486 Patient-09sk skeletal muscle | 5.4 | 25.9 |
| 97487 Patient-09ut uterus | 39.8 | 60.7 |
| 97488 Patient-09pl placenta | 13.2 | 13.2 |
| 97492 Patient-10ut uterus | 25.5 | 31.6 |
| 97493 Patient-10pl placenta | 45.4 | 95.3 |
| 97495 Patient-11go adipose | 30.1 | 30.8 |
| 97496 Patient-11sk skeletal muscle | 55.5 | 85.9 |
| 97497 Patient-11ut uterus | 46.0 | 96.6 |
| 97498 Patient-11pl placenta | 12.1 | 17.4 |
| 97500 Patient-12go adipose | 20.9 | 7.5 |
| 97501 Patient-12sk skeletal muscle | 100.0 | 100.0 |
| 97502 Patient-12ut uterus | 26.4 | 18.8 |
| 97503 Patient-12pl placenta | 27.4 | 48.3 |
| 94721 Donor 2 U - A | 6.4 | 9.7 |
| Mesenchymal Stem Cells | | |
| 94722 Donor 2 U - B | 6.6 | 10.1 |
| Mesenchymal Stem Cells | | |
| 94723 Donor 2 U - C | 7.4 | 5.3 |
| Mesenchymal Stem Cells | | |
| 94709 Donor 2 AM - A adipose | 8.5 | 7.6 |
| 94710 Donor 2 AM - B adipose | 3.1 | 5.0 |
| 94711 Donor 2 AM - C adipose | 4.5 | 7.5 |
| 94712 Donor 2 AD - A adipose | 14.1 | 9.9 |
| 94713 Donor 2 AD - B adipose | 10.6 | 26.2 |
| 94714 Donor 2 AD - C adipose | 25.5 | 20.9 |
| 94742 Donor 3 U - A | 2.2 | 3.4 |
| Mesenchymal Stem Cells | | |
| 94743 Donor 3 U - B | 3.2 | 13.3 |
| Mesenchymal Stem Cells | | |
| 94730 Donor 3 AM - A adipose | 6.2 | 9.1 |
| 94731 Donor 3 AM - B adipose | 3.9 | 4.0 |
| 94732 Donor 3 AM - C adipose | 2.9 | 3.4 |
| 94733 Donor 3 AD - A adipose | 10.8 | 23.2 |
| 94734 Donor 3 AD - B adipose | 4.0 | 3.0 |
| 94735 Donor 3 AD - C adipose | 3.7 | 2.6 |
| 77138 Liver HepG2untreated | 6.3 | 9.7 |
| 73556 Heart Cardiac stromal cells (primary) | 0.0 | 4.7 |
| 81735 Small Intestine | 30.1 | 40.9 |
| 72409 Kidney Proximal Convoluted Tubule | 6.0 | 14.0 |
| 82685 Small intestine Duodenum | 23.3 | 53.2 |
| 90650 Adrenal Adrenocortical adenoma | 16.5 | 33.2 |
| 72410 Kidney HRCE | 17.4 | 28.5 |
| 72411 Kidney HRE | 8.1 | 17.0 |
| 73139 Uterus Uterine smooth muscle cells | 3.6 | 3.8 |

Column A - Rel. Exp. (%) Ag2828, Run 253721040
Column B - Rel. Exp. (%) Ag2828, Run 254275034

General_screening_panel_v1.5 Summary: Ag2828 The highest expression of this gene was detected in cerebellum (CT=26). Generally this gene was ubiquitously expressed. Among tissues with metabolic or endocrine function, this gene was expressed at high to moderate levels in pancreas, adipose, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product are useful in the treatment of endocrine/metabolically related diseases, such as obesity and diabetes.

Panel 4D Summary: Ag2828 The highest expression of this gene was detected in resting primary Th1 cells (CT=30). This gene was expressed in thymus, colon, lung and kidney. The expression of this gene was downregulated in Crohn's disease and colitis.

Unstimulated T lymphocytes (Th1, Th2, and Tr1) expressed this gene at higher levels than anti-CD28+anti-CD3-stimulated T cells. The gene or protein product therefore is useful as a marker of resting vs activated T cells. Thus, this gene may be involved in T lymphocyte function. Therapeutic modulation of this gene, expressed protein and/or use of antibodies or small molecule drugs targeting the gene or gene product are useful in the treatment of T cell-mediated autoimmune and inflammatory diseases.

Panel 5 Islet Summary: Ag2828 The highest expression of this gene was detected in skeletal muscle from a diabetic patient (CT=30). It was also expressed in adipose, uterus and placenta from both diabetic and non-diabetic individuals. Therapeutic modulation of this gene, expressed protein and/or use of antibodies or small molecule drugs targeting the gene or gene product are useful in the treatment of endocrine/metabolically related diseases, such as obesity and diabetes.

E. NOV5, CG51117, Nephronectin

Expression of gene NOV5a through 5f, CG51117 were assessed using the primer-probe sets Ag2505, Ag2667, Ag2767, Ag2831 and Ag7237, described in Tables EA, EB, EC, ED and EE. Results of the RTQ-PCR runs are shown in Tables EF, EG, EH, EI, EJ, EK, EL and EM.

TABLE EA
Probe Name Ag2505

| | | | | |
|---|---|---|---|---|
| | | | Start | SEQ ID |
| Primers | Sequences | Length | Position | No |

| | | | | |
|---|---|---|---|---|
| Forward | 5′-aaagaaggataccagggt | 22 | 980 | 103 |
| | gatg-3′ | | | |
| Probe | TET-5′-atgattgaaccttc | 26 | 1031 | 104 |
| | aggtccaattca-3′-TAMRA | | | |
| Reverse | 5′-ggtaccatttccctttgg | 22 | 1057 | 105 |
| | taca-3′ | | | |

TABLE EB
Probe Name Ag2667

| | | | Start | SEQ ID |
|---|---|---|---|---|
| Primers | Sequences | Length | Position | No |
| Forward | 5′-gcagagaatagccaggat | 22 | 391 | 106 |
| | aagg-3′ | | | |
| Probe | TET-5′-caaccacgatgcaa | 25 | 434 | 107 |
| | acatggtgaat-3′-TAMRA | | | |
| Reverse | 5′-cacttgtttggcccgata | 19 | 459 | 108 |
| | c-3′ | | | |

TABLE EC
Probe Name Ag2767

| | | | Start | SEQ ID |
|---|---|---|---|---|
| Primers | Sequences | Length | Position | No |
| Forward | 5′-gcagagaatagccaggat | 22 | 391 | 109 |
| | aagg-3′ | | | |
| Probe | TET-5′-caaccacgatgcaa | 25 | 434 | 110 |
| | acatggtgaat-3′-TAMRA | | | |
| Reverse | 5′-cacttgtttggcccgata | 19 | 459 | 111 |
| | c-3′ | | | |

TABLE ED
Probe Name Ag2831

| | | | Start | SEQ ID |
|---|---|---|---|---|
| Primers | Sequences | Length | Position | No |
| Forward | 5′-gcagagaatagccaggat | 22 | 391 | 112 |
| | aagg-3′ | | | |
| Probe | TET-5′-caaccacgatgcaa | 25 | 434 | 113 |
| | acatggtgaat-3′-TAMRA | | | |
| Reverse | 5′-cacttgtttggcccgata | 19 | 459 | 114 |
| | c-3′ | | | |

TABLE EE
Probe Name Ag7237

| | | | Start | SEQ ID |
|---|---|---|---|---|
| Primers | Sequences | Length | Position | No |
| Forward | 5′-gtgttcattccacggcaa | 19 | 1406 | 115 |
| | c-3′ | | | |
| Probe | TET-5′-catcgtctgcactg | 27 | 1455 | 116 |
| | actcctctttcta-3′- | | | |
| | TAMRA | | | |
| Reverse | 5′-gtgtaccagaacacctgg | 22 | 492 | 117 |
| | atca-3′ | | | |

TABLE EF
AI_comprehensive panel_v1.0

| Tissue Name | A | B |
|---|---|---|
| 110967 COPD-F | 15.3 | 9.3 |
| 110980 COPD-F | 11.8 | 7.0 |
| 110968 COPD-M | 8.9 | 5.8 |
| 110977 COPD-M | 28.1 | 14.1 |
| 110989 Emphysema-F | 9.6 | 12.2 |
| 110992 Emphysema-F | 1.9 | 4.1 |
| 110993 Emphysema-F | 7.7 | 9.3 |
| 110994 Emphysema-F | 5.2 | 4.1 |
| 110995 Emphysema-F | 3.6 | 4.3 |
| 110996 Emphysema-F | 0.4 | 0.2 |
| 110997 Asthma-M | 4.6 | 3.0 |
| 111001 Asthma-F | 2.3 | 5.0 |
| 111002 Asthma-F | 3.5 | 7.2 |
| 111003 Atopic Asthma-F | 22.5 | 22.2 |
| 111004 Atopic Asthma-F | 10.4 | 11.4 |
| 111005 Atopic Asthma-F | 7.3 | 9.4 |
| 111006 Atopic Asthma-F | 1.6 | 1.4 |
| 111417 Allergy-M | 5.1 | 2.8 |
| 112347 Allergy-M | 1.4 | 0.2 |
| 112349 Normal Lung-F | 0.7 | 0.2 |
| 112357 Normal Lung-F | 7.1 | 6.4 |
| 112354 Normal Lung-M | 7.6 | 6.0 |
| 112374 Crohns-F | 9.0 | 3.2 |
| 112389 Match Control Crohns-F | 11.2 | 6.6 |
| 112375 Crohns-F | 10.2 | 6.0 |
| 112732 Match Control Crohns-F | 1.2 | 2.1 |
| 112725 Crohns-M | 0.9 | 1.6 |
| 112387 Match Control Crohns-M | 11.4 | 13.0 |
| 112378 Crohns-M | 1.3 | 2.0 |
| 112390 Match Control Crohns-M | 16.7 | 4.3 |
| 112726 Crohns-M | 21.8 | 17.0 |
| 112731 Match Control Crohns-M | 15.3 | 6.3 |
| 112380 Ulcer Col-F | 5.8 | 7.0 |
| 112734 Match Control Ulcer Col-F | 3.7 | 5.0 |
| 112384 Ulcer Col-F | 19.2 | 15.0 |
| 112737 Match Control Ulcer Col-F | 13.5 | 12.0 |
| 112386 Ulcer Col-F | 8.4 | 6.0 |
| 112738 Match Control Ulcer Col-F | 3.8 | 2.1 |
| 112381 Ulcer Col-M | 5.0 | 9.9 |
| 112735 Match Control Ulcer Col-M | 9.7 | 5.8 |
| 112382 Ulcer Col-M | 11.7 | 12.6 |
| 112394 Match Control Ulcer Col-M | 3.1 | 3.4 |
| 112383 Ulcer Col-M | 5.1 | 13.7 |
| 112736 Match Control Ulcer Col-M | 5.0 | 6.3 |
| 112423 Psoriasis-F | 14.0 | 10.3 |
| 112427 Match Control Psoriasis-F | 16.7 | 18.9 |
| 112418 Psoriasis-M | 14.0 | 13.9 |
| 112723 Match Control Psoriasis-M | 0.2 | 0.2 |
| 112419 Psoriasis-M | 18.2 | 8.7 |
| 112424 Match Control Psoriasis-M | 6.8 | 6.7 |
| 112420 Psoriasis-M | 13.9 | 15.5 |
| 112425 Match Control Psoriasis-M | 13.6 | 16.6 |
| 104689 (MF) OA Bone-Backus | 25.3 | 38.4 |
| 104690 (MF) Adj “Normal” Bone-Backus | 27.9 | 21.2 |
| 104691 (MF) OA Synovium-Backus | 2.9 | 3.0 |
| 104692 (BA) OA Cartilage-Backus | 0.0 | 0.0 |
| 104694 (BA) OA Bone-Backus | 5.8 | 18.7 |
| 104695 (BA) Adj “Normal” Bone-Backus | 14.1 | 19.8 |
| 104696 (BA) OA Synovium-Backus | 2.3 | 3.6 |
| 104700 (SS) OA Bone-Backus | 28.9 | 22.4 |
| 104701 (SS) Adj “Normal” Bone-Backus | 25.5 | 18.7 |
| 104702 (SS) OA Synovium-Backus | 11.7 | 7.1 |
| 117093 OA Cartilage Rep7 | 7.5 | 7.5 |
| 112672 OA Bone5 | 19.2 | 17.2 |
| 112673 OA Synovium5 | 6.4 | 3.8 |
| 112674 OA Synovial Fluid cells5 | 6.8 | 4.2 |
| 117100 OA Cartilage Rep14 | 1.9 | 2.1 |
| 112756 OA Bone9 | 26.4 | 31.6 |
| 112757 OA Synovium9 | 2.8 | 1.3 |
| 112758 OA Synovial Fluid Cells9 | 8.1 | 6.3 |
| 117125 RA Cartilage Rep2 | 14.8 | 9.2 |
| 113492 Bone2 RA | 84.7 | 47.0 |
| 113493 Synovium2 RA | 40.9 | 25.3 |
| 113494 Syn Fluid Cells RA | 61.1 | 49.3 |
| 113499 Cartilage4 RA | 90.1 | 73.2 |
| 113500 Bone4 RA | 100.0 | 100.0 |
| 113501 Synovium4 RA | 71.2 | 59.5 |
| 113502 Syn Fluid Cells4 RA | 48.6 | 37.9 |
| 113495 Cartilage3 RA | 77.9 | 47.0 |
| 113496 Bone3 RA | 92.0 | 41.8 |
| 113497 Synovium3 RA | 53.6 | 24.0 |
| 113498 Syn Fluid Cells3 RA | 98.6 | 57.0 |
| 117106 Normal Cartilage Rep20 | 3.0 | 1.6 |
| 113663 Bone3 Normal | 2.0 | 0.7 |
| 113664 Synovium3 Normal | 0.5 | 0.4 |
| 113665 Syn Fluid Cells3 Normal | 1.6 | 1.4 |
| 117107 Normal Cartilage Rep22 | 3.7 | 5.5 |
| 113667 Bone4 Normal | 4.3 | 6.0 |
| 113668 Synovium4 Normal | 9.1 | 7.4 |
| 113669 Syn Fluid Cells4 Normal | 6.6 | 6.6 |

Column A - Rel. Exp. (%) Ag2505, Run 248588456
Column B - Rel. Exp. (%) Ag2831, Run 244570250

TABLE EG
CNS_neurodegeneration_v1.0

| Tissue Name | A | B | C | D | E | F |
|---|---|---|---|---|---|---|
| AD 1 Hippo | 38.7 | 51.1 | 75.3 | 75.8 | 59.9 | 32.1 |
| AD 2 Hippo | 48.0 | 64.2 | 61.1 | 61.6 | 68.8 | 59.9 |
| AD 3 Hippo | 18.8 | 30.4 | 21.8 | 21.3 | 7.9 | 28.7 |
| AD 4 Hippo | 34.6 | 32.8 | 30.8 | 76.8 | 21.9 | 38.2 |
| AD 5 Hippo | 37.1 | 39.2 | 30.4 | 32.5 | 25.5 | 36.6 |
| AD 6 Hippo | 100.0 | 97.3 | 62.4 | 100.0 | 59.0 | 100.0 |
| Control 2 Hippo | 26.8 | 33.4 | 5.8 | 35.6 | 7.2 | 19.2 |
| Control 4 Hippo | 39.8 | 52.1 | 27.0 | 40.3 | 49.0 | 83.5 |
| Control (Path) 3 Hippo | 18.6 | 21.8 | 55.1 | 10.8 | 12.9 | 25.0 |
| AD 1 Temporal Ctx | 48.6 | 58.2 | 61.1 | 36.9 | 41.5 | 50.3 |
| AD 2 Temporal Ctx | 45.1 | 42.9 | 56.3 | 45.7 | 100.0 | 56.6 |
| AD 3 Temporal Ctx | 37.4 | 33.2 | 35.1 | 30.4 | 9.7 | 8.4 |
| AD 4 Temporal Ctx | 50.3 | 66.0 | 76.8 | 37.4 | 44.4 | 56.3 |
| AD 5 Inf Temporal Ctx | 23.2 | 26.4 | 20.9 | 22.5 | 29.5 | 29.7 |
| AD 5 Sup Temporal Ctx | 42.6 | 43.8 | 16.6 | 19.6 | 45.4 | 50.7 |
| AD 6 Inf Temporal Ctx | 88.9 | 100.0 | 68.8 | 72.2 | 79.6 | 100.0 |
| AD 6 Sup Temporal Ctx | 70.2 | 87.7 | 64.6 | 44.8 | 46.3 | 82.4 |
| Control 1 Temporal Ctx | 16.7 | 16.3 | 44.8 | 31.6 | 9.2 | 7.5 |
| Control 2 Temporal Ctx | 16.7 | 21.5 | 12.3 | 41.2 | 21.9 | 24.5 |
| Control 3 Temporal Ctx | 7.2 | 12.8 | 1.6 | 1.8 | 6.3 | 25.3 |
| Control 3 Temporal Ctx | 19.8 | 43.2 | 25.5 | 58.6 | 43.8 | 50.7 |
| AH3 3975 | 20.6 | 24.0 | 33.2 | 32.3 | 16.5 | 25.0 |
| AH3 3954 | 20.6 | 31.9 | 25.9 | 66.4 | 46.7 | 31.9 |
| AH3 4624 | 14.0 | 17.7 | 12.6 | 31.9 | 38.2 | 20.0 |
| AH3 4640 | 21.0 | 23.3 | 27.9 | 36.3 | 16.2 | 30.8 |
| AD 1 Occipital Ctx | 31.9 | 35.1 | 44.1 | 19.6 | 28.7 | 32.1 |
| AD 2 Occipital Ctx (Missing) | 5.2 | 4.8 | 27.9 | 30.8 | 31.4 | 8.7 |
| AD 3 Occipital Ctx | 16.7 | 23.3 | 31.2 | 2.9 | 30.6 | 12.4 |
| AD 4 Occipital Ctx | 39.8 | 46.3 | 24.3 | 20.3 | 63.3 | 56.3 |
| AD 5 Occipital Ctx | 24.7 | 22.7 | 26.4 | 34.6 | 18.2 | 27.2 |
| AD 5 Occipital Ctx | 59.9 | 87.1 | 50.7 | 56.6 | 20.2 | 66.9 |
| Control 1 Occipital Ctx | 19.9 | 14.9 | 46.7 | 3.7 | 49.0 | 14.3 |
| Control 2 Occipital Ctx | 26.2 | 29.7 | 1.8 | 36.6 | 15.9 | 39.0 |
| Control 3 Occipital Ctx | 15.3 | 15.7 | 44.4 | 11.8 | 11.7 | 20.2 |
| Control 4 Occipital Ctx | 34.2 | 36.3 | 33.7 | 35.1 | 47.6 | 64.2 |
| Control (Path) 1 Occipital Ctx | 25.7 | 24.1 | 21.9 | 27.0 | 31.4 | 30.1 |
| Control (Path) 2 Occipital Ctx | 15.1 | 20.2 | 12.9 | 16.2 | 19.1 | 15.7 |
| Control (Path) 3 Occipital Ctx | 9.5 | 14.1 | 100.0 | 21.5 | 3.8 | 14.0 |
| Control (Path) 4 Occipital Ctx | 30.6 | 37.4 | 29.9 | 32.3 | 22.8 | 27.2 |
| Control 1 Parietal Ctx | 13.3 | 13.9 | 25.0 | 88.3 | 19.2 | 13.5 |
| Control 2 Parietal Ctx | 46.7 | 57.8 | 42.0 | 44.8 | 53.6 | 66.9 |
| Control 3 Parietal Ctx | 10.1 | 12.8 | 1.6 | 8.4 | 6.3 | 12.9 |
| Control (Path) 1 Parietal Ctx | 24.5 | 29.3 | 17.0 | 23.3 | 33.2 | 31.6 |
| Control (Path) 2 Parietal Ctx | 28.3 | 35.4 | 55.9 | 22.7 | 53.2 | 37.1 |
| Control (Path) 3 Parietal Ctx | 12.8 | 18.9 | 49.7 | 19.6 | 11.1 | 11.9 |
| Control (Path) 4 Parietal Ctx | 28.3 | 32.3 | 44.1 | 28.7 | 49.0 | 33.9 |

Column A - Rel. Exp. (%) Ag2505, Run 208123723
Column B - Rel. Exp. (%) Ag2505, Run 224116291
Column C - Rel. Exp. (%) Ag2667, Run 206955569
Column D - Rel. Exp. (%) Ag2767, Run 206985756
Column E - Rel. Exp. (%) Ag2831, Run 208699692
Column F - Rel. Exp. (%) Ag7237, Run 296423778

TABLE EH
General_screening_panel_v1.6

| Tissue Name | A |
|---|---|
| Adipose | 19.9 |
| Melanoma* Hs688(A).T | 0.2 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 1.9 |
| Testis Pool | 5.0 |
| Prostate ca.* (bone met) PC-3 | 0.3 |
| Prostate Pool | 44.1 |
| Placenta | 1.0 |
| Uterus Pool | 2.0 |
| Ovarian ca. OVCAR-3 | 5.6 |
| Ovarian ca. SK-OV-3 | 18.0 |
| Ovarian ca. OVCAR-4 | 0.0 |
| Ovarian ca. OVCAR-5 | 4.7 |
| Ovarian ca. IGROV-1 | 10.9 |
| Ovarian ca. OVCAR-8 | 0.9 |
| Ovary | 2.3 |
| Breast ca. MCF-7 | 71.7 |
| Breast ca. MDA-MB-231 | 0.0 |
| Breast ca. BT 549 | 12.2 |
| Breast ca. T47D | 6.2 |
| Breast ca. MDA-N | 0.0 |
| Breast Pool | 7.5 |
| Trachea | 10.7 |
| Lung | 2.4 |
| Fetal Lung | 100.0 |
| Lung ca. NCI-N417 | 0.0 |
| Lung ca. LX-1 | 5.2 |
| Lung ca. NCI-H146 | 7.9 |
| Lung ca. SHP-77 | 0.7 |
| Lung ca. A549 | 0.7 |
| Lung ca. NCI-H526 | 0.3 |
| Lung ca. NCI-H23 | 4.5 |
| Lung ca. NCI-H460 | 0.5 |
| Lung ca. HOP-62 | 0.0 |
| Lung ca. NCI-H522 | 0.0 |
| Liver | 0.0 |
| Fetal Liver | 1.3 |
| Liver ca. HepG2 | 2.7 |
| Kidney Pool | 0.0 |
| Fetal Kidney | 23.2 |
| Renal ca. 786-0 | 0.0 |
| Renal ca. A498 | 0.0 |
| Renal ca. ACHN | 47.6 |
| Renal ca. UO-31 | 0.0 |
| Renal ca. TK-10 | 2.4 |
| Bladder | 19.2 |
| Gastric ca. (liver met.) NCI-N87 | 45.7 |
| Gastric ca. KATO III | 11.7 |
| Colon ca. SW-948 | 9.1 |
| Colon ca. SW480 | 0.5 |
| Colon ca.* (SW480 met) SW620 | 0.0 |
| Colon ca. HT29 | 12.4 |
| Colon ca. HCT-116 | 10.0 |
| Colon ca. CaCo-2 | 17.4 |
| Colon cancer tissue | 5.9 |
| Colon ca. SW1116 | 4.8 |
| Colon ca. Colo-205 | 4.2 |
| Colon ca. SW-48 | 10.1 |
| Colon Pool | 5.8 |
| Small Intestine Pool | 6.3 |
| Stomach Pool | 4.3 |
| Bone Marrow Pool | 6.4 |
| Fetal Heart | 6.0 |
| Heart Pool | 4.5 |
| Lymph Node Pool | 18.0 |
| Fetal Skeletal Muscle | 12.8 |
| Skeletal Muscle Pool | 0.6 |
| Spleen Pool | 5.7 |
| Thymus Pool | 6.9 |
| CNS cancer (glio/astro) U87-MG | 0.0 |
| CNS cancer (glio/astro) U-118-MG | 0.0 |
| CNS cancer (neuro; met) SK-N-AS | 0.1 |
| CNS cancer (astro) SF-539 | 1.7 |
| CNS cancer (astro) SNB-75 | 0.7 |
| CNS cancer (glio) SNB-19 | 12.7 |
| CNS cancer (glio) SF-295 | 0.2 |
| Brain (Amygdala) Pool | 3.0 |
| Brain (cerebellum) | 0.6 |
| Brain (fetal) | 24.8 |
| Brain (Hippocampus) Pool | 7.5 |
| Cerebral Cortex Pool | 3.7 |
| Brain (Substantia nigra) Pool | 1.9 |
| Brain (Thalamus) Pool | 5.5 |
| Brain (whole) | 6.1 |
| Spinal Cord Pool | 1.0 |
| Adrenal Gland | 3.1 |
| Pituitary gland Pool | 5.8 |
| Salivary Gland | 0.7 |
| Thyroid (female) | 47.3 |
| Pancreatic ca. CAPAN2 | 0.7 |
| Pancreas Pool | 9.6 |

Column A - Rel. Exp. (%) Ag7237, Run 296433071

TABLE EI
PGI1.0

| Tissue Name | A |
|---|---|
| 162191 Normal Lung 1 (IBS) | 9.2 |
| 160468 MD lung | 19.2 |
| 156629 MD Lung 13 | 7.9 |
| 162570 Normal Lung 4 (Aastrand) | 9.5 |
| 162571 Normal Lung 3 (Aastrand) | 5.2 |
| 162187 Fibrosis Lung 2 (Genomic Collaborative) | 89.5 |
| 151281 Fibrosis lung 11(Ardais) | 100.0 |
| 162186 Fibrosis Lung 1 (Genomic Collaborative) | 79.0 |
| 162190 Asthma Lung 4 (Genomic Collaborative) | 60.7 |
| 160467 Asthma Lung 13 (MD) | 11.8 |
| 137027 Emphysema Lung 1 (Ardais) | 11.3 |
| 137028 Emphysema Lung 2 (Ardais) | 11.7 |
| 137040 Emphysema Lung 3 (Ardais) | 26.4 |
| 137041 Emphysema Lung 4 (Ardais) | 23.7 |
| 137043 Emphysema Lung 5 (Ardais) | 29.3 |
| 142817 Emphysema Lung 6 (Ardais) | 48.0 |
| 142818 Emphysema Lung 7 (Ardais) | 33.4 |
| 142819 Emphysema Lung 8 (Ardais) | 50.3 |
| 142820 Emphysema Lung 9 (Ardais) | 9.5 |
| 142821 Emphysema Lung 10 (Ardais) | 27.4 |
| 162185 Emphysema Lung 12 (Ardais) | 48.0 |
| 162184 Emphysema Lung 13 (Ardais) | 14.5 |
| 162183 Emphysema Lung 14 (Ardais) | 59.5 |
| 162188 Emphysema Lung 15 (Genomic Collaborative) | 77.9 |
| 162177 NAT UC Colon 1(Ardais) | 4.8 |
| 162176 UC Colon 1(Ardais) | 2.3 |
| 162179 NAT UC Colon 2(Ardais) | 6.7 |
| 162178 UC Colon 2(Ardais) | 3.6 |
| 162181 NAT UC Colon 3(Ardais) | 4.1 |
| 162180 UC Colon 3(Ardais) | 1.8 |
| 162182 NAT UC Colon 4 (Ardais) | 7.5 |
| 137042 UC Colon 1108 | 1.2 |
| 137029 UC Colon 8215 | 1.8 |
| 137031 UC Colon 8217 | 1.4 |
| 137036 UC Colon 1137 | 3.3 |
| 137038 UC Colon 1491 | 2.7 |
| 137039 UC Colon 1546 | 5.8 |
| 162593 Crohn's 47751 (NDRI) | 0.4 |
| 162594 NAT Crohn's 47751 (NDRI) | 1.9 |

Column A - Rel. Exp. (%) Ag2505, Run 406107081

TABLE EJ
Panel 1.3D

| Tissue Name | A | B | C | D |
|---|---|---|---|---|
| Liver adenocarcinoma | 1.8 | 0.0 | 0.0 | 1.3 |
| Pancreas | 13.7 | 8.9 | 35.4 | 14.1 |
| Pancreatic ca. CAPAN 2 | 1.5 | 2.0 | 0.0 | 2.0 |
| Adrenal gland | 3.6 | 2.9 | 2.8 | 4.6 |
| Thyroid | 100.0 | 52.5 | 100.0 | 67.8 |
| Salivary gland | 4.1 | 2.3 | 9.3 | 2.5 |
| Pituitary gland | 37.6 | 9.9 | 18.7 | 19.8 |
| Brain (fetal) | 44.1 | 6.8 | 40.9 | 28.5 |
| Brain (whole) | 9.3 | 0.6 | 11.2 | 0.0 |
| Brain (amygdala) | 8.1 | 4.4 | 8.2 | 2.6 |
| Brain (cerebellum) | 1.8 | 0.8 | 0.0 | 4.4 |
| Brain (hippocampus) | 10.2 | 1.6 | 6.4 | 2.2 |
| Brain (substantia nigra) | 29.3 | 3.7 | 12.5 | 11.0 |
| Brain (thalamus) | 3.6 | 1.9 | 8.7 | 7.2 |
| Cerebral Cortex | 7.7 | 8.8 | 3.8 | 1.3 |
| Spinal cord | 15.2 | 14.4 | 13.3 | 10.4 |
| glio/astro U87-MG | 0.0 | 0.0 | 0.0 | 0.0 |
| glio/astro U-118-MG | 0.0 | 0.0 | 0.0 | 0.0 |
| astrocytoma SW1783 | 0.3 | 0.6 | 0.0 | 1.0 |
| neuro*; met SK-N-AS | 0.4 | 0.0 | 0.0 | 0.0 |
| astrocytoma SF-539 | 1.8 | 1.2 | 2.5 | 1.2 |
| astrocytoma SNB-75 | 2.7 | 0.6 | 0.0 | 2.0 |
| glioma SNB-19 | 0.0 | 0.0 | 0.0 | 0.0 |
| glioma U251 | 9.3 | 2.0 | 12.2 | 9.1 |
| glioma SF-295 | 0.4 | 0.0 | 2.6 | 1.3 |
| Heart (Fetal) | 10.0 | 24.7 | 9.6 | 7.4 |
| Heart | 3.1 | 0.0 | 2.4 | 2.4 |
| Skeletal muscle (Fetal) | 12.8 | 66.4 | 7.7 | 1.3 |
| Skeletal muscle | 20.9 | 2.1 | 7.5 | 13.1 |
| Bone marrow | 1.2 | 1.9 | 4.5 | 0.9 |
| Thymus | 6.0 | 24.0 | 17.8 | 4.9 |
| Spleen | 6.7 | 5.0 | 19.8 | 9.2 |
| Lymph node | 6.7 | 1.4 | 5.4 | 2.6 |
| Colorectal | 23.5 | 19.3 | 6.5 | 9.9 |
| Stomach | 12.0 | 1.8 | 4.5 | 2.5 |
| Small intestine | 54.3 | 13.3 | 69.7 | 43.2 |
| Colon ca. SW480 | 1.1 | 0.5 | 0.0 | 0.0 |
| Colon ca.* SW620 (SW480 met) | 1.4 | 0.0 | 2.8 | 3.1 |
| Colon ca. HT29 | 7.3 | 28.3 | 11.3 | 5.3 |
| Colon ca. HCT-116 | 7.3 | 9.0 | 12.4 | 10.2 |
| Colon ca. CaCo-2 | 10.7 | 29.7 | 19.6 | 11.1 |
| CC Well to Mod Diff (ODO3866) | 8.5 | 14.6 | 10.6 | 10.6 |
| Colon ca. HCC-2998 | 2.9 | 6.3 | 19.8 | 2.9 |
| Gastric ca. (liver met) NCI-N87 | 71.7 | 49.7 | 95.3 | 100.0 |
| Bladder | 14.8 | 44.4 | 29.7 | 20.4 |
| Trachea | 21.9 | 13.9 | 18.6 | 5.5 |
| Kidney | 38.2 | 74.2 | 56.3 | 67.4 |
| Kidney (fetal) | 27.5 | 37.1 | 40.1 | 33.9 |
| Renal ca. 786-0 | 0.0 | 0.0 | 0.0 | 0.0 |
| Renal ca. A498 | 0.2 | 1.7 | 3.7 | 2.9 |
| Renal ca. RXF 393 | 39.8 | 5.9 | 20.3 | 10.8 |
| Renal ca. ACHN | 51.1 | 6.8 | 20.2 | 7.2 |
| Renal ca. UO-31 | 0.2 | 0.0 | 0.0 | 0.0 |
| Renal ca. TK-10 | 0.0 | 0.0 | 0.0 | 0.0 |
| Liver | 1.4 | 0.7 | 0.0 | 3.5 |
| Liver (fetal) | 2.3 | 0.0 | 4.0 | 1.2 |
| Liver ca. (hepatoblast) HepG2 | 11.8 | 4.7 | 19.5 | 10.8 |
| Lung | 75.3 | 46.0 | 91.4 | 84.1 |
| Lung (fetal) | 54.7 | 100.0 | 92.0 | 64.6 |
| Lung ca. (small cell) LX-1 | 5.5 | 2.5 | 5.8 | 4.1 |
| Lung ca. (small cell) NCI-H69 | 5.6 | 6.2 | 10.1 | 5.3 |
| Lung ca. (s. cell var.) SHP-77 | 0.2 | 0.6 | 0.0 | 0.0 |
| Lung ca. (large cell)NCI-H460 | 1.2 | 0.0 | 0.0 | 0.0 |
| Lung ca. (non-sm. cell) A549 | 1.2 | 0.7 | 3.1 | 1.3 |
| Lung ca. (non-s. cell) NCI-H23 | 3.2 | 4.7 | 8.2 | 4.5 |
| Lung ca. (non-s. cell) HOP-62 | 0.0 | 0.0 | 0.0 | 0.0 |
| Lung ca. (non-s. cl) NCI-H522 | 0.0 | 0.0 | 0.0 | 0.0 |
| Lung ca. (squam.) SW 900 | 1.8 | 0.0 | 3.4 | 2.0 |
| Lung ca. (squam.) NCI-H596 | 14.6 | 10.9 | 31.6 | 53.6 |
| Mammary gland | 11.9 | 4.9 | 23.8 | 17.4 |
| Breast ca.* (pl. ef) MCF-7 | 89.5 | 92.7 | 84.1 | 80.1 |
| Breast ca.* (pl. ef) MDA-MB-231 | 0.0 | 0.0 | 0.0 | 0.0 |
| Breast ca.* (pl. ef) T47D | 24.7 | 7.6 | 20.3 | 9.9 |
| Breast ca. BT-549 | 2.3 | 0.0 | 0.0 | 1.2 |
| Breast ca. MDA-N | 0.0 | 0.0 | 0.0 | 0.0 |
| Ovary | 3.5 | 20.3 | 8.5 | 4.9 |
| Ovarian ca. OVCAR-3 | 6.4 | 2.6 | 8.2 | 4.5 |
| Ovarian ca. OVCAR-4 | 0.0 | 0.0 | 0.0 | 0.0 |
| Ovarian ca. OVCAR-5 | 0.0 | 0.0 | 0.0 | 0.0 |
| Ovarian ca. OVCAR-8 | 0.2 | 1.0 | 0.0 | 0.0 |
| Ovarian ca. IGROV-1 | 14.5 | 17.6 | 31.6 | 33.7 |
| Ovarian ca. (ascites) SK-OV-3 | 9.3 | 5.4 | 20.7 | 22.5 |
| Uterus | 27.7 | 3.5 | 39.0 | 46.3 |
| Placenta | 2.9 | 4.9 | 6.4 | 8.9 |
| Prostate | 25.0 | 8.8 | 16.7 | 16.7 |
| Prostate ca.* (bone met) PC-3 | 0.0 | 0.0 | 0.0 | 0.0 |
| Testis | 2.5 | 0.7 | 4.4 | 2.7 |
| Melanoma Hs688(A).T | 0.0 | 0.0 | 0.0 | 0.0 |
| Melanoma* (met) Hs688(B).T | 0.0 | 0.0 | 0.0 | 0.0 |
| Melanoma UACC-62 | 0.0 | 0.0 | 0.0 | 0.0 |
| Melanoma M14 | 0.0 | 0.0 | 0.0 | 0.0 |
| Melanoma LOX IMVI | 0.0 | 0.0 | 0.0 | 0.0 |
| Melanoma* (met) SK-MEL-5 | 0.0 | 0.0 | 0.0 | 0.0 |
| Adipose | 19.3 | 6.5 | 4.3 | 22.1 |

Column A - Rel. Exp. (%) Ag2505, Run 165531061
Column B - Rel. Exp. (%) Ag2667, Run 162554578
Column C - Rel. Exp. (%) Ag2767, Run 165527179
Column D - Rel. Exp. (%) Ag2831, Run 165517578

TABLE EK
Panel 2.2

| Tissue Name | A |
|---|---|
| Normal Colon | 4.7 |
| Colon cancer (OD06064) | 24.7 |
| Colon Margin (OD06064) | 12.0 |
| Colon cancer (OD06159) | 1.1 |
| Colon Margin (OD06159) | 6.2 |
| Colon cancer (OD06297-04) | 1.9 |
| Colon Margin (OD06297-05) | 6.9 |
| CC Gr. 2 ascend colon (ODO3921) | 0.4 |
| CC Margin (ODO3921) | 2.7 |
| Colon cancer metastasis (OD06104) | 2.4 |
| Lung Margin (OD06104) | 10.2 |
| Colon mets to lung (OD04451-01) | 7.0 |
| Lung Margin (OD04451-02) | 20.4 |
| Normal Prostate | 4.9 |
| Prostate Cancer (OD04410) | 5.9 |
| Prostate Margin (OD04410) | 8.3 |
| Normal Ovary | 1.9 |
| Ovarian cancer (OD06283-03) | 1.2 |
| Ovarian Margin (OD06283-07) | 3.6 |
| Ovarian Cancer | 7.8 |
| Ovarian cancer (OD06145) | 0.9 |
| Ovarian Margin (OD06145) | 0.9 |
| Ovarian cancer (OD06455-03) | 0.0 |
| Ovarian Margin (OD06455-07) | 7.3 |
| Normal Lung | 14.2 |
| Invasive poor diff. lung adeno (ODO4945-01 | 1.5 |
| Lung Margin (ODO4945-03) | 15.5 |
| Lung Malignant Cancer (OD03126) | 4.2 |
| Lung Margin (OD03126) | 8.3 |
| Lung Cancer (OD05014A) | 5.4 |
| Lung Margin (OD05014B) | 41.5 |
| Lung cancer (OD06081) | 3.8 |
| Lung Margin (OD06081) | 37.6 |
| Lung Cancer (OD04237-01) | 1.6 |
| Lung Margin (OD04237-02) | 33.2 |
| Ocular Mel Met to Liver (ODO4310) | 0.0 |
| Liver Margin (ODO4310) | 0.0 |
| Melanoma Metastasis | 0.0 |
| Lung Margin (OD04321) | 37.9 |
| Normal Kidney | 5.5 |
| Kidney Ca, Nuclear grade 2 (OD04338) | 26.6 |
| Kidney Margin (OD04338) | 0.9 |
| Kidney Ca Nuclear grade 1/2 (OD04339) | 2.0 |
| Kidney Margin (OD04339) | 10.3 |
| Kidney Ca, Clear cell type (OD04340) | 4.5 |
| Kidney Margin (OD04340) | 12.6 |
| Kidney Ca, Nuclear grade 3 (OD04348) | 1.4 |
| Kidney Margin (OD04348) | 100.0 |
| Kidney malignant cancer (OD06204B) | 0.0 |
| Kidney normal adjacent tissue (OD06204E) | 7.0 |
| Kidney Cancer (OD04450-01) | 1.2 |
| Kidney Margin (OD04450-03) | 16.6 |
| Kidney Cancer 8120613 | 1.8 |
| Kidney Margin 8120614 | 5.7 |
| Kidney Cancer 9010320 | 0.6 |
| Kidney Margin 9010321 | 2.6 |
| Kidney Cancer 8120607 | 6.2 |
| Kidney Margin 8120608 | 2.3 |
| Normal Uterus | 13.4 |
| Uterine Cancer 064011 | 0.8 |
| Normal Thyroid | 6.1 |
| Thyroid Cancer | 28.5 |
| Thyroid Cancer A302152 | 46.3 |
| Thyroid Margin A302153 | 21.0 |
| Normal Breast | 10.2 |
| Breast Cancer | 1.5 |
| Breast Cancer | 4.6 |
| Breast Cancer (OD04590-01) | 62.0 |
| Breast Cancer Mets (OD04590-03) | 98.6 |
| Breast Cancer Metastasis | 70.7 |
| Breast Cancer | 3.6 |
| Breast Cancer 9100266 | 3.4 |
| Breast Margin 9100265 | 2.9 |
| Breast Cancer A209073 | 1.7 |
| Breast Margin A209073 | 2.5 |
| Breast cancer (OD06083) | 49.7 |
| Breast cancer node metastasis (OD06083) | 64.2 |
| Normal Liver | 0.5 |
| Liver Cancer 1026 | 0.5 |
| Liver Cancer 1025 | 1.8 |
| Liver Cancer 6004-T | 0.0 |
| Liver Tissue 6004-N | 1.3 |
| Liver Cancer 6005-T | 0.5 |
| Liver Tissue 6005-N | 1.4 |
| Liver Cancer | 0.0 |
| Normal Bladder | 2.8 |
| Bladder Cancer | 2.5 |
| Bladder Cancer | 6.2 |
| Normal Stomach | 2.8 |
| Gastric Cancer 9060397 | 0.0 |
| Stomach Margin 9060396 | 1.4 |
| Gastric Cancer 9060395 | 2.3 |
| Stomach Margin 9060394 | 4.1 |
| Gastric Cancer 064005 | 5.7 |

Column A - Rel. Exp. (%) Ag2831, Run 175063921

TABLE EL
Panel 3D

| Tissue Name | A |
|---|---|
| 94905 Daoy Medulloblastoma/Cerebellum | 0.4 |
| 94906 TE671 Medulloblastom/Cerebellum | 3.7 |
| 94907 D283 Med Medulloblastoma/Cerebellum | 6.7 |
| 94908 PFSK-1 Primitive Neuroectodermal/Cerebellum | 0.0 |
| 94909 XF-498 CNS | 1.2 |
| 94910 SNB-78 CNS/glioma | 1.7 |
| 94911 SF-268 CNS/glioblastoma | 0.0 |
| 94912 T98G Glioblastoma | 0.0 |
| 96776 SK-N-SH Neuroblastoma (metastasis) | 0.0 |
| 94913 SF-295 CNS/glioblastoma | 0.0 |
| 94914 Cerebellum | 0.0 |
| 96777 Cerebellum | 0.0 |
| 94916 NCI-H292 Mucoepidermoid lung carcinoma | 23.7 |
| 94917 DMS-114 Small cell lung cancer | 0.0 |
| 94918 DMS-79 Small cell lung cancer/neuroendocrine | 1.1 |
| 94919 NCI-H146 Small cell lung | 100.0 |
| cancer/neuroendocrine | |
| 94920 NCI-H526 Small cell lung | 5.6 |
| cancer/neuroendocrine | |
| 94921 NCI-N417 Small cell lung | 0.8 |
| cancer/neuroendocrine | |
| 94923 NCI-H82 Small cell lung | 0.0 |
| cancer/neuroendocrine | |
| 94924 NCI-H157 Squamous cell lung cancer | 0.0 |
| (metastasis) | |
| 94925 NCI-H1155 Large cell lung | 14.6 |
| cancer/neuroendocrine | |
| 94926 NCI-H1299 Large cell lung | 0.0 |
| cancer/neuroendocrine | |
| 94927 NCI-H727 Lung carcinoid | 14.8 |
| 94928 NCI-UMC-11 Lung carcinoid | 84.1 |
| 94929 LX-1 Small cell lung cancer | 7.5 |
| 94930 Colo-205 Colon cancer | 18.7 |
| 94931 KM12 Colon cancer | 66.4 |
| 94932 KM20L2 Colon cancer | 8.4 |
| 94933 NCI-H716 Colon cancer | 23.2 |
| 94935 SW-48 Colon adenocarcinoma | 63.7 |
| 94936 SW1116 Colon adenocarcinoma | 15.5 |
| 94937 LS 174T Colon adenocarcinoma | 62.9 |
| 94938 SW-948 Colon adenocarcinoma | 2.7 |
| 94939 SW-480 Colon adenocarcinoma | 39.2 |
| 94940 NCI-SNU-5 Gastric carcinoma | 0.0 |
| KATO III- Gastric carcinoma | 33.0 |
| 94943 NCI-SNU-16 Gastric carcinoma | 0.0 |
| 94944 NCI-SNU-1 Gastric carcinoma | 35.6 |
| 94946 RF-1 Gastric adenocarcinoma | 0.0 |
| 94947 RF-48 Gastric adenocarcinoma | 0.7 |
| 96778 MKN-45 Gastric carcinoma | 96.6 |
| 94949 NCI-N87 Gastric carcinoma | 79.6 |
| 94951 OVCAR-5 Ovarian carcinoma | 0.0 |
| 94952 RL95-2 Uterine carcinoma | 0.0 |
| 94953 HelaS3 Cervical adenocarcinoma | 0.0 |
| 94954 Ca Ski Cervical epidermoid carcinoma | 9.4 |
| (metastasis | |
| 94955 ES-2 Ovarian clear cell carcinoma | 0.0 |
| 94957 Ramos Stimulated with PMA/ionomycin 6 h | 0.0 |
| 94958 Ramos Stimulated with PMA/ionomycin 14 h | 0.0 |
| 94962 MEG-01 Chronic myelogenous leukemia | 0.7 |
| (megokaryoblast ) | |
| 94963 Raji Burkitt's lymphoma | 0.0 |
| 94964 Daudi Burkitt's lymphoma | 0.0 |
| 94965 U266 B-cell plasmacytoma/myeloma | 0.0 |
| 94968 CA46 Burkitt's lymphoma | 0.0 |
| 94970 RL non-Hodgkin's B-cell lymphoma | 0.0 |
| 94972 JM1 pre-B-cell lymphoma/leukemia | 0.0 |
| 94973 Jurkat T cell leukemia | 0.0 |
| 94974 TF-1 Erythroleukemia | 0.0 |
| 94975 HUT 78 T-cell lymphoma | 0.0 |
| 94977 U937 Histiocytic lymphoma | 0.0 |
| 94980 KU-812 Myelogenous leukemia | 0.6 |
| 769-P- Clear cell renal carcinoma | 3.2 |
| 94983 Caki-2 Clear cell renal carcinoma | 0.8 |
| 94984 SW 839 Clear cell renal carcinoma | 0.9 |
| 94986 G401 Wilms' tumor | 0.0 |
| 94987 Hs766T Pancreatic carcinoma (LN metastasis) | 0.0 |
| 94988 CAPAN-1 Pancreatic adenocarcinoma | 0.0 |
| (liver metastasis) | |
| 94989 SU86.86 Pancreatic carcinoma (liver | 0.0 |
| metastasis) | |
| 94990 BxPC-3 Pancreatic adenocarcinoma | 0.0 |
| 94991 HPAC Pancreatic adenocarcinoma | 0.0 |
| 94992 MIA PaCa-2 Pancreatic carcinoma | 0.0 |
| 94993 CFPAC-1 Pancreatic ductal adenocarcinoma | 0.0 |
| 94994 PANC-1 Pancreatic epithelioid ductal | 0.6 |
| carcinoma | |
| 94996 T24 Bladder carcinma (transitional cell | 0.0 |
| 5637- Bladder carcinoma | 0.0 |
| 94998 HT-1197 Bladder carcinoma | 3.2 |
| 94999 UM-UC-3 Bladder carcinma (transitional cell) | 0.0 |
| 95000 A204 Rhabdomyosarcoma | 0.0 |
| 95001 HT-1080 Fibrosarcoma | 0.0 |
| 95002 MG-63 Osteosarcoma (bone) | 0.0 |
| 95003 SK-LMS-1 Leiomyosarcoma (vulva) | 0.0 |
| 95004 SJRH30 Rhabdomyosarcoma (met to bone marrow) | 27.7 |
| 95005 A431 Epidermoid carcinoma | 17.8 |
| 95007 WM266-4 Melanoma | 0.0 |
| DU 145- Prostate carcinoma (brain metastasis) | 0.0 |
| 95012 MDA-MB-468 Breast adenocarcinoma | 2.7 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| 95017 CAL 27 Squamous cell carcinoma of tongue | 1.2 |

Column A - Rel. Exp. (%) Ag2831, Run 164843468

TABLE EM
Panel 5 Islet

| Tissue Name | A |
|---|---|
| 97457 Patient-02go adipose | 32.3 |
| 97476 Patient-07sk skeletal muscle | 8.2 |
| 97477 Patient-07ut uterus | 31.4 |
| 97478 Patient-07pl placenta | 3.5 |
| 99167 Bayer Patient 1 | 9.5 |
| 97482 Patient-08ut uterus | 90.1 |
| 97483 Patient-08pl placenta | 7.4 |
| 97486 Patient-09sk skeletal muscle | 1.4 |
| 97487 Patient-09ut uterus | 78.5 |
| 97488 Patient-09pl placenta | 0.6 |
| 97492 Patient-10ut uterus | 66.0 |
| 97493 Patient-10pl placenta | 3.1 |
| 97495 Patient-11go adipose | 28.3 |
| 97496 Patient-11sk skeletal muscle | 5.8 |
| 97497 Patient-11ut uterus | 35.4 |
| 97498 Patient-11pl placenta | 2.0 |
| 97500 Patient-12go adipose | 35.1 |
| 97501 Patient-12sk skeletal muscle | 9.9 |
| 97502 Patient-12ut uterus | 100.0 |
| 97503 Patient-12pl placenta | 4.1 |
| 94721 Donor 2 U - A Mesenchymal Stem Cells | 0.0 |
| 94722 Donor 2 U - B Mesenchymal Stem Cells | 0.0 |
| 94723 Donor 2 U - C Mesenchymal Stem Cells | 0.0 |
| 94709 Donor 2 AM - A adipose | 0.0 |
| 94710 Donor 2 AM - B adipose | 0.0 |
| 94711 Donor 2 AM - C adipose | 0.0 |
| 94712 Donor 2 AD - A adipose | 0.0 |
| 94713 Donor 2 AD - B adipose | 0.0 |
| 94714 Donor 2 AD - C adipose | 0.0 |
| 94742 Donor 3 U - A Mesenchymal Stem Cells | 0.0 |
| 94743 Donor 3 U - B Mesenchymal Stem Cells | 0.0 |
| 94730 Donor 3 AM - A adipose | 0.0 |
| 94731 Donor 3 AM - B adipose | 0.0 |
| 94732 Donor 3 AM - C adipose | 0.2 |
| 94733 Donor 3 AD - A adipose | 0.0 |
| 94734 Donor 3 AD - B adipose | 0.0 |
| 94735 Donor 3 AD - C adipose | 0.0 |
| 77138 Liver HepG2untreated | 21.2 |
| 73556 Heart Cardiac stromal cells (primary) | 0.0 |
| 81735 Small Intestine | 36.9 |
| 72409 Kidney Proximal Convoluted Tubule | 4.6 |
| 82685 Small intestine Duodenum | 27.0 |
| 90650 Adrenal Adrenocortical adenoma | 0.3 |
| 72410 Kidney HRCE | 7.2 |
| 72411 Kidney HRE | 10.7 |
| 73139 Uterus Uterine smooth muscle cells | 0.0 |

Column A - Rel. Exp. (%) Ag2505, Run 248045752

AI_comprehensive panel_v1.0 Summary: Ag2505/Ag2831 The highest expression of this gene was seen in bone from a rheumatoid arthritis patient (CT=27-29). While the gene showed ubiquitous expression, expression was clearly higher in bone, synovium, cartilage and synovial fluid from RA patients as compared to expression in samples from OA patients, normal and diseased lung and therefore is useful for differentiating these disease states. Expression of this gene was modulated in Crohn's samples as compared to the corresponding control samples. This gene encodes a novel adhesion molecule which is homologous to mouse POEM (preosteoblast epidermal growth factor-like repeat protein with meprin)or nephronectin. Murine nephronectin functions in multiple biological processes including development of the kidney (Miner J H. J Cell Biol Jul. 23, 2001;154(2):257-9, PMID: 11470814) and bone (Morimura N et al., 2001, J. Biol. Chem. Nov. 9, 2000;276(45):42172-42181, PMID: 11546798) and contribute to liver and lung fibrosis (Levine et al., Am J Pathol June, 2000;156(6):1927-35, PMID: 10854216). Therapeutic modulation of this gene, expressed protein and/or use of antibodies or small molecule drugs targeting the gene or gene product are useful in the treatment of autoimmune and inflammatory diseases such as rheumatoid and osteoarthritis, Inflammatory bowel disease, COPD, asthma, psoriasis, liver and lung fibrosis.

CNS_neurodegeneration_v1.0

Summary: Ag2505/Ag2667/Ag2767/Ag2831I/Ag7237 This panel confirms the expression of this gene at low levels in the brain in an independent group of individuals. This gene was found to be upregulated in the temporal cortex of Alzheimer's disease patients. This gene codes for a homolog of mouse POEM (Nephronectin short isoform), a cell adhesion molecule with EGF domains. Alpha secretase activity, which is generally believed to be a beneficial processing alternative to beta secretase, is increased by EGF in neuronal cells (Slack B E, Breu J, Muchnicki L, Wurtman R J, 1997, Biochem J 327 (Pt 1):245-9). The increased expression of this gene reported here is a compensatory action in the brain to counter the mechanisms of Alzheimer's Disease. Therapeutic modulation of this gene, expressed protein and/or use of antibodies or small molecule drugs targeting the gene or gene product are useful in the treatment of Alzheimer's disease and other neurodegenerative diseases.

EGF is also known to facilitate long term potentiation (LTP) in the hippocampus, a process thought to underlie learning and memory (Abe K, Ishiyama J, Saito H, 1992, Brain Res 593(2):335-8). Therapeutic modulation of this gene, expressed protein and/or use of antibodies or small molecule drugs targeting the gene or gene product are useful in treating disorders of memory, such as neurodegenerative diseases and aging, when used alone or incombination with other growth factors such as but not limited to bFGF.

In addition, EGF supports the growth and differentiation of dopaminergic neurons (Storch A, Paul G, Csete M, Boehm B O, Carvey P M, Kupsch A, Schwarz J, 2001, Exp Neurol 170(2):317-25), which are selectively vulnerable to loss in Parkinson's disease. Therapeutic modulation of this gene, expressed protein and/or use of antibodies or small molecule drugs targeting the gene or gene product are useful in treating Parkinson's Disease.

General_screening_panel_v1.6 Summary: Ag7237 Highest expression of this gene was detected in fetal lung (CT=27) and was higher in fetal (CTs=27-33) than in corresponding adult lung, kidney, liver and skeletal muscle tissues (CT=32-40). The relative overexpression of this gene in fetal tissue suggests that the protein product may enhance growth or development of these tissues in the fetus and thus may also act in a regenerative capacity in the adult. Therapeutic modulation of this gene, expressed protein and/or use of antibodies or small molecule drugs targeting the gene or gene product are useful in the treatment of lung, liver, kidney and muscle related diseases.

Moderate to low levels of expression of this gene were also seen in cancer cell lines derived from squamous cell carcinoma, brain, colon, renal, lung, breast, and ovarian cancers. Expression of this gene is useful as diagnostic marker for detection of these cancers or for differentiating cancerous from normal tissue. Therapeutic modulation of this gene, expressed protein and/or use of antibodies or small molecule drugs targeting the gene or gene product are useful in the treatment of carcinomas including but not limited to: squamous cell carcinoma, brain, colon, renal, lung, breast, and ovarian cancers.

Moderate to low levels of expression of this gene was also seen in tissues with metabolic/endocrine functions and also in all the regions of brain.

PGI1.0 Summary: Ag2505 The highest expression of this gene was detected in a lung fibrosis sample (CT=22). This gene was upregulated in several lung fibrosis and emphysema samples, and also in one asthma sample. Therapeutic modulation of this gene, expressed protein and/or use of antibodies or small molecule drugs targeting the gene or gene product are useful in the treatment of lung diseases including fibrosis, emphysema, and asthma.

Panel 1.3D Summary: Ag2505/Ag2667/Ag2767/Ag2831 Highest expression of this gene was detected in the thyroid and fetal lung (CTs=29-31). Moderate to low levels of expression of this gene was also seen in other tissues with metabolic/endocrine functions, including skeletal muscle, fetal skeletal muscle, small intestine, stomach, pancreas, adipose and fetal heart. Very low levels were also seen in heart and placenta. Nephronectin is the ligand for the alpha8beta1 integrin. Integrins are known to mediate development and organogenesis. Nephronectin can bind integrins including alpha5beta3, alpha5beta5, alpha5beta6 and alpha4beta7, but not alpha4beta1, alpha3beta1, alpha2beta1 or alpha1beta1. Nephronectin interacts with integrins via the RGD sequence, but RGD alone is not sufficient for binding, the MAM domain is also required. Therapeutic modulation of this gene, expressed protein and/or use of antibodies or small molecule drugs targeting the gene or gene product are useful in the treatment of disorders involving alpha8beta1 integrin signaling including inflammatory diseases.

Obesity has also been linked to inflammatory condition (Das U N, 2001, Nutrition 17(11-12):953-66, PMD: 11744348) and thus humanized antibodies are therapeutically relevant in treating this condition and related complications such as type II diabetes.

Panel 2.2 Summary: Ag2831 Highest expression of this gene was detected in kidney (CT=30.3). Expression of this gene was down regulated in kidney, lung and colon cancer as compared to the corresponding normal adjacent tissue. Conversely, increased expression of this gene was seen in breast cancer samples. Therefore, expression of this gene may be used to distinguish between cancer and normal kidney, lung, colon and breast. Therapeutic modulation of this gene, expressed protein and/or use of antibodies or small molecule drugs targeting the gene or gene product are useful in the treatment of kidney, lung, colon and breast cancer.

Panel 3D Summary: Ag2831 Highest expression of this gene was detected in a small cell lung cancer NCI-H146 cell line (CT=29.7). Moderate to low levels of expression of this gene was also seen in cancer cell lines derived from epidermoid carcinoma, rhabodomyosacoma, gastric, colon and small cell lung cancers. The expression of this gene can be used as diagnostic marker for detection of these cancers or for differentiating cancerous from normal tissue. Therapeutic modulation of this gene, expressed protein and/or use of antibodies or small molecule drugs targeting the gene or gene product are useful in the treatment of epidermoid carcinoma, rhabodomyosacoma, gastric, colon and small cell lung cancers.

Panel 5 Islet Summary: Ag2505 The highest expression of this gene was detected in uterus (CT=30). Moderate expression of this gene was also seen in adipose and skeletal muscle of gestational diabetic patients requiring and not requiring daily injections of insulin. This gene was also expressed in samples derived from pregnant and a nondiabetic, but overweight patient. In addition, this gene is also expressed in islet beta cells (those that are insulin producing) and small intestine. Therapeutic modulation of this gene, expressed protein and/or use of antibodies or small molecule drugs targeting the gene or gene product are useful in the treatment of metabolically related diseases including obesity, Type I and Type II diabetes.

F. NOV6, CG51923: FAT Tumor Suppressor Homolog 2

Expression of gene NOV6a, 6m, 6n, CG51923 were assessed using the primer-probe sets Ag395, Ag706, Ag888, Ag944 and Ag945, described in Tables FA, FB, FC, FD and FE. Results of the RTQ-PCR runs are shown in Tables FF and FG.

TABLE FGA
Probe Name Ag395

| | | | Start | SEQ ID |
|---|---|---|---|---|
| Primers | Sequences | Length | Position | No |
| Forward | 5′-caggaagaaataagccaa | 23 | 13104 | 118 |
| | gtcca-3′ | | | |
| Probe | TET-5′-tccttggcctcccg | 19 | 13084 | 119 |
| | cctgc-3′-TAMRA | | | |
| Reverse | 5′-gaggtcatgttctagctt | 24 | 13049 | 120 |
| | cccatt-3′ | | | |

TABLE FB
Probe Name Ag706

| | | | Start | SEQ ID |
|---|---|---|---|---|
| Primers | Sequences | Length | Position | No |
| Forward | 5′-tatgtggagagcttcgag | 22 | 164 | 121 |
| | aaaa-3′ | | | |
| Probe | TET-5′-atctacctcgcgga | 23 | 191 | 122 |
| | gccacagtg-3′-TAMRA | | | |
| Reverse | 5′-agagatgatccggtacct | 22 | 217 | 123 |
| | cact-3′ | | | |

TABLE FC
Probe Name Ag888

| | | | Start | SEQ ID |
|---|---|---|---|---|
| Primers | Sequences | Length | Position | No |
| Forward | 5′-catagctgaccgcatctg | 20 | 11160 | 124 |
| | aa-3′ | | | |
| Probe | TET-5′-aatgctccatctcc | 26 | 11125 | 125 |
| | ttggctgagtga-3′-TAMRA | | | |
| Reverse | 5′-ggagctagcatccatcat | 21 | 11104 | 126 |
| | cac-3′ | | | |

TABLE FD
Probe Name Ag944

| | | | Start | SEQ ID |
|---|---|---|---|---|
| Primers | Sequences | Length | Position | No |
| Forward | 5′-agctcaactacagcacca | 22 | 10296 | 127 |
| | ctgt-3′ | | | |
| Probe | TET-5′-cagcaaagtcctgc | 25 | 10339 | 128 |
| | agctgatcctg-3′-TAMRA | | | |
| Reverse | 5′-ctctggagaatctgggtc | 21 | 10364 | 129 |
| | act-3′ | | | |

TABLE FE
Probe Name Ag945

| | | | Start | SEQ ID |
|---|---|---|---|---|
| Primers | Sequences | Length | Position | No |
| Forward | 5′-ccaagtcatcattcatgt | 22 | 5581 | 130 |
| | caga-3′ | | | |
| Probe | TET-5′-ttcccctcccagat | 26 | 5614 | 131 |
| | tctcagaacaga-3′-TAMRA | | | |
| Reverse | 5′-atggataggcccgactat | 20 | 5652 | 132 |
| | tg-3′ | | | |

TABLE FF
Panel 1.1

| Tissue Name | A |
|---|---|
| Adrenal gland | 0.1 |
| Bladder | 1.4 |
| Brain (amygdala) | 0.1 |
| Brain (cerebellum) | 100.0 |
| Brain (hippocampus) | 0.2 |
| Brain (substantia nigra) | 1.2 |
| Brain (thalamus) | 0.2 |
| Cerebral Cortex | 1.5 |
| Brain (fetal) | 0.9 |
| Brain (whole) | 4.5 |
| glio/astro U-118-MG | 0.1 |
| astrocytoma SF-539 | 0.3 |
| astrocytoma SNB-75 | 0.3 |
| astrocytoma SW1783 | 0.1 |
| glioma U251 | 0.1 |
| glioma SF-295 | 0.4 |
| glioma SNB-19 | 0.1 |
| glio/astro U87-MG | 0.8 |
| neuro*; met SK-N-AS | 1.2 |
| Mammary gland | 1.4 |
| Breast ca. BT-549 | 0.2 |
| Breast ca. MDA-N | 0.7 |
| Breast ca.* (pl. ef) T47D | 0.5 |
| Breast ca.* (pl. ef) MCF-7 | 0.3 |
| Breast ca.* (pl. ef) MDA-MB-231 | 0.1 |
| Small intestine | 0.6 |
| Colorectal | 0.2 |
| Colon ca. HT29 | 0.1 |
| Colon ca. CaCo-2 | 1.0 |
| Colon ca. HCT-15 | 0.3 |
| Colon ca. HCT-116 | 0.3 |
| Colon ca. HCC-2998 | 1.1 |
| Colon ca. SW480 | 0.3 |
| Colon ca.* SW620 (SW480 met) | 1.0 |
| Stomach | 0.3 |
| Gastric ca. (liver met) NCI-N87 | 0.5 |
| Heart | 0.4 |
| Skeletal muscle (Fetal) | 0.5 |
| Skeletal muscle | 0.8 |
| Endothelial cells | 0.2 |
| Heart (Fetal) | 0.0 |
| Kidney | 0.7 |
| Kidney (fetal) | 0.7 |
| Renal ca. 786-0 | 0.1 |
| Renal ca. A498 | 0.3 |
| Renal ca. ACHN | 0.3 |
| Renal ca. TK-10 | 0.5 |
| Renal ca. UO-31 | 0.1 |
| Renal ca. RXF 393 | 0.0 |
| Liver | 0.5 |
| Liver (fetal) | 0.5 |
| Liver ca. (hepatoblast) HepG2 | 0.0 |
| Lung | 0.1 |
| Lung (fetal) | 0.2 |
| Lung ca. (non-s. cell) HOP-62 | 1.0 |
| Lung ca. (large cell)NCI-H460 | 0.8 |
| Lung ca. (non-s. cell) NCI-H23 | 0.2 |
| Lung ca. (non-s. cl) NCI-H522 | 0.7 |
| Lung ca. (non-sm. cell) A549 | 0.3 |
| Lung ca. (s. cell var.) SHP-77 | 0.2 |
| Lung ca. (small cell) LX-1 | 1.2 |
| Lung ca. (small cell) NCI-H69 | 0.4 |
| Lung ca. (squam.) SW 900 | 0.1 |
| Lung ca. (squam.) NCI-H596 | 0.5 |
| Lymph node | 0.3 |
| Spleen | 0.1 |
| Thymus | 1.1 |
| Ovary | 0.0 |
| Ovarian ca. IGROV-1 | 0.1 |
| Ovarian ca. OVCAR-3 | 7.7 |
| Ovarian ca. OVCAR-4 | 6.4 |
| Ovarian ca. OVCAR-5 | 1.5 |
| Ovarian ca. OVCAR-8 | 0.5 |
| Ovarian ca. (ascites) SK-OV-3 | 0.7 |
| Pancreas | 0.9 |
| Pancreatic ca. CAPAN 2 | 0.0 |
| Pituitary gland | 0.5 |
| Placenta | 0.6 |
| Prostate | 2.4 |
| Prostate ca.* (bone met) PC-3 | 0.2 |
| Salivary gland | 2.4 |
| Trachea | 1.9 |
| Spinal cord | 0.4 |
| Testis | 2.0 |
| Thyroid | 0.1 |
| Uterus | 0.1 |
| Melanoma M14 | 0.4 |
| Melanoma LOX IMVI | 0.1 |
| Melanoma UACC-62 | 0.1 |
| Melanoma SK-MEL-28 | 1.6 |
| Melanoma* (met) SK-MEL-5 | 0.1 |
| Melanoma Hs688(A).T | 0.1 |
| Melanoma* (met) Hs688(B).T | 0.1 |

Column A - Rel. Exp. (%) Ag395, Run 109668522

TABLE FG
Panel 2D

| Tissue Name | A | B | C |
|---|---|---|---|
| Normal Colon | 20.2 | 10.7 | 5.6 |
| CC Well to Mod Diff (ODO3866) | 6.0 | 0.5 | 0.5 |
| CC Margin (ODO3866) | 5.8 | 0.0 | 0.0 |
| CC Gr. 2 rectosigmoid (ODO3868) | 1.8 | 0.7 | 0.2 |
| CC Margin (ODO3868) | 1.9 | 0.6 | 0.7 |
| CC Mod Diff (ODO3920) | 2.2 | 2.0 | 0.7 |
| CC Margin (ODO3920) | 5.6 | 1.1 | 1.1 |
| CC Gr. 2 ascend colon (ODO3921) | 1.2 | 0.3 | 0.0 |
| CC Margin (ODO3921) | 0.9 | 0.8 | 0.9 |
| CC from Partial Hepatectomy (ODO4309) | 0.9 | 0.7 | 0.2 |
| Mets | | | |
| Liver Margin (ODO4309) | 1.3 | 0.9 | 0.0 |
| Colon mets to lung (OD04451-01) | 2.2 | 0.7 | 0.4 |
| Lung Margin (OD04451-02) | 5.4 | 0.6 | 0.2 |
| Normal Prostate 6546-1 | 43.8 | 29.3 | 21.0 |
| Prostate Cancer (OD04410) | 17.3 | 9.3 | 5.2 |
| Prostate Margin (OD04410) | 15.7 | 8.9 | 12.2 |
| Prostate Cancer (OD04720-01) | 41.2 | 37.9 | 41.2 |
| Prostate Margin (OD04720-02) | 22.8 | 37.1 | 33.2 |
| Normal Lung | 2.8 | 4.5 | 3.0 |
| Lung Met to Muscle (ODO4286) | 0.0 | 1.3 | 1.3 |
| Muscle Margin (ODO4286) | 66.0 | 24.0 | 16:7 |
| Lung Malignant Cancer (OD03126) | 3.5 | 4.4 | 2.4 |
| Lung Margin (OD03126) | 2.9 | 1.8 | 0.2 |
| Lung Cancer (OD04404) | 46.0 | 100.0 | 30.4 |
| Lung Margin (OD04404) | 16.6 | 5.9 | 1.7 |
| Lung Cancer (OD04565) | 100.0 | 65.5 | 100.0 |
| Lung Margin (OD04565) | 3.0 | 0.8 | 2.0 |
| Lung Cancer (OD04237-01) | 2.6 | 0.9 | 1.2 |
| Lung Margin (OD04237-02) | 0.6 | 0.9 | 0.2 |
| Ocular Mel Met to Liver (ODO4310) | 1.0 | 0.7 | 0.9 |
| Liver Margin (ODO4310) | 0.0 | 0.0 | 0.0 |
| Melanoma Metastasis | 3.5 | 1.1 | 0.3 |
| Lung Margin (OD04321) | 0.8 | 1.2 | 0.5 |
| Normal Kidney | 11.3 | 10.3 | 3.0 |
| Kidney Ca, Nuclear grade 2 (OD04338) | 6.3 | 2.3 | 2.4 |
| Kidney Margin (OD04338) | 3.6 | 3.4 | 1.4 |
| Kidney Ca Nuclear grade 1/2 (OD04339) | 23.8 | 4.5 | 3.3 |
| Kidney Margin (OD04339) | 15.0 | 5.8 | 5.5 |
| Kidney Ca, Clear cell type (OD04340) | 3.2 | 1.0 | 2.4 |
| Kidney Margin (OD04340) | 11.9 | 9.8 | 4.0 |
| Kidney Ca, Nuclear grade 3 (OD04348) | 1.3 | 2.0 | 1.9 |
| Kidney Margin (OD04348) | 12.2 | 2.7 | 3.0 |
| Kidney Cancer (OD04622-01) | 4.9 | 2.5 | 4.8 |
| Kidney Margin (OD04622-03) | 3.1 | 3.2 | 4.4 |
| Kidney Cancer (OD04450-01) | 0.5 | 1.6 | 0.1 |
| Kidney Margin (OD04450-03) | 7.4 | 3.0 | 0.8 |
| Kidney Cancer 8120607 | 3.0 | 2.7 | 0.4 |
| Kidney Margin 8120608 | 1.1 | 0.4 | 1.9 |
| Kidney Cancer 8120613 | 0.9 | 0.0 | 0.4 |
| Kidney Margin 8120614 | 2.0 | 0.0 | 0.3 |
| Kidney Cancer 9010320 | 13.1 | 2.4 | 0.9 |
| Kidney Margin 9010321 | 11.5 | 2.3 | 3.1 |
| Normal Uterus | 2.9 | 0.1 | 1.1 |
| Uterine Cancer 064011 | 21.3 | 23.2 | 21.2 |
| Normal Thyroid | 0.8 | 0.7 | 0.7 |
| Thyroid Cancer | 2.5 | 3.2 | 1.5 |
| Thyroid Cancer A302152 | 3.0 | 0.7 | 1.5 |
| Thyroid Margin A302153 | 0.0 | 0.4 | 0.5 |
| Normal Breast | 44.1 | 9.2 | 16.6 |
| Breast Cancer | 5.3 | 1.7 | 3.8 |
| Breast Cancer (OD04590-01) | 10.8 | 1.2 | 1.2 |
| Breast Cancer Mets (OD04590-03) | 6.4 | 3.1 | 3.3 |
| Breast Cancer Metastasis | 1.4 | 0.2 | 1.4 |
| Breast Cancer | 13.1 | 13.7 | 5.5 |
| Breast Cancer | 62.0 | 55.9 | 23.3 |
| Breast Cancer 9100266 | 10.0 | 22.4 | 14.4 |
| Breast Margin 9100265 | 12.9 | 36.6 | 28.5 |
| Breast Cancer A209073 | 25.2 | 43.8 | 44.8 |
| Breast Margin A209073 | 61.1 | 100.0 | 20.7 |
| Normal Liver | 5.4 | 0.0 | 0.4 |
| Liver Cancer | 2.6 | 1.0 | 0.0 |
| Liver Cancer 1025 | 1.0 | 0.4 | 0.3 |
| Liver Cancer 1026 | 0.9 | 0.0 | 0.0 |
| Liver Cancer 6004-T | 9.7 | 0.0 | 0.0 |
| Liver Tissue 6004-N | 3.1 | 0.6 | 0.2 |
| Liver Cancer 6005-T | 0.0 | 0.3 | 0.3 |
| Liver Tissue 6005-N | 0.0 | 0.0 | 0.0 |
| Normal Bladder | 9.0 | 2.5 | 3.1 |
| Bladder Cancer | 2.4 | 0.4 | 0.3 |
| Bladder Cancer | 21.8 | 33.4 | 11.9 |
| Bladder Cancer (OD04718-01) | 46.7 | 75.3 | 68.3 |
| Bladder Normal Adjacent (OD04718-03) | 4.1 | 1.6 | 0.5 |
| Normal Ovary | 0.0 | 0.4 | 0.0 |
| Ovarian Cancer | 65.1 | 91.4 | 50.3 |
| Ovarian Cancer (OD04768-07) | 33.0 | 17.9 | 10.8 |
| Ovary Margin (OD04768-08) | 0.0 | 0.0 | 0.2 |
| Normal Stomach | 2.4 | 2.1 | 1.6 |
| Gastric Cancer 9060358 | 1.5 | 0.7 | 0.0 |
| Stomach Margin 9060359 | 1.4 | 0.4 | 0.4 |
| Gastric Cancer 9060395 | 2.3 | 0.4 | 0.2 |
| Stomach Margin 9060394 | 0.8 | 0.3 | 0.7 |
| Gastric Cancer 9060397 | 6.6 | 2.8 | 0.8 |
| Stomach Margin 9060396 | 0.0 | 0.0 | 0.2 |
| Gastric Cancer 064005 | 4.5 | 1.5 | 0.3 |

Column A - Rel. Exp. (%) Ag395, Run 144794701
Column B - Rel. Exp. (%) Ag888, Run 144791434
Column C - Rel. Exp. (%) Ag888, Run 145420466

Panel 1.1 Summary: Ag395 Highest expression of NOV6 was detected in cerebellum (CT=21). High to moderate levels of expression of this gene were also seen in all regions of the central nervous system examined, including amygdala, hippocampus, substantia nigra, thalamus, cerebral cortex, and spinal cord. This gene encodes protocadherin Fat 2 protein, a homolog of the Drosophila tumor suppressor gene fat. Protocadherins are transmembrane glycoproteins belonging to the cadherin superfamily of molecules, which are involved in many biological processes such as cell adhesion, cytoskeletal organization and morphogenesis. Protocadherins generally exhibit only moderate adhesive activity and are highly expressed in the nervous system. FAT2 is unique among the cadherin superfamily because it contains EGF domains together with the classical cadherin repeats (Nollet et al., 2000, J Mol Biol 299(3):551-72, PMID: 10835267). Cadherins can act as axon guidance and cell adhesion proteins, specifically during development and in the response to injury (Ranscht B., 2000, Int. J. Dev. Neurosci. 18: 643-651, PND: 10978842). Therapeutic modulation of this gene, expressed protein and/or use of antibodies or small molecule drugs targeting the gene or gene product is useful in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

Moderate to high levels of expression of this gene was also seen in certain cancer cell lines derived from gastric, colon, lung, renal, breast, ovarian, prostate, melanoma and brain. Therefore expression of this gene is useful in differentiating the cancer cells from normal counterparts. Therapeutic modulation of this gene, expressed protein and/or use of antibodies or small molecule-drugs targeting the gene or gene product is useful in the treatment of gastric, colon, lung, renal, breast, ovarian, prostate, melanoma and brain cancers.

Among tissues with metabolic or endocrine function, this gene was expressed at high to moderate levels in pancreas, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product is useful in the treatment of endocrine/metabolically related diseases, such as obesity obesity, diabetes, hypercholesterolemia and hypertension.

Panel 2D Summary: Ag395/Ag888 Highest expression of the CG51923-01 gene was detected in two lung cancer cell lines and a control breast sample (CTs=29-32). Moderate expression of this gene was also seen in samples derived from ovarian, bladder, breast, uterine, lung, and prostate cancers. Expression of this gene was higher in ovarian, bladder and lung cancers as compared to their corresponding control samples. Therefore, expression of this gene can be used to differentiate these cancers from the normal tissue counterparts. Furthermore, therapeutic modulation of this gene, expressed protein and/or use of antibodies or small molecule drugs targeting the gene or gene product is useful in the treatment of ovarian, bladder, breast, uterine, lung, and prostate cancers.

G. NOV7 CG52919: Secreted Sushi and CUB Sez-6

Expression of gene CG52919-06 was assessed using the primer-probe set Ag90, described in Table GA. Results of the RTQ-PCR runs are shown in Table HB.

TABLE GA
Probe Name Ag90

| | | | Start | SEQ ID |
|---|---|---|---|---|
| Primers | Sequences | Length | Position | No |
| Forward | 5′-ttggcctggactgcttct | 20 | 824 | 133 |
| | tc-3′ | | | |
| Probe | TET-5′-catctctgtctacc | 28 | 846 | 134 |
| | ctggctatggcgtg-3′- | | | |
| | TAMRA | | | |
| Reverse | 5′-aggctgatattctggacc | 24 | 876 | 135 |
| | ttgatt-3′ | | | |

TABLE GB
Panel 1

| Tissue Name | A |
|---|---|
| Endothelial cells | 0.0 |
| Endothelial cells (treated) | 0.0 |
| Pancreas | 0.1 |
| Pancreatic ca. CAPAN 2 | 0.0 |
| Adrenal gland | 0.0 |
| Thyroid | 0.0 |
| Salivary gland | 0.0 |
| Pituitary gland | 0.0 |
| Brain (fetal) | 37.1 |
| Brain (whole) | 22.5 |
| Brain (amygdala) | 24.8 |
| Brain (cerebellum) | 100.0 |
| Brain (hippocampus) | 29.5 |
| Brain (substantia nigra) | 7.6 |
| Brain (thalamus) | 13.7 |
| Brain (hypothalamus) | 7.7 |
| Spinal cord | 1.4 |
| glio/astro U87-MG | 0.0 |
| glio/astro U-118-MG | 0.0 |
| astrocytoma SW1783 | 0.0 |
| neuro*; met SK-N-AS | 0.4 |
| astrocytoma SF-539 | 0.0 |
| astrocytoma SNB-75 | 0.0 |
| glioma SNB-19 | 1.8 |
| glioma U251 | 0.4 |
| glioma SF-295 | 0.0 |
| Heart | 0.0 |
| Skeletal muscle | 0.0 |
| Bone marrow | 0.0 |
| Thymus | 0.1 |
| Spleen | 0.0 |
| Lymph node | 0.0 |
| Colon (ascending) | 0.1 |
| Stomach | 0.1 |
| Small intestine | 0.3 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| Colon ca. HCT-15 | 0.0 |
| Colon ca. HCC-2998 | 0.0 |
| Gastric ca. (liver met) NCI-N87 | 0.0 |
| Bladder | 0.0 |
| Trachea | 0.0 |
| Kidney | 0.0 |
| Kidney (fetal) | 0.0 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 0.0 |
| Lung ca. (small cell) LX-1 | 0.0 |
| Lung ca. (small cell) NCI-H69 | 33.7 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 20.0 |
| Mammary gland | 0.1 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca. (ascites) SK-OV-3 | 0.0 |
| Uterus | 0.0 |
| Placenta | 0.0 |
| Prostate | 0.0 |
| Prostate ca.* (bone met) PC-3 | 0.0 |
| Testis | 1.3 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Melanoma SK-MEL-28 | 0.0 |

Column A - Rel. Exp. (%) Ag90, Run 87586258

Panel 1 Summary: Ag90 Highest expression of this gene was detected in brain cerebellum (CT=25). High expression of this gene was seen in all the regions of brain including amygdala, hippocampus, substantia nigra, thalamus, cerebellum, cerebral cortex, and spinal cord. In addition, moderate levels of expression of this gene were also seen in two lung cancer cell lines and a glioma cell line. Differential NOV7 gene expression is useful for differentiating lung and glioma cancerous tissues or cells from normal counterparts. Therapeutic modulation of this gene, expressed protein and/or use of antibodies or small molecule drugs targeting the gene or gene product are useful in the treatment of central nervous system disorders such as Alzheimer's disease, Parkinson's disease, epilepsy, multiple sclerosis, schizophrenia, depression, lung and brain cancers.

H. NOV8 CG94946: Agrin Precursor

Expression of gene CG94946-01 was assessed using the primer-probe sets Ag3605 and Ag3974, described in Tables HA and HB. Results of the RTQ-PCR runs are shown in Tables HC and HD.

TABLE HA
Probe Name Ag3605

| | | | Start | SEQ ID |
|---|---|---|---|---|
| Primers | Sequences | Length | Position | No |
| Forward | 5′-gaccccaagtcagaactg | 21 | 3514 | 137 |
| | ttc-3′ | | | |
| Probe | TET-5′-attgagagcaccct | 26 | 3553 | 138 |
| | ggacgacctctt-3′-TAMRA | | | |
| Reverse | 5′-gaaatccttcttgacgtc | 22 | 3585 | 139 |
| | tgaa-3′ | | | |

TABLE HB
Probe Name Ag3974

| | | | Start | SEQ ID |
|---|---|---|---|---|
| Primers | Sequences | Length | Position | No |
| Forward | 5′-gacaccaggatcttctttgtga-3′ | 22 | 379 | 140 |
| Probe | TET-5′-catacctgtggccagcccacaag-3′- | 23 | 413 | 141 |
| | TAMRA | | | |
| Reverse | 5′-gagttgagcatcagctcgtt-3′ | 20 | 436 | 142 |

TABLE HC
General_screening_panel_v1.4

| Tissue Name | A | B |
|---|---|---|
| Adipose | 1.4 | 1.5 |
| Melanoma* Hs688(A).T | 2.6 | 3.2 |
| Melanoma* Hs688(B).T | 4.6 | 4.2 |
| Melanoma* M14 | 6.7 | 6.4 |
| Melanoma* LOXIMVI | 4.8 | 4.0 |
| Melanoma* SK-MEL-5 | 2.6 | 4.2 |
| Squamous cell carcinoma SCC-4 | 9.0 | 8.4 |
| Testis Pool | 1.3 | 1.1 |
| Prostate ca.* (bone met) PC-3 | 21.0 | 24.8 |
| Prostate Pool | 0.9 | 0.8 |
| Placenta | 0.9 | 1.3 |
| Uterus Pool | 0.4 | 0.4 |
| Ovarian ca. OVCAR-3 | 77.4 | 66.9 |
| Ovarian ca. SK-OV-3 | 42.0 | 36.3 |
| Ovarian ca. OVCAR-4 | 9.5 | 12.7 |
| Ovarian ca. OVCAR-5 | 39.2 | 44.4 |
| Ovarian ca. IGROV-1 | 22.1 | 27.7 |
| Ovarian ca. OVCAR-8 | 18.0 | 14.9 |
| Ovary | 1.5 | 1.9 |
| Breast ca. MCF-7 | 7.7 | 9.7 |
| Breast ca. MDA-MB-231 | 22.7 | 31.2 |
| Breast ca. BT 549 | 13.2 | 10.1 |
| Breast ca. T47D | 100.0 | 100.0 |
| Breast ca. MDA-N | 4.8 | 4.2 |
| Breast Pool | 1.6 | 1.6 |
| Trachea | 2.8 | 2.6 |
| Lung | 0.2 | 0.1 |
| Fetal Lung | 11.4 | 8.3 |
| Lung ca. NCI-N417 | 1.4 | 0.7 |
| Lung ca. LX-1 | 10.5 | 11.0 |
| Lung ca. NCI-H146 | 0.1 | 0.1 |
| Lung ca. SHP-77 | 1.1 | 0.8 |
| Lung ca. A549 | 15.6 | 10.4 |
| Lung ca. NCI-H526 | 5.4 | 1.6 |
| Lung ca. NCI-H23 | 18.9 | 20.6 |
| Lung ca. NCI-H460 | 11.5 | 9.3 |
| Lung ca. HOP-62 | 23.7 | 23.0 |
| Lung ca. NCI-H522 | 1.8 | 2.3 |
| Liver | 0.5 | 0.6 |
| Fetal Liver | 0.8 | 1.4 |
| Liver ca. HepG2 | 15.5 | 12.6 |
| Kidney Pool | 1.5 | 2.5 |
| Fetal Kidney | 5.8 | 4.6 |
| Renal ca. 786-0 | 46.3 | 39.5 |
| Renal ca. A498 | 13.8 | 7.9 |
| Renal ca. ACHN | 14.3 | 15.9 |
| Renal ca. UO-31 | 41.5 | 38.7 |
| Renal ca. TK-10 | 19.3 | 16.4 |
| Bladder | 8.4 | 9.0 |
| Gastric ca. (liver met.) NCI-N87 | 87.7 | 80.7 |
| Gastric ca. KATO III | 17.8 | 17.7 |
| Colon ca. SW-948 | 9.2 | 7.8 |
| Colon ca. SW480 | 25.0 | 32.3 |
| Colon ca.* (SW480 met) SW620 | 5.0 | 4.6 |
| Colon ca. HT29 | 26.8 | 30.6 |
| Colon ca. HCT-116 | 4.9 | 5.8 |
| Colon ca. CaCo-2 | 13.6 | 10.4 |
| Colon cancer tissue | 10.2 | 10.0 |
| Colon ca. SW1116 | 5.1 | 3.6 |
| Colon ca. Colo-205 | 1.8 | 1.5 |
| Colon ca. SW-48 | 1.2 | 0.7 |
| Colon Pool | 1.9 | 1.3 |
| Small Intestine Pool | 0.6 | 1.0 |
| Stomach Pool | 1.5 | 1.2 |
| Bone Marrow Pool | 0.6 | 0.5 |
| Fetal Heart | 1.4 | 1.0 |
| Heart Pool | 0.7 | 0.8 |
| Lymph Node Pool | 2.1 | 2.0 |
| Fetal Skeletal Muscle | 0.9 | 0.5 |
| Skeletal Muscle Pool | 0.4 | 0.5 |
| Spleen Pool | 0.7 | 0.7 |
| Thymus Pool | 1.8 | 2.2 |
| CNS cancer (glio/astro) U87-MG | 5.9 | 6.0 |
| CNS cancer (glio/astro) U-118-MG | 11.7 | 11.2 |
| CNS cancer (neuro; met) SK-N-AS | 1.2 | 0.9 |
| CNS cancer (astro) SF-539 | 6.7 | 5.0 |
| CNS cancer (astro) SNB-75 | 22.8 | 32.3 |
| CNS cancer (glio) SNB-19 | 25.0 | 20.2 |
| CNS cancer (glio) SF-295 | 35.1 | 38.2 |
| Brain (Amygdala) Pool | 1.7 | 1.3 |
| Brain (cerebellum) | 1.4 | 1.0 |
| Brain (fetal) | 7.0 | 2.8 |
| Brain (Hippocampus) Pool | 1.6 | 0.9 |
| Cerebral Cortex Pool | 1.9 | 0.9 |
| Brain (Substantia nigra) Pool | 2.8 | 1.7 |
| Brain (Thalamus) Pool | 2.7 | 1.6 |
| Brain (whole) | 3.4 | 1.1 |
| Spinal Cord Pool | 1.8 | 1.4 |
| Adrenal Gland | 0.2 | 0.4 |
| Pituitary gland Pool | 0.3 | 0.2 |
| Salivary Gland | 1.1 | 1.3 |
| Thyroid (female) | 3.3 | 3.7 |
| Pancreatic ca. CAPAN2 | 23.7 | 27.7 |
| Pancreas Pool | 3.0 | 4.1 |

Column A - Rel. Exp. (%) Ag3605, Run 213406184
Column B - Rel. Exp. (%) Ag3974, Run 217508632

TABLE HD
Panel 4.1D

| Tissue Name | A | B |
|---|---|---|
| Secondary Th1 act | 1.0 | 1.2 |
| Secondary Th2 act | 5.1 | 8.0 |
| Secondary Tr1 act | 2.5 | 3.5 |
| Secondary Th1 rest | 0.0 | 0.7 |
| Secondary Th2 rest | 0.4 | 0.2 |
| Secondary Tr1 rest | 0.4 | 1.2 |
| Primary Th1 act | 3.6 | 3.2 |
| Primary Th2 act | 1.1 | 2.0 |
| Primary Tr1 act | 3.4 | 2.9 |
| Primary Th1 rest | 0.9 | 0.4 |
| Primary Th2 rest | 0.5 | 0.2 |
| Primary Tr1 rest | 0.2 | 0.3 |
| CD45RA CD4 lymphocyte act | 43.5 | 22.7 |
| CD45RO CD4 lymphocyte act | 5.0 | 5.5 |
| CD8 lymphocyte act | 3.9 | 3.3 |
| Secondary CD8 lymphocyte rest | 3.7 | 3.3 |
| Secondary CD8 lymphocyte act | 3.3 | 3.5 |
| CD4 lymphocyte none | 0.3 | 0.1 |
| 2ry Th1/Th2/Tr1 anti-CD95 CH11 | 0.3 | 0.4 |
| LAK cells rest | 5.0 | 6.4 |
| LAK cells IL-2 | 2.8 | 1.7 |
| LAK cells IL-2 + IL-12 | 1.4 | 1.8 |
| LAK cells IL-2 + IFN gamma | 2.3 | 1.1 |
| LAK cells IL-2 + IL-18 | 2.9 | 1.6 |
| LAK cells PMA/ionomycin | 6.8 | 4.6 |
| NK Cells IL-2 rest | 1.6 | 1.9 |
| Two Way MLR 3 day | 10.7 | 12.4 |
| Two Way MLR 5 day | 6.5 | 5.3 |
| Two Way MLR 7 day | 4.3 | 4.0 |
| PBMC rest | 0.0 | 0.6 |
| PBMC PWM | 5.0 | 4.9 |
| PBMC PHA-L | 5.5 | 3.4 |
| Ramos (B cell) none | 0.4 | 0.4 |
| Ramos (B cell) ionomycin | 0.2 | 0.2 |
| B lymphocytes PWM | 2.4 | 3.0 |
| B lymphocytes CD40L and IL-4 | 1.5 | 3.7 |
| EOL-1 dbcAMP | 3.4 | 3.1 |
| EOL-1 dbcAMP PMA/ionomycin | 18.3 | 8.0 |
| Dendritic cells none | 14.8 | 9.0 |
| Dendritic cells LPS | 48.3 | 32.8 |
| Dendritic cells anti-CD40 | 9.7 | 8.8 |
| Monocytes rest | 0.9 | 1.4 |
| Monocytes LPS | 66.4 | 81.2 |
| Macrophages rest | 16.2 | 9.7 |
| Macrophages LPS | 54.7 | 43.8 |
| HUVEC none | 9.3 | 12.6 |
| HUVEC starved | 14.4 | 25.2 |
| HUVEC IL-1beta | 15.6 | 18.9 |
| HUVEC IFN gamma | 12.9 | 16.7 |
| HUVEC TNF alpha + IFN gamma | 37.6 | 34.9 |
| HUVEC TNF alpha + IL4 | 31.4 | 31.4 |
| HUVEC IL-11 | 14.9 | 13.9 |
| Lung Microvascular EC none | 79.0 | 100.0 |
| Lung Microvascular EC TNFalpha + IL-1beta | 100.0 | 97.9 |
| Microvascular Dermal EC none | 49.7 | 48.3 |
| Microsvasular Dermal EC TNFalpha + IL-1beta | 56.6 | 47.0 |
| Bronchial epithelium TNFalpha + IL1beta | 78.5 | 90.1 |
| Small airway epithelium none | 31.0 | 32.5 |
| Small airway epithelium TNFalpha + IL-1beta | 81.8 | 93.3 |
| Coronery artery SMC rest | 17.2 | 28.5 |
| Coronery artery SMC TNFalpha + IL-1beta | 22.2 | 28.7 |
| Astrocytes rest | 80.1 | 55.1 |
| Astrocytes TNFalpha + IL-1beta | 82.9 | 66.4 |
| KU-812 (Basophil) rest | 2.6 | 1.9 |
| KU-812 (Basophil) PMA/ionomycin | 0.6 | 2.8 |
| CCD1106 (Keratinocytes) none | 70.7 | 82.4 |
| CCD1106 (Keratinocytes) TNFalpha + IL-1beta | 77.4 | 72.7 |
| Liver cirrhosis | 13.2 | 14.4 |
| NCI-H292 none | 57.8 | 54.0 |
| NCI-H292 IL-4 | 62.4 | 78.5 |
| NCI-H292 IL-9 | 61.6 | 79.6 |
| NCI-H292 IL-13 | 53.6 | 59.9 |
| NCI-H292 IFN gamma | 67.4 | 71.7 |
| HPAEC none | 21.5 | 21.3 |
| HPAEC TNF alpha + IL-1 beta | 37.6 | 45.4 |
| Lung fibroblast none | 22.4 | 29.3 |
| Lung fibroblast TNF alpha + IL-1 beta | 71.2 | 87.7 |
| Lung fibroblast IL-4 | 16.2 | 23.3 |
| Lung fibroblast IL-9 | 31.9 | 30.4 |
| Lung fibroblast IL-13 | 18.7 | 36.6 |
| Lung fibroblast IFN gamma | 23.0 | 29.7 |
| Dermal fibroblast CCD1070 rest | 15.9 | 27.2 |
| Dermal fibroblast CCD1070 TNF alpha | 15.9 | 20.6 |
| Dermal fibroblast CCD1070 IL-1 beta | 17.2 | 22.4 |
| Dermal fibroblast IFN gamma | 7.2 | 10.3 |
| Dermal fibroblast IL-4 | 7.2 | 8.0 |
| Dermal Fibroblasts rest | 4.3 | 6.3 |
| Neutrophils TNFa + LPS | 0.0 | 0.9 |
| Neutrophils rest | 0.2 | 1.0 |
| Colon | 7.0 | 5.8 |
| Lung | 23.3 | 23.3 |
| Thymus | 5.8 | 7.3 |
| Kidney | 23.2 | 33.2 |

Column A - Rel. Exp. (%) Ag3605, Run 169943454
Column B - Rel. Exp. (%) Ag3974, Run 170739806

General_screening_panel_v1.4 Summary: Ag3605/Ag3974 The highest expression of the CG94946-01 gene was detected in breast cancer cell line T47D (CTs=22.5-25.3). In addition, there was substantial expression in other samples derived from breast, ovarian cancer, renal, lung, colon and brain cancer cell lines. Thus, the expression of this gene is useful as a marker for cancer and for differentiating cancerous from normal tissues or cells. Therapeutic modulation of this gene, expressed protein and/or use of antibodies or small molecule drugs targeting the gene or gene product is in the treatment of breast, ovarian, kidney, lung, colon and brain cancer. Among metabolic tissues, this gene showed low-to-moderate levels of expression in adrenal, pituitary, adult and fetal heart, adult and fetal liver, adult and fetal skeletal muscle, and adipose. High expression of this gene was detected (CT values=27) in pancreas and thyroid. Decreased glomerular expression of agrin has been observed in diabetic nephropathy (Yard B A, Exp Nephrol 2001;9(3):214-22). Thus, this gene product is useful for the differentiation, diagnosis and treatment of metabolic and endocrine diseases, including obesity, Types 1 and 2 diabetes and thyroidopathies. This gene was expressed at moderate levels in all regions of the central nervous system examined, including amygdala, hippocampus, substantia nigra, thalamus, cerebellum, cerebral cortex, and spinal cord. Therapeutic modulation of this gene, expressed protein and/or use of antibodies or small molecule drugs targeting the gene or gene product are useful in the treatment of central nervous system disorders such as Alzheimer's disease, Parkinson's disease, epilepsy, multiple sclerosis, schizophrenia and depression.

Panel 4.1D Summary: Ag3605/Ag3974 Highest expression of NOV9 was detected in lung microvascular endothelial cells (CTs=27.3-28.5), microvascular dermal endothelial cells, mucoepidermoid cell line NCI-H292, astrocytes, and keratinocytes. Thus therapeutic modulation of this gene, expressed protein and/or use of antibodies or small molecule drugs targeting the gene or gene product are useful in the treatment of symptoms/conditions associated with autoimmune and inflammatory disorders including psoriasis, allergy, asthma, inflammatory bowel disease, rheumatoid arthritis and osteoarthritis.

### Example D

### Gene Expression Analysis Using CuraChip in Human Tissues from Tumors and from Equivalent Normal Tissues

Background: CuraGen has developed a gene microarray (CuraChip 1.2) for target identification. It provides a high-throughput means of global mRNA expression analyses of CuraGen's collection of cDNA sequences representing the Pharmaceutically Tractable Genome (PTG). This sequence set includes genes which can be developed into protein therapeutics, or used to develop antibody or small molecule therapeutics. CuraChip 1.2 contains ˜11,000 oligos representing approximately 8,500 gene loci, including (but not restricted to) kinases, ion channels, G-protein coupled receptors (GPCRs), nuclear hormone receptors, proteases, transporters, metabolic enzymes, hormones, growth factors, chemokines, cytokines, complement and coagulation factors, and cell surface receptors.

The CuraChip cDNAs were represented as 30-mer oligodeoxyribonucleotides (oligos) on a glass microchip. Hybridization methods using the longer CuraChip oligos are more specific compared to methods using 25-mer oligos. CuraChip oligos were synthesized with a linker, purified to remove truncated oligos (which can influence hybridization strength and specificity), and spotted on a glass slide. Oligo-dT primers were used to generate cRNA probes for hybridization from samples of interest. A biotin-avidin conjugation system was used to detect hybridized probes with a fluorophore-labeled secondary antibody. Gene expression was analyzed using clustering and correlation bioinformatics tools such as Spotfire® (Spotfire, Inc., 212 Elm Street, Somerville, Mass. 02144) and statistical tools such as multivariate analysis (MVA).

Expression Analysis of NOV1, CG121992-01 using PITG Chip 1.2: Approximately 234 samples of RNA from tissues obtained from surgically dissected disease- and non-disease tissues, and treated and untreated cell lines, were used to generate labelled nucleic acid which was hybridized to PTG Chip 1.2. An oligo (optg2_{—}1002018, TTGGAGAGATGAGCTGTATCACCTGCAGAT (SEQ ID NO:143)) that corresponds to CG121992-01 on the PTG Chip 1.2 was analyzed for its expression profile.

| | Signal |
|---|---|
| Definition | value |
| G1C4D21B11-01_Lung cancer(35C) | 18.72 |
| G1C4D21B11-02_Lung NAT(36A) | 27.29 |
| G1C4D21B11-03_Lung cancer(35E) | 150.11 |
| G1C4D21B11-04_Lung cancer(365) | 47.21 |
| G1C4D21B11-05_Lung cancer(368) | 46.04 |
| G1C4D21B11-06_Lung cancer(369) | 33.28 |
| G1C4D21B11-07_Lung cancer(36E) | 20.46 |
| G1C4D21B11-08_Lung NAT(36F) | 121.31 |
| G1C4D21B11-09_Lung cancer(370) | 57.42 |
| G1C4D21B11-10_Lung cancer(376) | 24.03 |
| G1C4D21B11-11_Lung cancer(378) | 16.67 |
| G1C4D21B11-12_Lung cancer(37A) | 12.85 |
| G1C4D21B11-13_Normal Lung 4 | 61.34 |
| G1C4D21B11-14_Normal Lung 5 | 99.72 |
| G1C4D21B11-16_5.Melanoma | 52.01 |
| G1C4D21B11-17_6.Melanoma | 71.46 |
| G1C4D21B11-18_Melanoma (19585) | 28.82 |
| G1C4D21B11-19_Normal Lung 1 | 38.72 |
| G1C4D21B11-20_Lung cancer(372) | 34.2 |
| G1C4D21B11-21_Lung NAT(35D) | 73.14 |
| G1C4D21B11-22_Lung NAT(361) | 20.95 |
| G1C4D21B11-23_1.Melanoma | 42.94 |
| G1C4D21B11-24_Normal Lung 2 | 56.04 |
| G1C4D21B11-25_Lung cancer(374) | 76.78 |
| G1C4D21B11-26_Lung cancer(36B) | 12.72 |
| G1C4D21B11-27_Lung cancer(362) | 51.13 |
| G1C4D21B11-28_Lung cancer(358) | 101.83 |
| G1C4D21B11-29_2.Melanoma | 57.33 |
| G1C4D21B11-30_Normal Lung 3 | 42.9 |
| G1C4D21B11-31_Lung NAT(375) | 87.42 |
| G1C4D21B11-32_Lung cancer(36D) | 23.39 |
| G1C4D21B11-33_Lung NAT(363) | 26.15 |
| G1C4D21B11-34_Lung cancer(35A) | 35.39 |
| G1C4D21B11-35_4.Melanoma | 92.98 |
| G1C4E09B12-54_Prostate cancer(B8B) | 115.79 |
| G1C4E09B12-55_Prostate cancer(B88) | 66.13 |
| G1C4E09B12-56_Prostate NAT(B93) | 129.17 |
| G1C4E09B12-57_Prostate cancer(B8C) | 133.03 |
| G1C4E09B12-58_Prostate cancer(AD5) | 80.36 |
| G1C4E09B12-59_Prostate NAT(AD6) | 121.97 |
| G1C4E09B12-60_Prostate cancer(AD7) | 65.73 |
| G1C4E09B12-61_Prostate NAT(AD8) | 91.17 |
| G1C4E09B12-62_Prostate cancer(ADA) | 242.14 |
| G1C4E09B12-63_Prostate NAT(AD9) | 151.25 |
| G1C4E09B12-64_Prostate cancer(9E7) | 5.51 |
| G1C4E09B12-65_Prostate NAT(A0B) | 92.72 |
| G1C4E09B12-66_Prostate cancer(A0A) | 75.96 |
| G1C4E09B12-67_Prostate cancer(9E2) | 18.05 |
| G1C4E09B12-68_Pancreatic cancer(9E4) | 55.06 |
| G1C4E09B12-69_Pancreatic cancer(9D8) | 5.02 |
| G1C4E09B12-70_Pancreatic cancer(9D4) | 9.04 |
| G1C4E09B12-71_Pancreatic cancer(9BE) | 38.09 |
| G1C4E09B12-73_Pancreatic NAT(ADB) | 172.23 |
| G1C4E09B12-74_Pancreatic NAT(ADC) | 327.48 |
| G1C4E09B12-76_Pancreatic NAT(ADD) | 103.04 |
| G1C4E09B12-77_Pancreatic NAT(AED) | 31.82 |
| G1C4E19B13-10_Colon NAT(8B6) | 53.85 |
| G1C4E19B13-12_Colon NAT(9F1) | 61.04 |
| G1C4E19B13-13_Colon cancer(9F2) | 31.11 |
| G1C4E19B13-14_Colon NAT(A1D) | 122.69 |
| G1C4E19B13-15_Colon cancer(9DB) | 0 |
| G1C4E19B13-16_Colon NAT(A15) | 78.49 |
| G1C4E19B13-17_Colon cancer(A14) | 23.69 |
| G1C4E19B13-18_Colon NAT(ACB) | 57.87 |
| G1C4E19B13-19_Colon cancer(AC0) | 19.08 |
| G1C4E19B13-2_Colon cancer(8A4) | 94.14 |
| G1C4E19B13-20_Colon NAT(ACD) | 58.43 |
| G1C4E19B13-21_Colon cancer(AC4) | 17.46 |
| G1C4E19B13-22_Colon NAT(AC2) | 17.37 |
| G1C4E19B13-23_Colon cancer(AC1) | 24.09 |
| G1C4E19B13-24_Colon NAT(ACC) | 31.75 |
| G1C4E19B13-25_Colon cancer(AC3) | 12.67 |
| G1C4E19B13-26_Breast cancer(9B7) | 841.73 |
| G1C4E19B13-27_Breast NAT(9CF) | 33.19 |
| G1C4E19B13-28_Breast cancer(9B6) | 453.74 |
| G1C4E19B13-29_Breast cancer(9C7) | 5.87 |
| G1C4E19B13-3_Colon cancer(8A6) | 23.75 |
| G1C4E19B13-30_Breast NAT(A11) | 187.73 |
| G1C4E19B13-31_Breast cancer(A1A) | 52.65 |
| G1C4E19B13-32_Breast cancer(9F3) | 56.05 |
| G1C4E19B13-33_Breast cancer(9B8) | 13.06 |
| G1C4E19B13-34_Breast NAT(9C4) | 184.99 |
| G1C4E19B13-35_Breast cancer(9EF) | 139.47 |
| G1C4E19B13-36_Breast cancer(9F0) | 32.54 |
| G1C4E19B13-37_Breast cancer(9B4) | 77.88 |
| G1C4E19B13-38_Breast cancer(9EC) | 36.65 |
| G1C4E19B13-4_Colon cancer(8A7) | 5.41 |
| G1C4E19B13-44_Colon cancer(8B7) | 47.59 |
| G1C4E19B13-5_Colon cancer(8A9) | 12.73 |
| G1C4E19B13-6_Colon cancer(8AB) | 50.86 |
| G1C4E19B13-7_Colon cancer(8AC) | 9.22 |
| G1C4E19B13-8_Colon NAT(8AD) | 97.98 |
| G1C4E19B13-9_Colon cancer(8B5) | 43.83 |
| G1C4E21B14-1_Cervical cancer(B08) | 0 |
| G1C4E21B14-10_Brain cancer(9F8) | 0 |
| G1C4E21B14-11_Brain cancer(9C0) | 0 |
| G1C4E21B14-12_Brain cancer(9F7) | 0 |
| G1C4E21B14-13_Brain cancer(A00) | 0 |
| G1C4E21B14-14_Brain NAT(A01) | 0 |
| G1C4E21B14-15_Brain cancer(9DA) | 0 |
| G1C4E21B14-16_Brain cancer(9FE) | 0 |
| G1C4E21B14-17_Brain cancer(9C6) | 0 |
| G1C4E21B14-18_Brain cancer(9F6) | 0 |
| G1C4E21B14-2_Cervical NAT(AEB) | 0 |
| G1C4E21B14-21_Bladder NAT(23954) | 0 |
| G1C4E21B14-22_Urinary cancer(AF6) | 0 |
| G1C4E21B14-23_Urinary cancer(B0C) | 0 |
| G1C4E21B14-24_Urinary cancer(AE4) | 0 |
| G1C4E21B14-25_Urinary NAT(B20) | 0 |
| G1C4E21B14-26_Urinary cancer(AE6) | 0 |
| G1C4E21B14-27_Urinary NAT(B04) | 0 |
| G1C4E21B14-28_Urinary cancer(B07) | 0 |
| G1C4E21B14-29_Urinary NAT(AF8) | 0 |
| G1C4E21B14-3_Cervical cancer(AFF) | 0 |
| G1C4E21B14-30_Ovarian cancer(9D7) | 0 |
| G1C4E21B14-31_Urinary cancer(AF7) | 0 |
| G1C4E21B14-32_Ovarian cancer(9F5) | 0 |
| G1C4E21B14-33_Ovarian cancer(A05) | 0 |
| G1C4E21B14-34_Ovarian cancer(9BC) | 0 |
| G1C4E21B14-35_Ovarian cancer(9C2) | 0 |
| G1C4E21B14-36_Ovarian cancer(9D9) | 0 |
| G1C4E21B14-37_Ovarian NAT(AC7) | 0 |
| G1C4E21B14-38_Ovarian NAT(AC9) | 0 |
| G1C4E21B14-39_Ovarian NAT(ACA) | 0 |
| G1C4E21B14-4_Cervical N AT(B1E) | 0 |
| G1C4E21B14-40_Ovarian NAT(AC5) | 0 |
| G1C4E21B14-6_Cervical NAT(AFA) | 0 |
| G1C4E21B14-7_Cervical cancer(B1F) | 0 |
| G1C4E21B14-8_Cervical NAT(B1C) | 0 |
| G1C4E23B15-32_Breast cancer(D34) | 41.24 |
| G1C4E23B15-33_Breast cancer(D35) | 21.19 |
| G1C4E23B15-34_Breast cancer(D36) | 102.07 |
| G1C4E23B15-35_Breast cancer(D37) | 83.94 |
| G1C4E23B15-36_Breast cancer(D38) | 25.2 |
| G1C4E23B15-37_Breast cancer(D39) | 1.62 |
| G1C4E23B15-38_Breast cancer(D3A) | 96.28 |
| G1C4E23B15-39_Breast cancer(D3B) | 81.09 |
| G1C4E23B15-40_Breast cancer(D3C) | 71.32 |
| G1C4E23B15-41_Breast cancer(D3D) | 56.37 |
| G1C4E23B15-42_Breast cancer(D3E) | 283.29 |
| G1C4E23B15-43_Breast cancer(D3F) | 1141.66 |
| G1C4E23B15-44_Breast cancer(D40) | 157.32 |
| G1C4E23B15-45_Breast cancer(D42) | 58.84 |
| G1C4E23B15-46_Breast cancer(D43) | 223.65 |
| G1C4E23B15-47_Breast cancer(D44) | 778.17 |
| G1C4E23B15-48_Breast cancer(D45) | 278.05 |
| G1C4E23B15-49_Breast cancer(D46) | 1191.16 |
| G1C4E30B16-1_2.SK-MES | 0 |
| G1C4E30B16-10_40.HLaC-79 | 64.5 |
| G1C4E30B16-11_43.H226 | 0 |
| G1C4E30B16-12_45.HCT-116 | 94.63 |
| G1C4E30B16-13_53.IGROV-1 | 0 |
| G1C4E30B16-14_59.MX-1 | 0 |
| G1C4E30B16-15_63.C33A | 229.24 |
| G1C4E30B16-16_65.Daudi | 0 |
| G1C4E30B16-17_71.MV522 | 68.41 |
| G1C4E30B16-18_76.RWP-2 | 0 |
| G1C4E30B16-19_77.BON | 3.86 |
| G1C4E30B16-2_6.MiaPaCa | 0 |
| G1C4E30B16-20_82.H82 | 176.67 |
| G1C4E30B16-21_86.H69 | 0 |
| G1C4E30B16-22_95.Caki-2 | 0 |
| G1C4E30B16-23_100.LNCaP | 5.77 |
| G1C4E30B16-24_101.A549 | 0 |
| G1C4E30B16-25_1.DU145 | 96.91 |
| G1C4E30B16-26_6.OVCAR-3 | 0 |
| G1C4E30B16-27_11.HT-29 | 135.28 |
| G1C4E30B16-28_13.DLD-2 | 0 |
| G1C4E30B16-29_18.MCF-7 | 24.47 |
| G1C4E30B16-3_9.H460 | 0 |
| G1C4E30B16-4_15.SW620 | 3.28 |
| G1C4E30B16-5_20.SK-OV-3 | 0 |
| G1C4E30B16-6_23.MDA-231 | 0 |
| G1C4E30B16-7_27.Caki-1 | 0 |
| G1C4E30B16-8_31.PC-3 | 0 |
| G1C4E30B16-9_35.LoVo | 0 |
| G1C4I11B20-10_Kidney NAT(10B1) | 206.25 |
| G1C4I11B20-11_Kidney cancer(10B2) | 103.55 |
| G1C4I11B20-12_Kidney NAT(10B3) | 194.23 |
| G1C4I11B20-13_Kidney cancer(10B4) | 0 |
| G1C4I11B20-14_Kidney NAT(10B5) | 191.62 |
| G1C4I11B20-15_Kidney cancer(10B6) | 0.84 |
| G1C4I11B20-16_Kidney NAT(10B7) | 222.1 |
| G1C4I11B20-17_Kidney cancer(10BA) | 0 |
| G1C4I11B20-18_Kidney NAT(10BB) | 216.02 |
| G1C4I11B20-19_Kidney cancer(10C0) | 13.27 |
| G1C4I11B20-20_Kidney NAT(10C1) | 149.04 |
| G1C4I11B20-21_Kidney cancer(10C4) | 309.45 |
| G1C4I11B20-22_Kidney NAT(10C5) | 217.97 |
| G1C4I11B20-23_Kidney cancer(10A8) | 0 |
| G1C4I11B20-24_Kidney NAT(10A9) | 265 |
| G1C4I11B20-25_Kidney cancer(10AA) | 106.33 |
| G1C4I11B20-4_Kidney NAT(10AB) | 246.18 |
| G1C4I11B20-5_Kidney cancer(10AC) | 219.37 |
| G1C4I11B20-6_Kidney NAT(10AD) | 226.44 |
| G1C4I11B20-7_Kidney cancer(10AE) | 251.5 |
| G1C4I11B20-8_Kidney NAT(10AF) | 238.33 |
| G1C4I11B20-9_Kidney cancer(10B0) | 129.29 |
| G1C4I12B21-66_Ardais Lung 4 | 115.55 |
| G1C4I12B21-67_Ardais Lung 6 | 24.5 |
| G1C4I12B21-68_Ardais Lung 7 | 74.81 |
| G1C4I12B21-69_Ardais Lung 10 | 51.67 |
| G1C4I12B21-70_4169B1 normal lung | 1.11 |
| G1C4I12B21-71_4267B1 normal lung | 4.99 |
| G1C4I12B21-72_#689 Control Lung | 34.21 |
| G1C4I12B21-73_#812 Asthma Lung | 82.58 |
| G1C4I12B21-74_#1078 Control Lung | 104.81 |
| G1C4I17B22-10_Lymphoma(9BF) | 0 |
| G1C4I17B22-11_Lymphoma(9D2) | 0 |
| G1C4I17B22-12_Lymphoma(A04) | 0 |
| G1C4I17B22-13_Lymphoma(9DD) | 0 |
| G1C4I17B22-14_Lymphoma(F68) | 0 |
| G1C4I17B22-15_Lymphoma(F6A) | 0 |
| G1C4I17B22-16_Lymphoma(F6B) | 0 |
| G1C4I17B22-17_Lymphoma(F6C) | 0 |
| G1C4I17B22-18_Lymphoma(F6D) | 0 |
| G1C4I17B22-19_Lymphoma(F6E) | 0 |
| G1C4I17B22-20_Lymphoma(F6F) | 0 |
| G1C4I17B22-21_Lymphoma(F70) | 0 |
| G1C4I17B22-22_Lymphoma(F71) | 0 |
| G1C4I17B22-23_Lymphoma(F72) | 0 |
| G1C4I17B22-24_Lymphoma(F73) | 0 |
| G1C4I17B22-25_Lymphoma(F74) | 0 |
| G1C4I17B22-26_Lymphoma NAT(1002) | 41.6 |
| G1C4I17B22-28_Lymphoma NAT(1004) | 3.63 |
| G1C4I17B22-29_Lymphoma NAT(1005) | 0 |
| G1C4I17B22-30_Lymphoma NAT(1007) | 0 |
| G1C4I17B22-32_Lymphoma NAT(1003) | 0 |
| G1C4I17B22-4_Lymphoma(9E3) | 135.17 |
| G1C4I17B22-5_Lymphoma(9D0) | 0 |
| G1C4I17B22-6_Lymphoma(9E1) | 0 |
| G1C4I17B22-7_Lymphoma(A0D) | 65.22 |
| G1C4I17B22-8_Lymphoma(9B5) | 0 |
| G1C4I17B22-9_Lymphoma(9D3) | 0 |

Gene expression analysis using CuraChip revealed that the expression level of this gene was elevated in breast cancer tissues and reduced in kidney cancer tissues as compared with normal adjacent tissues. Therefore this gene is useful as a specific marker for differentiating cancerous from normal tissue in these disease states. Therapeutic modulation of this gene, expressed protein and/or use of antibodies or small molecule drugs targeting the gene or gene product would be useful in the treatment of breast cancer and kidney cancer.

### Other Embodiments

Although particular embodiments are disclosed herein in detail, this is done by way of example for purposes of illustration only, and is not intended to be limiting with respect to the scope of the appended claims, which follow. In particular, it is contemplated by the inventors that various substitutions, alterations, and modifications will be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be with in the scope of the following claims. The claims presented are representative of the inventions disclosed herein. Other, unclaimed inventions are also contemplated. Applicants reserve the right to pursue such inventions in later claims.

1. An isolated polypeptide comprising the mature form of an amino acid sequenced selected from the group consisting of SEQ ID NO:2n, wherein n is an integer between 1 and 38.

2. An isolated polypeptide comprising an amino acid sequence selected from the group consisting of SEQ ID NO:2n, wherein n is an integer between 1 and 38.

4. A composition comprising the polypeptide of claim 1 and a carrier.

5. A kit comprising, in one or more containers, the composition of claim 4.

6. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

7. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

8. A method of identifying an agent that binds to the polypeptide of claim 1, the method comprising:
(a) introducing said polypeptide to said agent; and
(b) determining whether said agent binds to said polypeptide.

9. The method of claim 8 wherein the agent is a cellular receptor or a downstream effector.

10. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
determining whether the substance alters the property or function ascribable to the polypeptide;
whereby, if an alteration observed in the presence of the substance is not observed when the cell is contacted with a composition in the absence of the substance, the substance is identified as a potential therapeutic agent.

11. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

12. The method of claim 11, wherein said test animal is a recombinant test animal that expresses a test protein transgene or expresses said transgene under the control of a promoter at an increased level relative to a wild-type test animal, and wherein said promoter is not the native gene promoter of said transgene.

13. A method of treating a pathological state in a mammal, the method comprising administering to the mammal a polypeptide in an amount that is sufficient to alleviate the pathological state, wherein the polypeptide is a polypeptide having an amino acid sequence comprising the amino acid sequence selected from the group consisting of SEQ ID NO:2n, wherein n is an integer between 1 and 38 or a biologically active fragment thereof.

14. An isolated nucleic acid molecule comprising a nucleic acid sequence selected from the group consisting of SEQ ID NO:2n-1, wherein n is an integer between 1 and 38.

15. An isolated nucleic acid molecule encoding the mature form of a polypeptide having an amino acid sequence selected from the group consisting of SEQ ID NO:2n, wherein n is an integer between 1 and 38.

16. A vector comprising the nucleic acid molecule of claim 14.

17. A cell comprising the vector of claim 16.

18. An antibody that immunospecifically binds to the polypeptide of claim 1.

19. The antibody of claim 18, wherein the antibody is a human monoclonal antibody.

20. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n-1, wherein n is an integer between 1 and 38.

21. The method of claim 36 wherein the cell is chosen from the group comprising a bacterial cell, an insect cell, a yeast cell and a mammalian cell.
